(19) United States
(12) Patent Application Publication
(10) Pub. No.: US 2022/0143084 A1
(43) Pub. Date: May 12, 2022

(54) MODIFIED NATURAL KILLER (NK) CELLS FOR IMMUNOTHERAPY

(71) Applicant: Editas Medicine, Inc., Cambridge, MA (US)

(72) Inventors: Gordon Grant Welstead, Newton, MA (US); Christopher Borges, Reading, MA (US); Karrie Ka Wai Wong, Somerville, MA (US)

(73) Assignee: Editas Medicine, Inc., Cambridge, MA (US)

(21) Appl. No.: 17/430,382

(22) PCT Filed: Feb. 14, 2020
(86) PCT No.: PCT/US2020/018443

§ 371 Date: Aug. 12, 2021

Related U.S. Application Data
(60) Provisional application No. 62/943,649, filed on Dec. 4, 2019.
(60) Provisional application No. 62/841,684, filed on May 1, 2019.
(60) Provisional application No. 62/841,066, filed on Apr. 30, 2019.
(60) Provisional application No. 62/806,457, filed on Feb. 15, 2019.

(51) Int. Cl. A61K 35/17; C12N 5/0783; C12N 15/113; C12N 9/22; A61P 35/00

(52) U.S. Cl. CPC A61K 35/17; C12N 5/0646; A61P 35/00; C12N 9/22; C12N 15/113

(57) ABSTRACT

The present disclosure is directed to the generation of NK cells (or other lymphocytes) from induced pluripotent cells that have been derived from cells, e.g., developmentally mature T cells, and uses thereof for immunotherapy.

### RELATED APPLICATIONS

This application claims priority to U.S. Provisional Application No. 62/806,457, filed on Feb. 15, 2019; U.S. Provisional Application No. 62/841,066, filed on Apr. 30, 2019; U.S. Provisional Application No. 62/841,684, filed on May 1, 2019; and U.S. Provisional Application No. 62/943,649, filed on Dec. 4, 2019, the entire contents of each of which are expressly incorporated herein by reference.

### BACKGROUND

NK cells are useful for immunotherapy approaches, for example, in the context of immuno-oncology. NK cells are a type of cytotoxic innate lymphocyte. NK cells play an important role in tumor immunity, and the cytotoxic activity of NK cells is tightly regulated by a network of activating and inhibitory pathways (see, e.g., Gras Navarro A, Bjorklund A T and Chekenya M (2015) Front. Immunol. 6:202; incorporated in its entirety herein by reference).

The use of naturally occurring or modified NK cells in immunotherapy approaches, e.g., via autologous or allogeneic NK cell transfer, has been reported, and while some success has been achieved, such approaches are typically characterized by a suboptimal NK cell response. In the context of immune-oncology, it is believed that this suboptimal response is, at least in part, to tumors harnessing NK cell inhibitory pathways to suppress cytotoxic NK cell activity, limit NK cell invasion, and/or inhibit NK cell proliferation and survival. Thus, application of NK cells in the therapy of solid tumors has seen limited success.

Initial work has been performed in trying to focus NK cell response on specific cells, e.g., by expressing a chimeric antigen receptor in NK cells that targets the NK cells to tumor cells, or by modulating activating or inhibitory NK cell pathways to achieve a stronger and/or more sustained NK cell response. See, e.g., Jing Y, et al. (2015) PLoS ONE 10(3):e0121788; and Oberschmidt O, Kloess S and Koehl U (2017) Front. Immunol. 8:654; incorporated in their entireties herein by reference.

In pursuit of an off-the shelf allogeneic NK cell therapy that could be used in combination with a therapeutic antibody, an induced pluripotent stem cell line has been developed in which cells express an enhanced version of CD16 (hnCD16), and NK cells have been derived from this iPSC line. See, e.g., Li et al., Cell Stem Cell. 2018 Aug. 2; 23(2):181-192.e5; incorporated in its entirety herein by reference.

However, to date all of these approaches have seen limited success. Therefore, there remains a need for the development of better therapeutic approaches for immunotherapy.

### SUMMARY

Some aspects of the present disclosure provide compositions, cells, cell populations, methods, strategies, and treatment modalities that are useful in the context of immunotherapeutic approaches, e.g., immunooncology therapeutic approaches. In some embodiments, the present disclosure provides modified NK cells (or other lymphocytes) that are useful in NK cell therapy, e.g., in the context of immunotherapeutic approaches. In some embodiments, the cells and cell populations provided herein are characterized by one or more modifications that enhance their efficacy in immunotherapeutic approaches. For example, in some embodiments, NK cells are provided that comprise one or more modifications that effect a loss-of-function in a gene or protein associated with inhibition of NK cell function in a therapeutic context, and/or one or more modifications that effect an expression of an exogenous nucleic acid or protein associated with an enhanced NK cell function in a therapeutic context. In some embodiments, the present disclosure provides modified NK cells that are derived from an induced pluripotent cell (iPSC). IPSC-derived NK cells are also referred to herein as iNK cells. In some embodiments, modified iNK cells are provided that are derived from a somatic cell, for example, and without limitation, from a fibroblast, a peripheral blood cell, or a developmentally mature T cell (T cell that have undergone thymic selection). In some embodiments, the NK or iNK cells provided herein comprise one or more genomic edits, e.g., indels or insertions of exogenous nucleic acid constructs resulting from cutting a genomic locus with an RNA-guided nuclease. The use of RNA-guided nuclease technology in the context of the generation of modified NK and iNK cells allows for the engineering of complex alterations with enhanced characteristics relevant for clinical applications.

Some aspects of the present disclosure provide complex editing strategies, and resulting NK cells having complex genomic alterations, that allow for the generation of advanced NK cell products for clinical applications, e.g., for immunooncology therapeutic approaches. In some embodiments, the modified NK cells provided herein can serve as an off-the-shelf clinical solution for patients having, or having been diagnosed with, a hyperproliferative disease, such as, for example, a cancer. In some embodiments, the modified NK cells exhibit an enhanced survival, proliferation, NK cell response level, NK cell response duration, resistance against NK cell exhaustion, and/or target recognition as compared to non-modified NK cells. For example, the modified NK cells provided herein may comprise genomic edits that result in: expression of a chimeric antigen receptor (CAR) of interest, e.g., a CAR targeting mesothelin, EGFR, HER2 and/or MICA/B; expression of a CD16 variant, e.g., a non-naturally occurring CD16 variant such as, for example, hnCD16 (see, e.g., Zhu et al., Blood 2017, 130:4452, the contents of which are incorporated herein in their entirety by reference); expression of an IL15/IL15RA fusion; a loss-of-function in TGF beta receptor 2 (TGFbetaR2); and/or expression of a dominant-negative variant of TGFbetaR2; a loss-of-function of ADORA2A; a loss-of-function of B2M; expression of HLA-G: a loss-of-function of a CIITA; a loss-of-function of a PD1; a loss-of-function of TIGIT; and/or a loss-of-function of CISH; or any combination of two or more thereof in the modified NK cell. In one embodiment, the modified NK cell comprises genomic edits that result in a loss-of-function of TGFbetaR2 and a loss-of-function of CISH. In one embodiment, the modified NK cell comprises genomic edits that result in a loss-of-function of TGFbetaR2 and a loss-of-function of TIGIT. In one embodiment, the modified NK cell comprises genomic edits that result in a loss-of-function of TGFbetaR2 and a loss-of-function of ADORA2A. In one embodiment, the modified NK cell comprises genomic edits that result in a loss-of-function of TGFbetaR2 and a loss-of-function of NKG2A. In one embodiment, the modified NK cell comprises genomic edits that result in a loss-of-function of CISH and a loss-of-function of TIGIT. In one embodiment, the modified NK cell comprises genomic edits that result in a loss-of-function of CISH and a loss-of-function of ADORA2A. In one embodiment, the modified NK cell comprises genomic edits that result in a loss-of-function of CISH and a loss-of-function of NKG2A. In one embodiment, the modified NK cell comprises genomic edits that result in a loss-of-function of TIGIT and a loss-of-function of ADORA2A. In one embodiment, the modified NK cell comprises genomic edits that result in a loss-of-function of TIGIT and a loss-of-function of NKG2A. In one embodiment, the modified NK cell comprises genomic edits that result in a loss-of-function of ADORA2A and a loss-of-function of NKG2A. In one embodiment, the modified NK cell comprises genomic edits that result in a loss-of-function of TGFbetaR2, a loss-of-function of CISH, and a loss-of-function of TIGIT. In one embodiment, the modified NK cell comprises genomic edits that result in a loss-of-function of TGFbetaR2, a loss-of-function of CISH, and a loss-of-function of ADORA2A. In one embodiment, the modified NK cell comprises genomic edits that result in a loss-of-function of TGFbetaR2, a loss-of-function of CISH, and a loss-of-function of NKG2A. In one embodiment, the modified NK cell comprises genomic edits that result in a loss-of-function of TGFbetaR2, a loss-of-function of TIGIT, and a loss-of-function of ADORA2A. In one embodiment, the modified NK cell comprises genomic edits that result in a loss-of-function of TGFbetaR2, a loss-of-function of TIGIT, and a loss-of-function of NKG2A. In one embodiment, the modified NK cell comprises genomic edits that result in a loss-of-function of TGFbetaR2, a loss-of-function of ADORA2A, and a loss-of-function of NKG2A. In one embodiment, the modified NK cell comprises genomic edits that result in a loss-of-function of CISH, a loss-of-function of TIGIT, and a loss-of-function of ADORA2A. In one embodiment, the modified NK cell comprises genomic edits that result in a loss-of-function of CISH, a loss-of-function of TIGIT, and a loss-of-function of NKG2A. In one embodiment, the modified NK cell comprises genomic edits that result in a loss-of-function of CISH, a loss-of-function of ADORA2A, and a loss-of-function of NKG2A. In one embodiment, the modified NK cell comprises genomic edits that result in a loss-of-function of TIGIT, a loss-of-function of ADORA2A, and a loss-of-function of NKG2A.

In some embodiments, the modified NK cells provided herein may comprise genomic edits that result in: expression of an exogenous a CD16 variant, e.g., hnCD16, expression of an exogenous IL15/IL15RA fusion, expression of an exogenous HLA-G, expression of an exogenous DN-TGFbetaR2, a loss of function in TGFbetaR2, a loss of function in B2M, a loss of function of PD1, a loss of function of TIGIT, and/or a loss of function of ADORA2A.

In some embodiments, the modified NK cells provided herein may comprise genomic edits that result in: expression of an exogenous a CD16 variant, e.g., hnCD16, expression of an exogenous IL15/IL15RA fusion, expression of an exogenous HLA-G, expression of an exogenous DN-TGFbetaR2, expression of a soluble MICA and/or MICB, a loss of function in TGFbetaR2, a loss of function in B2M, a loss of function of PD1, a loss of function of TIGIT, and/or a loss of function of ADORA2A.

In some embodiments, the modified NK cells provided herein may comprise genomic edits that result in: expression of an exogenous a CD16 variant, e.g., hnCD16, expression of an exogenous IL15/IL15RA fusion, expression of an exogenous HLA-G, expression of an exogenous DN-TGFbetaR2, expression of a soluble MICA and/or MICB, expression of an exogenous IL-12, expression of an exogenous IL-18, a loss of function in TGFbetaR2, a loss of function in B2M, a loss of function of PD1, a loss of function of TIGIT, and/or a loss of function of ADORA2A.

In some embodiments, the modified NK cells provided herein may comprise genomic edits that result in: expression of an exogenous a CD16 variant, e.g., hnCD16, expression of an exogenous IL15/IL15RA fusion, expression of an exogenous HLA-G, expression of an exogenous DN-TGFbetaR2, expression of an exogenous IL-12, expression of an exogenous IL-18, a loss of function in TGFbetaR2, a loss of function in B2M, a loss of function of PD1, a loss of function of TIGIT, and/or a loss of function of ADORA2A.

In one aspect the disclosure features a modified lymphocyte, wherein the modified lymphocyte does not express endogenous CD3, CD4, and/or CD8; and expresses at least one endogenous gene encoding: (i) CD56 (NCAM), CD49, and/or CD45; (ii) NK cell receptor (cluster of differentiation 16 (CD16)); (iii) natural killer group-2 member D (NKG2D); (iv) CD69; (v) a natural cytotoxicity receptor; or any combination of two or more thereof; wherein the modified lymphocyte further: (1) comprises at least one exogenous nucleic acid construct encoding: (i) a chimeric antigen receptor (CAR); (ii) a non-naturally occurring variant of immunoglobulin gamma Fc region receptor III (FcγRIII, CD16); (iii) interleukin 15 (IL-15); (iv) IL-15 receptor (IL-15R), or a variant thereof; (v) interleukin 12 (IL-12); (vi) interleukin-12 receptor (IL-12R), or a variant thereof; (vii) human leukocyte antigen G (HLA-G); (viii) human leukocyte antigen E (HLA-E); (ix) a nucleic acid sequence encoding leukocyte surface antigen cluster of differentiation CD47 (CD47); or any combination of two or more thereof; and/or (2) exhibits a loss of function of at least one of: (i) transforming growth factor beta receptor 2 (TGFβR2); (ii) adenosine A2a receptor (ADORA2A); (iii) T cell immunoreceptor with Ig and ITIM domains (TIGIT); (iv) β-2 microgobulin (B2M); (v) programmed cell death protein 1 (PD-1); (vi) cytokine inducible SH2 containing protein (CISH); (vii) class II, major histocompatibility complex, transactivator (CIITA); (viii) natural killer cell receptor NKG2A (natural killer group 2A); (ix) two or more HLA class II histocompatibility antigen alpha chain genes, and/or two or more HLA class II histocompatibility antigen beta chain genes; (x) cluster of differentiation 32B (CD32B, FCGR2B); (xi) T cell receptor alpha constant (TRAC); or any combination of two or more thereof. In one embodiment, the modified lymphocyte exhibits a loss of function of TGFβR2 and a loss-of-function of CISH. In one embodiment, the modified lymphocyte exhibits a loss-of-function of TGFbetaR2 and a loss-of-function of TIGIT. In one embodiment, the modified lymphocyte exhibits a loss-of-function of TGFbetaR2 and a loss-of-function of ADORA2A. In one embodiment, the modified lymphocyte exhibits a loss-of-function of TGFbetaR2 and a loss-of-function of NKG2A. In one embodiment, the modified lymphocyte exhibits a loss-of-function of CISH and a loss-of-function of TIGIT. In one embodiment, the modified lymphocyte exhibits a loss-of-function of CISH and a loss-of-function of ADORA2A. In one embodiment, the modified lymphocyte exhibits a loss-of-function of CISH and a loss-of-function of NKG2A. In one embodiment, the modified lymphocyte exhibits a loss-of-function of TIGIT and a loss-of-function of ADORA2A. In one embodiment, the modified lymphocyte exhibits a loss-of-function of TIGIT and a loss-of-function of NKG2A. In one embodiment, the modified lymphocyte exhibits a loss-of-function of ADORA2A and a loss-of-function of NKG2A. In one embodiment, the modified lymphocyte exhibits a loss-of-function of TGFbetaR2, a loss-of-function of CISH, and a loss-of-function of TIGIT. In one embodiment, the modified lymphocyte exhibits a loss-of-function of TGFbetaR2, a loss-of-function of CISH, and a loss-of-function of ADORA2A. In one embodiment, the modified lymphocyte exhibits a loss-of-function of TGFbetaR2, a loss-of-function of CISH, and a loss-of-function of NKG2A. In one embodiment, the modified lymphocyte exhibits a loss-of-function of TGFbetaR2, a loss-of-function of TIGIT, and a loss-of-function of ADORA2A. In one embodiment, the modified lymphocyte exhibits a loss-of-function of TGFbetaR2, a loss-of-function of TIGIT, and a loss-of-function of NKG2A. In one embodiment, the modified lymphocyte exhibits a loss-of-function of TGFbetaR2, a loss-of-function of ADORA2A, and a loss-of-function of NKG2A. In one embodiment, the modified lymphocyte exhibits a loss-of-function of CISH, a loss-of-function of TIGIT, and a loss-of-function of ADORA2A. In one embodiment, the modified lymphocyte exhibits a loss-of-function of CISH, a loss-of-function of TIGIT, and a loss-of-function of NKG2A. In one embodiment, the modified lymphocyte exhibits a loss-of-function of CISH, a loss-of-function of ADORA2A, and a loss-of-function of NKG2A. In one embodiment, the modified lymphocyte exhibits a loss-of-function of TIGIT, a loss-of-function of ADORA2A, and a loss-of-function of NKG2A.

In one embodiment, the modified lymphocyte does not express endogenous CD3, CD4, and/or CD8; and expresses at least one endogenous gene encoding: (i) CD56 (NCAM), CD49, and/or CD45; (ii) NK cell receptor (cluster of differentiation 16 (CD16)); (iii) natural killer group-2 member D (NKG2D); (iv) CD69; (v) a natural cytotoxicity receptor; or any combination of two or more thereof; wherein the modified lymphocyte further: (1) comprises at least one exogenous nucleic acid construct encoding: (i) a chimeric antigen receptor (CAR); (ii) a non-naturally occurring variant of immunoglobulin gamma Fc region receptor III (FcγRIII, CD16); (iii) interleukin 15 (IL-15); (iv) IL-15 receptor (IL-15R), or a variant thereof; (v) interleukin 12 (IL-12); (vi) interleukin-12 receptor (IL-12R), or a variant thereof; (vii) human leukocyte antigen G (HLA-G); (viii) human leukocyte antigen E (HLA-E); (ix) a nucleic acid sequence encoding leukocyte surface antigen cluster of differentiation CD47 (CD47); or any combination of two or more thereof; and/or (2) exhibits a loss of function of transforming growth factor beta receptor 2 (TGFβR2), cytokine inducible SH2 containing protein (CISH), or a combination thereof.

In some embodiments, the nucleic acid construct is an expression construct comprising a nucleic acid sequence encoding the gene product listed under (1)(i)-(1(ix), or any combination thereof, operably linked to a promoter driving expression of the nucleic acid sequence in a target cell, e.g., in a modified lymphocyte, for example, a modified NK cell provided herein. In some embodiments, the promoter is specifically expressed in the target cell, e.g., the promoter is a lymphocyte- or NK-cell-specific promoter. In some embodiments, the promoter is a CD56 (NCAM) promoter. In some embodiments, the promoter is a CD49 promoter. In some embodiments, the promoter is a CD45 promoter. In some embodiments, the promoter is an FcγRIII promoter. In some embodiments, the promoter is an NKG2D promoter. In some embodiments, the promoter is a CD69 promoter.

In some embodiments, the exogenous nucleic acid construct encoding a gene product listed under (1) is knocked into a genomic locus encoding a gene product listed under (2), resulting in a loss-of-function of the gene product listed under (2) and expression of a gene product encoded by the exogenous nucleic acid construct, either driven by a heterologous promoter, or driven by the endogenous promoter of the genomic locus that the exogenous nucleic acid construct is knocked into.

In some embodiments, the exogenous nucleic acid construct encoding a gene product listed under (1) is knocked into a “safe harbor” locus, e.g., a ROSA26 locus, a collagen locus, or an AAVSI genomic locus.

In some embodiments, the two or more HLA class II histocompatibility antigen alpha chain genes are selected from HLA-DQA1, HLA-DRA, HLA-DPA1, HLA-DMA, HLA-DQA2, and HLA-DOA. In some embodiments, the two or more HLA class II histocompatibility antigen beta chain genes are selected from HLA-DMB, HLA-DOB, HLA-DPB1, HLA-DQB1, HLA-DQB3, HLA-DQB2, HLA-DRB1, HLA-DRB3, HLA-DRB4, and HLA-DRB5.

In some embodiments, the modified lymphocyte comprises a rearranged endogenous T-cell receptor (TCR) locus. In some embodiments, the rearranged TCR comprises TCRα VJ and/or TCRβ V(D)J section rearrangements and complete V-domain exons.

In some embodiments, the natural cytotoxicity receptor is NKp30, NKp44, NKp46, and/or CD158b.

In some embodiments, the IL-15R variant is a constitutively active IL-15R variant. In some embodiments, the constitutively active IL-15R variant is a fusion between IL-15R and an IL-15R agonist, e.g., an IL-15 protein or IL-15R-binding fragment thereof. In some embodiments, the IL-15R agonist is IL-15, or an IL-15R-binding variant thereof. Exemplary suitable IL-15R variants include, without limitation, those described, e.g., in Mortier E et al, 2006; The Journal of Biological Chemistry 2006 281: 1612-1619; or in Bessard-A et al., Mol Cancer Ther. 2009 September; 8(9):2736-45, the entire contents of each of which are incorporated by reference herein. Additional suitable variants will be apparent to those of ordinary skill in the art based on the present disclosure and the knowledge in the art. The disclosure is not limited in this respect.

In some embodiments, the TGFβR2 is a dominant-negative variant of TGFβ receptor II (DN-TGFβR2).

In some embodiments, the CAR is capable of binding mesothelin, EGFR, HER2, MICA/B, BCMA, CD19, CD22, CD20, CD33, CD123, androgen receptor, PSMA, PSCA, Muc1, HPV viral peptides (ie. E7), EBV viral peptides, CD70, WT1, CEA, EGFRvIII, IL13Ra2, GD2, CA125, CD7, EpCAM, Muc16, and/or CD30,

In some embodiments, the modified lymphocyte is derived from a pluripotent or multipotent stem cell. In some embodiments, the multipotent stem cell is a hematopoietic stem cell (HSC). In some embodiments, the pluripotent stem cell is an induced pluripotent stem cell (iPSC). In some embodiments, the pluripotent stem cell is an embryonic stem cell (ESC).

In some embodiments, the modified lymphocyte is derived from a pluripotent or multipotent stem cell that comprises at least one or more exogenous nucleic acid constructs encoding any of (1)(i)-(1)(ix), or any combination thereof; and/or at least one genomic alteration that effects the loss-of-function of any of (2)(i)-(2)(xi), or any combination thereof, in the lymphocyte.

In some embodiments, the modified lymphocyte is derived from a pluripotent or multipotent stem cell that comprises at least one genomic alteration that effects the loss-of-function of any of (2)(i)-(2)(xi), or any combination thereof, in the lymphocyte.

In some embodiments, the at least one genomic alteration that effects the loss-of-function of one or more (2)(i)-(2)(xi) in the lymphocyte comprises an insertion of an exogenous nucleic acid construct.

In some embodiments, the exogenous nucleic acid construct encodes any of (1)(i)-(1)(ix), or any combination thereof.

In some embodiments, the modified lymphocyte exhibits a loss-of-function in two or more of the genes/proteins listed under (2).

In some embodiments, the modified lymphocyte comprises an indel or an insertion of an exogenous nucleotide construct into a genomic locus harboring a gene or encoding a protein under (2).

In some embodiments, the modified lymphocyte comprises an indel or an insertion of an exogenous nucleotide construct into two or more genomic loci harboring a gene or encoding a protein under (2).

In some embodiments, the modified lymphocyte was obtained by editing a genomic locus with an RNA-guided nuclease. In some embodiments, the RNA-guided nuclease is a CRISPR/Cas nuclease. In some embodiments, the RNA-guided nuclease is selected from the group consisting of SpCas9, SaCas9, (KKH) SaCas9, AsCpf1 (AsCas12a), LbCpf1, (LbCas12a), CasX, CasY, Cas12h1, Cas12i1, Cas12c1, Cas12c2, eSpCas9, Cas9-HF1, HypaCas9, dCas9-Fokl, Sniper-Cas9, xCas9, AaCas12b, evoCas9, SpCas9-NG, VRQR, VRER, NmeCas9, CjCas9, BhCas12b, and BhCas12b V4.

In some embodiments, the modified lymphocyte is obtained by editing two or more genomic loci harboring genes encoding any of the proteins under (2). In some embodiments, at least two of the two or more genomic loci harboring genes encoding any of the proteins under (2) have been edited by a different RNA-guided nuclease. In some embodiments, at least one of the two or more genomic loci harboring genes encoding any of the proteins under (2) has been edited by Cas9, and wherein at least one of the loci has been edited by Cpf1.

In some embodiments, the modified lymphocyte expresses endogenous CD56, CD49, and CD45.

In some embodiments, the modified lymphocyte is a natural killer (NK) cell.

In another aspect the disclosure features a modified cell, wherein the modified cell (1) comprises at least one exogenous nucleic acid construct encoding: (i) a chimeric antigen receptor (CAR); (ii) a non-naturally occurring variant of immunoglobulin gamma Fc region receptor III (FcγRIII, cluster of differentiation 16 ((CD16); (iii) interleukin 15 (IL-15); (iv) IL-15 receptor (IL-15R), or a variant thereof; (v) interleukin 12 (IL-12); (vi) IL-12 receptor (IL-12R), or a variant thereof; (vii) human leukocyte antigen G (HLA-G); (viii) human leukocyte antigen E (HLA-E); (ix) leukocyte surface antigen cluster of differentiation CD47 (CD47); or any combination of two or more thereof; and/or (2) exhibits a loss of function of at least one of: (i) transforming growth factor beta receptor 2 (TGFβR2); (ii) adenosine A2a receptor (ADORA2A); (iii) T cell immunoreceptor with Ig and ITIM domains (TIGIT); (iv) β-2 microgobulin (B2M); (v) programmed cell death protein 1 (PD-1); (vi) cytokine inducible SH2 containing protein (CISH); (vii) class II, major histocompatibility complex, transactivator (CIITA); (viii) natural killer cell receptor NKG2A (natural killer group 2A); (ix) two or more HLA class II histocompatibility antigen alpha chain genes, and/or two or more HLA class II histocompatibility antigen beta chain genes; (x) cluster of differentiation 32B (CD32B, FCGR2B); (xi) T cell receptor alpha constant (TRAC); or any combination of two or more thereof. In one embodiment, the modified cell exhibits a loss of function of TGFβR2 and a loss-of-function of CISH. In one embodiment, the modified cell exhibits a loss-of-function of TGFbetaR2 and a loss-of-function of TIGIT. In one embodiment, the modified cell exhibits a loss-of-function of TGFbetaR2 and a loss-of-function of ADORA2A. In one embodiment, the modified cell exhibits a loss-of-function of TGFbetaR2 and a loss-of-function of NKG2A. In one embodiment, the modified cell exhibits a loss-of-function of CISH and a loss-of-function of TIGIT. In one embodiment, the modified cell exhibits a loss-of-function of CISH and a loss-of-function of ADORA2A. In one embodiment, the modified cell exhibits a loss-of-function of CISH and a loss-of-function of NKG2A. In one embodiment, the modified cell exhibits a loss-of-function of TIGIT and a loss-of-function of ADORA2A. In one embodiment, the modified cell exhibits a loss-of-function of TIGIT and a loss-of-function of NKG2A. In one embodiment, the modified cell exhibits a loss-of-function of ADORA2A and a loss-of-function of NKG2A. In one embodiment, the modified cell exhibits a loss-of-function of TGFbetaR2, a loss-of-function of CISH, and a loss-of-function of TIGIT. In one embodiment, the modified cell exhibits a loss-of-function of TGFbetaR2, a loss-of-function of CISH, and a loss-of-function of ADORA2A. In one embodiment, the modified cell exhibits a loss-of-function of TGFbetaR2, a loss-of-function of CISH, and a loss-of-function of NKG2A. In one embodiment, the modified cell exhibits a loss-of-function of TGFbetaR2, a loss-of-function of TIGIT, and a loss-of-function of ADORA2A. In one embodiment, the modified cell exhibits a loss-of-function of TGFbetaR2, a loss-of-function of TIGIT, and a loss-of-function of NKG2A. In one embodiment, the modified cell exhibits a loss-of-function of TGFbetaR2, a loss-of-function of ADORA2A, and a loss-of-function of NKG2A. In one embodiment, the modified cell exhibits a loss-of-function of CISH, a loss-of-function of TIGIT, and a loss-of-function of ADORA2A. In one embodiment, the modified cell exhibits a loss-of-function of CISH, a loss-of-function of TIGIT, and a loss-of-function of NKG2A. In one embodiment, the modified cell exhibits a loss-of-function of CISH, a loss-of-function of ADORA2A, and a loss-of-function of NKG2A. In one embodiment, the modified cell exhibits a loss-of-function of TIGIT, a loss-of-function of ADORA2A, and a loss-of-function of NKG2A.

In one embodiment, the modified cell (1) comprises at least one exogenous nucleic acid construct encoding: (i) a chimeric antigen receptor (CAR); (ii) a non-naturally occurring variant of immunoglobulin gamma Fc region receptor III (FcγRIII, cluster of differentiation 16 ((CD16); (iii) interleukin 15 (IL-15); (iv) IL-15 receptor (IL-15R), or a variant thereof; (v) interleukin 12 (IL-12); (vi) IL-12 receptor (IL-12R), or a variant thereof; (vii) human leukocyte antigen G (HLA-G); (viii) human leukocyte antigen E (HLA-E); (ix) leukocyte surface antigen cluster of differentiation CD47 (CD47); or any combination of two or more thereof; and/or (2) exhibits a loss of function of transforming growth factor beta receptor 2 (TGFβR2), cytokine inducible SH2 containing protein (CISH), or a combination thereof.

In some embodiments of modified cells comprising an exogenous nucleic acid construct, e.g., modified lymphocytes provided herein, the exogenous nucleic acid construct is an expression construct comprising a nucleic acid sequence encoding the gene product listed under (1)(i)-(1(x), or any combination thereof, operably linked to a promoter driving expression of the nucleic acid sequence in a target cell, e.g., in a modified lymphocyte, for example, a modified NK cell provided herein. In some embodiments, the promoter is specifically expressed in the target cell, e.g., the promoter is a lymphocyte- or NK-cell-specific promoter. In some embodiments, the promoter is a CD56 (NCAM) promoter. In some embodiments, the promoter is a CD49 promoter. In some embodiments, the promoter is a CD45 promoter. In some embodiments, the promoter is an FcγRIII promoter. In some embodiments, the promoter is an NKG2D promoter. In some embodiments, the promoter is a CD69 promoter.

In some embodiments of modified cells, e.g., modified lymphocytes provided herein, the exogenous nucleic acid construct encoding a gene product listed under (1) is knocked into a genomic locus encoding a gene product listed under (2), resulting in a loss-of-function of the gene product listed under (2) and expression of a gene product encoded by the exogenous nucleic acid construct, either driven by a heterologous promoter, or driven by the endogenous promoter of the genomic locus that the exogenous nucleic acid construct is knocked into.

In some embodiments of modified cells, e.g., modified lymphocytes provided herein, comprising a loss of function in two or more HLA class II histocompatibility antigen alpha chain genes, and/or two or more HLA class II histocompatibility antigen beta chain genes, the two or more HLA class II histocompatibility antigen alpha chain genes are selected from HLA-DQA1, HLA-DRA, HLA-DPA1, HLA-DMA, HLA-DQA2, and HLA-DOA. In some embodiments, the two or more HLA class II histocompatibility antigen beta chain genes are selected from HLA-DMB, HLA-DOB, HLA-DPB1, HLA-DQB1, HLA-DQB3, HLA-DQB2, HLA-DRB1, HLA-DRB3, HLA-DRB4, and HLA-DRB5.

In some embodiments, the modified cell is an immune cell. In some embodiments, the immune cell is a lymphocyte. In some embodiments, the lymphocyte is an NK cell. In some embodiments, the lymphocyte is an iNK cell.

In some embodiments, the modified cell is a multipotent or pluripotent stem cell, e.g., an iPS cell, or a hematopoietic stem cell, or a differentiated cell derived from such a multipotent or pluripotent stem cell, e.g., an iNK cell.

In some embodiments, the modified cell does not express an endogenous T-cell co-receptor.

In some embodiments, the lymphocyte is a T cell.

In some embodiments, the modified cell comprises a rearranged endogenous TCR locus, wherein the rearranged TCR comprises TCRα VJ and/or TCRβ V(D)J section rearrangements and complete V-domain exons.

In some embodiments, the modified cell expresses at least one endogenous gene encoding: (i) CD56 (NCAM), CD49, and/or CD45; (ii) NK cell receptor (cluster of differentiation 16 (CD16)); (iii) natural killer group-2 member D (NKG2D); (iv) CD69; (v) a natural cytotoxicity receptor; or any combination of two or more thereof.

In some embodiments, the natural cytotoxicity receptor is NKp30, NKp44, NKp46, and/or CD158b.

In some embodiments, the modified cell expresses at least one NK cell biomarker. In some embodiments, the NK cell biomarker is CD56, CD49, and/or CD45.

In one aspect, disclosed herein is a population of cells comprising the modified lymphocyte described herein, or the modified cell described herein.

In one aspect, disclosed herein is a pharmaceutical composition comprising the population of cells disclosed herein.

In another aspect, the disclosure provides an isolated population of lymphocytes, wherein the population comprises at least 1×10³, at least 1×10⁴, at least 1×10⁵, at least 2×10⁵, at least 3×10⁵, at least 4×10⁵, at least 5×10⁵, at least 1×10⁶, at least 2×10⁶, at least 3×10⁶, at least 4×10⁶, at least 5×10⁶, at least 1×10⁷, at least 1×10⁷, at least 2×10⁷, at least 3×10⁷, at least 4×10⁷, at least 5×10⁷, at least 1×10⁸, at least 2×10⁸, at least 3×10⁸, at least 4×10⁸, at least 5×10⁸, at least 1×10⁹, at least 1×10⁹, at least 2×10⁹, at least 3×10⁹, at least 4×10⁹, at least 5×10⁹, at least 1×10¹⁰, at least 2×10¹⁰, at least 3×10¹⁰, at least 4×10¹⁰, at least 5×10¹⁰, at least 1×10¹¹, or at least 1×10¹² cells, and wherein at least 50%, at least 60%, at least 70%, at least 80%, at least 90%, at least 95%, at least 98%, at least 99%, at least 99.9%, at least 99.99%, at least 99.999%, or virtually 100% of the lymphocytes in the population: (a) comprise a rearranged T-cell receptor (TCR) locus; (b) do not express endogenous CD3; (c) express endogenous CD56 (NCAM), CD49, and/or CD45; and (d) expresses at least endogenous gene encoding: (i) NK cell receptor (cluster of differentiation 16 (CD16)); (ii) natural killer group-2 member D (NKG2D); (iii) CD69; (iv) a natural cytotoxicity receptor; or any combination of two or more thereof; and wherein the modified lymphocyte further: (1) comprises at least one exogenous nucleic acid construct encoding: (i) chimeric antigen receptor (CAR); (ii) non-naturally occurring variant of immunoglobulin gamma Fc region receptor III (FcγRIII, CD16); (iii) interleukin 15 (IL-15); (iv) IL-15 receptor (IL-15R), or a variant thereof; (v) interleukin 12 (IL-12); (vi) IL-12 receptor (IL-12R), or a variant thereof; (vii) human leukocyte antigen G (HLA-G); (viii) human leukocyte antigen E (HLA-E); (ix) leukocyte surface antigen cluster of differentiation CD47 (CD47); or any combination of two or more thereof; and/or (2) exhibits a loss of function of at least one of: (i) transforming growth factor beta receptor 2 (TGFβR2); (ii) adenosine A2a receptor (ADORA2A); (iii) T cell immunoreceptor with Ig and ITIM domains (TIGIT); (iv) β-2 microgobulin (B2M); (v) programmed cell death protein 1 (PD-1); (vi) cytokine inducible SH2 containing protein (CISH); (vii) class II, major histocompatibility complex, transactivator (CIITA); (viii) natural killer cell receptor NKG2A (natural killer group 2A); (ix) two or more HLA class II histocompatibility antigen alpha chain genes, and/or two or more HLA class II histocompatibility antigen beta chain genes; (x) cluster of differentiation 32B (CD32B, FCGR2B); (xi) T cell receptor alpha constant (TRAC); or any combination of two or more thereof. In one embodiment, the modified lymphocyte exhibits a loss of function of TGFβR2 and a loss-of-function of CISH. In one embodiment, the modified lymphocyte exhibits a loss-of-function of TGFbetaR2 and a loss-of-function of TIGIT. In one embodiment, the modified lymphocyte exhibits a loss-of-function of TGFbetaR2 and a loss-of-function of ADORA2A. In one embodiment, the modified lymphocyte exhibits a loss-of-function of TGFbetaR2 and a loss-of-function of NKG2A. In one embodiment, the modified lymphocyte exhibits a loss-of-function of CISH and a loss-of-function of TIGIT. In one embodiment, the modified lymphocyte exhibits a loss-of-function of CISH and a loss-of-function of ADORA2A. In one embodiment, the modified lymphocyte exhibits a loss-of-function of CISH and a loss-of-function of NKG2A. In one embodiment, the modified lymphocyte exhibits a loss-of-function of TIGIT and a loss-of-function of ADORA2A. In one embodiment, the modified lymphocyte exhibits a loss-of-function of TIGIT and a loss-of-function of NKG2A. In one embodiment, the modified lymphocyte exhibits a loss-of-function of ADORA2A and a loss-of-function of NKG2A. In one embodiment, the modified lymphocyte exhibits a loss-of-function of TGFbetaR2, a loss-of-function of CISH, and a loss-of-function of TIGIT. In one embodiment, the modified lymphocyte exhibits a loss-of-function of TGFbetaR2, a loss-of-function of CISH, and a loss-of-function of ADORA2A. In one embodiment, the modified lymphocyte exhibits a loss-of-function of TGFbetaR2, a loss-of-function of CISH, and a loss-of-function of NKG2A. In one embodiment, the modified lymphocyte exhibits a loss-of-function of TGFbetaR2, a loss-of-function of TIGIT, and a loss-of-function of ADORA2A. In one embodiment, the modified lymphocyte exhibits a loss-of-function of TGFbetaR2, a loss-of-function of TIGIT, and a loss-of-function of NKG2A. In one embodiment, the modified lymphocyte exhibits a loss-of-function of TGFbetaR2, a loss-of-function of ADORA2A, and a loss-of-function of NKG2A. In one embodiment, the modified lymphocyte exhibits a loss-of-function of CISH, a loss-of-function of TIGIT, and a loss-of-function of ADORA2A. In one embodiment, the modified lymphocyte exhibits a loss-of-function of CISH, a loss-of-function of TIGIT, and a loss-of-function of NKG2A. In one embodiment, the modified lymphocyte exhibits a loss-of-function of CISH, a loss-of-function of ADORA2A, and a loss-of-function of NKG2A. In one embodiment, the modified lymphocyte exhibits a loss-of-function of TIGIT, a loss-of-function of ADORA2A, and a loss-of-function of NKG2A.

In one embodiment, the isolated population of myphocytes comprises at least 1×10³, at least 1×10⁴, at least 1×10⁵, at least 2×10⁵, at least 3×10⁵, at least 4×10⁵, at least 5×10⁵, at least 1×10⁶, at least 2×10⁶, at least 3×10⁶, at least 4×10⁶, at least 5×10⁶, at least 1×10⁷, at least 1×10⁷, at least 2×10⁷, at least 3×10⁷, at least 4×10⁷, at least 5×10⁷, at least 1×10⁸, at least 2×10⁸, at least 3×10⁸, at least 4×10⁸, at least 5×10⁸, at least 1×10⁹, at least 1×10⁹, at least 2×10⁹, at least 3×10⁹, at least 4×10⁹, at least 5×10⁹, at least 1×10¹⁰, at least 2×10¹⁰, at least 3×10¹⁰, at least 4×10¹⁰, at least 5×10¹⁰, at least 1×10¹¹, or at least 1×10¹² cells, and wherein at least 50%, at least 60%, at least 70%, at least 80%, at least 90%, at least 95%, at least 98%, at least 99%, at least 99.9%, at least 99.99%, at least 99.999%, or virtually 100% of the lymphocytes in the population: (a) comprise a rearranged T-cell receptor (TCR) locus; (b) do not express endogenous CD3; (c) express endogenous CD56 (NCAM), CD49, and/or CD45; and (d) expresses at least endogenous gene encoding: (i) NK cell receptor (cluster of differentiation 16 (CD16)); (ii) natural killer group-2 member D (NKG2D); (iii) CD69; (iv) a natural cytotoxicity receptor; or any combination of two or more thereof; and wherein the modified lymphocyte further: (1) comprises at least one exogenous nucleic acid construct encoding: (i) chimeric antigen receptor (CAR); (ii) non-naturally occurring variant of immunoglobulin gamma Fc region receptor III (FcγRIII, CD16); (iii) interleukin 15 (IL-15); (iv) IL-15 receptor (IL-15R), or a variant thereof; (v) interleukin 12 (IL-12); (vi) IL-12 receptor (IL-12R), or a variant thereof; (vii) human leukocyte antigen G (HLA-G); (viii) human leukocyte antigen E (HLA-E); (ix) leukocyte surface antigen cluster of differentiation CD47 (CD47); or any combination of two or more thereof; and/or (2) exhibits a loss of function of transforming growth factor beta receptor 2 (TGFβR2), cytokine inducible SH2 containing protein (CISH), or a combination thereof.

In some embodiments, the rearranged TCR locus comprises of TCRα VJ and/or TCRβ V(D)J section rearrangements and complete V-domain exons. In some embodiments, the rearranged endogenous TCR locus consists of no more than two rearranged alleles.

In some embodiments, the natural cytotoxicity receptor is NKp30, NKp44, NKp46, and/or CD158b.

In some embodiments, the in vitro population of lymphocytes does not comprise more than 1%, more than 0.1%, more than 0.001%, more than 0.0001%, more than 0.00001%, more than 0.000001%, more than 0.0000001%, more than 0.00000001%, more than 0.000000001%, more than 0.0000000001%, or more than more than 0.00000000001% of cells expressing a reprogramming factor from an exogenous nucleic acid construct.

In some embodiments, the in vitro population of lymphocytes does not comprise a cell expressing a reprogramming factor from an exogenous nucleic acid construct. In some embodiments, the reprogramming factor is Oct-4 and/or Sox-2.

In some embodiments, the in vitro population of lymphocytes does not comprise cells harboring episomal expression constructs encoding a reprogramming factor.

In some embodiments, each cell in in vitro population of lymphocytes comprises the same combination of an exogenous nucleic acid construct listed under (1) and a loss of function listed (2).

In some embodiments, the in vitro population of lymphocytes comprises less than 0.001%, less than 0.002%, less than 0.003%, less than 0.004%, less than 0.005%, less than 0.006%, less than 0.007%, less than 0.008%, less than 0.009%, less than 0.01%, less than 0.02%, less than 0.03%, less than 0.04%, less than 0.05%, less than 0.06%, less than 0.07%, less than 0.08%, less than 0.09%, less than 0.1%, less than 0.2%, less than 0.3%, less than 0.4%, less than 0.5%, less than 0.6%, less than 0.7%, less than 0.8%, less than 0.9%, less than 1%, less than 2%, less than 3%, less than 4%, less than 5%, less than 6%, less than 7%, less than 8%, less than 9%, or less than 10% cell that harbor a chromosomal translocation.

In another aspect, the disclosure provides a method of treating a subject, the method comprising administering any modified lymphocyte, any modified cell, any pharmaceutical composition, or the isolated in vitro population of cells as described in the disclosure, to a subject in need thereof. In some embodiments, the subject has, or is diagnosed with, a proliferative disease. In some embodiments, the proliferative disease is cancer. In some embodiments, the cancer is breast cancer, colorectal cancer, gastric cancer, renal cell carcinoma (RCC), or non-small cell lung cancer (NSCLC), solid tumors, bladder cancer, hepatocellular carcinoma, prostate cancer, ovarian/uterine cancer, pancreatic cancer, mesothelioma, melanoma, glioblastoma, HPV-associated and/or HPV-positive cancers such as cervical and HPV+ head and neck cancer, oral cavity cancer, cancer of the pharynx, thyroid cancer, gallbladder cancer, soft tissue sarcomas, and hematological cancers like ALL, CLL, NHL, DLBCL, AML, CML, multiple myeloma (MM).

In some embodiments, the method of generating the modified lymphocyte, the modified cell, the population of cells, or the isolated in vitro population of lymphocytes of the disclosure comprises: (a) obtaining an induced pluripotent stem cell (iPSC); (b) modifying the iPSC, or an undifferentiated or differentiated daughter cell thereof, to comprise express at least one exogenous gene of (1) and/or to comprise a loss of function in at least one gene of (2); (c) directing differentiation of the iPSC to hematopoietic lineage cells, wherein the hematopoietic lineage cells retain the edited genetic loci comprised in the iPSCs.

In some embodiments, directing differentiation comprises: (i) contacting iPSCs with a composition comprising a BMP pathway activator, and optionally bFGF, to obtain mesodermal cells; and (ii) contacting the mesodermal cells with a composition comprising a BMP pathway activator, bFGF, and a WNT pathway activator, to obtain mesodermal cells having definitive hemogenic endothelium (HE) potential, wherein the mesodermal cells having definitive hemogenic endothelium (HE) potential are capable of providing hematopoietic lineage cells; wherein mesodermal cells and mesodermal cells having definitive HE potential are obtained in steps (i) and (ii) without the step of forming embryoid bodies; wherein the hematopoietic lineage cells comprise definitive hemogenic endothelium cells, hematopoietic stem and progenitor cells (HSC), hematopoietic multipotent progenitor cell (MPP), pre-T cell progenitor cells, pre-NK cell progenitor cells, T cell progenitor cells, NK cell progenitor cells, T cells, NK cells, NKT cells, or B cells.

In some embodiments, the method of directing differentiation of iPSCs to hematopoietic lineage cells further comprises: contacting the mesodermal cells having definitive HE potential with a composition comprising bFGF and a ROCK inhibitor to obtain definitive HE cells.

In some embodiments, the method of directing differentiation further comprises: contacting the definitive HE cells with a composition comprising a BMP activator, and optionally a ROCK inhibitor, and one or more growth factors and cytokines selected from the group consisting of TPO, IL3, GMCSF, EPO, bFGF, VEGF, SCF, IL6, Flt3L and IL11 to obtain hematopoietic multipotent progenitor cells (MPP).

In some embodiments, the method of directing differentiation further comprises: contacting the definitive HE cells with a composition comprising one or more growth factors and cytokines selected from the group consisting of SCF, Flt3L, and IL7; and optionally one or more of a BMP activator, a ROCK inhibitor, TPO, VEGF and bFGF to obtain pre-T cell progenitors, T cell progenitors, and/or T cells.

In some embodiments, the method of directing differentiation further comprises: contacting the definitive HE cells with a composition comprising one or more growth factors and cytokines selected from the group consisting of SCF, Flt3L, TPO, IL7 and IL15, and optionally one or more of a BMP activator, a ROCK inhibitor, VEGF and bFGF to obtain pre-NK cell progenitors, NK cell progenitors, and/or NK cells.

In some embodiments, the method of generating the modified lymphocyte, the modified cell, the population of cells, or the isolated in vitro population of lymphocytes of the disclosure further comprises: prior to step c), contacting the pluripotent stem cells with a composition comprising a MEK inhibitor, a GSK3 inhibitor, and a ROCK inhibitor, to seed and expand the cells.

In some embodiments, the method of generating the modified lymphocyte, the modified cell, the population of cells, or the isolated in vitro population of lymphocytes of the disclosure further comprises: detecting a rearranged T-cell receptor (TCR) locus in the hematopoietic lineage cells. In some embodiments, the method further comprises selecting the hematopoietic lineage cells comprising the rearranged TCR locus based on the TCR encoded by the rearranged TCR locus binding an antigen of interest. In some embodiments, the antigen of interest is a tumor antigen.

In another aspect, the disclosure provides a method, the method comprising: reprogramming a donor cell to a pluripotent state; editing a target locus in the donor cell genome; and differentiating the reprogrammed donor cell into a lymphocyte. In some embodiments, the editing is performed before or during the step of reprogramming of the donor cell to a pluripotent state. In some embodiments, the donor cell is a fibroblast, a peripheral blood cell, a lymphocyte, or a T cell.

In another aspect, the disclosure provides a method, the method comprising: differentiating a genetically modified pluripotent stem cell into a lymphocyte, wherein the genetically modified pluripotent stem cell comprises: (1) an exogenous nucleic acid comprising: (i) a nucleic acid encoding a chimeric antigen receptor (CAR); (ii) a nucleic acid encoding a non-naturally occurring variant of immunoglobulin gamma Fc region receptor III (FcγRIII, CD16); (iii) a nucleic acid encoding interleukin 15 (IL-15); (iv) a nucleic acid encoding IL-15R, or a variant thereof; (v) a nucleic acid encoding interleukin 12 (IL-12); (vi) a nucleic acid encoding IL-12R, or a variant thereof; (vii) a nucleic acid encoding human leukocyte antigen G (HLA-G); (viii) human leukocyte antigen E (HLA-E); (ix) leukocyte surface antigen cluster of differentiation CD47 (CD47); or any combination of two or more thereof; and (2) an indel, or an insertion of an exogenous nucleic acid in one or more of the following genetic loci: (i) transforming growth factor beta receptor 2 (TGFβR2); (ii) adenosine A2a receptor (ADORA2A); (iii) T cell immunoreceptor with Ig and ITIM domains (TIGIT); (iv) β-2 microgobulin (B2M); (v) programmed cell death protein 1 (PD-1, CD279); (vi) cytokine inducible SH2 containing protein (CISH); (vii) class II, major histocompatibility complex, transactivator (CIITA); (viii) natural killer cell receptor NKG2A (natural killer group 2A); (ix) two or more HLA class II histocompatibility antigen alpha chain genes, and/or two or more HLA class II histocompatibility antigen beta chain genes; (x) cluster of differentiation 32B (CD32B, FCGR2B); (xi) T cell receptor alpha constant (TRAC); or any combination of two or more thereof, wherein the indel or insertion results in a loss-of-function of a gene product encoded by the respective genetic locus or loci. In one embodiment, the method comprises differentiating a genetically modified pluripotent stem cell into a lymphocyte, wherein the genetically modified pluripotent stem cell comprises an indel, or an insertion of an exogenous nucleic acid in TGFβR2 and CISH, wherein the indel or insertion results in a loss-of-function of a gene product encoded by TGFβR2 and/or CISH. In one embodiment, the method comprises differentiating a genetically modified pluripotent stem cell into a lymphocyte, wherein the genetically modified pluripotent stem cell comprises an indel, or an insertion of an exogenous nucleic acid in TGFβR2 and TIGIT, wherein the indel or insertion results in a loss-of-function of a gene product encoded by TGFβR2 and/or TIGIT. In one embodiment, the method comprises differentiating a genetically modified pluripotent stem cell into a lymphocyte, wherein the genetically modified pluripotent stem cell comprises an indel, or an insertion of an exogenous nucleic acid in TGFβR2 and ADORA2A, wherein the indel or insertion results in a loss-of-function of a gene product encoded by TGFβR2 and/or ADORA2A. In one embodiment, the method comprises differentiating a genetically modified pluripotent stem cell into a lymphocyte, wherein the genetically modified pluripotent stem cell comprises an indel, or an insertion of an exogenous nucleic acid in TGFβR2 and NKG2A, wherein the indel or insertion results in a loss-of-function of a gene product encoded by TGFβR2 and/or NKG2A. In one embodiment, the method comprises differentiating a genetically modified pluripotent stem cell into a lymphocyte, wherein the genetically modified pluripotent stem cell comprises an indel, or an insertion of an exogenous nucleic acid in CISH and TIGIT, wherein the indel or insertion results in a loss-of-function of a gene product encoded by CISH and/or TIGIT. In one embodiment, the method comprises differentiating a genetically modified pluripotent stem cell into a lymphocyte, wherein the genetically modified pluripotent stem cell comprises an indel, or an insertion of an exogenous nucleic acid in CISH and ADORA2A, wherein the indel or insertion results in a loss-of-function of a gene product encoded by CISH and/or ADORA2A. In one embodiment, the method comprises differentiating a genetically modified pluripotent stem cell into a lymphocyte, wherein the genetically modified pluripotent stem cell comprises an indel, or an insertion of an exogenous nucleic acid in CISH and NKG2A, wherein the indel or insertion results in a loss-of-function of a gene product encoded by CISH and/or NKG2A. In one embodiment, the method comprises differentiating a genetically modified pluripotent stem cell into a lymphocyte, wherein the genetically modified pluripotent stem cell comprises an indel, or an insertion of an exogenous nucleic acid in TIGIT and ADORA2A, wherein the indel or insertion results in a loss-of-function of a gene product encoded by TIGIT and/or ADORA2A. In one embodiment, the method comprises differentiating a genetically modified pluripotent stem cell into a lymphocyte, wherein the genetically modified pluripotent stem cell comprises an indel, or an insertion of an exogenous nucleic acid in TIGIT and NKG2A, wherein the indel or insertion results in a loss-of-function of a gene product encoded by TIGIT and/or NKG2A. In one embodiment, the method comprises differentiating a genetically modified pluripotent stem cell into a lymphocyte, wherein the genetically modified pluripotent stem cell comprises an indel, or an insertion of an exogenous nucleic acid in ADORA2A and NKG2A, wherein the indel or insertion results in a loss-of-function of a gene product encoded by ADORA2A and/or NKG2A. In one embodiment, the method comprises differentiating a genetically modified pluripotent stem cell into a lymphocyte, wherein the genetically modified pluripotent stem cell comprises an indel, or an insertion of an exogenous nucleic acid in TGFβR2, CISH and TIGIT, wherein the indel or insertion results in a loss-of-function of a gene product encoded by TGFβR2, CISH and/or TIGIT. In one embodiment, the method comprises differentiating a genetically modified pluripotent stem cell into a lymphocyte, wherein the genetically modified pluripotent stem cell comprises an indel, or an insertion of an exogenous nucleic acid in TGFβR2, CISH and ADORA2A, wherein the indel or insertion results in a loss-of-function of a gene product encoded by TGFβR2, CISH and/or ADORA2A. In one embodiment, the method comprises differentiating a genetically modified pluripotent stem cell into a lymphocyte, wherein the genetically modified pluripotent stem cell comprises an indel, or an insertion of an exogenous nucleic acid in TGFβR2, CISH and NKG2A, wherein the indel or insertion results in a loss-of-function of a gene product encoded by TGFβR2, CISH and/or NKG2A. In one embodiment, the method comprises differentiating a genetically modified pluripotent stem cell into a lymphocyte, wherein the genetically modified pluripotent stem cell comprises an indel, or an insertion of an exogenous nucleic acid in TGFβR2, TIGIT and ADORA2A, wherein the indel or insertion results in a loss-of-function of a gene product encoded by TGFβR2, TIGIT and/or ADORA2A. In one embodiment, the method comprises differentiating a genetically modified pluripotent stem cell into a lymphocyte, wherein the genetically modified pluripotent stem cell comprises an indel, or an insertion of an exogenous nucleic acid in TGFβR2, TIGIT and NKG2A, wherein the indel or insertion results in a loss-of-function of a gene product encoded by TGFβR2, TIGIT and/or NKG2A. In one embodiment, the method comprises differentiating a genetically modified pluripotent stem cell into a lymphocyte, wherein the genetically modified pluripotent stem cell comprises an indel, or an insertion of an exogenous nucleic acid in TGFβR2, ADORA2A and NKG2A, wherein the indel or insertion results in a loss-of-function of a gene product encoded by TGFβR2, ADORA2A and/or NKG2A. In one embodiment, the method comprises differentiating a genetically modified pluripotent stem cell into a lymphocyte, wherein the genetically modified pluripotent stem cell comprises an indel, or an insertion of an exogenous nucleic acid in CISH, TIGIT and ADORA2A, wherein the indel or insertion results in a loss-of-function of a gene product encoded by CISH, TIGIT and/or ADORA2A. In one embodiment, the method comprises differentiating a genetically modified pluripotent stem cell into a lymphocyte, wherein the genetically modified pluripotent stem cell comprises an indel, or an insertion of an exogenous nucleic acid in CISH, TIGIT and NKG2A, wherein the indel or insertion results in a loss-of-function of a gene product encoded by CISH, TIGIT and/or NKG2A. In one embodiment, the method comprises differentiating a genetically modified pluripotent stem cell into a lymphocyte, wherein the genetically modified pluripotent stem cell comprises an indel, or an insertion of an exogenous nucleic acid in CISH, ADORA2A and NKG2A, wherein the indel or insertion results in a loss-of-function of a gene product encoded by CISH, ADORA2A and/or NKG2A. In one embodiment, the method comprises differentiating a genetically modified pluripotent stem cell into a lymphocyte, wherein the genetically modified pluripotent stem cell comprises an indel, or an insertion of an exogenous nucleic acid in TIGIT, ADORA2A and NKG2A, wherein the indel or insertion results in a loss-of-function of a gene product encoded by TIGIT, ADORA2A and/or NKG2A.

In some embodiments, the exogenous nucleic acid of (2) is the exogenous nucleic acid of (1). In some embodiments, the pluripotent stem cell is an iPS cell. In some embodiments, the differentiating comprises contacting the pluripotent stem cell with a differentiation medium or a sequence of differentiation media.

### BRIEF DESCRIPTION OF THE DRAWINGS

FIGS. 1A and 1B depict that robust single and double-gene editing of TGFBR2 and CISH was achieved in NK cells. Both single and simultaneous targeting of TGFBR2 and CISH in NK cells using CRISPR-Cpf1 produced in/dels at both targets in greater than 80% of NK cells, with greater than 90% of edited NK cells viable at 72 hours post-editing.

FIGS. 2A and 2B depict that normalization of spheroid curves maintains the same efficacy patterns as observed in the non-normalized data, as analyzed across 3 unique donors and 5 independent experiments. Each single knockout (SKO) NK group was significantly more efficient at reducing the SK-OV-3 spheroid size than the control NK, and the double knockout (DKO) NK group was significantly more efficient at reducing the SK-OV-3 spheroid size than the SKO NK groups. FIG. 2A depicts SK-OV-3 spheriod analysis at 10:1 E:T with 10 ng/mL TGFbeta (3 donors, 5 independent experiments). FIG. 2B has error bars that are SEM. Statistical significance is a result of 2-way ANOVA analysis. 2-way ANOVA analysis excludes time points greater than 104 hours due to missing time points in some experiments. Mixed model analysis yields same or improved statistical significance between groups when all time points are considered.

FIGS. 3A and 3B depict that the CISH/TGFBR2 double knockout NK cells demonstrate superior effector function over the single knockout NK cells or the control NK cells in the SK-OV-3 spheroid assay, even at lower NK effector cell to target cell (E:T) ratios. FIG. 3A depicts the SK-OV-3 spheroid analysis at 20:1 E:T, with 10 ng/mL TGF-β, as analyzed across 3 unique donors and 5 independent experiments. FIG. 3B depicts the SK-OV-3 spheroid analysis at 10:1 E:T, with 10 ng/mL TGF-β, as analyzed across 4 unique donors and 7 independent experiments. These marginal differences between the different E:T ratios at all conditions suggests that the effector cell phenotype is driven by the knockout rather than NK cell to target ratio.

FIGS. 4A and 4B depict that the CISH/TGFBR2 double knockout NK cells demonstrate superior effector function over the single knockout NK cells or the control NK cells in the PC-3 spheroid assay, even at lower NK effector cell to target cell (E:T) ratios. FIG. 4A depicts the PC-3 spheroid analysis at 20:1 E:T, with 10 ng/mL TGF-β, as analyzed across 3 unique donors and 5 independent experiments. FIG. 4B depicts the PC-3 spheroid analysis at 10:1 E:T, with 10 ng/mL TGF-β, as analyzed across 4 unique donors and 7 independent experiments. These marginal differences between the different E:T ratios at all conditions suggests that the effector cell phenotype is driven by the knockout rather than NK cell to target ratio.

FIGS. 5A and 5B depict that CISH/TGFBR2 double knockout NK cells demonstrate superior effector function over the single knockout NK cells or the control NK cells in the SK-OV-3 and PC-3 spheroid assays, in the absence of any exogenous cytokine. FIG. 5A depicts the SK-OV-3 spheroid analysis at 10:1 E:T, in the absence of any exogenous cytokine, as analyzed across 4 unique donors and 7 independent experiments. FIG. 5B depicts the PC-3 spheroid analysis at 10:1 E:T, in the absence of any exogenous cytokine, as analyzed across 4 unique donors and 7 independent experiments.

FIG. 6A depicts that IFN-γ concentrations correlate with NK cell efficacy in the spheroid assay. The SK-OV-3 spheroid analysis was performed across different E:Ts with 10 ng/mL TGF-β and 5 ng/mL IL-15. The analysis for 5:1 and 10:1 E:T was performed across 4 unique donors and 7 independent experiments. The analysis for 20:1 E:T was performed across 3 unique donors, and 5 independent experiments.

FIG. 6B depicts that TNF-α concentrations correlate with NK cell efficacy in the spheroid assay. The SK-OV-3 spheroid analysis was performed across different E:Ts with 10 ng/mL TGF-β and 5 ng/mL IL-15. The analysis for 5:1 and 10:1 E:T was performed across 4 unique donors and 7 independent experiments. The analysis for 20:1 E:T was performed across 3 unique donors, and 5 independent experiments.

FIG. 6C depicts marker expression in CISH/TGFBR2 double knockout (DKO) NK cells. Control (non-edited) and double knockout NK cells were harvested for staining at 72 hours post editing. Expression of the NK activation markers CD25 and CD69 were quantified. Double KO NK cells expressed significantly higher levels of activation markers CD25 and CD69 as compared to control NK cells.

FIG. 6D depicts anti-tumor activity of NK cells was measured in an in vivo model. NSG mice received an intraperitoneal injection of 500,000 SKOV3 tumor cells labeled with luciferase. Seven days post-tumor implantation, 10 million edited (CISH/TGFBR2 double-knockout) or unedited (control) NK cells were injected into the peritoneal cavity of the tumor-bearing mice. Tumor burden was monitored weekly by IP administration of luciferin and IVIS imaging. Two-way ANOVA analysis was performed at day 34 to determine statistical significance between control and DKO NK cell groups (****, P<0.0001)

FIG. 7A depicts the robust single gene editing of TIGIT achieved in NK cells, across 2 independent experiments and 3 unique donors.

FIG. 7B depicts the robust single gene editing of NKG2A achieved in NK cells, across 2 independent experiments and 3 unique donors.

FIG. 7C depicts the robust single gene editing of ADORA2A achieved in NK cells, across 3 independent experiments and 3 unique donors.

FIGS. 8A and 8B depict that TIGIT single knockout NK cells demonstrate superior effector function over the unedited control NK cells in the in vitro spheroid assay, at different effector cell to target cell (E:T) ratios. FIG. 8A depicts the tumor spheroid analysis at 20:1 E:T, as analyzed across 2 unique donors and 2 independent experiments. Red object intensity was measured every two hours for 6 days on an Incucyte imaging system. FIG. 8B depicts the tumor spheroid analysis at 1.25:1, 2.5:1, 5:1, 10:1 and 20:1 effector to target ratios, as analyzed across 2 unique donors and 2 independent experiments. Red object intensity is shown at 100 hours following NK cell addition.

FIGS. 9A and 9B depict that NKG2A single knockout NK cells demonstrate superior effector function over the unedited control NK cells in the in vitro spheroid assay, at different effector cell to target cell (E:T) ratios. FIG. 9A depicts the tumor spheroid analysis at 20:1 E:T, as analyzed across 2 unique donors and 2 independent experiments. Red object intensity was measured every two hours for 6 days on an Incucyte imaging system. FIG. 9B depicts the tumor spheroid analysis at 1.25:1, 2.5:1, 5:1, 10:1 and 20:1 E:T, as analyzed across 2 unique donors and 2 independent experiments. Red object intensity is shown at 100 hours following NK cell addition.

FIGS. 10A and 10B depict that ADORA2A single knockout NK cells demonstrate superior effector function over the unedited control NK cells in the in vitro spheroid assay, at different effector cell to target cell (E:T) ratios. FIG. 10A depicts the tumor spheroid analysis at 20:1 E:T, as analyzed across 2 unique donors and 2 independent experiments. Red object intensity was measured every two hours for 6 days on an Incucyte imaging system. FIG. 10B depicts the tumor spheroid analysis at 1.25:1, 2.5:1, 5:1, 10:1 and 20:1 E:T, as analyzed across 2 unique donors and 2 independent experiments. Red object intensity is shown at 100 hours following NK cell addition.

FIG. 11 depicts triple gene editing of TGFbR2/CISH/TIGIT achieved in NK cells.

FIGS. 12A and 12B depict that TGFbR2/CISH/TIGIT triple knockout NK cells demonstrate superior effector function over the unedited control NK cells in the in vitro spheroid assay, at different effector cell to target cell (E:T) ratios. FIG. 12A depicts the tumor spheroid analysis at 20:1 E:T. Red object intensity was measured every two hours for 6 days on an Incucyte imaging system. FIG. 12B depicts the tumor spheroid analysis at 5:1, 10:1 and 20:1 E:T. Red object intensity is shown at 100 hours following NK cell addition.

### DETAILED DESCRIPTION

Some aspects of the present disclosure provide strategies, compositions, and methods useful for engineering “off the shelf” allogeneic cells that can be used in clinical applications. Some aspects of the present disclosure provide strategies, compositions, and methods useful for engineering pluripotent or multipotent stem cells (e.g., induced pluripotent stem cells (iPSCs) or hematopoietic stem cells (HSCs) that can be used to derive differentiated daughter cells, e.g., modified lymphocytes, such as iNK cells. Immunoreactivity, both graft-versus-host and host-versus-graft, is a major challenge for clinical applications of allogeneic cells. Some aspects of the present disclosure provide strategies, compositions, and methods for engineering cells that address various aspect of immunoreactivity typically encountered by non-modified cell grafts in allogeneic settings.

Some aspects of this disclosure provide strategies, compositions, and methods useful for overcoming “nonself” host-versus-graft immunoreactivity, e.g., by removing MHC Class I and II functionality in target cells for allogeneic clinical applications. For example, in some embodiments, MHC Class I and II functionality is achieved by effecting a loss-of-function of B2M (Class I) and of CIITA (Class II) and/or two or more MHC Class II alpha and/or beta chains, as described in more detail elsewhere herein.

Some aspects of the present disclosure provide strategies, compositions, and methods useful for overcoming “missing self” host-versus-graft immunoreactivity, e.g., by introducing an exogenous expression construct comprising a nucleic acid sequence encoding an NK inhibitory modality into target cells for allogeneic clinical applications. For example, in some embodiments, such “missing self” immunoreactivity is addressed by effecting transgenic expression of HLA-G, HLA-E, and/or CD47 in target cells for allogeneic clinical applications.

Some aspects of the present disclosure provide strategies, compositions, and methods useful for overcoming graft-versus-host T-cell receptor (TCR) alloreactivity by removing endogenous TCR functionality. For example, in some embodiments, strategies, compositions, and methods useful for the generation of modified cells for allogeneic clinical applications from multipotent or pluripotent stem cells are provided herein that include engineering the stem cells to comprise the immunomodulatory modifications described herein, and then differentiating the stem cells into a cell type for administration to a patient in need thereof, e.g., into lymphocytes, such as, e.g., iNK cells, for immunotherapy. In some embodiments, the pluripotent or multipotent stem cells are derived from a cell expressing a TCR or comprising a rearranged TCR locus, e.g., from a T-cell, and in some such embodiments, a differentiated lymphocyte derived from such engineered stem cells may express the TCR and be the target of TCR alloreactivity. In some such embodiments, it is advantageous to effect a loss-of-function of the endogenous TCR expression products, and the present disclosure provides strategies, compositions, and methods useful for achieving such a loss-of-function in the respective cells, e.g., by effecting a loss-of-function of TRAC as described in more detail elsewhere herein.

Some aspects of the present disclosure relate to the generation of modified NK cells (or other lymphocytes) that are useful as therapeutic agents, e.g., in the context of immunooncology. For example, at least some of the modified NK cells provided herein exhibit enhanced NK cell response characteristics as compared to non-modified NK cells, e.g., enhanced target recognition, enhanced NK cell response level and/or duration, improved NK cell survival, delayed NK cell exhaustion, enhanced target recognition, and/or recognition of a target not typically recognized by non-modified NK cells.

Some aspects of the present disclosure provide compositions, methods, and strategies for the generation of modified NK cells. In some embodiments, such modified NK cells are generated by editing the genome of mature NK cells. In some embodiments, modified NK cells are generated by editing the genome of a cell from which an NK cell is derived, either in vitro or in vivo. In some embodiments, the cell from which and NK cell is derived is a stem cell, for example, a hematopoietic stem cell (HSC), or a pluripotent stem cells, such as, e.g., an embryonic stem cell (ES cell) or an induced pluripotent stem cell (iPS cell). For example, in some embodiments, modified NK cells are generated by editing the genome of an ES cell, an iPS cell, or a hematopoietic stem cell, and subsequently differentiating the edited stem cell into an NK cell. In some embodiments, where the generation of modified NK cells involves differentiation of the modified NK cell from an iPS cell, the editing of the genome may take place at any suitable time during the generation, maintenance, or differentiation of the iPS cell. For example, where a donor cell is reprogrammed into an iPS cell, the donor cell, e.g., a somatic cell such as, for example, a fibroblast cell or a T lymphocyte, may be subjected to the gene editing approaches described herein before reprogramming to an iPS cell, during the reprogramming procedure, or after the donor cell has been reprogrammed to an iPS cell.

NK cells derived from iPS cells are also referred to herein as iNK cells. In some embodiments, the present disclosure provides compositions, methods, and strategies for generating iNK cells that have been derived from developmentally mature cells, also referred to as somatic cells, such as, for example, fibroblasts or peripheral blood cells.

In some embodiments, the present disclosure provides compositions, methods, and strategies for generating iNK cells that have been derived from developmentally mature T cells (T cells that have undergone thymic selection). One hallmark of developmentally mature T cells is a rearranged T cell receptor locus. During T cell maturation, the TCR locus undergoes V(D)J rearrangements to generate complete V-domain exons. These rearrangements are retained throughout reprogramming of a T cells to an induced pluripotent stem (iPS) cell, and throughout differentiation of the resulting iPS cell to a somatic cell.

One advantage of using T cells for the generation of iPS cells is that T cells can be edited with relative ease, e.g., by CRISPR-based methods or other gene-editing methods.

Another advantage of using T cells for the generation of iPS cells is that the rearranged TCR locus allows for genetic tracking of individual cells and their daughter cells. If the reprogramming, expansion, culture, and/or differentiation strategies involved in the generation of NK cells a clonal expansion of a single cell, the rearranged TCR locus can be used as a genetic marker unambiguously identifying a cell and its daughter cells. This, in turn, allows for the characterization of a cell population as truly clonal, or for the identification of mixed populations, or contaminating cells in a clonal population.

A third advantage of using T cells in generating iNK cells carrying multiple edits is that certain karyotypic aberrations associated with chromosomal translocations are selected against in T cell culture. Such aberrations pose a concern when editing cells by CRISPR technology, and in particular when generating cells carrying multiple edits.

A fourth advantage of using T cell derived iPS cells as a starting point for the derivation of therapeutic lymphocytes is that it allows for the expression of a pre-screened TCR in the lymphocytes, e.g., via selecting the T cells for binding activity against a specific antigen, e.g., a tumor antigen, reprogramming the selected T cells to iPS cells, and then deriving lymphocytes from these iPS cells that express the TCR (e.g., T cells). This strategy would also allow for activating the TCR in other cell types, e.g., by genetic or epigenetic strategies.

A fifth advantage of using T cell derived iPS cells as a starting point for iNK differentiation is that the T cells retain at least part of their “epigenetic memory” throughout the reprogramming process, and thus subsequent differentiation of the same or a closely related cell type, such as iNK cells will be more efficient and/or result in higher quality cell populations as compared to approaches using non-related cells, such as fibroblasts, as a starting point for iNK derivation.

### Definitions and Abbreviations

Unless otherwise specified, each of the following terms have the meaning set forth in this section.

The indefinite articles “a” and “an” refer to at least one of the associated noun, and are used interchangeably with the terms “at least one” and “one or more.”

The conjunctions “or” and “and/or” are used interchangeably as non-exclusive disjunctions.

“Subject” means a human or non-human animal. A human subject can be any age (e.g., an infant, child, young adult, or adult), and may suffer from a disease, or may be in need of alteration of a gene or a combination of specific genes. Alternatively, the subject may be an animal, which term includes, but is not limited to, a mammal, and, more particularly, a non-human primate, a rodent (e.g., a mouse, rat, hamster, etc.), a rabbit, a guinea pig, a dog, a cat, and so on. In certain embodiments of this disclosure, the subject is livestock, e.g., a cow, a horse, a sheep, or a goat. In certain embodiments, the subject is poultry.

The terms “treatment,” “treat,” and “treating,” refer to a clinical intervention aimed to reverse, alleviate, delay the onset of, or inhibit the progress, and/or prevent or delay the recurrence of a disease or disorder, or one or more symptoms thereof, as described herein. Treatment, e.g., in the form of a modified NK cell or a population of modified NK cells as described herein, may be administered to a subject after one or more symptoms have developed and/or after a disease has been diagnosed. Treatment may be administered in the absence of symptoms, e.g., to prevent or delay onset of a symptom or inhibit onset or progression of a disease. For example, treatment may be administered to a susceptible individual prior to the onset of symptoms (e.g., in light of genetic or other susceptibility factors). Treatment may also be continued after symptoms have resolved, for example to prevent or delay their recurrence.

“Prevent,” “preventing,” and “prevention” refer to the prevention of a disease in a mammal, e.g., in a human, including (a) avoiding or precluding the disease; (b) affecting the predisposition toward the disease; or (c) preventing or delaying the onset of at least one symptom of the disease.

The terms “polynucleotide”, “nucleotide sequence”, “nucleic acid”, “nucleic acid molecule”, “nucleic acid sequence”, and “oligonucleotide” refer to a series of nucleotide bases (also called “nucleotides”) in DNA and RNA, and mean any chain of two or more nucleotides. The polynucleotides, nucleotide sequences, nucleic acids etc. can be chimeric mixtures or derivatives or modified versions thereof, single-stranded or double-stranded. They can be modified at the base moiety, sugar moiety, or phosphate backbone, for example, to improve stability of the molecule, its hybridization parameters, etc. A nucleotide sequence typically carries genetic information, including, but not limited to, the information used by cellular machinery to make proteins and enzymes. These terms include double- or single-stranded genomic DNA, RNA, any synthetic and genetically manipulated polynucleotide, and both sense and antisense polynucleotides. These terms also include nucleic acids containing modified bases.

Conventional IUPAC notation is used in nucleotide sequences presented herein, as shown in Table 1, below (see also Cornish-Bowden A, Nucleic Acids Res. 1985 May 10; 13(9):3021-30, incorporated by reference herein). It should be noted, however, that “T” denotes “Thymine or Uracil” in those instances where a sequence may be encoded by either DNA or RNA, for example in gRNA targeting domains.

TABLE 1
IUPAC nucleic acid notation

| Character | Base |
|---|---|
| A | Adenine |
| T | Thymine or Uracil |
| G | Guanine |
| C | Cytosine |
| U | Uracil |
| K | G or T/U |
| M | A or C |
| R | A or G |
| Y | C or T/U |
| S | C or G |
| W | A or T/U |
| B | C, G or T/U |
| V | A, C or G |
| H | A, C or T/U |
| D | A, G or T/U |
| N | A, C, G or T/U |

The terms “protein,” “peptide” and “polypeptide” are used interchangeably to refer to a sequential chain of amino acids linked together via peptide bonds. The terms include individual proteins, groups or complexes of proteins that associate together, as well as fragments or portions, variants, derivatives and analogs of such proteins. Peptide sequences are presented herein using conventional notation, beginning with the amino or N-terminus on the left, and proceeding to the carboxyl or C-terminus on the right. Standard one-letter or three-letter abbreviations can be used.

The term “variant” refers to an entity such as a polypeptide, polynucleotide or small molecule that shows significant structural identity with a reference entity but differs structurally from the reference entity in the presence or level of one or more chemical moieties as compared with the reference entity. In many embodiments, a variant also differs functionally from its reference entity. In general, whether a particular entity is properly considered to be a “variant” of a reference entity is based on its degree of structural identity with the reference entity.

The term “endogenous,” as used herein in the context of nucleic acids (e.g., genes, protein-encoding genomic regions, promoters), refers to a native nucleic acid or protein in its natural location, e.g., within the genome of a cell. In contrast, the term “exogenous,” as used herein in the context of nucleic acids, e.g., expression constructs, cDNAs, indels, and nucleic acid vectors, refers to nucleic acids that have artificially been introduced into the genome of a cell using, for example, gene-editing or genetic engineering techniques, e.g., CRISPR-based editing techniques.

The terms “RNA-guided nuclease” and “RNA-guided nuclease molecule” are used interexchangably herein. In some embodiments, the RNA-guided nuclease is a RNA-guided DNA endonuclease enzyme. In some embodiments, the RNA-guided nuclease is a CRISPR nuclease. Non-limiting examples of RNA-guided nucleases are listed in Table 2 below, and the methods and compositions disclosed herein can use any combination of RNA-guided nucleases disclosed herein, or known to those of ordinary skill in the art. Those of ordinary skill in the art will be aware of additional nucleases and nuclease variants suitable for use in the context of the present disclosure, and it will be understood that the present disclosure is not limited in this respect.

TABLE 2
RNA-Guided Nucleases

| | Length | | |
|---|---|---|---|
| Nuclease | (a.a.) | PAM | Reference |
| SpCas9 | 1368 | NGG | Cong et al., Science. 2013;339(6121):819-23 |
| SaCas9 | 1053 | NNGRRT | Ran et al., Nature. 2015;520(7546):186-91. |
| (KKH) | 1067 | NNNRRT | Kleinstiver et al., Nat Biotechnol. |
| SaCas9 | | | 2015;33(12):1293-1298 |
| AsCpf1 | 1353 | TTTV | Zetsche et al., Nat Biotechnol. 2017;35(1):31-34. |
| (AsCas12a) | | | |
| LbCpf1 | 1274 | TTTV | Zetsche et al., Cell. 2015;163(3):759-71. |
| (LbCas12a) | | | |
| CasX | 980 | TTC | Burstein et al., Nature. 2017;542(7640):237-241. |
| CasY | 1200 | TA | Burstein et al., Nature. 2017;542(7640):237-241. |
| Cas12h1 | 870 | RTR | Yan et al., Science. 2019;363(6422):88-91. |
| Cas12i1 | 1093 | TTN | Yan et al., Science. 2019;363(6422):88-91. |
| Cas12c1 | unknown | TG | Yan et al., Science. 2019;363(6422):88-91. |
| Cas12c2 | unknown | TN | Yan et al., Science. 2019;363(6422):88-91. |
| eSpCas9 | 1423 | NGG | Chen et al., Nature. 2017;550(7676):407-410. |
| Cas9-HF1 | 1367 | NGG | Chen et al., Nature. 2017;550(7676):407-410. |
| HypaCas9 | 1404 | NGG | Chen et al., Nature. 2017;550(7676):407-410. |
| dCas9-Fok1 | 1623 | NGG | U.S. Pat. No. 9,322,037 |
| Sniper-Cas9 | 1389 | NGG | Lee et al., Nat Commun. 2018;9(1):3048. |
| xCas9 | 1786 | NGG, NG, | Wang et al., Plant Biotechnol J. 2018; pbi.13053. |
| | | GAA, | |
| | | GAT | |
| AaCas12b | 1129 | TTN | Teng et al. Cell Discov. 2018;4:63. |
| evoCas9 | 1423 | NGG | Casini et al., Nat Biotechnol. 2018;36(3):265-271. |
| SpCas9-NG | 1423 | NG | Nishimasu et al., Science. 2018;361(6408):1259- |
| | | | 1262. |
| VRQR | 1368 | NGA | Li et al., The CRISPR Journal, 2018; 01:01 |
| VRER | 1372 | NGCG | Kleinstiver et al., Nature. 2016;529(7587):490-5. |
| NmeCas9 | 1082 | NNNNGA | Amrani et al., Genome Biol. 2018;19(1):214. |
| | | TT | |
| CjCas9 | 984 | NNNNRY | Kim et al., Nat Commun. 2017;8:14500. |
| | | AC | |
| BhCas12b | 1108 | ATTN | Strecker et al., Nat Commun. 2019 Jan. |
| | | | 22;10(1):212. |
| BhCas12b | 1108 | ATTN | Strecker et al., Nat Commun. 2019 Jan. |
| V4 | | | 22;10(1):212. |

Additional suitable RNA-guided nucleases, e.g., Cas9 and Cas12 nucleases, will be apparent to the skilled artisan in view of the present disclosure, and the disclosure is not limited by the exemplary suitable nucleases provided herein. In some embodiment, a suitable nuclease is a Cas9 or Cpf1 (Cas12a) nuclease. In some embodiments, the disclosure also embraces nuclease variants, e.g., Cas9 or Cpf1 nuclease variants. A nuclease variant refers to a nuclease comprising an amino acid sequence characterized by one or more amino acid substitutions, deletions, or additions as compared to the wild type amino acid sequence of the nuclease. Suitable nucleases and nuclease variants may also include purification tags (e.g., polyhistidine tags) and signaling peptides, e.g., comprising or consisting of a nuclear localization signal sequence. Some non-limiting examples of suitable nucleases and nuclease variants are described in more detail elsewhere herein, and also include those described in PCT application PCT/US2019/22374, filed Mar. 14, 2019, and entitled “Systems and Methods for the Treatment of Hemoglobinopathies,” the entire contents of which are incorporated herein by reference.

In some embodiments, the RNA-guided nuclease is an Acidaminococcus sp. Cpf1 variant (AsCpf1 variant). Suitable Cpf1 nuclease variants, including suitable AsCpf1 variants will be known or apparent to those of ordinary skill in the art based on the present disclosure, and include, but are not limited to, the Cpf1 variants disclosed herein or otherwise known in the art. For example, in some embodiments, the RNA-guided nuclease is a Acidaminococcus sp. Cpf1 RR variant (AsCpf1-RR). In another embodiment, the RNA-guided nuclease is a Cpf1 RVR variant. For example, suitable Cpf1 variants include those having an M537R substitution, an H800A substitution, and/or an F870L substitution, or any combination thereof (numbering scheme according to AsCpf1 wild-type sequence).

The term “hematopoietic stem cell,” or “definitive hematopoietic stem cell” as used herein, refers to CD34+ stem cells capable of giving rise to both mature myeloid and lymphoid cell types including T cells, natural killer cells and B cells.

As used herein, the terms “reprogramming” or “dedifferentiation” or “increasing cell potency” or “increasing developmental potency” refers to a method of increasing the potency of a cell or dedifferentiating the cell to a less differentiated state. For example, a cell that has an increased cell potency has more developmental plasticity (i.e., can differentiate into more cell types) compared to the same cell in the non-reprogrammed state. In other words, a reprogrammed cell is one that is in a less differentiated state than the same cell in a non-reprogrammed state. In some embodiments, the term “reprogramming” refers to de-differentiating a somatic cell, or a multipotent stem cell, into a pluripotent stem cell, also referred to as an induced pluripotent stem cell, or iPS cell. Suitable methods for the generation of iPS cells from somatic or multipotent stem cells are well known to those of skill in the art.

As used herein, the term “differentiation” is the process by which an unspecialized (“uncommitted”) or less specialized cell acquires the features of a specialized cell such as, for example, a blood cell or a muscle cell. A differentiated or differentiation-induced cell is one that has taken on a more specialized (“committed”) position within the lineage of a cell. For example, an iPS cell can be differentiated into various more differentiated cell types, for example, a neural or a hematopoietic stem cell, a lymphocyte, a cardiomyocyte, and other cell types, upon treatment with suitable differentiation factors in the cell culture medium. Suitable methods, differentiation factors, and cell culture media for the differentiation of pluri- and multipotent cell types into more differentiated cell types are well known to those of skill in the art. The term “committed”, when applied to the process of differentiation, refers to a cell that has proceeded in the differentiation pathway to a point where, under normal circumstances, it will continue to differentiate into a specific cell type or subset of cell types, and cannot, under normal circumstances, differentiate into a different cell type or revert to a less differentiated cell type.

As used herein, the terms “differentiation marker,” “differentiation marker gene,” or “differentiation gene,” refers to genes or proteins whose expression are indicative of cell differentiation occurring within a cell, such as a pluripotent cell. Differentiation marker genes include, but are not limited to, the following genes: CD34, CD4, CD8, CD3, CD56 (NCAM), CD49, CD45; NK cell receptor (cluster of differentiation 16 (CD16)), natural killer group-2 member D (NKG2D), CD69, NKp30, NKp44, NKp46, CD158b, FOXA2, FGF5, SOX17, XIST, NODAL, COL3A1, OTX2, DUSP6, EOMES, NR2F2, NROB1, CXCR4, CYP2B6, GAT A3, GATA4, ERBB4, GATA6, HOXC6, INHA, SMAD6, RORA, NIPBL, TNFSF11, CDH11, ZIC4, GAL, SOX3, PITX2, APOA2, CXCL5, CER1, FOXQ1, MLL5, DPP10, GSC, PCDH10, CTCFL, PCDH20, TSHZ1, MEGF10, MYC, DKK1, BMP2, LEFTY2, HES1, CDX2, GNAS, EGR1, COL3A1, TCF4, HEPH, KDR, TOX, FOXA1, LCK, PCDH7, CD1D FOXG1, LEFTY1, TUJ1, T gene (Brachyury), ZIC1, GATA1, GATA2, HDAC4, HDAC5, HDAC7, HDAC9, NOTCH1, NOTCH2, NOTCH4, PAX5, RBPJ, RUNX1, STAT1 and STATS.

As used herein, the term “differentiation marker gene profile,” or “differentiation gene profile,” “differentiation gene expression profile,” “differentiation gene expression signature,” “differentiation gene expression panel,” “differentiation gene panel,” or “differentiation gene signature” refers to the expression or levels of expression of a plurality of differentiation marker genes.

As used herein in the context of cellular developmental potential, the term “potency” or “developmental potency” refers to the sum of all developmental options accessible to the cell (i.e., the developmental potency). The continuum of cell potency includes, but is not limited to, totipotent cells, pluripotent cells, multipotent cells, oligopotent cells, unipotent cells, and terminally differentiated cells.

As used herein, the term “pluripotent” refers to the ability of a cell to form all lineages of the body or soma (i.e., the embryo proper). For example, embryonic stem cells are a type of pluripotent stem cells that are able to form cells from each of the three germs layers, the ectoderm, the mesoderm, and the endoderm. Pluripotency is a continuum of developmental potencies ranging from the incompletely or partially pluripotent cell (e.g., an epiblast stem cell or EpiSC), which is unable to give rise to a complete organism to the more primitive, more pluripotent cell, which is able to give rise to a complete organism (e.g., an embryonic stem cell or an induced pluripotent stem cell).

As used herein, the term “induced pluripotent stem cell” or, iPS cell refers to a stem cell obtained from a differentiated somatic, e.g., adult, neonatal, or fetal cell by a process referred to as reprogramming into cells capable of differentiating into tissues of all three germ or dermal layers: mesoderm, endoderm, and ectoderm. IPS cells are not found in nature.

As used herein, the term “embryonic stem cell” refers to pluripotent stem cells derived from the inner cell mass of the embryonic blastocyst. Embryonic stem cells are pluripotent and give rise during development to all derivatives of the three primary germ layers: ectoderm, endoderm and mesoderm. They do not contribute to the extra-embryonic membranes or the placenta, i.e., are not totipotent.

As used herein, the term “multipotent stem cell” refers to a cell that has the developmental potential to differentiate into cells of one or more germ layers (ectoderm, mesoderm and endoderm), but not all three. Thus, a multipotent cell can also be termed a “partially differentiated cell.” Multipotent cells are well known in the art, and examples of multipotent cells include adult stem cells, such as for example, hematopoietic stem cells and neural stem cells. “Multipotent” indicates that a cell may form many types of cells in a given lineage, but not cells of other lineages. For example, a multipotent hematopoietic cell can form the many different types of blood cells (red, white, platelets, etc.), but it cannot form neurons. Accordingly, the term “multipotency” refers to a state of a cell with a degree of developmental potential that is less than totipotent and pluripotent.

Pluripotency can be determined, in part, by assessing pluripotency characteristics of the cells. Pluripotency characteristics include, but are not limited to: (i) pluripotent stem cell morphology; (ii) the potential for unlimited self-renewal; (iii) expression of pluripotent stem cell markers including, but not limited to SSEA1 (mouse only), SSEA3/4, SSEA5, TRA1-60/81, TRA1-85, TRA2-54, GCTM-2, TG343, TG30, CD9, CD29, CD133/prominin, CD140a, CD56, CD73, CD90, CD105, OCT4, NANOG, SOX2, CD30 and/or CD50; (tv) ability to differentiate to all three somatic lineages (ectoderm, mesoderm and endoderm); (v) teratoma formation consisting of the three somatic lineages; and (vi) formation of embryoid bodies consisting of cells from the three somatic lineages.

As used herein, the term “pluripotent stem cell morphology” refers to the classical morphological features of an embryonic stem cell. Normal embryonic stem cell morphology is characterized by being round and small in shape, with a high nucleus-to-cytoplasm ratio, the notable presence of nucleoli, and typical intercell spacing.

### Genome Editing Systems

The present disclosure relates to the generation of modified NK cells, e.g., NK cells the genome of which has been modified, or that are derived from a multipotent or pluripotent stem cell, e.g., an HSC, ES cell, or iPS cell, the genome of which has been modified. The NK cells and stem cells provided herein can be modified using any gene-editing technology known to those of ordinary skill in the art, including, for example, by using genome editing systems, e.g., CRISPR.

The term “genome editing system” refers to any system having RNA-guided DNA editing activity. Genome editing systems of the present disclosure include at least two components adapted from naturally occurring CRISPR systems: a guide RNA (gRNA) and an RNA-guided nuclease. These two components form a complex that is capable of associating with a specific nucleic acid sequence and editing the DNA in or around that nucleic acid sequence, for instance by making one or more of a single-strand break (an SSB or nick), a double-strand break (a DSB) and/or a point mutation.

Naturally occurring CRISPR systems are organized evolutionarily into two classes and five types (Makarova et al. Nat Rev Microbiol. 2011 June; 9(6): 467-477 (Makarova), incorporated by reference herein), and while genome editing systems of the present disclosure may adapt components of any type or class of naturally occurring CRISPR system, the embodiments presented herein are generally adapted from Class 2, and type II or V CRISPR systems. Class 2 systems, which encompass types II and V, are characterized by relatively large, multidomain RNA-guided nuclease proteins (e.g., Cas9 or Cpf1) and one or more guide RNAs (e.g., a crRNA and, optionally, a tracrRNA) that form ribonucleoprotein (RNP) complexes that associate with (i.e. target) and cleave specific loci complementary to a targeting (or spacer) sequence of the crRNA. Genome editing systems according to the present disclosure similarly target and edit cellular DNA sequences, but differ significantly from CRISPR systems occurring in nature. For example, the unimolecular guide RNAs described herein do not occur in nature, and both guide RNAs and RNA-guided nucleases according to this disclosure may incorporate any number of non-naturally occurring modifications.

Genome editing systems can be implemented (e.g. administered or delivered to a cell or a subject) in a variety of ways, and different implementations may be suitable for distinct applications. For instance, a genome editing system is implemented, in certain embodiments, as a protein/RNA complex (a ribonucleoprotein, or RNP), which can be included in a pharmaceutical composition that optionally includes a pharmaceutically acceptable carrier and/or an encapsulating agent, such as a lipid or polymer micro- or nano-particle, micelle, liposome, etc. In certain embodiments, a genome editing system is implemented as one or more nucleic acids encoding the RNA-guided nuclease and guide RNA components described above (optionally with one or more additional components); in certain embodiments, the genome editing system is implemented as one or more vectors comprising such nucleic acids, for instance a viral vector such as an adeno-associated virus; and in certain embodiments, the genome editing system is implemented as a combination of any of the foregoing. Additional or modified implementations that operate according to the principles set forth herein will be apparent to the skilled artisan and are within the scope of this disclosure.

It should be noted that the genome editing systems of the present disclosure can be targeted to a single specific nucleotide sequence, or may be targeted to—and capable of editing in parallel—two or more specific nucleotide sequences through the use of two or more guide RNAs. The use of multiple gRNAs is referred to as “multiplexing” throughout this disclosure, and can be employed to target multiple, unrelated target sequences of interest, or to form multiple SSBs or DSBs within a single target domain and, in some cases, to generate specific edits within such target domain. For example, International Patent Publication No. WO 2015/138510 by Maeder et al. (Maeder), which is incorporated by reference herein, describes a genome editing system for correcting a point mutation (C.2991+1655A to G) in the human CEP290 gene that results in the creation of a cryptic splice site, which in turn reduces or eliminates the function of the gene. The genome editing system of Maeder utilizes two guide RNAs targeted to sequences on either side of (i.e. flanking) the point mutation, and forms DSBs that flank the mutation. This, in turn, promotes deletion of the intervening sequence, including the mutation, thereby eliminating the cryptic splice site and restoring normal gene function.

As another example, WO 2016/073990 by Cotta-Ramusino, et al. (“Cotta-Ramusino”), incorporated by reference herein, describes a genome editing system that utilizes two gRNAs in combination with a Cas9 nickase (a Cas9 that makes a single strand nick such as S. pyogenes D10A), an arrangement termed a “dual-nickase system.” The dual-nickase system of Cotta-Ramusino is configured to make two nicks on opposite strands of a sequence of interest that are offset by one or more nucleotides, which nicks combine to create a double strand break having an overhang (5′ in the case of Cotta-Ramusino, though 3′ overhangs are also possible). The overhang, in turn, can facilitate homology directed repair events in some circumstances. And, as another example, WO 2015/070083 by Palestrant et al. (“Palestrant”, incorporated by reference herein) describes a gRNA targeted to a nucleotide sequence encoding Cas9 (referred to as a “governing RNA”), which can be included in a genome editing system comprising one or more additional gRNAs to permit transient expression of a Cas9 that might otherwise be constitutively expressed, for example in some virally transduced cells. These multiplexing applications are intended to be exemplary, rather than limiting, and the skilled artisan will appreciate that other applications of multiplexing are generally compatible with the genome editing systems described here.

Genome editing systems can, in some instances, form double strand breaks that are repaired by cellular DNA double-strand break mechanisms such as NHEJ or HDR. These mechanisms are described throughout the literature, for example by Davis & Maizels, PNAS, 111(10):E924-932, Mar. 11, 2014 (Davis) (describing Alt-HDR); Frit et al. DNA Repair 17(2014) 81-97 (Frit) (describing Alt-NHEJ); and Iyama and Wilson III, DNA Repair (Amst.) 2013-August; 12(8): 620-636 (Iyama) (describing canonical HDR and NHEJ pathways generally).

Where genome editing systems operate by forming DSBs, such systems optionally include one or more components that promote or facilitate a particular mode of double-strand break repair or a particular repair outcome. For instance, Cotta-Ramusino also describes genome editing systems in which a single stranded oligonucleotide “donor template” is added; the donor template is incorporated into a target region of cellular DNA that is cleaved by the genome editing system, and can result in a change in the target sequence.

In certain embodiments, genome editing systems modify a target sequence, or modify expression of a gene in or near the target sequence, without causing single- or double-strand breaks. For example, a genome editing system may include an RNA-guided nuclease fused to a functional domain that acts on DNA, thereby modifying the target sequence or its expression. As one example, an RNA-guided nuclease can be connected to (e.g. fused to) a cytidine deaminase functional domain, and may operate by generating targeted C-to-A substitutions. Exemplary nuclease/deaminase fusions are described in Komor et al. Nature 533, 420-424 (19 May 2016) (“Komor”), which is incorporated by reference. Alternatively, a genome editing system may utilize a cleavage-inactivated (i.e. a “dead”) nuclease, such as a dead Cas9 (dCas9), and may operate by forming stable complexes on one or more targeted regions of cellular DNA, thereby interfering with functions involving the targeted region(s) including, without limitation, mRNA transcription, chromatin remodeling, etc.

Guide RNA (gRNA) Molecules

The terms “guide RNA” and “gRNA” refer to any nucleic acid that promotes the specific association (or “targeting”) of an RNA-guided nuclease such as a Cas9 or a Cpf1 to a target sequence such as a genomic or episomal sequence in a cell. gRNAs can be unimolecular (comprising a single RNA molecule, and referred to alternatively as chimeric), or modular (comprising more than one, and typically two, separate RNA molecules, such as a crRNA and a tracrRNA, which are usually associated with one another, for instance by duplexing). gRNAs and their component parts are described throughout the literature, for instance in Briner et al. (Molecular Cell 56(2), 333-339, Oct. 23, 2014 (Briner), which is incorporated by reference), and in Cotta-Ramusino.

In bacteria and archaea, type II CRISPR systems generally comprise an RNA-guided nuclease protein such as Cas9, a CRISPR RNA (crRNA) that includes a 5′ region that is complementary to a foreign sequence, and a trans-activating crRNA (tracrRNA) that includes a 5′ region that is complementary to, and forms a duplex with, a 3′ region of the crRNA. While not intending to be bound by any theory, it is thought that this duplex facilitates the formation of—and is necessary for the activity of—the Cas9/gRNA complex. As type II CRISPR systems were adapted for use in gene editing, it was discovered that the crRNA and tracrRNA could be joined into a single unimolecular or chimeric guide RNA, in one non-limiting example, by means of a four nucleotide (e.g. GAAA) “tetraloop” or “linker” sequence bridging complementary regions of the crRNA (at its 3′ end) and the tracrRNA (at its 5′ end). (Mali et al. Science. 2013 Feb. 15; 339(6121): 823-826 (“Mali”); Jiang et al. Nat Biotechnol. 2013 March; 31(3): 233-239 (“Jiang”); and Jinek et al., 2012 Science August 17; 337(6096): 816-821 (“Jinek”), all of which are incorporated by reference herein.)

Guide RNAs, whether unimolecular or modular, include a “targeting domain” that is fully or partially complementary to a target domain within a target sequence, such as a DNA sequence in the genome of a cell where editing is desired. Targeting domains are referred to by various names in the literature, including without limitation “guide sequences” (Hsu et al., Nat Biotechnol. 2013 September; 31(9): 827-832, (“Hsu”), incorporated by reference herein), “complementarity regions” (Cotta-Ramusino), “spacers” (Briner) and generically as “crRNAs” (Jiang). Irrespective of the names they are given, targeting domains are typically 10-30 nucleotides in length, and in certain embodiments are 16-24 nucleotides in length (for instance, 16, 17, 18, 19, 20, 21, 22, 23 or 24 nucleotides in length), and are at or near the 5′ terminus of in the case of a Cas9 gRNA, and at or near the 3′ terminus in the case of a Cpf1 gRNA.

In addition to the targeting domains, gRNAs typically (but not necessarily, e.g., as discussed below) include a plurality of domains that may influence the formation or activity of gRNA/Cas9 complexes. For instance, as mentioned above, the duplexed structure formed by first and secondary complementarity domains of a gRNA (also referred to as a repeat:anti-repeat duplex) interacts with the recognition (REC) lobe of Cas9 and can mediate the formation of Cas9/gRNA complexes. (Nishimasu et al., Cell 156, 935-949, Feb. 27, 2014 (Nishimasu 2014) and Nishimasu et al., Cell 162, 1113-1126, Aug. 27, 2015 (Nishimasu 2015), both incorporated by reference herein). It should be noted that the first and/or second complementarity domains may contain one or more poly-A tracts, which can be recognized by RNA polymerases as a termination signal. The sequence of the first and second complementarity domains are, therefore, optionally modified to eliminate these tracts and promote the complete in vitro transcription of gRNAs, for instance through the use of A-G swaps as described in Briner, or A-U swaps. These and other similar modifications to the first and second complementarity domains are within the scope of the present disclosure.

Along with the first and second complementarity domains, Cas9 gRNAs typically include two or more additional duplexed regions that are involved in nuclease activity in vivo but not necessarily in vitro. (Nishimasu 2015). A first stem-loop one near the 3′ portion of the second complementarity domain is referred to variously as the “proximal domain,” (Cotta-Ramusino) “stem loop 1” (Nishimasu 2014 and 2015) and the “nexus” (Briner). One or more additional stem loop structures are generally present near the 3′ end of the gRNA, with the number varying by species: S. pyogenes gRNAs typically include two 3′ stem loops (for a total of four stem loop structures including the repeat:anti-repeat duplex), while S. aureus and other species have only one (for a total of three stem loop structures). A description of conserved stem loop structures (and gRNA structures more generally) organized by species is provided in Briner.

While the foregoing description has focused on gRNAs for use with Cas9, it should be appreciated that other RNA-guided nucleases have been (or may in the future be) discovered or invented which utilize gRNAs that differ in some ways from those described to this point. For instance, Cpf1 (“CRISPR from Prevotella and Franciscella 1”) is a recently discovered RNA-guided nuclease that does not require a tracrRNA to function. (Zetsche et al., 2015, Cell 163, 759-771 Oct. 22, 2015 (Zetsche I), incorporated by reference herein). A gRNA for use in a Cpf1 genome editing system generally includes a targeting domain and a complementarity domain (alternately referred to as a “handle”). It should also be noted that, in gRNAs for use with Cpf1, the targeting domain is usually present at or near the 3′ end, rather than the 5′ end as described above in connection with Cas9 gRNAs (the handle is at or near the 5′ end of a Cpf1 gRNA).

Those of skill in the art will appreciate that, although structural differences may exist between gRNAs from different prokaryotic species, or between Cpf1 and Cas9 gRNAs, the principles by which gRNAs operate are generally consistent. Because of this consistency of operation, gRNAs can be defined, in broad terms, by their targeting domain sequences, and skilled artisans will appreciate that a given targeting domain sequence can be incorporated in any suitable gRNA, including a unimolecular or chimeric gRNA, or a gRNA that includes one or more chemical modifications and/or sequential modifications (substitutions, additional nucleotides, truncations, etc.). Thus, for economy of presentation in this disclosure, gRNAs may be described solely in terms of their targeting domain sequences.

More generally, skilled artisans will appreciate that some aspects of the present disclosure relate to systems, methods and compositions that can be implemented using multiple RNA-guided nucleases. For this reason, unless otherwise specified, the term gRNA should be understood to encompass any suitable gRNA that can be used with any RNA-guided nuclease, and not only those gRNAs that are compatible with a particular species of Cas9 or Cpf1. By way of illustration, the term gRNA can, in certain embodiments, include a gRNA for use with any RNA-guided nuclease occurring in a Class 2 CRISPR system, such as a type II or type V or CRISPR system, or an RNA-guided nuclease derived or adapted therefrom.

In some embodiments, the guide RNA used comprises a modification as compared to the standard gRNA scaffold. Such modifications may comprise, for example, chemical modifications of a part of the gRNA, e.g., of a nucleobase or backbone moiety. In some embodiments, such a modification may also include the presence of a DNA nucleotide within the gRNA, e.g., within or outside of the targeting domain. In some embodiments, the modification may include an extension of the gRNA scaffold, e.g., by addition of 1-100 nucleotides, including RNA and/or DNA nucleotides at the 3′ or the 5′ terminus of the guide RNA, e.g., at the terminus distal to the targeting domain.

Generally, gRNAs include the sugar group ribose, which is a 5-membered ring having an oxygen. Exemplary modified gRNAs can include, without limitation, replacement of the oxygen in ribose (e.g., with sulfur (S), selenium (Se), or alkylene, such as, e.g., methylene or ethylene); addition of a double bond (e.g., to replace ribose with cyclopentenyl or cyclohexenyl); ring contraction of ribose (e.g., to form a 4-membered ring of cyclobutane or oxetane); ring expansion of ribose (e.g., to form a 6- or 7-membered ring having an additional carbon or heteroatom, such as for example, anhydrohexitol, altritol, mannitol, cyclohexanyl, cyclohexenyl, and morpholino that also has a phosphoramidate backbone). Although the majority of sugar analog alterations are localized to the 2′ position, other sites are amenable to modification, including the 4′ position. In certain embodiments, a gRNA comprises a 4′-S, 4′-Se or a 4′-C-aminomethyl-2′-O-Me modification.

In certain embodiments, deaza nucleotides, e.g., 7-deaza-adenosine, can be incorporated into the gRNA. In certain embodiments, 0- and N-alkylated nucleotides, e.g., N6-methyl adenosine, can be incorporated into the gRNA. In certain embodiments, one or more or all of the nucleotides in a gRNA are deoxynucleotides.

In certain embodiments, gRNAs as used herein may be modified or unmodified gRNAs. In certain embodiments, a gRNA may include one or more modifications. In certain embodiments, the one or more modifications may include a phosphorothioate linkage modification, a phosphorodithioate (PS2) linkage modification, a 2′-O-methyl modification, or combinations thereof. In certain embodiments, the one or more modifications may be at the 5′ end of the gRNA, at the 3′ end of the gRNA, or combinations thereof.

In certain embodiments, a gRNA modification may comprise one or more phosphorodithioate (PS2) linkage modifications.

In some embodiments, a gRNA used herein includes one or more or a stretch of deoxyribonucleic acid (DNA) bases, also referred to herein as a “DNA extension.” In some embodiments, a gRNA used herein includes a DNA extension at the 5′ end of the gRNA, the 3′ end of the gRNA, or a combination thereof. In certain embodiments, the DNA extension may be 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 DNA bases long. For example, in certain embodiments, the DNA extension may be 1, 2, 3, 4, 5, 10, 15, 20, or 25 DNA bases long. In certain embodiments, the DNA extension may include one or more DNA bases selected from adenine (A), guanine (G), cytosine (C), or thymine (T). In certain embodiments, the DNA extension includes the same DNA bases. For example, the DNA. extension may include a stretch of adenine (A) bases. In certain embodiments, the DNA extension may include a stretch of thymine (T) bases. In certain embodiments, the DNA extension includes a combination of different DNA bases. In certain embodiments, a DNA extension may comprise a sequence set forth in Table 3. In certain embodiments, a gRNA used herein includes a DNA extension as well as one or more phosphorothioate linkage modifications, one or more phosphorodithioate (PS2) linkage modifications, one or more 2′-O-methyl modifications, or combinations thereof. In certain embodiments, the one or more modifications may be at the 5′ end of the gRNA, at the 3′ end of the gRNA, or combinations thereof. In certain embodiments, a gRNA including a DNA extension may comprise a sequence set forth in Table 3 that includes a DNA extension. Without wishing to be bound by theory, it is contemplated that any DNA extension may be used herein, so long as it does not hybridize to the target nucleic acid being targeted by the gRNA and it also exhibits an increase in editing at the target nucleic acid site relative to a gRNA which does not include such a DNA extension.

In some embodiments, a gRNA used herein includes one or more or a stretch of ribonucleic acid (RNA) bases, also referred to herein as an “RNA extension.” In some embodiments, a gRNA used herein includes an RNA extension at the 5′ end of the gRNA, the 3′ end of the gRNA, or a combination thereof. In certain embodiments, the RNA extension may be 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 RNA bases long. For example, in certain embodiments, the RNA extension may be 1, 2, 3, 4, 5, 10, 15, 20, or 25 RNA bases long. In certain embodiments, the RNA extension may include one or more RNA bases selected from adenine (rA), guanine (rG), cytosine (rC), or uracil (rU), in which the “r” represents RNA, 2′-hydroxy. In certain embodiments, the RNA extension includes the same RNA bases. For example, the RNA extension may include a stretch of adenine (rA) bases. In certain embodiments, the RNA extension includes a combination of different RNA bases. In certain embodiments, an RNA extension may comprise a sequence set forth in Table 3. In certain embodiments, a gRNA used herein includes an RNA extension as well as one or more phosphorothioate linkage modifications, one or more phosphorodithioate (PS2) linkage modifications, one or more 2′-O-methyl modifications, or combinations thereof. In certain embodiments, the one or more modifications may be at the 5′ end of the gRNA, at the 3′ end of the gRNA, or combinations thereof. In certain embodiments, a gRNA including a RNA extension may comprise a sequence set forth in Table 3 that includes an RNA extension. gRNAs including an RNA extension at the 5′ end of the gRNA may comprise a sequence disclosed herein. gRNAs including an RNA extension at the 3′ end of the gRNA may comprise a sequence disclosed herein.

It is contemplated that gRNAs used herein may also include an RNA extension and a DNA extension. In certain embodiments, the RNA extension and DNA extension may both be at the 5′ end of the gRNA, the 3′ end of the gRNA, or a combination thereof. In certain embodiments, the RNA extension is at the 5′ end of the gRNA and the DNA extension is at the 3′ end of the gRNA. In certain embodiments, the RNA extension is at the 3′ end of the gRNA and the DNA extension is at the 5′ end of the gRNA.

In some embodiments, a gRNA which includes a modification, e.g., a DNA extension at the 5′ end, is complexed with a RNA-guided nuclease, e.g., an AsCpf1 nuclease, to form an RNP, which is then employed to edit a target cell, e.g., an NK cell.

Exemplary suitable 5′ extensions for Cpf1 guide RNAs are provided in the table below:

TABLE 3
gRNA 5′ Extensions

| 5′ extension | | |
|---|---|---|
| Sequence ID | | 5′ |
| No: | 5′ extension sequence | modification |
| | rCrUrUrUrU | +5 RNA |
| | rArArGrArCrCrUrUrUrU | +10 RNA |
| | rArUrGrUrGrUrUrUrUrUrGrUrCrArArArArGrArCrCrUrUrUrU | +25 RNA |
| | rArGrGrCrCrArGrCrUrUrGrCrCrGrGrUrUrUrUrUrUrArGrUrCrG | |
| | rUrGrCrUrGrCrUrUrCrArUrGrUrGrUrUrUrUrUrGrUrCrArArAr | +60 RNA |
| | ArGrArCrCrUrUrUrU | |
| | CTTTT | +5 DNA |
| | AAGACCTTTT | +10 DNA |
| | ATGTGTTTTTGTCAAAAGACCTTTT | +25 DNA |
| | AGGCCAGCTTGCCGGTTTTTTAGTCGTGCTGCTTCATGTG | |
| | TTTTTGTCAAAAGACCTTTT | +60 DNA |
| | TTTTTGTCAAAAGACCTTTT | +20 DNA |
| | GCTTCATGTGTTTTTGTCAAAAGACCTTTT | +30 DNA |
| | GCCGGTTTTTTAGTCGTGCTGCTTCATGTGTTTTTGTCAAA | |
| | AGACCTTTT | +50 DNA |
| | TAGTCGTGCTGCTTCATGTGTTTTTGTCAAAAGACCTTTT | +40 DNA |
| | C*C*GAAGTTTTCTTCGGTTTT | +20 DNA + 2xPS |
| | T*T*TTTCCGAAGTTTTCTTCGGTTTT | +25 DNA + 2xPS |
| | A*A*CGCTTTTTCCGAAGTTTTCTTCGGTTTT | +30 DNA + 2xPS |
| | G*C*GTTGTTTTCAACGCTTTTTCCGAAGTTTTCTTCGGTT | |
| | TT | +41 DNA + 2xPS |
| | G*G*CTTCTTTTGAAGCCTTTTTGCGTTGTTTTCAACGCTT | |
| | TTTCCGAAGTTTTCTTCGGTTTT | +62 DNA + 2xPS |
| | A*T*GTGTTTTTGTCAAAAGACCTTTT | +25 DNA + 2xPS |
| | AAAAAAAAAAAAAAAAAAAAAAAAA | +25 A |
| | TTTTTTTTTTTTTTTTTTTTTTTTT | +25 T |
| | mA*mU*rGrUrGrUrUrUrUrUrGrUrCrArArArArGrArCrCrUrUrU | +25 RNA + 2xPS |
| | rU | |
| | mA*mA*rArArArArArArArArArArArArArArArArArArArArAr | PolyA RNA + |
| | ArA | 2xPS |
| | mU*mU*rUrUrUrUrUrUrUrUrUrUrUrUrUrUrUrUrUrUrUrUrUr | PolyU RNA + |
| | UrU | 2xPS |

All bases are in upper case
Lowercase “r” represents RNA, 2′-hydroxy; bases not modified by an “r” are DNA
All bases are linked via standard phosphodiester bonds except as noted:
“*” represents phosphorothioate modification
“PS” represents phosphorothioate modification

Additional suitable gRNA modifications will be apparent to those of ordinary skill in the art based on the present disclosure. Suitable gRNA modifications include, for example, those described in PCT application PCT/US2018/054027, filed on Oct. 2, 2018, and entitled “MODIFIED CPF1 GUIDE RNA;” in PCT application PCT/US2015/000143, filed on Dec. 3, 2015, and entitled “GUIDE RNA WITH CHEMICAL MODIFICATIONS;” in PCT application PCT/US2016/026028, filed Apr. 5, 2016, and entitled “CHEMICALLY MODIFIED GUIDE RNAS FOR CRISPR/CAS-MEDIATED GENE REGULATION;” and in PCT application PCT/US2016/053344, filed on Sep. 23, 2016, and entitled “NUCLEASE-MEDIATED GENOME EDITING OF PRIMARY CELLS AND ENRICHMENT THEREOF;” the entire contents of each of which are incorporated herein by reference.

gRNA Design

Methods for selection and validation of target sequences as well as off-target analyses have been described previously, e.g., in Mali; Hsu; Fu et al., 2014 Nat biotechnol 32(3): 279-84, Heigwer et al., 2014 Nat methods 11(2):122-3; Bae et al. (2014) Bioinformatics 30(10): 1473-5; and Xiao A et al. (2014) Bioinformatics 30(8): 1180-1182. Each of these references is incorporated by reference herein. As a non-limiting example, gRNA design may involve the use of a software tool to optimize the choice of potential target sequences corresponding to a user's target sequence, e.g., to minimize total off-target activity across the genome. While off-target activity is not limited to cleavage, the cleavage efficiency at each off-target sequence can be predicted, e.g., using an experimentally-derived weighting scheme. These and other guide selection methods are described in detail in Maeder and Cotta-Ramusino.

In certain embodiments, one or more or all of the nucleotides in a gRNA are modified. Strategies for modifying a gRNA are described in WO2019/152519, published Aug. 8, 2019, the entire contents of which are expressly incorporated herein by reference.

Non-limiting examples of guide RNAs suitable for certain embodiments embraced by the present disclosure are provided herein, for example, in the Tables below. Those of ordinary skill in the art will be able to envision suitable guide RNA sequences for a specific nuclease, e.g., a Cas9 or Cpf-1 nuclease, from the disclosure of the targeting domain sequence, either as a DNA or RNA sequence. For example, a guide RNA comprising a targeting sequence consisting of RNA nucleotides would include the RNA sequence corresponding to the targeting domain sequence provided as a DNA sequence, and this contain uracil instead of thymidine nucleotides. For example, a guide RNA comprising a targeting domain sequence consisting of RNA nucleotides, and described by the DNA sequence TCTGCAGAAATGTTCCCCGT (SEQ ID NO: ______) would have a targeting domain of the corresponding RNA sequence UCUGCAGAAAUGUUCCCCGU (SEQ ID NO: ______). As will be apparent to the skilled artisan, such a targeting sequence would be linked to a suitable guide RNA scaffold, e.g., a crRNA scaffold sequence or a chimeric crRNA/tracerRNA scaffold sequence. Suitable gRNA scaffold sequences are known to those of ordinary skill in the art. For AsCpf1, for example, a suitable scaffold sequence comprises the sequence UAAUUUCUACUCUUGUAGAU (SEQ ID NO: ______), added to the 5′-terminus of the targeting domain. In the example above, this would result in a Cpf1 guide RNA of the sequence UAAUUUCUACUCUUGUAGAUUCUGCAGAAAUGUUCCCCGU (SEQ ID NO: ______). Those of skill in the art would further understand how to modify such a guide RNA, e.g., by adding a DNA extension (e.g., in the example above, adding a 25-mer DNA extension as described herein would result, for example, in a guide RNA of the sequence ATGTGTTTTTGTCAAAAGACCTTTTrUrArArUrUrUrCrUrArCrUrCrUrUrGrUrArGrArU rUrCrUrGrCrArGrArArArUrGrUrUrCrCrCrCrGrU (SEQ ID NO: ______). It will be understood that the exemplary targeting sequences provided herein are not limiting, and additional suitable sequences, e.g., variants of the specific sequences disclosed herein, will be apparent to the skilled artisan based on the present disclosure in view of the general knowledge in the art.

In some embodiments the gRNA for use in the disclosure is a gRNA targeting TIGIT (TIGIT gRNA). In some embodiments, the gRNA targeting TIGIT is one or more of the gRNAs described in Table 4.

TABLE 4
TIGIT gRNAs

| | gRNA Targeting Domain | | |
|---|---|---|---|
| Name | Sequence (DNA) | Length | Enzyme |
| TIGIT4170 | TCTGCAGAAATGTTCCCCGT | 20 | AsCpf1 |
| TIGIT4171 | TGCAGAGAAAGGTGGCTCTA | 20 | AsCpf1 |
| TIGIT4172 | TAATGCTGACTTGGGGTGGC | 20 | AsCpf1 |
| TIGIT4173 | TAGGACCTCCAGGAAGATTC | 20 | AsCpf1 |
| TIGIT4174 | TAGTCAACGCGACCACCACG | 20 | AsCpf1 |
| TIGIT4175 | TCCTGAGGTCACCTTCCACA | 20 | AsCpf1 |
| TIGIT4176 | TATTGTGCCTGTCATCATTC | 20 | AsCpf1 |
| TIGIT4177 | TGACAGGCACAATAGAAACAA | 21 | SauCas9 |
| TIGIT4178 | GACAGGCACAATAGAAACAAC | 21 | SauCas9 |
| TIGIT4179 | AAACAACGGGGAACATTTCTG | 21 | SauCas9 |
| TIGIT4180 | ACAACGGGGAACATTTCTGCA | 21 | SauCas9 |
| TIGIT4181 | TGATAGAGCCACCTTTCTCTG | 21 | SauCas9 |
| TIGIT4182 | GGGTCACTTGTGCCGTGGTGG | 21 | SauCas9 |
| TIGIT4183 | GGCACAAGTGACCCAGGTCAA | 21 | SauCas9 |
| TIGIT4184 | GTCCTGCTGCTCCCAGTTGAC | 21 | SauCas9 |
| TIGIT4185 | TGGCCATTTGTAATGCTGACT | 21 | SauCas9 |
| TIGIT4186 | TGGCACATCTCCCCATCCTTC | 21 | SauCas9 |
| TIGIT4187 | CATCTCCCCATCCTTCAAGGA | 21 | SauCas9 |
| TIGIT4188 | CCACTCGATCCTTGAAGGATG | 21 | SauCas9 |
| TIGIT4189 | GGCCACTCGATCCTTGAAGGA | 21 | SauCas9 |
| TIGIT4190 | CCTGGGGCCACTCGATCCTTG | 21 | SauCas9 |
| TIGIT4191 | GACTGGAGGGTGAGGCCCAGG | 21 | SauCas9 |
| TIGIT4192 | ATCGTTCACGGTCAGCGACTG | 21 | SauCas9 |
| TIGIT4193 | GTCGCTGACCGTGAACGATAC | 21 | SauCas9 |
| TIGIT4194 | CGCTGACCGTGAACGATACAG | 21 | SauCas9 |
| TIGIT4195 | GCATCTATCACACCTACCCTG | 21 | SauCas9 |
| TIGIT4196 | CCTACCCTGATGGGACGTACA | 21 | SauCas9 |
| TIGIT4197 | TACCCTGATGGGACGTACACT | 21 | SauCas9 |
| TIGIT4198 | CCCTGATGGGACGTACACTGG | 21 | SauCas9 |
| TIGIT4199 | TTCTCCCAGTGTACGTCCCAT | 21 | SauCas9 |
| TIGIT4200 | GGAGAATCTTCCTGGAGGTCC | 21 | SauCas9 |
| TIGIT4201 | CATGGCTCCAAGCAATGGAAT | 21 | SauCas9 |
| TIGIT4202 | CGCGGCCATGGCTCCAAGCAA | 21 | SauCas9 |
| TIGIT4203 | TCGCGGCCATGGCTCCAAGCA | 21 | SauCas9 |
| TIGIT4204 | CATCGTGGTGGTCGCGTTGAC | 21 | SauCas9 |
| TIGIT4205 | AAAGCCCTCAGAATCCATTCT | 21 | SauCas9 |
| TIGIT4206 | CATTCTGTGGAAGGTGACCTC | 21 | SauCas9 |
| TIGIT4207 | TTCTGTGGAAGGTGACCTCAG | 21 | SauCas9 |
| TIGIT4208 | CCTGAGGTCACCTTCCACAGA | 21 | SauCas9 |
| TIGIT4209 | TTCTCCTGAGGTCACCTTCCA | 21 | SauCas9 |
| TIGIT4210 | AGGAGAAAATCAGCTGGACAG | 21 | SauCas9 |
| TIGIT4211 | GGAGAAAATCAGCTGGACAGG | 21 | SauCas9 |
| TIGIT4212 | GCCCCAGTGCTCCCTCACCCC | 21 | SauCas9 |
| TIGIT4213 | TGGACACAGCTTCCTGGGGGT | 21 | SauCas9 |
| TIGIT4214 | TCTGCCTGGACACAGCTTCCT | 21 | SauCas9 |
| TIGIT4215 | AGCTGCACCTGCTGGGCTCTG | 21 | SauCas9 |
| TIGIT4216 | GCTGGGCTCTGTGGAGAGCAG | 21 | SauCas9 |
| TIGIT4217 | TGGGCTCTGTGGAGAGCAGCG | 21 | SauCas9 |
| TIGIT4218 | CTGCATGACTACTTCAATGTC | 21 | SauCas9 |
| TIGIT4219 | AATGTCCTGAGTTACAGAAGC | 21 | SauCas9 |
| TIGIT4220 | TGGGTAACTGCAGCTTCTTCA | 21 | SauCas9 |
| TIGIT4221 | GACAGGCACAATAGAAACAA | 20 | SpyCas9 |
| TIGIT4222 | ACAGGCACAATAGAAACAAC | 20 | SpyCas9 |
| TIGIT4223 | CAGGCACAATAGAAACAACG | 20 | SpyCas9 |
| TIGIT4224 | GGGAACATTTCTGCAGAGAA | 20 | SpyCas9 |
| TIGIT4225 | AACATTTCTGCAGAGAAAGG | 20 | SpyCas9 |
| TIGIT4226 | ATGTCACCTCTCCTCCACCA | 20 | SpyCas9 |
| TIGIT4227 | CTTGTGCCGTGGTGGAGGAG | 20 | SpyCas9 |
| TIGIT4228 | GGTCACTTGTGCCGTGGTGG | 20 | SpyCas9 |
| TIGIT4229 | CACCACGGCACAAGTGACCC | 20 | SpyCas9 |
| TIGIT4230 | CTGGGTCACTTGTGCCGTGG | 20 | SpyCas9 |
| TIGIT4231 | GACCTGGGTCACTTGTGCCG | 20 | SpyCas9 |
| TIGIT4232 | CACAAGTGACCCAGGTCAAC | 20 | SpyCas9 |
| TIGIT4233 | ACAAGTGACCCAGGTCAACT | 20 | SpyCas9 |
| TIGIT4234 | CCAGGTCAACTGGGAGCAGC | 20 | SpyCas9 |
| TIGIT4235 | CTGCTGCTCCCAGTTGACCT | 20 | SpyCas9 |
| TIGIT4236 | CCTGCTGCTCCCAGTTGACC | 20 | SpyCas9 |
| TIGIT4237 | GGAGCAGCAGGACCAGCTTC | 20 | SpyCas9 |
| TIGIT4238 | CATTACAAATGGCCAGAAGC | 20 | SpyCas9 |
| TIGIT4239 | GGCCATTTGTAATGCTGACT | 20 | SpyCas9 |
| TIGIT4240 | GCCATTTGTAATGCTGACTT | 20 | SpyCas9 |
| TIGIT4241 | CCATTTGTAATGCTGACTTG | 20 | SpyCas9 |
| TIGIT4242 | TTTGTAATGCTGACTTGGGG | 20 | SpyCas9 |
| TIGIT4243 | CCCCAAGTCAGCATTACAAA | 20 | SpyCas9 |
| TIGIT4244 | GCACATCTCCCCATCCTTCA | 20 | SpyCas9 |
| TIGIT4245 | CCCATCCTTCAAGGATCGAG | 20 | SpyCas9 |
| TIGIT4246 | CACTCGATCCTTGAAGGATG | 20 | SpyCas9 |
| TIGIT4247 | CCACTCGATCCTTGAAGGAT | 20 | SpyCas9 |
| TIGIT4248 | GCCACTCGATCCTTGAAGGA | 20 | SpyCas9 |
| TIGIT4249 | TTCAAGGATCGAGTGGCCCC | 20 | SpyCas9 |
| TIGIT4250 | TGGGGCCACTCGATCCTTGA | 20 | SpyCas9 |
| TIGIT4251 | GATCGAGTGGCCCCAGGTCC | 20 | SpyCas9 |
| TIGIT4252 | AGTGGCCCCAGGTCCCGGCC | 20 | SpyCas9 |
| TIGIT4253 | GTGGCCCCAGGTCCCGGCCT | 20 | SpyCas9 |
| TIGIT4254 | GAGGCCCAGGCCGGGACCTG | 20 | SpyCas9 |
| TIGIT4255 | TGAGGCCCAGGCCGGGACCT | 20 | SpyCas9 |
| TIGIT4256 | GTGAGGCCCAGGCCGGGACC | 20 | SpyCas9 |
| TIGIT4257 | TGGAGGGTGAGGCCCAGGCC | 20 | SpyCas9 |
| TIGIT4258 | CTGGAGGGTGAGGCCCAGGC | 20 | SpyCas9 |
| TIGIT4259 | GCGACTGGAGGGTGAGGCCC | 20 | SpyCas9 |
| TIGIT4260 | CGGTCAGCGACTGGAGGGTG | 20 | SpyCas9 |
| TIGIT4261 | GTTCACGGTCAGCGACTGGA | 20 | SpyCas9 |
| TIGIT4262 | CGTTCACGGTCAGCGACTGG | 20 | SpyCas9 |
| TIGIT4263 | TATCGTTCACGGTCAGCGAC | 20 | SpyCas9 |
| TIGIT4264 | TCGCTGACCGTGAACGATAC | 20 | SpyCas9 |
| TIGIT4265 | CGCTGACCGTGAACGATACA | 20 | SpyCas9 |
| TIGIT4266 | GCTGACCGTGAACGATACAG | 20 | SpyCas9 |
| TIGIT4267 | GTACTCCCCTGTATCGTTCA | 20 | SpyCas9 |
| TIGIT4268 | ATCTATCACACCTACCCTGA | 20 | SpyCas9 |
| TIGIT4269 | TCTATCACACCTACCCTGAT | 20 | SpyCas9 |
| TIGIT4270 | TACCCTGATGGGACGTACAC | 20 | SpyCas9 |
| TIGIT4271 | ACCCTGATGGGACGTACACT | 20 | SpyCas9 |
| TIGIT4272 | AGTGTACGTCCCATCAGGGT | 20 | SpyCas9 |
| TIGIT4273 | TCCCAGTGTACGTCCCATCA | 20 | SpyCas9 |
| TIGIT4274 | CTCCCAGTGTACGTCCCATC | 20 | SpyCas9 |
| TIGIT4275 | GTACACTGGGAGAATCTTCC | 20 | SpyCas9 |
| TIGIT4276 | CACTGGGAGAATCTTCCTGG | 20 | SpyCas9 |
| TIGIT4277 | CTGAGCTTTCTAGGACCTCC | 20 | SpyCas9 |
| TIGIT4278 | AGGTTCCAGATTCCATTGCT | 20 | SpyCas9 |
| TIGIT4279 | AAGCAATGGAATCTGGAACC | 20 | SpyCas9 |
| TIGIT4280 | GATTCCATTGCTTGGAGCCA | 20 | SpyCas9 |
| TIGIT4281 | TGGCTCCAAGCAATGGAATC | 20 | SpyCas9 |
| TIGIT4282 | GCGGCCATGGCTCCAAGCAA | 20 | SpyCas9 |
| TIGIT4283 | TGGAGCCATGGCCGCGACGC | 20 | SpyCas9 |
| TIGIT4284 | AGCCATGGCCGCGACGCTGG | 20 | SpyCas9 |
| TIGIT4285 | GACCACCAGCGTCGCGGCCA | 20 | SpyCas9 |
| TIGIT4286 | GCAGATGACCACCAGCGTCG | 20 | SpyCas9 |
| TIGIT4287 | CATCTGCACAGCAGTCATCG | 20 | SpyCas9 |
| TIGIT4288 | CTGCACAGCAGTCATCGTGG | 20 | SpyCas9 |
| TIGIT4289 | AGCCCTCAGAATCCATTCTG | 20 | SpyCas9 |
| TIGIT4290 | CTCAGAATCCATTCTGTGGA | 20 | SpyCas9 |
| TIGIT4291 | TTCCACAGAATGGATTCTGA | 20 | SpyCas9 |
| TIGIT4292 | CTTCCACAGAATGGATTCTG | 20 | SpyCas9 |
| TIGIT4293 | ATTCTGTGGAAGGTGACCTC | 20 | SpyCas9 |
| TIGIT4294 | TGAGGTCACCTTCCACAGAA | 20 | SpyCas9 |
| TIGIT4295 | GACCTCAGGAGAAAATCAGC | 20 | SpyCas9 |
| TIGIT4296 | CAGGAGAAAATCAGCTGGAC | 20 | SpyCas9 |
| TIGIT4297 | GTCCAGCTGATTTTCTCCTG | 20 | SpyCas9 |
| TIGIT4298 | GAGAAAATCAGCTGGACAGG | 20 | SpyCas9 |
| TIGIT4299 | AATCAGCTGGACAGGAGGAA | 20 | SpyCas9 |
| TIGIT4300 | CCCAGTGCTCCCTCACCCCC | 20 | SpyCas9 |
| TIGIT4301 | CTGGGGGTGAGGGAGCACTG | 20 | SpyCas9 |
| TIGIT4302 | CCTGGGGGTGAGGGAGCACT | 20 | SpyCas9 |
| TIGIT4303 | TCCTGGGGGTGAGGGAGCAC | 20 | SpyCas9 |
| TIGIT4304 | ACACAGCTTCCTGGGGGTGA | 20 | SpyCas9 |
| TIGIT4305 | GACACAGCTTCCTGGGGGTG | 20 | SpyCas9 |
| TIGIT4306 | ACCCCCAGGAAGCTGTGTCC | 20 | SpyCas9 |
| TIGIT4307 | GCCTGGACACAGCTTCCTGG | 20 | SpyCas9 |
| TIGIT4308 | TGCCTGGACACAGCTTCCTG | 20 | SpyCas9 |
| TIGIT4309 | CTGCCTGGACACAGCTTCCT | 20 | SpyCas9 |
| TIGIT4310 | TCTGCCTGGACACAGCTTCC | 20 | SpyCas9 |
| TIGIT4311 | CAGGCAGAAGCTGCACCTGC | 20 | SpyCas9 |
| TIGIT4312 | AGGCAGAAGCTGCACCTGCT | 20 | SpyCas9 |
| TIGIT4313 | CAGCAGGTGCAGCTTCTGCC | 20 | SpyCas9 |
| TIGIT4314 | GCTGCACCTGCTGGGCTCTG | 20 | SpyCas9 |
| TIGIT4315 | TGCTCTCCACAGAGCCCAGC | 20 | SpyCas9 |
| TIGIT4316 | CTGGGCTCTGTGGAGAGCAG | 20 | SpyCas9 |
| TIGIT4317 | TGGGCTCTGTGGAGAGCAGC | 20 | SpyCas9 |
| TIGIT4318 | GGGCTCTGTGGAGAGCAGCG | 20 | SpyCas9 |
| TIGIT4319 | CTGTGGAGAGCAGCGGGGAG | 20 | SpyCas9 |
| TIGIT4320 | ATTGAAGTAGTCATGCAGCT | 20 | SpyCas9 |
| TIGIT4321 | TGTCCTGAGTTACAGAAGCC | 20 | SpyCas9 |
| TIGIT4322 | GTCCTGAGTTACAGAAGCCT | 20 | SpyCas9 |
| TIGIT4323 | TACCCAGGCTTCTGTAACTC | 20 | SpyCas9 |
| TIGIT4324 | TGAAGAAGCTGCAGTTACCC | 20 | SpyCas9 |
| TIGIT4325 | TGCAGCTTCTTCACAGAGAC | 20 | SpyCas9 |
| TIGIT5053 | GTTGTTTCTATTGTGCCTGT | 20 | AsCpf1 RR |
| TIGIT5054 | CGTTGTTTCTATTGTGCCTG | 20 | AsCpf1 RR |
| TIGIT5055 | CCGTTGTTTCTATTGTGCCT | 20 | AsCpf1 RR |
| TIGIT5056 | CCACGGCACAAGTGACCCAG | 20 | AsCpf1 RR |
| TIGIT5057 | AGTTGACCTGGGTCACTTGT | 20 | AsCpf1 RR |
| TIGIT5058 | AAGTCAGCATTACAAATGGC | 20 | AsCpf1 RR |
| TIGIT5059 | CATCCTTCAAGGATCGAGTG | 20 | AsCpf1 RR |
| TIGIT5060 | ATCCTTCAAGGATCGAGTGG | 20 | AsCpf1 RR |
| TIGIT5061 | AGGATCGAGTGGCCCCAGGT | 20 | AsCpf1 RR |
| TIGIT5062 | AGGTCCCGGCCTGGGCCTCA | 20 | AsCpf1 RR |
| TIGIT5063 | GGCCTGGGCCTCACCCTCCA | 20 | AsCpf1 RR |
| TIGIT5064 | CGGTCAGCGACTGGAGGGTG | 20 | AsCpf1 RR |
| TIGIT5065 | GTCGCTGACCGTGAACGATA | 20 | AsCpf1 RR |
| TIGIT5066 | TGTATCGTTCACGGTCAGCG | 20 | AsCpf1 RR |
| TIGIT5067 | CTGTATCGTTCACGGTCAGC | 20 | AsCpf1 RR |
| TIGIT5068 | ATCAGGGTAGGTGTGATAGA | 20 | AsCpf1 RR |
| TIGIT5069 | AGTGTACGTCCCATCAGGGT | 20 | AsCpf1 RR |
| TIGIT5070 | GGAAGATTCTCCCAGTGTAC | 20 | AsCpf1 RR |
| TIGIT5071 | TGGAGGTCCTAGAAAGCTCA | 20 | AsCpf1 RR |
| TIGIT5072 | AGCAATGGAATCTGGAACCT | 20 | AsCpf1 RR |
| TIGIT5073 | AGATTCCATTGCTTGGAGCC | 20 | AsCpf1 RR |
| TIGIT5074 | GATTCCATTGCTTGGAGCCA | 20 | AsCpf1 RR |
| TIGIT5075 | ATTGCTTGGAGCCATGGCCG | 20 | AsCpf1 RR |
| TIGIT5076 | TTGCTTGGAGCCATGGCCGC | 20 | AsCpf1 RR |
| TIGIT5077 | CAGAATGGATTCTGAGGGCT | 20 | AsCpf1 RR |
| TIGIT5078 | ACAGAATGGATTCTGAGGGC | 20 | AsCpf1 RR |
| TIGIT5079 | TTCTGTGGAAGGTGACCTCA | 20 | AsCpf1 RR |
| TIGIT5080 | GCTGATTTTCTCCTGAGGTC | 20 | AsCpf1 RR |
| TIGIT5081 | TCCTGTCCAGCTGATTTTCT | 20 | AsCpf1 RR |
| TIGIT5082 | TTCCTCCTGTCCAGCTGATT | 20 | AsCpf1 RR |
| TIGIT5083 | TGGGGGTGAGGGAGCACTGG | 20 | AsCpf1 RR |
| TIGIT5084 | AGTGCTCCCTCACCCCCAGG | 20 | AsCpf1 RR |
| TIGIT5085 | TCACCCCCAGGAAGCTGTGT | 20 | AsCpf1 RR |
| TIGIT5086 | CAGGAAGCTGTGTCCAGGCA | 20 | AsCpf1 RR |
| TIGIT5087 | AGGAAGCTGTGTCCAGGCAG | 20 | AsCpf1 RR |
| TIGIT5088 | GGCAGAAGCTGCACCTGCTG | 20 | AsCpf1 RR |
| TIGIT5089 | CAGAGCCCAGCAGGTGCAGC | 20 | AsCpf1 RR |
| TIGIT5090 | GCTGCTCTCCACAGAGCCCA | 20 | AsCpf1 RR |
| TIGIT5091 | CGCTGCTCTCCACAGAGCCC | 20 | AsCpf1 RR |
| TIGIT5092 | ATGTCCTGAGTTACAGAAGC | 20 | AsCpf1 RR |

In some embodiments the gRNA for use in the disclosure is a gRNA targeting ADORA2a (ADORA2a gRNA). In some embodiments, the gRNA targeting ADORA2a is one or more of the gRNAs described in Table 5.

TABLE 5
ADORA2a gRNAs

| | gRNA Targeting Domain | | |
|---|---|---|---|
| Name | Sequence (DNA) | Length | Enzyme |
| ADORA2A337 | GAGCACACCCACTGCGATGT | 20 | SpyCas9 |
| ADORA2A338 | GATGGCCAGGAGACTGAAGA | 20 | SpyCas9 |
| ADORA2A339 | CTGCTCACCGGAGCGGGATG | 20 | SpyCas9 |
| ADORA2A340 | GTCTGTGGCCATGCCCATCA | 20 | SpyCas9 |
| ADORA2A341 | TCACCGGAGCGGGATGCGGA | 20 | SpyCas9 |
| ADORA2A342 | GTGGCAGGCAGCGCAGAACC | 20 | SpyCas9 |
| ADORA2A343 | AGCACACCAGCACATTGCCC | 20 | SpyCas9 |
| ADORA2A344 | CAGGTTGCTGTTGAGCCACA | 20 | SpyCas9 |
| ADORA2A345 | CTTCATTGCCTGCTTCGTCC | 20 | SpyCas9 |
| ADORA2A346 | GTACACCGAGGAGCCCATGA | 20 | SpyCas9 |
| ADORA2A347 | GATGGCAATGTAGCGGTCAA | 20 | SpyCas9 |
| ADORA2A348 | CTCCTCGGTGTACATCACGG | 20 | SpyCas9 |
| ADORA2A349 | CGAGGAGCCCATGATGGGCA | 20 | SpyCas9 |
| ADORA2A350 | GGGCTCCTCGGTGTACATCA | 20 | SpyCas9 |
| ADORA2A351 | CTTTGTGGTGTCACTGGCGG | 20 | SpyCas9 |
| ADORA2A352 | CCGCTCCGGTGAGCAGGGCC | 20 | SpyCas9 |
| ADORA2A353 | GGGTTCTGCGCTGCCTGCCA | 20 | SpyCas9 |
| ADORA2A354 | GGACGAAGCAGGCAATGAAG | 20 | SpyCas9 |
| ADORA2A355 | GTGCTGATGGTGATGGCAAA | 20 | SpyCas9 |
| ADORA2A356 | AGCGCAGAACCCGGTGCTGA | 20 | SpyCas9 |
| ADORA2A357 | GAGCTCCATCTTCAGTCTCC | 20 | SpyCas9 |
| ADORA2A358 | TGCTGATGGTGATGGCAAAG | 20 | SpyCas9 |
| ADORA2A359 | GGCGGCGGCCGACATCGCAG | 20 | SpyCas9 |
| ADORA2A360 | AATGAAGAGGCAGCCGTGGC | 20 | SpyCas9 |
| ADORA2A361 | GGGCAATGTGCTGGTGTGCT | 20 | SpyCas9 |
| ADORA2A362 | CATGCCCATCATGGGCTCCT | 20 | SpyCas9 |
| ADORA2A363 | AATGTAGCGGTCAATGGCGA | 20 | SpyCas9 |
| ADORA2A364 | AGTAGTTGGTGACGTTCTGC | 20 | SpyCas9 |
| ADORA2A365 | AGCGGTCAATGGCGATGGCC | 20 | SpyCas9 |
| ADORA2A366 | CGCATCCCGCTCCGGTGAGC | 20 | SpyCas9 |
| ADORA2A367 | GCATCCCGCTCCGGTGAGCA | 20 | SpyCas9 |
| ADORA2A368 | TGGGCAATGTGCTGGTGTGC | 20 | SpyCas9 |
| ADORA2A369 | CAACTACTTTGTGGTGTCAC | 20 | SpyCas9 |
| ADORA2A370 | CGCTCCGGTGAGCAGGGCCG | 20 | SpyCas9 |
| ADORA2A371 | GATGGTGATGGCAAAGGGGA | 20 | SpyCas9 |
| ADORA2A372 | GGTGTACATCACGGTGGAGC | 20 | SpyCas9 |
| ADORA2A373 | GAACGTCACCAACTACTTTG | 20 | SpyCas9 |
| ADORA2A374 | CAGTGACACCACAAAGTAGT | 20 | SpyCas9 |
| ADORA2A375 | GGCCATCCTGGGCAATGTGC | 20 | SpyCas9 |
| ADORA2A376 | CCCGGCCCTGCTCACCGGAG | 20 | SpyCas9 |
| ADORA2A377 | CACCAGCACATTGCCCAGGA | 20 | SpyCas9 |
| ADORA2A378 | TTTGCCATCACCATCAGCAC | 20 | SpyCas9 |
| ADORA2A379 | CTCCACCGTGATGTACACCG | 20 | SpyCas9 |
| ADORA2A380 | GGAGCTGGCCATTGCTGTGC | 20 | SpyCas9 |
| ADORA2A381 | CAGGATGGCCAGCACAGCAA | 20 | SpyCas9 |
| ADORA2A382 | GAACCCGGTGCTGATGGTGA | 20 | SpyCas9 |
| ADORA2A383 | TGGAGCTCTGCGTGAGGACC | 20 | SpyCas9 |
| ADORA2A384 | CCCGCTCCGGTGAGCAGGGC | 20 | SpyCas9 |
| ADORA2A385 | AGGCAATGAAGAGGCAGCCG | 20 | SpyCas9 |
| ADORA2A386 | CCGGCCCTGCTCACCGGAGC | 20 | SpyCas9 |
| ADORA2A387 | GCGGCGGCCGACATCGCAGT | 20 | SpyCas9 |
| ADORA2A388 | GGTGCTGATGGTGATGGCAA | 20 | SpyCas9 |
| ADORA2A389 | CTACTTTGTGGTGTCACTGG | 20 | SpyCas9 |
| ADORA2A390 | TACACCGAGGAGCCCATGAT | 20 | SpyCas9 |
| ADORA2A391 | TCTGTGGCCATGCCCATCAT | 20 | SpyCas9 |
| ADORA2A392 | ATTGCTGTGCTGGCCATCCT | 20 | SpyCas9 |
| ADORA2A393 | CGTGAGGACCAGGACGAAGC | 20 | SpyCas9 |
| ADORA2A394 | TTGCCATCACCATCAGCACC | 20 | SpyCas9 |
| ADORA2A395 | GGATGCGGATGGCAATGTAG | 20 | SpyCas9 |
| ADORA2A396 | TTGCCATCCGCATCCCGCTC | 20 | SpyCas9 |
| ADORA2A397 | TGAAGATGGAGCTCTGCGTG | 20 | SpyCas9 |
| ADORA2A398 | CATTGCTGTGCTGGCCATCC | 20 | SpyCas9 |
| ADORA2A399 | TGCTGGTGTGCTGGGCCGTG | 20 | SpyCas9 |
| ADORA2A820 | GGCTCCTCGGTGTACATCACG | 21 | SauCas9 |
| ADORA2A821 | GAGCTCTGCGTGAGGACCAGG | 21 | SauCas9 |
| ADORA2A822 | GATGGAGCTCTGCGTGAGGAC | 21 | SauCas9 |
| ADORA2A823 | CCAGCACACCAGCACATTGCC | 21 | SauCas9 |
| ADORA2A824 | AGGACCAGGACGAAGCAGGCA | 21 | SauCas9 |
| ADORA2A825 | TGCCATCCGCATCCCGCTCCG | 21 | SauCas9 |
| ADORA2A826 | GTGTGGCTCAACAGCAACCTG | 21 | SauCas9 |
| ADORA2A827 | AGCTCCACCGTGATGTACACC | 21 | SauCas9 |
| ADORA2A828 | GTAGCGGTCAATGGCGATGGC | 21 | SauCas9 |
| ADORA2A829 | CGGTGCTGATGGTGATGGCAA | 21 | SauCas9 |
| ADORA2A830 | CCCTGCTCACCGGAGCGGGAT | 21 | SauCas9 |
| ADORA2A831 | GTGACGTTCTGCAGGTTGCTG | 21 | SauCas9 |
| ADORA2A832 | GCTCCACCGTGATGTACACCG | 21 | SauCas9 |
| ADORA2A833 | ACTGAAGATGGAGCTCTGCGT | 21 | SauCas9 |
| ADORA2A834 | CCAGCTCCACCGTGATGTACA | 21 | SauCas9 |
| ADORA2A835 | CCTTTGCCATCACCATCAGCA | 21 | SauCas9 |
| ADORA2A836 | CCGGTGCTGATGGTGATGGCA | 21 | SauCas9 |
| ADORA2A837 | CCTGGGCAATGTGCTGGTGTG | 21 | SauCas9 |
| ADORA2A838 | AGGCAGCCGTGGCAGGCAGCG | 21 | SauCas9 |
| ADORA2A839 | GCGATGGCCAGGAGACTGAAG | 21 | SauCas9 |
| ADORA2A840 | CGATGGCCAGGAGACTGAAGA | 21 | SauCas9 |
| ADORA2A841 | TCCCGCTCCGGTGAGCAGGGC | 21 | SauCas9 |
| ADORA2A842 | TGCTTCGTCCTGGTCCTCACG | 21 | SauCas9 |
| ADORA2A843 | ACCAGGACGAAGCAGGCAATG | 21 | SauCas9 |
| ADORA2A844 | ATGTACACCGAGGAGCCCATG | 21 | SauCas9 |
| ADORA2A845 | TCGTCTGTGGCCATGCCCATC | 21 | SauCas9 |
| ADORA2A846 | TCAATGGCGATGGCCAGGAGA | 21 | SauCas9 |
| ADORA2A847 | GGTGCTGATGGTGATGGCAAA | 21 | SauCas9 |
| ADORA2A848 | TAGCGGTCAATGGCGATGGCC | 21 | SauCas9 |
| ADORA2A849 | TCCGCATCCCGCTCCGGTGAG | 21 | SauCas9 |
| ADORA2A850 | CTGGCGGCGGCCGACATCGCA | 21 | SauCas9 |
| ADORA2A851 | GCCATTGCTGTGCTGGCCATC | 21 | SauCas9 |
| ADORA2A852 | ATCCCGCTCCGGTGAGCAGGG | 21 | SauCas9 |
| ADORA2A853 | AGACTGAAGATGGAGCTCTGC | 21 | SauCas9 |
| ADORA2A854 | CCCCGGCCCTGCTCACCGGAG | 21 | SauCas9 |
| ADORA2A855 | ATGGTGATGGCAAAGGGGATG | 21 | SauCas9 |
| ADORA2A856 | GCTCCTCGGTGTACATCACGG | 21 | SauCas9 |
| ADORA2A248 | TGTCGATGGCAATAGCCAAG | 20 | SpyCas9 |
| ADORA2A249 | AGAAGTTGGTGACGTTCTGC | 20 | SpyCas9 |
| ADORA2A250 | TTCGCCATCACCATCAGCAC | 20 | SpyCas9 |
| ADORA2A251 | GAAGAAGAGGCAGCCATGGC | 20 | SpyCas9 |
| ADORA2A252 | CACAAGCACGTTACCCAGGA | 20 | SpyCas9 |
| ADORA2A253 | CAACTTCTTCGTGGTATCTC | 20 | SpyCas9 |
| ADORA2A254 | CAGGATGGCCAGCACAGCAA | 20 | SpyCas9 |
| ADORA2A255 | AATTCCACTCCGGTGAGCCA | 20 | SpyCas9 |
| ADORA2A256 | AGCGCAGAAGCCAGTGCTGA | 20 | SpyCas9 |
| ADORA2A257 | GTGCTGATGGTGATGGCGAA | 20 | SpyCas9 |
| ADORA2A258 | GGAGCTGGCCATTGCTGTGC | 20 | SpyCas9 |
| ADORA2A259 | AATAGCCAAGAGGCTGAAGA | 20 | SpyCas9 |
| ADORA2A260 | CTCCTCGGTGTACATCATGG | 20 | SpyCas9 |
| ADORA2A261 | GGACAAAGCAGGCGAAGAAG | 20 | SpyCas9 |
| ADORA2A262 | TCTGGCGGCGGCTGACATCG | 20 | SpyCas9 |
| ADORA2A263 | TGGGTAACGTGCTTGTGTGC | 20 | SpyCas9 |
| ADORA2A264 | GATGTACACCGAGGAGCCCA | 20 | SpyCas9 |
| ADORA2A265 | TAACCCCTGGCTCACCGGAG | 20 | SpyCas9 |
| ADORA2A266 | TCACCGGAGTGGAATTCGGA | 20 | SpyCas9 |
| ADORA2A267 | GCGGCGGCTGACATCGCGGT | 20 | SpyCas9 |
| ADORA2A268 | GATGGTGATGGCGAATGGGA | 20 | SpyCas9 |
| ADORA2A269 | GGCTTCTGCGCTGCCTGCCA | 20 | SpyCas9 |
| ADORA2A270 | ATTCCACTCCGGTGAGCCAG | 20 | SpyCas9 |
| ADORA2A271 | GGTGTACATCATGGTGGAGC | 20 | SpyCas9 |
| ADORA2A272 | ATTGCTGTGCTGGCCATCCT | 20 | SpyCas9 |
| ADORA2A273 | CTCCACCATGATGTACACCG | 20 | SpyCas9 |
| ADORA2A274 | GGCGGCGGCTGACATCGCGG | 20 | SpyCas9 |
| ADORA2A275 | TACACCGAGGAGCCCATGGC | 20 | SpyCas9 |
| ADORA2A276 | GGGTAACGTGCTTGTGTGCT | 20 | SpyCas9 |
| ADORA2A277 | CAGGTTGCTGTTGATCCACA | 20 | SpyCas9 |
| ADORA2A278 | TGAAGATGGAACTCTGCGTG | 20 | SpyCas9 |
| ADORA2A279 | GATGGCGATGTATCTGTCGA | 20 | SpyCas9 |
| ADORA2A280 | CTTCTTCGCCTGCTTTGTCC | 20 | SpyCas9 |
| ADORA2A281 | AGGCGAAGAAGAGGCAGCCA | 20 | SpyCas9 |
| ADORA2A282 | TGCTTGTGTGCTGGGCCGTG | 20 | SpyCas9 |
| ADORA2A283 | GAAGCCAGTGCTGATGGTGA | 20 | SpyCas9 |
| ADORA2A284 | CGTGAGGACCAGGACAAAGC | 20 | SpyCas9 |
| ADORA2A285 | TGGAACTCTGCGTGAGGACC | 20 | SpyCas9 |
| ADORA2A286 | CATTGCTGTGCTGGCCATCC | 20 | SpyCas9 |
| ADORA2A287 | TTCTCCCGCCATGGGCTCCT | 20 | SpyCas9 |
| ADORA2A288 | TGGCTCACCGGAGTGGAATT | 20 | SpyCas9 |
| ADORA2A289 | TGCTGATGGTGATGGCGAAT | 20 | SpyCas9 |
| ADORA2A290 | CTTCGTGGTATCTCTGGCGG | 20 | SpyCas9 |
| ADORA2A291 | AGCACACAAGCACGTTACCC | 20 | SpyCas9 |
| ADORA2A292 | GGGCTCCTCGGTGTACATCA | 20 | SpyCas9 |
| ADORA2A293 | GTACACCGAGGAGCCCATGG | 20 | SpyCas9 |
| ADORA2A294 | GAACGTCACCAACTTCTTCG | 20 | SpyCas9 |
| ADORA2A295 | TCGCCATCCGAATTCCACTC | 20 | SpyCas9 |
| ADORA2A296 | GAGTTCCATCTTCAGCCTCT | 20 | SpyCas9 |
| ADORA2A297 | GAATTCCACTCCGGTGAGCC | 20 | SpyCas9 |
| ADORA2A298 | CAGAGATACCACGAAGAAGT | 20 | SpyCas9 |
| ADORA2A299 | CTTCTTCGTGGTATCTCTGG | 20 | SpyCas9 |
| ADORA2A695 | CAGTGCTGATGGTGATGGCGA | 21 | SauCas9 |
| ADORA2A696 | CGAATTCCACTCCGGTGAGCC | 21 | SauCas9 |
| ADORA2A697 | CCGAATTCCACTCCGGTGAGC | 21 | SauCas9 |
| ADORA2A698 | GCTGAAGATGGAACTCTGCGT | 21 | SauCas9 |
| ADORA2A699 | CGTGCTTGTGTGCTGGGCCGT | 21 | SauCas9 |
| ADORA2A700 | GTGAGGACCAGGACAAAGCAG | 21 | SauCas9 |
| ADORA2A701 | TCGATGGCAATAGCCAAGAGG | 21 | SauCas9 |
| ADORA2A702 | CATCGACAGATACATCGCCAT | 21 | SauCas9 |
| ADORA2A703 | GTACACCGAGGAGCCCATGGC | 21 | SauCas9 |
| ADORA2A704 | GCTCCACCATGATGTACACCG | 21 | SauCas9 |
| ADORA2A705 | AAGCCAGTGCTGATGGTGATG | 21 | SauCas9 |
| ADORA2A706 | CACCGCGATGTCAGCCGCCGC | 21 | SauCas9 |
| ADORA2A707 | AGGCTGAAGATGGAACTCTGC | 21 | SauCas9 |
| ADORA2A708 | GCCGCCGCCAGAGATACCACG | 21 | SauCas9 |
| ADORA2A709 | AGCTCCACCATGATGTACACC | 21 | SauCas9 |
| ADORA2A710 | AGGCAGCCATGGCAGGCAGCG | 21 | SauCas9 |
| ADORA2A711 | CCTGGCTCACCGGAGTGGAAT | 21 | SauCas9 |
| ADORA2A712 | CCAGCTCCACCATGATGTACA | 21 | SauCas9 |
| ADORA2A713 | ACCAGGACAAAGCAGGCGAAG | 21 | SauCas9 |
| ADORA2A714 | CCTGGGTAACGTGCTTGTGTG | 21 | SauCas9 |
| ADORA2A715 | AGGACCAGGACAAAGCAGGCG | 21 | SauCas9 |
| ADORA2A716 | TCAGCCGCCGCCAGAGATACC | 21 | SauCas9 |
| ADORA2A717 | GGCTCCTCGGTGTACATCATG | 21 | SauCas9 |
| ADORA2A718 | CTGGCGGCGGCTGACATCGCG | 21 | SauCas9 |
| ADORA2A719 | GATGGAACTCTGCGTGAGGAC | 21 | SauCas9 |
| ADORA2A720 | GCTCCTCGGTGTACATCATGG | 21 | SauCas9 |
| ADORA2A721 | TGTACACCGAGGAGCCCATGG | 21 | SauCas9 |
| ADORA2A722 | GCCATTGCTGTGCTGGCCATC | 21 | SauCas9 |
| ADORA2A723 | CAATAGCCAAGAGGCTGAAGA | 21 | SauCas9 |
| ADORA2A724 | ATGGTGATGGCGAATGGGATG | 21 | SauCas9 |
| ADORA2A725 | ATGTACACCGAGGAGCCCATG | 21 | SauCas9 |
| ADORA2A726 | GTGTGGATCAACAGCAACCTG | 21 | SauCas9 |
| ADORA2A727 | TGCTTTGTCCTGGTCCTCACG | 21 | SauCas9 |
| ADORA2A728 | GTAACCCCTGGCTCACCGGAG | 21 | SauCas9 |
| ADORA2A729 | CCAGCACACAAGCACGTTACC | 21 | SauCas9 |
| ADORA2A730 | TATCTGTCGATGGCAATAGCC | 21 | SauCas9 |
| ADORA2A731 | GCAATAGCCAAGAGGCTGAAG | 21 | SauCas9 |
| ADORA2A732 | AGTGCTGATGGTGATGGCGAA | 21 | SauCas9 |
| ADORA2A733 | ACACCGAGGAGCCCATGGCGG | 21 | SauCas9 |
| ADORA2A734 | CGCCATCCGAATTCCACTCCG | 21 | SauCas9 |
| ADORA2A4111 | TGGTGTCACTGGCGGCGGCC | 20 | AsCpf1 |
| ADORA2A4112 | CCATCACCATCAGCACCGGG | 20 | AsCpf1 |
| ADORA2A4113 | CCATCGGCCTGACTCCCATG | 20 | AsCpf1 |
| ADORA2A4114 | GCTGACCGCAGTTGTTCCAA | 20 | AsCpf1 |
| ADORA2A4115 | AGGATGTGGTCCCCATGAAC | 20 | AsCpf1 |
| ADORA2A4116 | CCTGTGTGCTGGTGCCCCTG | 20 | AsCpf1 |
| ADORA2A4117 | CGGATCTTCCTGGCGGCGCG | 20 | AsCpf1 |
| ADORA2A4118 | CCCTCTGCTGGCTGCCCCTA | 20 | AsCpf1 |
| ADORA2A4119 | TTCTGCCCCGACTGCAGCCA | 20 | AsCpf1 |
| ADORA2A4120 | AAGGCAGCTGGCACCAGTGC | 20 | AsCpf1 |
| ADORA2A4121 | TAAGGGCATCATTGCCATCTG | 21 | SauCas9 |
| ADORA2A4122 | CGGCCTGACTCCCATGCTAGG | 21 | SauCas9 |
| ADORA2A4123 | GCAGTTGTTCCAACCTAGCAT | 21 | SauCas9 |
| ADORA2A4124 | CCGCAGTTGTTCCAACCTAGC | 21 | SauCas9 |
| ADORA2A4125 | CAAGAACCACTCCCAGGGCTG | 21 | SauCas9 |
| ADORA2A4126 | CTTGGCCCTCCCCGCAGCCCT | 21 | SauCas9 |
| ADORA2A4127 | CACTTGGCCCTCCCCGCAGCC | 21 | SauCas9 |
| ADORA2A4128 | GGCCAAGTGGCCTGTCTCTTT | 21 | SauCas9 |
| ADORA2A4129 | TTCATGGGGACCACATCCTCA | 21 | SauCas9 |
| ADORA2A4130 | TGAAGTACACCATGTAGTTCA | 21 | SauCas9 |
| ADORA2A4131 | CTGGTGCCCCTGCTGCTCATG | 21 | SauCas9 |
| ADORA2A4132 | GCTCATGCTGGGTGTCTATTT | 21 | SauCas9 |
| ADORA2A4133 | CTTCAGCTGTCGTCGCGCCGC | 21 | SauCas9 |
| ADORA2A4134 | CGCGACGACAGCTGAAGCAGA | 21 | SauCas9 |
| ADORA2A4135 | GATGGAGAGCCAGCCTCTGCC | 21 | SauCas9 |
| ADORA2A4136 | GCGTGGCTGCAGTCGGGGCAG | 21 | SauCas9 |
| ADORA2A4137 | ACGATGGCCAGGTACATGAGC | 21 | SauCas9 |
| ADORA2A4138 | CTCTCCCACACCAATTCGGTT | 21 | SauCas9 |
| ADORA2A4139 | GATTCACAACCGAATTGGTGT | 21 | SauCas9 |
| ADORA2A4140 | GGGATTCACAACCGAATTGGT | 21 | SauCas9 |
| ADORA2A4141 | CGTAGATGAAGGGATTCACAA | 21 | SauCas9 |
| ADORA2A4142 | GGATACGGTAGGCGTAGATGA | 21 | SauCas9 |
| ADORA2A4143 | TCATCTACGCCTACCGTATCC | 21 | SauCas9 |
| ADORA2A4144 | CGGATACGGTAGGCGTAGATG | 21 | SauCas9 |
| ADORA2A4145 | GCGGAAGGTCTGGCGGAACTC | 21 | SauCas9 |
| ADORA2A4146 | AATGATCTTGCGGAAGGTCTG | 21 | SauCas9 |
| ADORA2A4147 | GACGTGGCTGCGAATGATCTT | 21 | SauCas9 |
| ADORA2A4148 | TTGCTGCCTCAGGACGTGGCT | 21 | SauCas9 |
| ADORA2A4149 | CAAGGCAGCTGGCACCAGTGC | 21 | SauCas9 |
| ADORA2A4150 | CGGGCACTGGTGCCAGCTGCC | 21 | SauCas9 |
| ADORA2A4151 | CTTGGCAGCTCATGGCAGTGA | 21 | SauCas9 |
| ADORA2A4152 | CCGTCTCAACGGCCACCCGCC | 21 | SauCas9 |
| ADORA2A4153 | CACACTCCTGGCGGGTGGCCG | 21 | SauCas9 |
| ADORA2A4154 | TGCCGTTGGCCCACACTCCTG | 21 | SauCas9 |
| ADORA2A4155 | CCATTGGGCCTCCGCTCAGGG | 21 | SauCas9 |
| ADORA2A4156 | CATAGCCATTGGGCCTCCGCT | 21 | SauCas9 |
| ADORA2A4157 | AATGGCTATGCCCTGGGGCTG | 21 | SauCas9 |
| ADORA2A4158 | ATGCCCTGGGGCTGGTGAGTG | 21 | SauCas9 |
| ADORA2A4159 | GCCCTGGGGCTGGTGAGTGGA | 21 | SauCas9 |
| ADORA2A4160 | TGGTGAGTGGAGGGAGTGCCC | 21 | SauCas9 |
| ADORA2A4161 | GAGGGAGTGCCCAAGAGTCCC | 21 | SauCas9 |
| ADORA2A4162 | AGGGAGTGCCCAAGAGTCCCA | 21 | SauCas9 |
| ADORA2A4163 | GTCTGGGAGGCCCGTGTTCCC | 21 | SauCas9 |
| ADORA2A4164 | CATGGCTAAGGAGCTCCACGT | 21 | SauCas9 |
| ADORA2A4165 | GAGCTCCTTAGCCATGAGCTC | 21 | SauCas9 |
| ADORA2A4166 | GCTCCTTAGCCATGAGCTCAA | 21 | SauCas9 |
| ADORA2A4167 | GGCCTAGATGACCCCCTGGCC | 21 | SauCas9 |
| ADORA2A4168 | CCCCCTGGCCCAGGATGGAGC | 21 | SauCas9 |
| ADORA2A4169 | CTCCTGCTCCATCCTGGGCCA | 21 | SauCas9 |
| ADORA2A4416 | CCGTGATGTACACCGAGGAG | 20 | AsCpf1 RR |
| ADORA2A4417 | CTTTGCCATCACCATCAGCA | 20 | AsCpf1 RR |
| ADORA2A4418 | TTTGCCATCACCATCAGCAC | 20 | AsCpf1 RR |
| ADORA2A4419 | TTGCCTGCTTCGTCCTGGTC | 20 | AsCpf1 RR |
| ADORA2A4420 | TCCTGGTCCTCACGCAGAGC | 20 | AsCpf1 RR |
| ADORA2A4421 | TCTTCAGTCTCCTGGCCATC | 20 | AsCpf1 RR |
| AD0RA2A4422 | GTCTCCTGGCCATCGCCATT | 20 | AsCpf1 RR |
| AD0RA2A4423 | ACCTAGCATGGGAGTCAGGC | 20 | AsCpf1 RR |
| AD0RA2A4424 | AACCTAGCATGGGAGTCAGG | 20 | AsCpf1 RR |
| AD0RA2A4425 | ATGCTAGGTTGGAACAACTG | 20 | AsCpf1 RR |
| AD0RA2A4426 | GCAGCCCTGGGAGTGGTTCT | 20 | AsCpf1 RR |
| AD0RA2A4427 | CGCAGCCCTGGGAGTGGTTC | 20 | AsCpf1 RR |
| AD0RA2A4428 | AGGGCTGCGGGGAGGGCCAA | 20 | AsCpf1 RR |
| AD0RA2A4429 | TGGGGACCACATCCTCAAAG | 20 | AsCpf1 RR |
| ADORA2A4430 | CATGAACTACATGGTGTACT | 20 | AsCpf1 RR |
| ADORA2A4431 | ATGAACTACATGGTGTACTT | 20 | AsCpf1 RR |
| AD0RA2A4432 | ACTTCTTTGCCTGTGTGCTG | 20 | AsCpf1 RR |
| AD0RA2A4433 | TGCTGCTCATGCTGGGTGTC | 20 | AsCpf1 RR |
| AD0RA2A4434 | CAAATAGACACCCAGCATGA | 20 | AsCpf1 RR |
| AD0RA2A4435 | GCTGTCGTCGCGCCGCCAGG | 20 | AsCpf1 RR |
| AD0RA2A4436 | TGGCGGCGCGACGACAGCTG | 20 | AsCpf1 RR |
| AD0RA2A4437 | TCTGCTTCAGCTGTCGTCGC | 20 | AsCpf1 RR |
| AD0RA2A4438 | GGCAGAGGCTGGCTCTCCAT | 20 | AsCpf1 RR |
| AD0RA2A4439 | CGGCAGAGGCTGGCTCTCCA | 20 | AsCpf1 RR |
| ADORA2A4440 | CCGGCAGAGGCTGGCTCTCC | 20 | AsCpf1 RR |
| ADORA2A4441 | CACTGCAGAAGGAGGTCCAT | 20 | AsCpf1 RR |
| AD0RA2A4442 | TGCTGCCAAGTCACTGGCCA | 20 | AsCpf1 RR |
| AD0RA2A4443 | ACAATGATGGCCAGTGACTT | 20 | AsCpf1 RR |
| AD0RA2A4444 | TACACATCATCAACTGCTTC | 20 | AsCpf1 RR |
| AD0RA2A4445 | CTTTCTTCTGCCCCGACTGC | 20 | AsCpf1 RR |
| AD0RA2A4446 | GACTGCAGCCACGCCCCTCT | 20 | AsCpf1 RR |
| AD0RA2A4447 | TCTCTGGCTCATGTACCTGG | 20 | AsCpf1 RR |
| AD0RA2A4448 | CAACCGAATTGGTGTGGGAG | 20 | AsCpf1 RR |
| AD0RA2A4449 | ACACCAATTCGGTTGTGAAT | 20 | AsCpf1 RR |
| ADORA2A4450 | GTTGTGAATCCCTTCATCTA | 20 | AsCpf1 RR |
| ADORA2A4451 | TTCATCTACGCCTACCGTAT | 20 | AsCpf1 RR |
| AD0RA2A4452 | TCTACGCCTACCGTATCCGC | 20 | AsCpf1 RR |
| AD0RA2A4453 | CGAGTTCCGCCAGACCTTCC | 20 | AsCpf1 RR |
| AD0RA2A4454 | GCCAGACCTTCCGCAAGATC | 20 | AsCpf1 RR |
| AD0RA2A4455 | CCAGACCTTCCGCAAGATCA | 20 | AsCpf1 RR |
| AD0RA2A4456 | GCAAGATCATTCGCAGCCAC | 20 | AsCpf1 RR |
| AD0RA2A4457 | CAAGATCATTCGCAGCCACG | 20 | AsCpf1 RR |
| AD0RA2A4458 | CAGCCACGTCCTGAGGCAGC | 20 | AsCpf1 RR |
| AD0RA2A4459 | AGGCAGCTGGCACCAGTGCC | 20 | AsCpf1 RR |
| ADORA2A4460 | TCACTGCCATGAGCTGCCAA | 20 | AsCpf1 RR |
| ADORA2A4461 | TCTCAACGGCCACCCGCCAG | 20 | AsCpf1 RR |
| AD0RA2A4462 | CTCAGGGTGGGGAGCACTGC | 20 | AsCpf1 RR |
| AD0RA2A4463 | CACCCTGAGCGGAGGCCCAA | 20 | AsCpf1 RR |
| AD0RA2A4464 | ACCCTGAGCGGAGGCCCAAT | 20 | AsCpf1 RR |
| AD0RA2A4465 | AGGGCATAGCCATTGGGCCT | 20 | AsCpf1 RR |
| AD0RA2A4466 | CTCACCAGCCCCAGGGCATA | 20 | AsCpf1 RR |
| AD0RA2A4467 | TCCACTCACCAGCCCCAGGG | 20 | AsCpf1 RR |
| AD0RA2A4468 | TGGGACTCTTGGGCACTCCC | 20 | AsCpf1 RR |
| AD0RA2A4469 | CTGGGACTCTTGGGCACTCC | 20 | AsCpf1 RR |
| ADORA2A4470 | CCTGGGACTCTTGGGCACTC | 20 | AsCpf1 RR |
| ADORA2A4471 | AGGGGAACACGGGCCTCCCA | 20 | AsCpf1 RR |
| AD0RA2A4472 | CGTCTGGGAGGCCCGTGTTC | 20 | AsCpf1 RR |
| AD0RA2A4473 | AGACGTGGAGCTCCTTAGCC | 20 | AsCpf1 RR |
| AD0RA2A4474 | TTGAGCTCATGGCTAAGGAG | 20 | AsCpf1 RR |
| AD0RA2A4475 | CTGGCCTAGATGACCCCCTG | 20 | AsCpf1 RR |
| AD0RA2A4476 | TGGCCTAGATGACCCCCTGG | 20 | AsCpf1 RR |
| AD0RA2A4477 | TCCTGGGCCAGGGGGTCATC | 20 | AsCpf1 RR |
| AD0RA2A4478 | CTGGCCCAGGATGGAGCAGG | 20 | AsCpf1 RR |
| AD0RA2A4479 | TGGCCCAGGATGGAGCAGGA | 20 | AsCpf1 RR |
| ADORA2A4480 | CGCGAGTTCCGCCAGACCTT | 20 | AsCpf1 RVR |
| ADORA2A4481 | CCCTGGGGCTGGTGAGTGGA | 20 | AsCpf1 RVR |

In some embodiments the gRNA for use in the disclosure is a gRNA targeting TGFbetaR2 (TGFbetaR2 gRNA). In some embodiments, the gRNA targeting TGFbetaR2 is one or more of the gRNAs described in Table 6.

TABLE 6
TGFbetaR2 gRNAs

| | gRNA Targeting | | |
|---|---|---|---|
| Name | Domain Sequence (DNA) | Length | Enzyme |
| TGFBR24326 | CAGGACGATGTGCAGCGGCC | 20 | AsCpf1 RR |
| TGFBR24327 | ACCGCACGTTCAGAAGTCGG | 20 | AsCpf1 RR |
| TGFBR24328 | ACAACTGTGTAAATTTTGTG | 20 | AsCpf1 RR |
| TGFBR24329 | CAACTGTGTAAATTTTGTGA | 20 | AsCpf1 RR |
| TGFBR24330 | ACCTGTGACAACCAGAAATC | 20 | AsCpf1 RR |
| TGFBR24331 | CCTGTGACAACCAGAAATCC | 20 | AsCpf1 RR |
| TGFBR24332 | TGTGGCTTCTCACAGATGGA | 20 | AsCpf1 RR |
| TGFBR24333 | TCTGTGAGAAGCCACAGGAA | 20 | AsCpf1 RR |
| TGFBR24334 | AAGCTCCCCTACCATGACTT | 20 | AsCpf1 RR |
| TGFBR24335 | GAATAAAGTCATGGTAGGGG | 20 | AsCpf1 RR |
| TGFBR24336 | AGAATAAAGTCATGGTAGGG | 20 | AsCpf1 RR |
| TGFBR24337 | CTACCATGACTTTATTCTGG | 20 | AsCpf1 RR |
| TGFBR24338 | TACCATGACTTTATTCTGGA | 20 | AsCpf1 RR |
| TGFBR24339 | TAATGCACTTTGGAGAAGCA | 20 | AsCpf1 RR |
| TGFBR24340 | TTCATAATGCACTTTGGAGA | 20 | AsCpf1 RR |
| TGFBR24341 | AAGTGCATTATGAAGGAAAA | 20 | AsCpf1 RR |
| TGFBR24342 | TGTGTTCCTGTAGCTCTGAT | 20 | AsCpf1 RR |
| TGFBR24343 | TGTAGCTCTGATGAGTGCAA | 20 | AsCpf1 RR |
| TGFBR24344 | AGTGACAGGCATCAGCCTCC | 20 | AsCpf1 RR |
| TGFBR24345 | AGTGGTGGCAGGAGGCTGAT | 20 | AsCpf1 RR |
| TGFBR24346 | AGGTTGAACTCAGCTTCTGC | 20 | AsCpf1 RR |
| TGFBR24347 | CAGGTTGAACTCAGCTTCTG | 20 | AsCpf1 RR |
| TGFBR24348 | ACCTGGGAAACCGGCAAGAC | 20 | AsCpf1 RR |
| TGFBR24349 | CGTCTTGCCGGTTTCCCAGG | 20 | AsCpf1 RR |
| TGFBR24350 | GCGTCTTGCCGGTTTCCCAG | 20 | AsCpf1 RR |
| TGFBR24351 | TGAGCTTCCGCGTCTTGCCG | 20 | AsCpf1 RR |
| TGFBR24352 | GCGAGCACTGTGCCATCATC | 20 | AsCpf1 RR |
| TGFBR24353 | GGATGATGGCACAGTGCTCG | 20 | AsCpf1 RR |
| TGFBR24354 | AGGATGATGGCACAGTGCTC | 20 | AsCpf1 RR |
| TGFBR24355 | CGTGTGCCAACAACATCAAC | 20 | AsCpf1 RR |
| TGFBR24356 | GCTCAATGGGCAGCAGCTCT | 20 | AsCpf1 RR |
| TGFBR24357 | ACCAGGGTGTCCAGCTCAAT | 20 | AsCpf1 RR |
| TGFBR24358 | CACCAGGGTGTCCAGCTCAA | 20 | AsCpf1 RR |
| TGFBR24359 | CCACCAGGGTGTCCAGCTCA | 20 | AsCpf1 RR |
| TGFBR24360 | GCTTGGCCTTATAGACCTCA | 20 | AsCpf1 RR |
| TGFBR24361 | GAGCAGTTTGAGACAGTGGC | 20 | AsCpf1 RR |
| TGFBR24362 | AGAGGCATACTCCTCATAGG | 20 | AsCpf1 RR |
| TGFBR24363 | CTATGAGGAGTATGCCTCTT | 20 | AsCpf1 RR |
| TGFBR24364 | AAGAGGCATACTCCTCATAG | 20 | AsCpf1 RR |
| TGFBR24365 | TATGAGGAGTATGCCTCTTG | 20 | AsCpf1 RR |
| TGFBR24366 | GATTGATGTCTGAGAAGATG | 20 | AsCpf1 RR |
| TGFBR24367 | CTCCTCAGCCGTCAGGAACT | 20 | AsCpf1 RR |
| TGFBR24368 | GTTCCTGACGGCTGAGGAGC | 20 | AsCpf1 RR |
| TGFBR24369 | GCTCCTCAGCCGTCAGGAAC | 20 | AsCpf1 RR |
| TGFBR24370 | TGACGGCTGAGGAGCGGAAG | 20 | AsCpf1 RR |
| TGFBR24371 | TCTTCCGCTCCTCAGCCGTC | 20 | AsCpf1 RR |
| TGFBR24372 | AACTCCGTCTTCCGCTCCTC | 20 | AsCpf1 RR |
| TGFBR24373 | CAACTCCGTCTTCCGCTCCT | 20 | AsCpf1 RR |
| TGFBR24374 | CCAACTCCGTCTTCCGCTCC | 20 | AsCpf1 RR |
| TGFBR24375 | ACGCCAAGGGCAACCTACAG | 20 | AsCpf1 RR |
| TGFBR24376 | CGCCAAGGGCAACCTACAGG | 20 | AsCpf1 RR |
| TGFBR24377 | AGCTGATGACATGCCGCGTC | 20 | AsCpf1 RR |
| TGFBR24378 | GGGCGAGGGAGCTGCCCAGC | 20 | AsCpf1 RR |
| TGFBR24379 | CGGGCGAGGGAGCTGCCCAG | 20 | AsCpf1 RR |
| TGFBR24380 | CCGGGCGAGGGAGCTGCCCA | 20 | AsCpf1 RR |
| TGFBR24381 | TCGCCCGGGGGATTGCTCAC | 20 | AsCpf1 RR |
| TGFBR24382 | ACATGGAGTGTGATCACTGT | 20 | AsCpf1 RR |
| TGFBR24383 | CAGTGATCACACTCCATGTG | 20 | AsCpf1 RR |
| TGFBR24384 | TGTGGGAGGCCCAAGATGCC | 20 | AsCpf1 RR |
| TGFBR24385 | TGTGCACGATGGGCATCTTG | 20 | AsCpf1 RR |
| TGFBR24386 | CGAGGATATTGGAGCTCTTG | 20 | AsCpf1 RR |
| TGFBR24387 | ATATCCTCGTGAAGAACGAC | 20 | AsCpf1 RR |
| TGFBR24388 | GACGCAGGGAAAGCCCAAAG | 20 | AsCpf1 RR |
| TGFBR24389 | CTGCGTCTGGACCCTACTCT | 20 | AsCpf1 RR |
| TGFBR24390 | TGCGTCTGGACCCTACTCTG | 20 | AsCpf1 RR |
| TGFBR24391 | CAGACAGAGTAGGGTCCAGA | 20 | AsCpf1 RR |
| TGFBR24392 | GCCAGCACGATCCCACCGCA | 20 | AsCpf1 RVR |
| TGFBR24393 | AAGGAAAAAAAAAAGCCTGG | 20 | AsCpf1 RVR |
| TGFBR24394 | ACACCAGCAATCCTGACTTG | 20 | AsCpf1 RVR |
| TGFBR24395 | ACTAGCAACAAGTCAGGATT | 20 | AsCpf1 RVR |
| TGFBR24396 | GCAACTCCCAGTGGTGGCAG | 20 | AsCpf1 RVR |
| TGFBR24397 | TGTCATCATCATCTTCTACT | 20 | AsCpf1 RVR |
| TGFBR24398 | GACCTCAGCAAAGCGACCTT | 20 | AsCpf1 RVR |
| TGFBR24399 | AGGCCAAGCTGAAGCAGAAC | 20 | AsCpf1 RVR |
| TGFBR24400 | AGGAGTATGCCTCTTGGAAG | 20 | AsCpf1 RVR |
| TGFBR24401 | CCTCTTGGAAGACAGAGAAG | 20 | AsCpf1 RVR |
| TGFBR24402 | TTCTCATGCTTCAGATTGAT | 20 | AsCpf1 RVR |
| TGFBR24403 | CTCGTGAAGAACGACCTAAC | 20 | AsCpf1 RVR |
| TGFbR2036 | GGCCGCTGCACATCGTCCTG | 20 | SpyCas9 |
| TGFbR2037 | GCGGGGTCTGCCATGGGTCG | 20 | SpyCas9 |
| TGFbR2038 | AGTTGCTCATGCAGGATTTC | 20 | SpyCas9 |
| TGFbR2039 | CCAGAATAAAGTCATGGTAG | 20 | SpyCas9 |
| TGFbR2040 | CCCCTACCATGACTTTATTC | 20 | SpyCas9 |
| TGFbR2041 | AAGTCATGGTAGGGGAGCTT | 20 | SpyCas9 |
| TGFbR2042 | AGTCATGGTAGGGGAGCTTG | 20 | SpyCas9 |
| TGFbR2043 | ATTGCACTCATCAGAGCTAC | 20 | SpyCas9 |
| TGFbR2044 | CCTAGAGTGAAGAGATTCAT | 20 | SpyCas9 |
| TGFbR2045 | CCAATGAATCTCTTCACTCT | 20 | SpyCas9 |
| TGFbR2046 | AAAGTCATGGTAGGGGAGCT | 20 | SpyCas9 |
| TGFbR2047 | GTGAGCAATCCCCCGGGCGA | 20 | SpyCas9 |
| TGFbR2048 | GTCGTTCTTCACGAGGATAT | 20 | SpyCas9 |
| TGFbR2049 | GCCGCGTCAGGTACTCCTGT | 20 | SpyCas9 |
| TGFbR2050 | GACGCGGCATGTCATCAGCT | 20 | SpyCas9 |
| TGFbR2051 | GCTTCTGCTGCCGGTTAACG | 20 | SpyCas9 |
| TGFbR2052 | GTGGATGACCTGGCTAACAG | 20 | SpyCas9 |
| TGFbR2053 | GTGATCACACTCCATGTGGG | 20 | SpyCas9 |
| TGFbR2054 | GCCCATTGAGCTGGACACCC | 20 | SpyCas9 |
| TGFbR2055 | GCGGTCATCTTCCAGGATGA | 20 | SpyCas9 |
| TGFbR2056 | GGGAGCTGCCCAGCTTGCGC | 20 | SpyCas9 |
| TGFbR2057 | GTTGATGTTGTTGGCACACG | 20 | SpyCas9 |
| TGFbR2058 | GGCATCTTGGGCCTCCCACA | 20 | SpyCas9 |
| TGFbR2059 | GCGGCATGTCATCAGCTGGG | 20 | SpyCas9 |
| TGFbR2060 | GCTCCTCAGCCGTCAGGAAC | 20 | SpyCas9 |
| TGFbR2061 | GCTGGTGTTATATTCTGATG | 20 | SpyCas9 |
| TGFbR2062 | CCGACTTCTGAACGTGCGGT | 20 | SpyCas9 |
| TGFbR2063 | TGCTGGCGATACGCGTCCAC | 20 | SpyCas9 |
| TGFbR2064 | CCCGACTTCTGAACGTGCGG | 20 | SpyCas9 |
| TGFbR2065 | CCACCGCACGTTCAGAAGTC | 20 | SpyCas9 |
| TGFbR2066 | TCACCCGACTTCTGAACGTG | 20 | SpyCas9 |
| TGFbR2067 | CCCACCGCACGTTCAGAAGT | 20 | SpyCas9 |
| TGFbR2068 | CGAGCAGCGGGGTCTGCCAT | 20 | SpyCas9 |
| TGFbR2069 | ACGAGCAGCGGGGTCTGCCA | 20 | SpyCas9 |
| TGFbR2070 | AGCGGGGTCTGCCATGGGTC | 20 | SpyCas9 |
| TGFbR2071 | CCTGAGCAGCCCCCGACCCA | 20 | SpyCas9 |
| TGFbR2072 | CCATGGGTCGGGGGCTGCTC | 20 | SpyCas9 |
| TGFbR2073 | AACGTGCGGTGGGATCGTGC | 20 | SpyCas9 |
| TGFbR2074 | GGACGATGTGCAGCGGCCAC | 20 | SpyCas9 |
| TGFbR2075 | GTCCACAGGACGATGTGCAG | 20 | SpyCas9 |
| TGFbR2076 | CATGGGTCGGGGGCTGCTCA | 20 | SpyCas9 |
| TGFbR2077 | CAGCGGGGTCTGCCATGGGT | 20 | SpyCas9 |
| TGFbR2078 | ATGGGTCGGGGGCTGCTCAG | 20 | SpyCas9 |
| TGFbR2079 | CGGGGTCTGCCATGGGTCGG | 20 | SpyCas9 |
| TGFbR2080 | AGGAAGTCTGTGTGGCTGTA | 20 | SpyCas9 |
| TGFbR2081 | CTCCATCTGTGAGAAGCCAC | 20 | SpyCas9 |
| TGFbR2082 | ATGATAGTCACTGACAACAA | 20 | SpyCas9 |
| TGFbR2083 | GATGCTGCAGTTGCTCATGC | 20 | SpyCas9 |
| TGFbR2084 | ACAGCCACACAGACTTCCTG | 20 | SpyCas9 |
| TGFbR2085 | GAAGCCACAGGAAGTCTGTG | 20 | SpyCas9 |
| TGFbR2086 | TTCCTGTGGCTTCTCACAGA | 20 | SpyCas9 |
| TGFbR2087 | CTGTGGCTTCTCACAGATGG | 20 | SpyCas9 |
| TGFbR2088 | TCACAAAATTTACACAGTTG | 20 | SpyCas9 |
| TGFbR2089 | GACAACATCATCTTCTCAGA | 20 | SpyCas9 |
| TGFbR2090 | TCCAGAATAAAGTCATGGTA | 20 | SpyCas9 |
| TGFbR2091 | GGTAGGGGAGCTTGGGGTCA | 20 | SpyCas9 |
| TGFbR2092 | TTCTCCAAAGTGCATTATGA | 20 | SpyCas9 |
| TGFbR2093 | CATCTTCCAGAATAAAGTCA | 20 | SpyCas9 |
| TGFbR2094 | CACATGAAGAAAGTCTCACC | 20 | SpyCas9 |
| TGFbR2095 | TTCCAGAATAAAGTCATGGT | 20 | SpyCas9 |
| TGFbR2096 | TTTTCCTTCATAATGCACTT | 20 | SpyCas9 |
| TGFBR24024 | CACAGTTGTGGAAACTTGAC | 20 | AsCpf1 |
| TGFBR24039 | CCCAACTCCGTCTTCCGCTC | 20 | AsCpf1 |
| TGFBR24040 | GGCTTTCCCTGCGTCTGGAC | 20 | AsCpf1 |
| TGFBR24036 | CTGAGGTCTATAAGGCCAAG | 20 | AsCpf1 |
| TGFBR24026 | TGATGTGAGATTTTCCACCT | 20 | AsCpf1 |
| TGFBR24038 | CCTATGAGGAGTATGCCTCT | 20 | AsCpf1 |
| TGFBR24033 | AAGTGACAGGCATCAGCCTC | 20 | AsCpf1 |
| TGFBR24028 | CCATGACCCCAAGCTCCCCT | 20 | AsCpf1 |
| TGFBR24031 | CTTCATAATGCACTTTGGAG | 20 | AsCpf1 |
| TGFBR24032 | TTCATGTGTTCCTGTAGCTC | 20 | AsCpf1 |
| TGFBR24029 | TTCTGGAAGATGCTGCTTCT | 20 | AsCpf1 |
| TGFBR24035 | CCCACCAGGGTGTCCAGCTC | 20 | AsCpf1 |
| TGFBR24037 | AGACAGTGGCAGTCAAGATC | 20 | AsCpf1 |
| TGFBR24041 | CCTGCGTCTGGACCCTACTC | 20 | AsCpf1 |
| TGFBR24025 | CACAACTGTGTAAATTTTGT | 20 | AsCpf1 |
| TGFBR24030 | GAGAAGCAGCATCTTCCAGA | 20 | AsCpf1 |
| TGFBR24027 | TGGTTGTCACAGGTGGAAAA | 20 | AsCpf1 |
| TGFBR24034 | CCAGGTTGAACTCAGCTTCT | 20 | AsCpf1 |
| TGFBR24043 | ATCACAAAATTTACACAGTTG | 21 | SauCas9 |
| TGFBR24065 | GGCATCAGCCTCCTGCCACCA | 21 | SauCas9 |
| TGFBR24110 | GTTAGCCAGGTCATCCACAGA | 21 | SauCas9 |
| TGFBR24099 | GCTGGGCAGCTCCCTCGCCCG | 21 | SauCas9 |
| TGFBR24064 | CAGGAGGCTGATGCCTGTCAC | 21 | SauCas9 |
| TGFBR24094 | GAGGAGCGGAAGACGGAGTTG | 21 | SauCas9 |
| TGFBR24108 | CGTCTGGACCCTACTCTGTCT | 21 | SauCas9 |
| TGFBR24058 | TTTTTCCTTCATAATGCACTT | 21 | SauCas9 |
| TGFBR24075 | CCATTGAGCTGGACACCCTGG | 21 | SauCas9 |
| TGFBR24057 | CTTCTCCAAAGTGCATTATGA | 21 | SauCas9 |
| TGFBR24103 | GCCCAAGATGCCCATCGTGCA | 21 | SauCas9 |
| TGFBR24060 | TCATGTGTTCCTGTAGCTCTG | 21 | SauCas9 |
| TGFBR24048 | GTGATGCTGCAGTTGCTCATG | 21 | SauCas9 |
| TGFBR24087 | TCTCATGCTTCAGATTGATGT | 21 | SauCas9 |
| TGFBR24081 | TCCCTATGAGGAGTATGCCTC | 21 | SauCas9 |
| TGFBR24044 | CATCACAAAATTTACACAGTT | 21 | SauCas9 |
| TGFBR24077 | ATTGAGCTGGACACCCTGGTG | 21 | SauCas9 |
| TGFBR24080 | CAGTCAAGATCTTTCCCTATG | 21 | SauCas9 |
| TGFBR24046 | AGGATTTCTGGTTGTCACAGG | 21 | SauCas9 |
| TGFBR24101 | TCCACAGTGATCACACTCCAT | 21 | SauCas9 |
| TGFBR24079 | AGCAGAACACTTCAGAGCAGT | 21 | SauCas9 |
| TGFBR24072 | CCGGCAAGACGCGGAAGCTCA | 21 | SauCas9 |
| TGFBR24074 | GATGTCAGAGCGGTCATCTTC | 21 | SauCas9 |
| TGFBR24062 | TCATTGCACTCATCAGAGCTA | 21 | SauCas9 |
| TGFBR24054 | CTTCCAGAATAAAGTCATGGT | 21 | SauCas9 |
| TGFBR24045 | AGATTTTCCACCTGTGACAAC | 21 | SauCas9 |
| TGFBR24049 | ACTGCAGCATCACCTCCATCT | 21 | SauCas9 |
| TGFBR24098 | AGCTGGGCAGCTCCCTCGCCC | 21 | SauCas9 |
| TGFBR24090 | TGACGGCTGAGGAGCGGAAGA | 21 | SauCas9 |
| TGFBR24076 | CATTGAGCTGGACACCCTGGT | 21 | SauCas9 |
| TGFBR24078 | AGCAAAGCGACCTTTCCCCAC | 21 | SauCas9 |
| TGFBR24067 | CGCGTTAACCGGCAGCAGAAG | 21 | SauCas9 |
| TGFBR24063 | GAAATATGACTAGCAACAAGT | 21 | SauCas9 |
| TGFBR24107 | AGACAGAGTAGGGTCCAGACG | 21 | SauCas9 |
| TGFBR24047 | CAGGATTTCTGGTTGTCACAG | 21 | SauCas9 |
| TGFBR24096 | CTCCTGTAGGTTGCCCTTGGC | 21 | SauCas9 |
| TGFBR24105 | ACAGAGTAGGGTCCAGACGCA | 21 | SauCas9 |
| TGFBR24056 | GCTTCTCCAAAGTGCATTATG | 21 | SauCas9 |
| TGFBR24068 | GCAGCAGAAGCTGAGTTCAAC | 21 | SauCas9 |
| TGFBR24093 | TGAGGAGCGGAAGACGGAGTT | 21 | SauCas9 |
| TGFBR24055 | CTTTGGAGAAGCAGCATCTTC | 21 | SauCas9 |
| TGFBR24053 | CTCCCCTACCATGACTTTATT | 21 | SauCas9 |
| TGFBR24106 | GACAGAGTAGGGTCCAGACGC | 21 | SauCas9 |
| TGFBR24092 | CTGAGGAGCGGAAGACGGAGT | 21 | SauCas9 |
| TGFBR24102 | GGGCATCTTGGGCCTCCCACA | 21 | SauCas9 |
| TGFBR24082 | CCAAGAGGCATACTCCTCATA | 21 | SauCas9 |
| TGFBR24051 | AGAATGACGAGAACATAACAC | 21 | SauCas9 |
| TGFBR24097 | CCTGACGCGGCATGTCATCAG | 21 | SauCas9 |
| TGFBR24073 | AGCGAGCACTGTGCCATCATC | 21 | SauCas9 |
| TGFBR24104 | GCAGGTTAGGTCGTTCTTCAC | 21 | SauCas9 |
| TGFBR24050 | ACCTCCATCTGTGAGAAGCCA | 21 | SauCas9 |
| TGFBR24052 | TAAAGTCATGGTAGGGGAGCT | 21 | SauCas9 |
| TGFBR24061 | TCAGAGCTACAGGAACACATG | 21 | SauCas9 |
| TGFBR24086 | TCTCAGACATCAATCTGAAGC | 21 | SauCas9 |
| TGFBR24066 | CATCAGCCTCCTGCCACCACT | 21 | SauCas9 |
| TGFBR24089 | CGCTCCTCAGCCGTCAGGAAC | 21 | SauCas9 |
| TGFBR24071 | AACCTGGGAAACCGGCAAGAC | 21 | SauCas9 |
| TGFBR24095 | TCCACGCCAAGGGCAACCTAC | 21 | SauCas9 |
| TGFBR24100 | GAGGTGAGCAATCCCCCGGGC | 21 | SauCas9 |
| TGFBR24069 | CAGCAGAAGCTGAGTTCAACC | 21 | SauCas9 |
| TGFBR24083 | TCCAAGAGGCATACTCCTCAT | 21 | SauCas9 |
| TGFBR24070 | AGCAGAAGCTGAGTTCAACCT | 21 | SauCas9 |
| TGFBR24088 | CCAGTTCCTGACGGCTGAGGA | 21 | SauCas9 |
| TGFBR24085 | AGGAGTATGCCTCTTGGAAGA | 21 | SauCas9 |
| TGFBR24084 | TTCCAAGAGGCATACTCCTCA | 21 | SauCas9 |
| TGFBR24042 | CAACTGTGTAAATTTTGTGAT | 21 | SauCas9 |
| TGFBR24059 | TGAAGGAAAAAAAAAAGCCTG | 21 | SauCas9 |
| TGFBR24091 | CGTCTTCCGCTCCTCAGCCGT | 21 | SauCas9 |
| TGFBR24109 | CCAGGTCATCCACAGACAGAG | 21 | SauCas9 |
| TGFBR2736 | GCCTAGAGTGAAGAGATTCAT | 21 | SpyCas9 |
| TGFBR2737 | GTTCTCCAAAGTGCATTATGA | 21 | SpyCas9 |
| TGFBR2738 | GCATCTTCCAGAATAAAGTCA | 21 | SpyCas9 |

In some embodiments the gRNA for use in the disclosure is a gRNA targeting CISH (CISH gRNA). In some embodiments, the gRNA targeting CISH is one or more of the gRNAs described in Table 7.

TABLE 7
CISH gRNAs

| | gRNA Targeting Domain | | |
|---|---|---|---|
| Name | Sequence (DNA) | Length | Enzyme |
| CISH0873 | CAACCGTCTGGTGGCCGACG | 20 | SpyCas9 |
| CISH0874 | CAGGATCGGGGCTGTCGCTT | 20 | SpyCas9 |
| CISH0875 | TCGGGCCTCGCTGGCCGTAA | 20 | SpyCas9 |
| CISH0876 | GAGGTAGTCGGCCATGCGCC | 20 | SpyCas9 |
| CISH0877 | CAGGTGTTGTCGGGCCTCGC | 20 | SpyCas9 |
| CISH0878 | GGAGGTAGTCGGCCATGCGC | 20 | SpyCas9 |
| CISH0879 | GGCATACTCAATGCGTACAT | 20 | SpyCas9 |
| CISH0880 | CCGCCTTGTCATCAACCGTC | 20 | SpyCas9 |
| CISH0881 | AGGATCGGGGCTGTCGCTTC | 20 | SpyCas9 |
| CISH0882 | CCTTGTCATCAACCGTCTGG | 20 | SpyCas9 |
| CISH0883 | TACTCAATGCGTACATTGGT | 20 | SpyCas9 |
| CISH0884 | GGGTTCCATTACGGCCAGCG | 20 | SpyCas9 |
| CISH0885 | GGCACTGCTTCTGCGTACAA | 20 | SpyCas9 |
| CISH0886 | GGTTGATGACAAGGCGGCAC | 20 | SpyCas9 |
| CISH0887 | TGCTGGGGCCTTCCTCGAGG | 20 | SpyCas9 |
| CISH0888 | TTGCTGGCTGTGGAGCGGAC | 20 | SpyCas9 |
| CISH0889 | TTCTCCTACCTTCGGGAATC | 20 | SpyCas9 |
| CISH0890 | GACTGGCTTGGGCAGTTCCA | 20 | SpyCas9 |
| CISH0891 | CATGCAGCCCTTGCCTGCTG | 20 | SpyCas9 |
| CISH0892 | AGCAAAGGACGAGGTCTAGA | 20 | SpyCas9 |
| CISH0893 | GCCTGCTGGGGCCTTCCTCG | 20 | SpyCas9 |
| CISH0894 | CAGACTCACCAGATTCCCGA | 20 | SpyCas9 |
| CISH0895 | ACCTCGTCCTTTGCTGGCTG | 20 | SpyCas9 |
| CISH0896 | CTCACCAGATTCCCGAAGGT | 20 | SpyCas9 |
| CISH7048 | TACGCAGAAGCAGTGCCCGC | 20 | AsCpf1 |
| CISH7049 | AGGTGTACAGCAGTGGCTGG | 20 | AsCpf1 |
| CISH7050 | GGTGTACAGCAGTGGCTGGT | 20 | AsCpf1 |
| CISH7051 | CGGATGTGGTCAGCCTTGTG | 20 | AsCpf1 |
| CISH7052 | CACTGACAGCGTGAACAGGT | 20 | AsCpf1 |
| CISH7053 | ACTGACAGCGTGAACAGGTA | 20 | AsCpf1 |
| CISH7054 | GCTCACTCTCTGTCTGGGCT | 20 | AsCpf1 |
| CISH7055 | CTGGCTGTGGAGCGGACTGG | 20 | AsCpf1 |
| CISH7056 | GCTCTGACTGTACGGGGCAA | 20 | AsCpf1 RR |
| CISH7057 | AGCTCTGACTGTACGGGGCA | 20 | AsCpf1 RR |
| CISH7058 | ACAGTACCCCTTCCAGCTCT | 20 | AsCpf1 RR |
| CISH7059 | CGTCGGCCACCAGACGGTTG | 20 | AsCpf1 RR |
| CISH7060 | CCAGCCACTGCTGTACACCT | 20 | AsCpf1 RR |
| CISH7061 | ACCCCGGCCCTGCCTATGCC | 20 | AsCpf1 RR |
| CISH7062 | GGTATCAGCAGTGCAGGAGG | 20 | AsCpf1 RR |
| CISH7063 | GATGTGGTCAGCCTTGTGCA | 20 | AsCpf1 RR |
| CISH7064 | GGATGTGGTCAGCCTTGTGC | 20 | AsCpf1 RR |
| CISH7065 | GGCCACGCATCCTGGCCTTT | 20 | AsCpf1 RR |
| CISH7066 | GAAAGGCCAGGATGCGTGGC | 20 | AsCpf1 RR |
| CISH7067 | ACTGCTTGTCCAGGCCACGC | 20 | AsCpf1 RR |
| CISH7068 | TCTGGACTCCAACTGCTTGT | 20 | AsCpf1 RR |
| CISH7069 | GTCTGGACTCCAACTGCTTG | 20 | AsCpf1 RR |
| CISH7070 | GCTTCCGTCTGGACTCCAAC | 20 | AsCpf1 RR |
| CISH7071 | GACGGAAGCTGGAGTCGGCA | 20 | AsCpf1 RR |
| CISH7072 | CGCTGTCAGTGAAAACCACT | 20 | AsCpf1 RR |
| CISH7073 | CTGACAGCGTGAACAGGTAG | 20 | AsCpf1 RR |
| CISH7074 | TTACGGCCAGCGAGGCCCGA | 20 | AsCpf1 RR |
| CISH7075 | ATTACGGCCAGCGAGGCCCG | 20 | AsCpf1 RR |
| CISH7076 | GGAATCTGGTGAGTCTGAGG | 20 | AsCpf1 RR |
| CISH7077 | CCCTCAGACTCACCAGATTC | 20 | AsCpf1 RR |
| CISH7078 | CGAAGGTAGGAGAAGGTCTT | 20 | AsCpf1 RR |
| CISH7079 | GAAGGTAGGAGAAGGTCTTG | 20 | AsCpf1 RR |
| CISH7080 | GCACCTTTGGCTCACTCTCT | 20 | AsCpf1 RR |
| CISH7081 | TCGAGGAGGTGGCAGAGGGT | 20 | AsCpf1 RR |
| CISH7082 | TGGAACTGCCCAAGCCAGTC | 20 | AsCpf1 RR |
| CISH7083 | AGGGACGGGGCCCACAGGGG | 20 | AsCpf1 RR |
| CISH7084 | GGGACGGGGCCCACAGGGGC | 20 | AsCpf1 RR |
| CISH7085 | CTCCACAGCCAGCAAAGGAC | 20 | AsCpf1 RR |
| CISH7086 | CAGCCAGCAAAGGACGAGGT | 20 | AsCpf1 RR |
| CISH7087 | CTGCCTTCTAGACCTCGTCC | 20 | AsCpf1 RR |
| CISH7088 | CCTAAGGAGGATGCGCCTAG | 20 | AsCpf1 RVR |
| CISH7089 | TGGCCTCCTGCACTGCTGAT | 20 | AsCpf1 RVR |
| CISH7090 | AGCAGTGCAGGAGGCCACAT | 20 | AsCpf1 RVR |
| CISH7091 | CCGACTCCAGCTTCCGTCTG | 20 | AsCpf1 RVR |
| CISH7092 | GGGGTTCCATTACGGCCAGC | 20 | AsCpf1 RVR |
| CISH7093 | CACAGCAGATCCTCCTCTGG | 20 | AsCpf1 RVR |
| CISH7094 | ATTGCCCCGTACAGTCAGAG | 21 | SauCas9 |
| CISH7095 | CCCGTACAGTCAGAGCTGGA | 21 | SauCas9 |
| CISH7096 | TGGTGGAGGAGCAGGCAGTG | 21 | SauCas9 |
| CISH7097 | TCCTTAGGCATAGGCAGGGC | 21 | SauCas9 |
| CISH7098 | CGGCCCTGCCTATGCCTAAG | 21 | SauCas9 |
| CISH7099 | TAGGCATAGGCAGGGCCGGG | 21 | SauCas9 |
| CISH7100 | AGGCAGGGCCGGGGTGGGAG | 21 | SauCas9 |
| CISH7101 | GCAGGATCGGGGCTGTCGCT | 21 | SauCas9 |
| CISH7102 | CTGCACAAGGCTGACCACAT | 21 | SauCas9 |
| CISH7103 | TGCACAAGGCTGACCACATC | 21 | SauCas9 |
| CISH7104 | CTGACCACATCCGGAAAGGC | 21 | SauCas9 |
| CISH7105 | GGCCACGCATCCTGGCCTTT | 21 | SauCas9 |
| CISH7106 | GCGTGGCCTGGACAAGCAGT | 21 | SauCas9 |
| CISH7107 | GACAAGCAGTTGGAGTCCAG | 21 | SauCas9 |
| CISH7108 | GTTGGAGTCCAGACGGAAGC | 21 | SauCas9 |
| CISH7109 | ATGCGTACATTGGTGGGGCC | 21 | SauCas9 |
| CISH7110 | TGGCCCCACCAATGTACGCA | 21 | SauCas9 |
| CISH7111 | GCTACCTGTTCACGCTGTCA | 21 | SauCas9 |
| CISH7112 | TGACAGCGTGAACAGGTAGC | 21 | SauCas9 |
| CISH7113 | GTCGGGCCTCGCTGGCCGTA | 21 | SauCas9 |
| CISH7114 | GCACTTGCCTAGGCTGGTAT | 21 | SauCas9 |
| CISH7115 | GGGAATCTGGTGAGTCTGAG | 21 | SauCas9 |
| CISH7116 | CTCACCAGATTCCCGAAGGT | 21 | SauCas9 |
| CISH7117 | CTCCTACCTTCGGGAATCTG | 21 | SauCas9 |
| CISH7118 | CAAGACCTTCTCCTACCTTC | 21 | SauCas9 |
| CISH7119 | CCAAGACCTTCTCCTACCTT | 21 | SauCas9 |
| CISH7120 | GCCAAGACCTTCTCCTACCT | 21 | SauCas9 |
| CISH7121 | TATGCACAGCAGATCCTCCT | 21 | SauCas9 |
| CISH7122 | CAAAGGTGCTGGACCCAGAG | 21 | SauCas9 |
| CISH7123 | GGCTCACTCTCTGTCTGGGC | 21 | SauCas9 |
| CISH7124 | AGGGTACCCCAGCCCAGACA | 21 | SauCas9 |
| CISH7125 | AGAGGGTACCCCAGCCCAGA | 21 | SauCas9 |
| CISH7126 | GTACCCTCTGCCACCTCCTC | 21 | SauCas9 |
| CISH7127 | CCTTCCTCGAGGAGGTGGCA | 21 | SauCas9 |
| CISH7128 | ATGACTGGCTTGGGCAGTTC | 21 | SauCas9 |
| CISH7129 | GGCCCCTGTGGGCCCCGTCC | 21 | SauCas9 |
| CISH7130 | AGGACGAGGTCTAGAAGGCA | 21 | SauCas9 |

In some embodiments the gRNA for use in the disclosure is a gRNA targeting B2M (B2M gRNA). In some embodiments, the gRNA targeting B2M is one or more of the gRNAs described in Table 8.

TABLE 8
B2M gRNAs

| gRNA | gRNA Targeting Domain | | |
|---|---|---|---|
| name | Target sequence (DNA) | Length | Enzyme |
| B2M1 | TATAAGTGGAGGCGTCGCGC | 20 | SpyCas9 |
| B2M2 | GGGCACGCGTTTAATATAAG | 20 | SpyCas9 |
| B2M3 | ACTCACGCTGGATAGCCTCC | 20 | SpyCas9 |
| B2M4 | GGCCGAGATGTCTCGCTCCG | 20 | SpyCas9 |
| B2M5 | CACGCGTTTAATATAAGTGG | 20 | SpyCas9 |
| B2M6 | AAGTGGAGGCGTCGCGCTGG | 20 | SpyCas9 |
| B2M7 | GAGTAGCGCGAGCACAGCTA | 20 | SpyCas9 |
| B2M8 | AGTGGAGGCGTCGCGCTGGC | 20 | SpyCas9 |
| B2M9 | GCCCGAATGCTGTCAGCTTC | 20 | SpyCas9 |
| B2M10 | CGCGAGCACAGCTAAGGCCA | 20 | SpyCas9 |
| B2M11 | CTCGCGCTACTCTCTCTTTC | 20 | SpyCas9 |
| B2M12 | GGCCACGGAGCGAGACATCT | 20 | SpyCas9 |
| B2M13 | CGTGAGTAAACCTGAATCTT | 20 | SpyCas9 |
| B2M14 | AGTCACATGGTTCACACGGC | 20 | SpyCas9 |
| B2M15 | AAGTCAACTTCAATGTCGGA | 20 | SpyCas9 |
| B2M16 | CAGTAAGTCAACTTCAATGT | 20 | SpyCas9 |
| B2M17 | ACCCAGACACATAGCAATTC | 20 | SpyCas9 |
| B2M18 | GCATACTCATCTTTTTCAGT | 20 | SpyCas9 |
| B2M19 | ACAGCCCAAGATAGTTAAGT | 20 | SpyCas9 |
| B2M20 | GGCATACTCATCTTTTTCAG | 20 | SpyCas9 |
| B2M21 | TTCCTGAAGCTGACAGCATT | 20 | SpyCas9 |
| B2M22 | TCACGTCATCCAGCAGAGAA | 20 | SpyCas9 |
| B2M23 | CAGCCCAAGATAGTTAAGTG | 20 | SpyCas9 |
| B2M-c1 | AAUUCUCUCUCCAUUCUU | 18 | AsCpf1 |
| B2M-c2 | AAUUCUCUCUCCAUUCUUC | 19 | AsCpf1 |
| B2M-c3 | AAUUCUCUCUCCAUUCUUCA | 20 | AsCpf1 |
| B2M-c4 | AAUUCUCUCUCCAUUCUUCAG | 21 | AsCpf1 |
| B2M-c5 | AAUUCUCUCUCCAUUCUUCAGU | 22 | AsCpf1 |
| B2M-c6 | AAUUCUCUCUCCAUUCUUCAGUA | 23 | AsCpf1 |
| B2M-c7 | AAUUCUCUCUCCAUUCUUCAGUAA | 24 | AsCpf1 |
| B2M-c8 | ACUUUCCAUUCUCUGCUG | 18 | AsCpf1 |
| B2M-c9 | ACUUUCCAUUCUCUGCUGG | 19 | AsCpf1 |
| B2M-c10 | ACUUUCCAUUCUCUGCUGGA | 20 | AsCpf1 |
| B2M-c11 | ACUUUCCAUUCUCUGCUGGAU | 21 | AsCpf1 |
| B2M-c12 | ACUUUCCAUUCUCUGCUGGAUG | 22 | AsCpf1 |
| B2M-c13 | ACUUUCCAUUCUCUGCUGGAUGA | 23 | AsCpf1 |
| B2M-c14 | ACUUUCCAUUCUCUGCUGGAUGAC | 24 | AsCpf1 |
| B2M-c15 | AGCAAGGACUGGUCUUUC | 18 | AsCpf1 |
| B2M-c16 | AGCAAGGACUGGUCUUUCU | 19 | AsCpf1 |
| B2M-c17 | AGCAAGGACUGGUCUUUCUA | 20 | AsCpf1 |
| B2M-c18 | AGCAAGGACUGGUCUUUCUAU | 21 | AsCpf1 |
| B2M-c19 | AGCAAGGACUGGUCUUUCUAUC | 22 | AsCpf1 |
| B2M-c20 | AGCAAGGACUGGUCUUUCUAUCU | 23 | AsCpf1 |
| B2M-c21 | AGCAAGGACUGGUCUUUCUAUCUC | 24 | AsCpf1 |
| B2M-c22 | AGUGGGGGUGAAUUCAGU | 18 | AsCpf1 |
| B2M-c23 | AGUGGGGGUGAAUUCAGUG | 19 | AsCpf1 |
| B2M-c24 | AGUGGGGGUGAAUUCAGUGU | 20 | AsCpf1 |
| B2M-c25 | AGUGGGGGUGAAUUCAGUGUA | 21 | AsCpf1 |
| B2M-c26 | AGUGGGGGUGAAUUCAGUGUAG | 22 | AsCpf1 |
| B2M-c27 | AGUGGGGGUGAAUUCAGUGUAGU | 23 | AsCpf1 |
| B2M-c28 | AGUGGGGGUGAAUUCAGUGUAGUA | 24 | AsCpf1 |
| B2M-c29 | AUCCAUCCGACAUUGAAG | 18 | AsCpf1 |
| B2M-c30 | AUCCAUCCGACAUUGAAGU | 19 | AsCpf1 |
| B2M-c31 | AUCCAUCCGACAUUGAAGUU | 20 | AsCpf1 |
| B2M-c32 | AUCCAUCCGACAUUGAAGUUG | 21 | AsCpf1 |
| B2M-c33 | AUCCAUCCGACAUUGAAGUUGA | 22 | AsCpf1 |
| B2M-c34 | AUCCAUCCGACAUUGAAGUUGAC | 23 | AsCpf1 |
| B2M-c35 | AUCCAUCCGACAUUGAAGUUGACU | 24 | AsCpf1 |
| B2M-c36 | CAAUUCUCUCUCCAUUCU | 18 | AsCpf1 |
| B2M-c37 | CAAUUCUCUCUCCAUUCUU | 19 | AsCpf1 |
| B2M-c38 | CAAUUCUCUCUCCAUUCUUC | 20 | AsCpf1 |
| B2M-c39 | CAAUUCUCUCUCCAUUCUUCA | 21 | AsCpf1 |
| B2M-c40 | CAAUUCUCUCUCCAUUCUUCAG | 22 | AsCpf1 |
| B2M-c41 | CAAUUCUCUCUCCAUUCUUCAGU | 23 | AsCpf1 |
| B2M-c42 | CAAUUCUCUCUCCAUUCUUCAGUA | 24 | AsCpf1 |
| B2M-c43 | CAGUGGGGGUGAAUUCAG | 18 | AsCpf1 |
| B2M-c44 | CAGUGGGGGUGAAUUCAGU | 19 | AsCpf1 |
| B2M-c45 | CAGUGGGGGUGAAUUCAGUG | 20 | AsCpf1 |
| B2M-c46 | CAGUGGGGGUGAAUUCAGUGU | 21 | AsCpf1 |
| B2M-c47 | CAGUGGGGGUGAAUUCAGUGUA | 22 | AsCpf1 |
| B2M-c48 | CAGUGGGGGUGAAUUCAGUGUAG | 23 | AsCpf1 |
| B2M-c49 | CAGUGGGGGUGAAUUCAGUGUAGU | 24 | AsCpf1 |
| B2M-c50 | CAUUCUCUGCUGGAUGAC | 18 | AsCpf1 |
| B2M-c51 | CAUUCUCUGCUGGAUGACG | 19 | AsCpf1 |
| B2M-c52 | CAUUCUCUGCUGGAUGACGU | 20 | AsCpf1 |
| B2M-c53 | CAUUCUCUGCUGGAUGACGUG | 21 | AsCpf1 |
| B2M-c54 | CAUUCUCUGCUGGAUGACGUGA | 22 | AsCpf1 |
| B2M-c55 | CAUUCUCUGCUGGAUGACGUGAG | 23 | AsCpf1 |
| B2M-c56 | CAUUCUCUGCUGGAUGACGUGAGU | 24 | AsCpf1 |
| B2M-c57 | CCCGAUAUUCCUCAGGUA | 18 | AsCpf1 |
| B2M-c58 | CCCGAUAUUCCUCAGGUAC | 19 | AsCpf1 |
| B2M-c59 | CCCGAUAUUCCUCAGGUACU | 20 | AsCpf1 |
| B2M-c60 | CCCGAUAUUCCUCAGGUACUC | 21 | AsCpf1 |
| B2M-c61 | CCCGAUAUUCCUCAGGUACUCC | 22 | AsCpf1 |
| B2M-c62 | CCCGAUAUUCCUCAGGUACUCCA | 23 | AsCpf1 |
| B2M-c63 | CCCGAUAUUCCUCAGGUACUCCAA | 24 | AsCpf1 |
| B2M-c64 | CCGAUAUUCCUCAGGUAC | 18 | AsCpf1 |
| B2M-c65 | CCGAUAUUCCUCAGGUACU | 19 | AsCpf1 |
| B2M-c66 | CCGAUAUUCCUCAGGUACUC | 20 | AsCpf1 |
| B2M-c67 | CCGAUAUUCCUCAGGUACUCC | 21 | AsCpf1 |
| B2M-c68 | CCGAUAUUCCUCAGGUACUCCA | 22 | AsCpf1 |
| B2M-c69 | CCGAUAUUCCUCAGGUACUCCAA | 23 | AsCpf1 |
| B2M-c70 | CCGAUAUUCCUCAGGUACUCCAAA | 24 | AsCpf1 |
| B2M-c71 | CUCACGUCAUCCAGCAGA | 18 | AsCpf1 |
| B2M-c72 | CUCACGUCAUCCAGCAGAG | 19 | AsCpf1 |
| B2M-c73 | CUCACGUCAUCCAGCAGAGA | 20 | AsCpf1 |
| B2M-c74 | CUCACGUCAUCCAGCAGAGAA | 21 | AsCpf1 |
| B2M-c75 | CUCACGUCAUCCAGCAGAGAAU | 22 | AsCpf1 |
| B2M-c76 | CUCACGUCAUCCAGCAGAGAAUG | 23 | AsCpf1 |
| B2M-c77 | CUCACGUCAUCCAGCAGAGAAUGG | 24 | AsCpf1 |
| B2M-c78 | CUGAAUUGCUAUGUGUCU | 18 | AsCpf1 |
| B2M-c79 | CUGAAUUGCUAUGUGUCUG | 19 | AsCpf1 |
| B2M-c80 | CUGAAUUGCUAUGUGUCUGG | 20 | AsCpf1 |
| B2M-c81 | CUGAAUUGCUAUGUGUCUGGG | 21 | AsCpf1 |
| B2M-c82 | CUGAAUUGCUAUGUGUCUGGGU | 22 | AsCpf1 |
| B2M-c83 | CUGAAUUGCUAUGUGUCUGGGUU | 23 | AsCpf1 |
| B2M-c84 | CUGAAUUGCUAUGUGUCUGGGUUU | 24 | AsCpf1 |
| B2M-c85 | GAGUACCUGAGGAAUAUC | 18 | AsCpf1 |
| B2M-c86 | GAGUACCUGAGGAAUAUCG | 19 | AsCpf1 |
| B2M-c87 | GAGUACCUGAGGAAUAUCGG | 20 | AsCpf1 |
| B2M-c88 | GAGUACCUGAGGAAUAUCGGG | 21 | AsCpf1 |
| B2M-c89 | GAGUACCUGAGGAAUAUCGGGA | 22 | AsCpf1 |
| B2M-c90 | GAGUACCUGAGGAAUAUCGGGAA | 23 | AsCpf1 |
| B2M-c91 | GAGUACCUGAGGAAUAUCGGGAAA | 24 | AsCpf1 |
| B2M-c92 | UAUCUCUUGUACUACACU | 18 | AsCpf1 |
| B2M-c93 | UAUCUCUUGUACUACACUG | 19 | AsCpf1 |
| B2M-c94 | UAUCUCUUGUACUACACUGA | 20 | AsCpf1 |
| B2M-c95 | UAUCUCUUGUACUACACUGAA | 21 | AsCpf1 |
| B2M-c96 | UAUCUCUUGUACUACACUGAAU | 22 | AsCpf1 |
| B2M-c97 | UAUCUCUUGUACUACACUGAAUU | 23 | AsCpf1 |
| B2M-c98 | UAUCUCUUGUACUACACUGAAUUC | 24 | AsCpf1 |
| B2M-c99 | UCAAUUCUCUCUCCAUUC | 18 | AsCpf1 |
| B2M-c100 | UCAAUUCUCUCUCCAUUCU | 19 | AsCpf1 |
| B2M-c101 | UCAAUUCUCUCUCCAUUCUU | 20 | AsCpf1 |
| B2M-c102 | UCAAUUCUCUCUCCAUUCUUC | 21 | AsCpf1 |
| B2M-c103 | UCAAUUCUCUCUCCAUUCUUCA | 22 | AsCpf1 |
| B2M-c104 | UCAAUUCUCUCUCCAUUCUUCAG | 23 | AsCpf1 |
| B2M-c105 | UCAAUUCUCUCUCCAUUCUUCAGU | 24 | AsCpf1 |
| B2M-c106 | UCACAGCCCAAGAUAGUU | 18 | AsCpf1 |
| B2M-c107 | UCACAGCCCAAGAUAGUUA | 19 | AsCpf1 |
| B2M-c108 | UCACAGCCCAAGAUAGUUAA | 20 | AsCpf1 |
| B2M-c109 | UCACAGCCCAAGAUAGUUAAG | 21 | AsCpf1 |
| B2M-c110 | UCACAGCCCAAGAUAGUUAAGU | 22 | AsCpf1 |
| B2M-c111 | UCACAGCCCAAGAUAGUUAAGUG | 23 | AsCpf1 |
| B2M-c112 | UCACAGCCCAAGAUAGUUAAGUGG | 24 | AsCpf1 |
| B2M-c113 | UCAGUGGGGGUGAAUUCA | 18 | AsCpf1 |
| B2M-c114 | UCAGUGGGGGUGAAUUCAG | 19 | AsCpf1 |
| B2M-c115 | UCAGUGGGGGUGAAUUCAGU | 20 | AsCpf1 |
| B2M-c116 | UCAGUGGGGGUGAAUUCAGUG | 21 | AsCpf1 |
| B2M-c117 | UCAGUGGGGGUGAAUUCAGUGU | 22 | AsCpf1 |
| B2M-c118 | UCAGUGGGGGUGAAUUCAGUGUA | 23 | AsCpf1 |
| B2M-c119 | UCAGUGGGGGUGAAUUCAGUGUAG | 24 | AsCpf1 |
| B2M-c120 | UGGCCUGGAGGCUAUCCA | 18 | AsCpf1 |
| B2M-c121 | UGGCCUGGAGGCUAUCCAG | 19 | AsCpf1 |
| B2M-c122 | UGGCCUGGAGGCUAUCCAGC | 20 | AsCpf1 |
| B2M-c123 | UGGCCUGGAGGCUAUCCAGCG | 21 | AsCpf1 |
| B2M-c124 | UGGCCUGGAGGCUAUCCAGCGU | 22 | AsCpf1 |
| B2M-c125 | UGGCCUGGAGGCUAUCCAGCGUG | 23 | AsCpf1 |
| B2M-c126 | UGGCCUGGAGGCUAUCCAGCGUGA | 24 | AsCpf1 |
| B2M-c127 | AUAGAUCGAGACAUGUAA | 18 | AsCpf1 |
| B2M-c128 | AUAGAUCGAGACAUGUAAG | 19 | AsCpf1 |
| B2M-c129 | AUAGAUCGAGACAUGUAAGC | 20 | AsCpf1 |
| B2M-c130 | AUAGAUCGAGACAUGUAAGCA | 21 | AsCpf1 |
| B2M-c131 | AUAGAUCGAGACAUGUAAGCAG | 22 | AsCpf1 |
| B2M-c132 | AUAGAUCGAGACAUGUAAGCAGC | 23 | AsCpf1 |
| B2M-c133 | AUAGAUCGAGACAUGUAAGCAGCA | 24 | AsCpf1 |
| B2M-c134 | CAUAGAUCGAGACAUGUA | 18 | AsCpf1 |
| B2M-c135 | CAUAGAUCGAGACAUGUAA | 19 | AsCpf1 |
| B2M-c136 | CAUAGAUCGAGACAUGUAAG | 20 | AsCpf1 |
| B2M-c137 | CAUAGAUCGAGACAUGUAAGC | 21 | AsCpf1 |
| B2M-c138 | CAUAGAUCGAGACAUGUAAGCA | 22 | AsCpf1 |
| B2M-c139 | CAUAGAUCGAGACAUGUAAGCAG | 23 | AsCpf1 |
| B2M-c140 | CAUAGAUCGAGACAUGUAAGCAGC | 24 | AsCpf1 |
| B2M-c141 | CUCCACUGUCUUUUUCAU | 18 | AsCpf1 |
| B2M-c142 | CUCCACUGUCUUUUUCAUA | 19 | AsCpf1 |
| B2M-c143 | CUCCACUGUCUUUUUCAUAG | 20 | AsCpf1 |
| B2M-c144 | CUCCACUGUCUUUUUCAUAGA | 21 | AsCpf1 |
| B2M-c145 | CUCCACUGUCUUUUUCAUAGAU | 22 | AsCpf1 |
| B2M-c146 | CUCCACUGUCUUUUUCAUAGAUC | 23 | AsCpf1 |
| B2M-c147 | CUCCACUGUCUUUUUCAUAGAUCG | 24 | AsCpf1 |
| B2M-c148 | UCAUAGAUCGAGACAUGU | 18 | AsCpf1 |
| B2M-c149 | UCAUAGAUCGAGACAUGUA | 19 | AsCpf1 |
| B2M-c150 | UCAUAGAUCGAGACAUGUAA | 20 | AsCpf1 |
| B2M-c151 | UCAUAGAUCGAGACAUGUAAG | 21 | AsCpf1 |
| B2M-c152 | UCAUAGAUCGAGACAUGUAAGC | 22 | AsCpf1 |
| B2M-c153 | UCAUAGAUCGAGACAUGUAAGCA | 23 | AsCpf1 |
| B2M-c154 | UCAUAGAUCGAGACAUGUAAGCAG | 24 | AsCpf1 |
| B2M-c155 | UCCACUGUCUUUUUCAUA | 18 | AsCpf1 |
| B2M-c156 | UCCACUGUCUUUUUCAUAG | 19 | AsCpf1 |
| B2M-c157 | UCCACUGUCUUUUUCAUAGA | 20 | AsCpf1 |
| B2M-c158 | UCCACUGUCUUUUUCAUAGAU | 21 | AsCpf1 |
| B2M-c159 | UCCACUGUCUUUUUCAUAGAUC | 22 | AsCpf1 |
| B2M-c160 | UCCACUGUCUUUUUCAUAGAUCG | 23 | AsCpf1 |
| B2M-c161 | UCCACUGUCUUUUUCAUAGAUCGA | 24 | AsCpf1 |
| B2M-c162 | UCUCCACUGUCUUUUUCA | 18 | AsCpf1 |
| B2M-c163 | UCUCCACUGUCUUUUUCAU | 19 | AsCpf1 |
| B2M-c164 | UCUCCACUGUCUUUUUCAUA | 20 | AsCpf1 |
| B2M-c165 | UCUCCACUGUCUUUUUCAUAG | 21 | AsCpf1 |
| B2M-c166 | UCUCCACUGUCUUUUUCAUAGA | 22 | AsCpf1 |
| B2M-c167 | UCUCCACUGUCUUUUUCAUAGAU | 23 | AsCpf1 |
| B2M-c168 | UCUCCACUGUCUUUUUCAUAGAUC | 24 | AsCpf1 |
| B2M-c169 | UUCUCCACUGUCUUUUUC | 18 | AsCpf1 |
| B2M-c170 | UUCUCCACUGUCUUUUUCA | 19 | AsCpf1 |
| B2M-c171 | UUCUCCACUGUCUUUUUCAU | 20 | AsCpf1 |
| B2M-c172 | UUCUCCACUGUCUUUUUCAUA | 21 | AsCpf1 |
| B2M-c173 | UUCUCCACUGUCUUUUUCAUAG | 22 | AsCpf1 |
| B2M-c174 | UUCUCCACUGUCUUUUUCAUAGA | 23 | AsCpf1 |
| B2M-c175 | UUCUCCACUGUCUUUUUCAUAGAU | 24 | AsCpf1 |
| B2M-c176 | UUUCUCCACUGUCUUUUU | 18 | AsCpf1 |
| B2M-c177 | UUUCUCCACUGUCUUUUUC | 19 | AsCpf1 |
| B2M-c178 | UUUCUCCACUGUCUUUUUCA | 20 | AsCpf1 |
| B2M-c179 | UUUCUCCACUGUCUUUUUCAU | 21 | AsCpf1 |
| B2M-c180 | UUUCUCCACUGUCUUUUUCAUA | 22 | AsCpf1 |
| B2M-c181 | UUUCUCCACUGUCUUUUUCAUAG | 23 | AsCpf1 |
| B2M-c182 | UUUCUCCACUGUCUUUUUCAUAGA | 24 | AsCpf1 |
| B2M-c183 | UUUUCUCCACUGUCUUUU | 18 | AsCpf1 |
| B2M-c184 | UUUUCUCCACUGUCUUUUU | 19 | AsCpf1 |
| B2M-c185 | UUUUCUCCACUGUCUUUUUC | 20 | AsCpf1 |
| B2M-c186 | UUUUCUCCACUGUCUUUUUCA | 21 | AsCpf1 |
| B2M-c187 | UUUUCUCCACUGUCUUUUUCAU | 22 | AsCpf1 |
| B2M-c188 | UUUUCUCCACUGUCUUUUUCAUA | 23 | AsCpf1 |
| B2M-c189 | UUUUCUCCACUGUCUUUUUCAUAG | 24 | AsCpf1 |

In some embodiments the gRNA for use in the disclosure is a gRNA targeting NKG2A (NKG2A gRNA). In some embodiments, the gRNA targeting NKG2A is one or more of the gRNAs described in Table 9.

TABLE 9
NKG2A gRNAs

| | gRNA Targeting Domain | | |
|---|---|---|---|
| Name | Sequence (DNA) | Length | Enzyme |
| NKG2A55 | GAGGTAAAGCGTTTGCATTTG | 21 | AsCpf1 |
| NKG2A56 | CCTCTAAAGCTTATGCTTACA | 21 | AsCpf1 |
| NKG2A57 | AGTCGATTTACTTGTAGCACT | 21 | AsCpf1 |
| NKG2A58 | CTTGTAGCACTGCACAGTTAA | 21 | AsCpf1 |
| NKG2A59 | TCCATTACAGGATAAAAGACT | 21 | AsCpf1 |
| NKG2A60 | CTCCATTACAGGATAAAAGAC | 21 | AsCpf1 |
| NKG2A61 | TCTCCATTACAGGATAAAAGA | 21 | AsCpf1 |
| NKG2A62 | ATCCTGTAATGGAGAAAAATC | 21 | AsCpf1 |
| NKG2A63 | TCCTGTAATGGAGAAAAATCC | 21 | AsCpf1 |
| NKG2A136 | AAACATGAGTAAGTTGTTTTG | 21 | AsCpf1 |
| NKG2A137 | GCTTTCAAACATGAGTAAGTT | 21 | AsCpf1 |
| NKG2A138 | AAAGCCAAACCATTCATTGTC | 21 | AsCpf1 |
| NKG2A139 | GTAACAGCAGTCATCATCCAT | 21 | AsCpf1 |
| NKG2A140 | ACCATCCTCATGGATTGGTGT | 21 | AsCpf1 |
| NKG2A141 | TGTCCATCATTTCACCATCCT | 21 | AsCpf1 |
| NKG2A142 | GAAATTTCTGTCCATCATTTC | 21 | AsCpf1 |
| NKG2A143 | AGAAATTTCTGTCCATCATTT | 21 | AsCpf1 |
| NKG2A144 | TTTTAGAAATTTCTGTCCATC | 21 | AsCpf1 |
| NKG2A145 | CTTTTAGAAATTTCTGTCCAT | 21 | AsCpf1 |
| NKG2A146 | TTTTCTTTTAGAAATTTCTGT | 21 | AsCpf1 |
| NKG2A147 | TAAAAGAAAAGAAAGAATTTT | 21 | AsCpf1 |
| NKG2A270 | AAACATTTACATCTTACCATT | 21 | AsCpf1 |
| NKG2A271 | CATCTTACCATTTCTTCTTCA | 21 | AsCpf1 |
| NKG2A272 | TATAGATAATGAAGAAGAAAT | 21 | AsCpf1 |
| NKG2A273 | TTCTTCATTATCTATAGAAAG | 21 | AsCpf1 |
| NKG2A274 | CTGGCCTGTACTTCGAAGAAC | 21 | AsCpf1 |
| NKG2A275 | CTTACCAATGTAGTAACAACT | 21 | AsCpf1 |
| NKG2A276 | GCACGTCATTGTGGCCATTGT | 21 | AsCpf1 |
| NKG2A277 | TTTAGCACGTCATTGTGGCCA | 21 | AsCpf1 |
| NKG2A414 | CCATCAGCTCCAGAGAAGCTC | 21 | AsCpf1 |
| NKG2A415 | TCTCCCTGCAGATTTACCATC | 21 | AsCpf1 |
| NKG2A437 | AAATGCTTTACCTTTGCAGTG | 21 | AsCpf1 |
| NKG2A438 | AATGCTTTACCTTTGCAGTGA | 21 | AsCpf1 |
| NKG2A439 | CCTTTGCAGTGATAGGTTTTG | 21 | AsCpf1 |
| NKG2A440 | CAGTGATAGGTTTTGTCATTC | 21 | AsCpf1 |
| NKG2A441 | AAGGGAATGACAAAACCTATC | 21 | AsCpf1 |
| NKG2A442 | CAAGGGAATGACAAAACCTAT | 21 | AsCpf1 |
| NKG2A443 | GTCATTCCCTTGAAAATCCTG | 21 | AsCpf1 |
| NKG2A444 | TCATTCCCTTGAAAATCCTGA | 21 | AsCpf1 |
| NKG2A445 | TGAAGGTTTAATTCCGCATAG | 21 | AsCpf1 |
| NKG2A446 | GAAGGTTTAATTCCGCATAGG | 21 | AsCpf1 |
| NKG2A447 | AAGGTTTAATTCCGCATAGGT | 21 | AsCpf1 |
| NKG2A448 | ATTCCGCATAGGTTATTTCCT | 21 | AsCpf1 |
| NKG2A449 | GCAACTGAACAGGAAATAACC | 21 | AsCpf1 |
| NKG2A450 | AGCAACTGAACAGGAAATAAC | 21 | AsCpf1 |
| NKG2A451 | CTGTTCAGTTGCTAAAATGGA | 21 | AsCpf1 |
| NKG2A452 | TATTGCCTTTAGGTTTTCGTT | 21 | AsCpf1 |
| NKG2A453 | ATTGCCTTTAGGTTTTCGTTG | 21 | AsCpf1 |
| NKG2A454 | TTGCCTTTAGGTTTTCGTTGC | 21 | AsCpf1 |
| NKG2A455 | GGTTTTCGTTGCTGCCTCTTT | 21 | AsCpf1 |
| NKG2A456 | CGTTGCTGCCTCTTTGGGTTT | 21 | AsCpf1 |
| NKG2A457 | GTTGCTGCCTCTTTGGGTTTG | 21 | AsCpf1 |
| NKG2A458 | GGTTTGGGGGCAGATTCAGGT | 21 | AsCpf1 |
| NKG2A459 | GGGGCAGATTCAGGTCTGAGT | 21 | AsCpf1 |

In some embodiments the gRNA for use in the disclosure is a gRNA targeting PD1. In some embodiments the gRNA for use in the disclosure is a gRNA targeting PD1. The gRNAs garneting B2M and PD1 for use in the disclosure are further described in WO2015161276 and WO2017152015 by Welstead et al. (“Welstead”); both incorporated in their entirety herein by reference.

RNA-Guided Nucleases

RNA-guided nucleases according to the present disclosure include, but are not limited to, naturally-occurring Class 2 CRISPR nucleases such as Cas9, and Cpf1, as well as other nucleases derived or obtained therefrom. In functional terms, RNA-guided nucleases are defined as those nucleases that: (a) interact with (e.g., complex with) a gRNA; and (b) together with the gRNA, associate with, and optionally cleave or modify, a target region of a DNA that includes (i) a sequence complementary to the targeting domain of the gRNA and, optionally, (ii) an additional sequence referred to as a “protospacer adjacent motif,” or “PAM,” which is described in greater detail below. As the following examples will illustrate, RNA-guided nucleases can be defined, in broad terms, by their PAM specificity and cleavage activity, even though variations may exist between individual RNA-guided nucleases that share the same PAM specificity or cleavage activity. Skilled artisans will appreciate that some aspects of the present disclosure relate to systems, methods and compositions that can be implemented using any suitable RNA-guided nuclease having a certain PAM specificity and/or cleavage activity. For this reason, unless otherwise specified, the term RNA-guided nuclease should be understood as a generic term, and not limited to any particular type (e.g. Cas9 vs. Cpf1), species (e.g. S. pyogenes vs. S. aureus) or variation (e.g., full-length vs. truncated or split; naturally-occurring PAM specificity vs. engineered PAM specificity, etc.) of RNA-guided nuclease.

The PAM sequence takes its name from its sequential relationship to the “protospacer” sequence that is complementary to gRNA targeting domains (or “spacers”). Together with protospacer sequences, PAM sequences define target regions or sequences for specific RNA-guided nuclease/gRNA combinations.

Various RNA-guided nucleases may require different sequential relationships between PAMs and protospacers. For example, Cas9 nucleases recognize PAM sequences that are 3′ of the protospacer, while

Cpf1, on the other hand, generally recognizes PAM sequences that are 5′ of the protospacer.

In addition to recognizing specific sequential orientations of PAMs and protospacers, RNA-guided nucleases can also recognize specific PAM sequences. S. aureus Cas9, for instance, recognizes a PAM sequence of NNGRRT or NNGRRV, wherein the N residues are immediately 3′ of the region recognized by the gRNA targeting domain. S. pyogenes Cas9 recognizes NGG PAM sequences. And F. novicida Cpf1 recognizes a TTN PAM sequence. PAM sequences have been identified for a variety of RNA-guided nucleases, and a strategy for identifying novel PAM sequences has been described by Shmakov et al., 2015, Molecular Cell 60, 385-397, Nov. 5, 2015. It should also be noted that engineered RNA-guided nucleases can have PAM specificities that differ from the PAM specificities of reference molecules (for instance, in the case of an engineered RNA-guided nuclease, the reference molecule may be the naturally occurring variant from which the RNA-guided nuclease is derived, or the naturally occurring variant having the greatest amino acid sequence homology to the engineered RNA-guided nuclease).

In addition to their PAM specificity, RNA-guided nucleases can be characterized by their DNA cleavage activity: naturally-occurring RNA-guided nucleases typically form DSBs in target nucleic acids, but engineered variants have been produced that generate only SSBs (discussed above) Ran & Hsu, et al., Cell 154(6), 1380-1389, Sep. 12, 2013 (Ran), incorporated by reference herein), or that that do not cut at all.

Cas9

Crystal structures have been determined for S. pyogenes Cas9 (Jinek 2014), and for S. aureus Cas9 in complex with a unimolecular guide RNA and a target DNA (Nishimasu 2014; Anders 2014; and Nishimasu 2015).

A naturally occurring Cas9 protein comprises two lobes: a recognition (REC) lobe and a nuclease (NUC) lobe; each of which comprise particular structural and/or functional domains. The REC lobe comprises an arginine-rich bridge helix (BH) domain, and at least one REC domain (e.g. a REC1 domain and, optionally, a REC2 domain). The REC lobe does not share structural similarity with other known proteins, indicating that it is a unique functional domain. While not wishing to be bound by any theory, mutational analyses suggest specific functional roles for the BH and REC domains: the BH domain appears to play a role in gRNA:DNA recognition, while the REC domain is thought to interact with the repeat:anti-repeat duplex of the gRNA and to mediate the formation of the Cas9/gRNA complex.

The NUC lobe comprises a RuvC domain, an HNH domain, and a PAM-interacting (PI) domain. The RuvC domain shares structural similarity to retroviral integrase superfamily members and cleaves the non-complementary (i.e. bottom) strand of the target nucleic acid. It may be formed from two or more split RuvC motifs (such as RuvC I, RuvCII, and RuvCIII in S. pyogenes and S. aureus). The HNH domain, meanwhile, is structurally similar to HNN endonuclease motifs, and cleaves the complementary (i.e. top) strand of the target nucleic acid. The PI domain, as its name suggests, contributes to PAM specificity.

While certain functions of Cas9 are linked to (but not necessarily fully determined by) the specific domains set forth above, these and other functions may be mediated or influenced by other Cas9 domains, or by multiple domains on either lobe. For instance, in S. pyogenes Cas9, as described in Nishimasu 2014, the repeat:antirepeat duplex of the gRNA falls into a groove between the REC and NUC lobes, and nucleotides in the duplex interact with amino acids in the BH, PI, and REC domains. Some nucleotides in the first stem loop structure also interact with amino acids in multiple domains (PI, BH and REC1), as do some nucleotides in the second and third stem loops (RuvC and PI domains).

Cpf1

The crystal structure of Acidaminococcus sp. Cpf1 in complex with crRNA and a double-stranded (ds) DNA target including a TTTN PAM sequence has been solved by Yamano et al. (Cell. 2016 May 5; 165(4): 949-962 (Yamano), incorporated by reference herein). Cpf1, like Cas9, has two lobes: a REC (recognition) lobe, and a NUC (nuclease) lobe. The REC lobe includes REC1 and REC2 domains, which lack similarity to any known protein structures. The NUC lobe, meanwhile, includes three RuvC domains (RuvC-I, —II and -III) and a BH domain. However, in contrast to Cas9, the Cpf1 REC lobe lacks an HNH domain, and includes other domains that also lack similarity to known protein structures: a structurally unique PI domain, three Wedge (WED) domains (WED-I, —II and —III), and a nuclease (Nuc) domain.

While Cas9 and Cpf1 share similarities in structure and function, it should be appreciated that certain Cpf1 activities are mediated by structural domains that are not analogous to any Cas9 domains. For instance, cleavage of the complementary strand of the target DNA appears to be mediated by the Nuc domain, which differs sequentially and spatially from the HNH domain of Cas9. Additionally, the non-targeting portion of Cpf1 gRNA (the handle) adopts a pseudoknot structure, rather than a stem loop structure formed by the repeat:antirepeat duplex in Cas9 gRNAs.

Modifications of RNA-Guided Nucleases

The RNA-guided nucleases described above have activities and properties that can be useful in a variety of applications, but the skilled artisan will appreciate that RNA-guided nucleases can also be modified in certain instances, to alter cleavage activity, PAM specificity, or other structural or functional features.

Turning first to modifications that alter cleavage activity, mutations that reduce or eliminate the activity of domains within the NUC lobe have been described above. Exemplary mutations that may be made in the RuvC domains, in the Cas9 HNH domain, or in the Cpf1 Nuc domain are described in Ran and Yamano, as well as in Cotta-Ramusino. In general, mutations that reduce or eliminate activity in one of the two nuclease domains result in RNA-guided nucleases with nickase activity, but it should be noted that the type of nickase activity varies depending on which domain is inactivated. As one example, inactivation of a RuvC domain or of a Cas9 HNH domain results in a nickase.

Modifications of PAM specificity relative to naturally occurring Cas9 reference molecules has been described by Kleinstiver et al. for both S. pyogenes (Kleinstiver et al., Nature. 2015 Jul. 23; 523(7561):481-5 (Kleinstiver I) and S. aureus (Kleinstiver et al., Nat Biotechnol. 2015 December; 33(12): 1293-1298 (Klienstiver II)). Kleinstiver et al. have also described modifications that improve the targeting fidelity of Cas9 (Nature, 2016 Jan. 28; 529, 490-495 (Kleinstiver III)). Each of these references is incorporated by reference herein.

RNA-guided nucleases have been split into two or more parts, as described by Zetsche et al. (Nat Biotechnol. 2015 February; 33(2):139-42 (Zetsche II), incorporated by reference), and by Fine et al. (Sci Rep. 2015 Jul. 1; 5:10777 (Fine), incorporated by reference).

RNA-guided nucleases can be, in certain embodiments, size-optimized or truncated, for instance via one or more deletions that reduce the size of the nuclease while still retaining gRNA association, target and PAM recognition, and cleavage activities. In certain embodiments, RNA guided nucleases are bound, covalently or non-covalently, to another polypeptide, nucleotide, or other structure, optionally by means of a linker. Exemplary bound nucleases and linkers are described by Guilinger et al., Nature Biotechnology 32, 577-582 (2014), which is incorporated by reference for all purposes herein.

RNA-guided nucleases also optionally include a tag, such as, but not limited to, a nuclear localization signal to facilitate movement of RNA-guided nuclease protein into the nucleus. In certain embodiments, the RNA-guided nuclease can incorporate C- and/or N-terminal nuclear localization signals. Nuclear localization sequences are known in the art and are described in Maeder and elsewhere.

The foregoing list of modifications is intended to be exemplary in nature, and the skilled artisan will appreciate, in view of the instant disclosure, that other modifications may be possible or desirable in certain applications. For brevity, therefore, exemplary systems, methods and compositions of the present disclosure are presented with reference to particular RNA-guided nucleases, but it should be understood that the RNA-guided nucleases used may be modified in ways that do not alter their operating principles. Such modifications are within the scope of the present disclosure.

Exemplary suitable nuclease variants include, but are not limited to, AsCpf1 variants comprising an M537R substitution, an H800A substitution, and/or an F870L substitution, or any combination thereof (numbering scheme according to AsCpf1 wild-type sequence). Other suitable modifications of the AsCpf1 amino acid sequence are known to those of ordinary skill in the art. Some exemplary sequences of wild-type AsCpf1 and AsCpf1 variants are provided below.

His-AsCpf1-sNLS-sNLS H800A amino acid sequence
(SEQ ID NO: [XX])
MGHHHHHHGSTQFEGFTNLYQVSKTLRFELIPQGKTLKHIQEQGFIEEDKAR
NDHYKELKPIIDRIYKTYADQCLQLVQLDWENLSAAIDSYRKEKTEETRNALIEEQATYRNA
IHDYFIGRTDNLTDAINKRHAEIYKGLFKAELFNGKVLKQLGTVTTTEHENALLRSFDKFTT
YFSGFYENRKNVFSAEDISTAIPHRIVQDNFPKFKENCHIFTRLITAVPSLREHFENVKKAI
GIFVSTSIEEVFSFPFYNQLLTQTQIDLYNQLLGGISREAGTEKIKGLNEVLNLAIQKNDET
AHIIASLPHRFIPLFKQILSDRNTLSFILEEFKSDEEVIQSFCKYKTLLRNENVLETAEALF
NELNSIDLTHIFISHKKLETISSALCDHWDTLRNALYERRISELTGKITKSAKEKVQRSLKH
EDINLQEIISAAGKELSEAFKQKTSEILSHAHAALDQPLPTTLKKQEEKEILKSQLDSLLGL
YHLLDWFAVDESNEVDPEFSARLTGIKLEMEPSLSFYNKARNYATKKPYSVEKFKLNFQMPT
LASGWDVNKEKNNGAILFVKNGLYYLGIMPKQKGRYKALSFEPTEKTSEGFDKMYYDYFPDA
AKMIPKCSTQLKAVTAHFQTHTTPILLSNNFIEPLEITKEIYDLNNPEKEPKKFQTAYAKKT
GDQKGYREALCKWIDFTRDFLSKYTKTTSIDLSSLRPSSQYKDLGEYYAELNPLLYHISFQR
IAEKEIMDAVETGKLYLFQIYNKDFAKGHHGKPNLHTLYWTGLFSPENLAKTSIKLNGQAEL
FYRPKSRMKRMAARLGEKMLNKKLKDQKTPIPDTLYQELYDYVNHRLSHDLSDEARALLPNV
ITKEVSHEIIKDRRFTSDKFFFHVPITLNYQAANSPSKFNQRVNAYLKEHPETPIIGIDRGE
RNLIYITVIDSTGKILEQRSLNTIQQFDYQKKLDNREKERVAARQAWSVVGTIKDLKQGYLS
QVIHEIVDLMIHYQAVVVLENLNFGFKSKRTGIAEKAVYQQFEKMLIDKLNCLVLKDYPAEK
VGGVLNPYQLTDQFTSFAKMGTQSGFLFYVPAPYTSKIDPLTGFVDPFVWKTIKNHESRKHF
LEGFDFLHYDVKTGDFILHFKMNRNLSFQRGLPGFMPAWDIVFEKNETQFDAKGTPFIAGKR
IVPVIENHRFTGRYRDLYPANELIALLEEKGIVFRDGSNILPKLLENDDSHAIDTMVALIRS
VLQMRNSNAATGEDYINSPVRDLNGVCFDSRFQNPEWPMDADANGAYHIALKGQLLLNHLKE
SKDLKLQNGISNQDWLAYIQELRNGSPKKKRKVGSPKKKRKV
Cpf1 variant 1 amino acid sequence
(SEQ ID NO: [XX])
MTQFEGFTNLYQVSKTLRFELIPQGKTLKHIQEQGFIEEDKARNDHYKELKPIIDRI
YKTYADQCLQLVQLDWENLSAAIDSYRKEKTEETRNALIEEQATYRNAIHDYFIGRTDNLTD
AINKRHAEIYKGLFKAELFNGKVLKQLGTVTTTEHENALLRSFDKFTTYFSGFYENRKNVFS
AEDISTAIPHRIVQDNFPKFKENCHIFTRLITAVPSLREHFENVKKAIGIFVSTSIEEVFSF
PFYNQLLTQTQIDLYNQLLGGISREAGTEKIKGLNEVLNLAIQKNDETAHIIASLPHRFIPL
FKQILSDRNTLSFILEEFKSDEEVIQSFCKYKTLLRNENVLETAEALFNELNSIDLTHIFIS
HKKLETISSALCDHWDTLRNALYERRISELTGKITKSAKEKVQRSLKHEDINLQEIISAAGK
ELSEAFKQKTSEILSHAHAALDQPLPTTLKKQEEKEILKSQLDSLLGLYHLLDWFAVDESNE
VDPEFSARLTGIKLEMEPSLSFYNKARNYATKKPYSVEKFKLNFQRPTLASGWDVNKEKNNG
AILFVKNGLYYLGIMPKQKGRYKALSFEPTEKTSEGFDKMYYDYFPDAAKMIPKCSTQLKAV
TAHFQTHTTPILLSNNFIEPLEITKEIYDLNNPEKEPKKFQTAYAKKTGDQKGYREALCKWI
DFTRDFLSKYTKTTSIDLSSLRPSSQYKDLGEYYAELNPLLYHISFQRIAEKEIMDAVETGK
LYLFQIYNKDFAKGHHGKPNLHTLYWTGLFSPENLAKTSIKLNGQAELFYRPKSRMKRMAHR
LGEKMLNKKLKDQKTPIPDTLYQELYDYVNHRLSHDLSDEARALLPNVITKEVSHEIIKDRR
FTSDKFLFHVPITLNYQAANSPSKFNQRVNAYLKEHPETPIIGIDRGERNLIYITVIDSTGK
ILEQRSLNTIQQFDYQKKLDNREKERVAARQAWSVVGTIKDLKQGYLSQVIHEIVDLMIHYQ
AVVVLENLNFGFKSKRTGIAEKAVYQQFEKMLIDKLNCLVLKDYPAEKVGGVLNPYQLTDQF
TSFAKMGTQSGFLFYVPAPYTSKIDPLTGFVDPFVWKTIKNHESRKHFLEGFDFLHYDVKTG
DFILHFKMNRNLSFQRGLPGFMPAWDIVFEKNETQFDAKGTPFIAGKRIVPVIENHRFTGRY
RDLYPANELIALLEEKGIVFRDGSNILPKLLENDDSHAIDTMVALIRSVLQMRNSNAATGED
YINSPVRDLNGVCFDSRFQNPEWPMDADANGAYHIALKGQLLLNHLKESKDLKLQNGISNQD
WLAYIQELRNGRSSDDEATADSQHAAPPKKKRKVGGSGGSGGSGGSGGSGGSGGSGGSLEHH
HHHH
Cpf1 variant 2 amino acid sequence
(SEQ ID NO: [XX])
MTQFEGFTNLYQVSKTLRFELIPQGKTLKHIQEQGFIEEDKARNDHYKELKP
IIDRIYKTYADQCLQLVQLDWENLSAAIDSYRKEKTEETRNALIEEQATYRNAIHDYFIGRT
DNLTDAINKRHAEIYKGLFKAELFNGKVLKQLGTVTTTEHENALLRSFDKFTTYFSGFYENR
KNVFSAEDISTAIPHRIVQDNFPKFKENCHIFTRLITAVPSLREHFENVKKAIGIFVSTSIE
EVFSFPFYNQLLTQTQIDLYNQLLGGISREAGTEKIKGLNEVLNLAIQKNDETAHIIASLPH
RFIPLFKQILSDRNTLSFILEEFKSDEEVIQSFCKYKTLLRNENVLETAEALFNELNSIDLT
HIFISHKKLETISSALCDHWDTLRNALYERRISELTGKITKSAKEKVQRSLKHEDINLQEII
SAAGKELSEAFKQKTSEILSHAHAALDQPLPTTLKKQEEKEILKSQLDSLLGLYHLLDWFAV
DESNEVDPEFSARLTGIKLEMEPSLSFYNKARNYATKKPYSVEKFKLNFQMPTLASGWDVNK
EKNNGAILFVKNGLYYLGIMPKQKGRYKALSFEPTEKTSEGFDKMYYDYFPDAAKMIPKCST
QLKAVTAHFQTHTTPILLSNNFIEPLEITKEIYDLNNPEKEPKKFQTAYAKKTGDQKGYREA
LCKWIDFTRDFLSKYTKTTSIDLSSLRPSSQYKDLGEYYAELNPLLYHISFQRIAEKEIMDA
VETGKLYLFQIYNKDFAKGHHGKPNLHTLYWTGLFSPENLAKTSIKLNGQAELFYRPKSRMK
RMAHRLGEKMLNKKLKDQKTPIPDTLYQELYDYVNHRLSHDLSDEARALLPNVITKEVSHEI
IKDRRFTSDKFFFHVPITLNYQAANSPSKFNQRVNAYLKEHPETPIIGIDRGERNLIYITVI
DSTGKILEQRSLNTIQQFDYQKKLDNREKERVAARQAWSVVGTIKDLKQGYLSQVIHEIVDL
MIHYQAVVVLENLNFGFKSKRTGIAEKAVYQQFEKMLIDKLNCLVLKDYPAEKVGGVLNPYQ
LTDQFTSFAKMGTQSGFLFYVPAPYTSKIDPLTGFVDPFVWKTIKNHESRKHFLEGFDFLHY
DVKTGDFILHFKMNRNLSFQRGLPGFMPAWDIVFEKNETQFDAKGTPFIAGKRIVPVIENHR
FTGRYRDLYPANELIALLEEKGIVFRDGSNILPKLLENDDSHAIDTMVALIRSVLQMRNSNA
ATGEDYINSPVRDLNGVCFDSRFQNPEWPMDADANGAYHIALKGQLLLNHLKESKDLKLQNG
ISNQDWLAYIQELRNGRSSDDEATADSQHAAPPKKKRKVGGSGGSGGSGGSGGSGGSGGSGG
SLEHHHHHH
Cpf1 variant 3 amino acid sequence
(SEQ ID NO: 1096)
MTQFEGFTNLYQVSKTLRFELIPQGKTLKHIQEQGFIEEDKARNDHYKELKP
IIDRIYKTYADQCLQLVQLDWENLSAAIDSYRKEKTEETRNALIEEQATYRNAIHDYFIGRT
DNLTDAINKRHAEIYKGLFKAELFNGKVLKQLGTVTTTEHENALLRSFDKFTTYFSGFYENR
KNVFSAEDISTAIPHRIVQDNFPKFKENCHIFTRLITAVPSLREHFENVKKAIGIFVSTSIE
EVFSFPFYNQLLTQTQIDLYNQLLGGISREAGTEKIKGLNEVLNLAIQKNDETAHIIASLPH
RFIPLFKQILSDRNTLSFILEEFKSDEEVIQSFCKYKTLLRNENVLETAEALFNELNSIDLT
HIFISHKKLETISSALCDHWDTLRNALYERRISELTGKITKSAKEKVQRSLKHEDINLQEII
SAAGKELSEAFKQKTSEILSHAHAALDQPLPTTLKKQEEKEILKSQLDSLLGLYHLLDWFAV
DESNEVDPEFSARLTGIKLEMEPSLSFYNKARNYATKKPYSVEKFKLNFQRPTLASGWDVNK
EKNNGAILFVKNGLYYLGIMPKQKGRYKALSFEPTEKTSEGFDKMYYDYFPDAAKMIPKCST
QLKAVTAHFQTHTTPILLSNNFIEPLEITKEIYDLNNPEKEPKKFQTAYAKKTGDQKGYREA
LCKWIDFTRDFLSKYTKTTSIDLSSLRPSSQYKDLGEYYAELNPLLYHISFQRIAEKEIMDA
VETGKLYLFQIYNKDFAKGHHGKPNLHTLYWTGLFSPENLAKTSIKLNGQAELFYRPKSRMK
RMAARLGEKMLNKKLKDQKTPIPDTLYQELYDYVNHRLSHDLSDEARALLPNVITKEVSHEI
IKDRRFTSDKFLFHVPITLNYQAANSPSKFNQRVNAYLKEHPETPIIGIDRGERNLIYITVI
DSTGKILEQRSLNTIQQFDYQKKLDNREKERVAARQAWSVVGTIKDLKQGYLSQVIHEIVDL
MIHYQAVVVLENLNFGFKSKRTGIAEKAVYQQFEKMLIDKLNCLVLKDYPAEKVGGVLNPYQ
LTDQFTSFAKMGTQSGFLFYVPAPYTSKIDPLTGFVDPFVWKTIKNHESRKHFLEGFDFLHY
DVKTGDFILHFKMNRNLSFQRGLPGFMPAWDIVFEKNETQFDAKGTPFIAGKRIVPVIENHR
FTGRYRDLYPANELIALLEEKGIVFRDGSNILPKLLENDDSHAIDTMVALIRSVLQMRNSNA
ATGEDYINSPVRDLNGVCFDSRFQNPEWPMDADANGAYHIALKGQLLLNHLKESKDLKLQNG
ISNQDWLAYIQELRNGRSSDDEATADSQHAAPPKKKRKVGGSGGSGGSGGSGGSGGSGGSGG
SLEHHHHHH
Cpf1 variant 4 amino acid sequence
(SEQ ID NO: 1097)
MTQFEGFTNLYQVSKTLRFELIPQGKTLKHIQEQGFIEEDKARNDHYKELKP
IIDRIYKTYADQCLQLVQLDWENLSAAIDSYRKEKTEETRNALIEEQATYRNAIHDYFIGRT
DNLTDAINKRHAEIYKGLFKAELFNGKVLKQLGTVTTTEHENALLRSFDKFTTYFSGFYENR
KNVFSAEDISTAIPHRIVQDNFPKFKENCHIFTRLITAVPSLREHFENVKKAIGIFVSTSIE
EVFSFPFYNQLLTQTQIDLYNQLLGGISREAGTEKIKGLNEVLNLAIQKNDETAHIIASLPH
RFIPLFKQILSDRNTLSFILEEFKSDEEVIQSFCKYKTLLRNENVLETAEALFNELNSIDLT
HIFISHKKLETISSALCDHWDTLRNALYERRISELTGKITKSAKEKVQRSLKHEDINLQEII
SAAGKELSEAFKQKTSEILSHAHAALDQPLPTTLKKQEEKEILKSQLDSLLGLYHLLDWFAV
DESNEVDPEFSARLTGIKLEMEPSLSFYNKARNYATKKPYSVEKFKLNFQRPTLASGWDVNK
EKNNGAILFVKNGLYYLGIMPKQKGRYKALSFEPTEKTSEGFDKMYYDYFPDAAKMIPKCST
QLKAVTAHFQTHTTPILLSNNFIEPLEITKEIYDLNNPEKEPKKFQTAYAKKTGDQKGYREA
LCKWIDFTRDFLSKYTKTTSIDLSSLRPSSQYKDLGEYYAELNPLLYHISFQRIAEKEIMDA
VETGKLYLFQIYNKDFAKGHHGKPNLHTLYWTGLFSPENLAKTSIKLNGQAELFYRPKSRMK
RMAARLGEKMLNKKLKDQKTPIPDTLYQELYDYVNHRLSHDLSDEARALLPNVITKEVSHEI
IKDRRFTSDKFLFHVPITLNYQAANSPSKFNQRVNAYLKEHPETPIIGIDRGERNLIYITVI
DSTGKILEQRSLNTIQQFDYQKKLDNREKERVAARQAWSVVGTIKDLKQGYLSQVIHEIVDL
MIHYQAVVVLENLNFGFKSKRTGIAEKAVYQQFEKMLIDKLNCLVLKDYPAEKVGGVLNPYQ
LTDQFTSFAKMGTQSGFLFYVPAPYTSKIDPLTGFVDPFVWKTIKNHESRKHFLEGFDFLHY
DVKTGDFILHFKMNRNLSFQRGLPGFMPAWDIVFEKNETQFDAKGTPFIAGKRIVPVIENHR
FTGRYRDLYPANELIALLEEKGIVFRDGSNILPKLLENDDSHAIDTMVALIRSVLQMRNSNA
ATGEDYINSPVRDLNGVCFDSRFQNPEWPMDADANGAYHIALKGQLLLNHLKESKDLKLQNG
ISNQDWLAYIQELRNGRSSDDEATADSQHAAPPKKKRKV
Cpf1 variant 5 amino acid sequence
(SEQ ID NO: 1107)
MTQFEGFTNLYQVSKTLRFELIPQGKTLKHIQEQGFIEEDKARNDHYKELKP
IIDRIYKTYADQCLQLVQLDWENLSAAIDSYRKEKTEETRNALIEEQATYRNAIHDYFIGRT
DNLTDAINKRHAEIYKGLFKAELFNGKVLKQLGTVTTTEHENALLRSFDKFTTYFSGFYENR
KNVFSAEDISTAIPHRIVQDNFPKFKENCHIFTRLITAVPSLREHFENVKKAIGIFVSTSIE
EVFSFPFYNQLLTQTQIDLYNQLLGGISREAGTEKIKGLNEVLNLAIQKNDETAHIIASLPH
RFIPLFKQILSDRNTLSFILEEFKSDEEVIQSFCKYKTLLRNENVLETAEALFNELNSIDLT
HIFISHKKLETISSALCDHWDTLRNALYERRISELTGKITKSAKEKVQRSLKHEDINLQEII
SAAGKELSEAFKQKTSEILSHAHAALDQPLPTTLKKQEEKEILKSQLDSLLGLYHLLDWFAV
DESNEVDPEFSARLTGIKLEMEPSLSFYNKARNYATKKPYSVEKFKLNFQRPTLASGWDVNK
EKNNGAILFVKNGLYYLGIMPKQKGRYKALSFEPTEKTSEGFDKMYYDYFPDAAKMIPKCST
QLKAVTAHFQTHTTPILLSNNFIEPLEITKEIYDLNNPEKEPKKFQTAYAKKTGDQKGYREA
LCKWIDFTRDFLSKYTKTTSIDLSSLRPSSQYKDLGEYYAELNPLLYHISFQRIAEKEIMDA
VETGKLYLFQIYNKDFAKGHHGKPNLHTLYWTGLFSPENLAKTSIKLNGQAELFYRPKSRMK
RMAHRLGEKMLNKKLKDQKTPIPDTLYQELYDYVNHRLSHDLSDEARALLPNVITKEVSHEI
IKDRRFTSDKFLFHVPITLNYQAANSPSKFNQRVNAYLKEHPETPIIGIDRGERNLIYITVI
DSTGKILEQRSLNTIQQFDYQKKLDNREKERVAARQAWSVVGTIKDLKQGYLSQVIHEIVDL
MIHYQAVVVLENLNFGFKSKRTGIAEKAVYQQFEKMLIDKLNCLVLKDYPAEKVGGVLNPYQ
LTDQFTSFAKMGTQSGFLFYVPAPYTSKIDPLTGFVDPFVWKTIKNHESRKHFLEGFDFLHY
DVKTGDFILHFKMNRNLSFQRGLPGFMPAWDIVFEKNETQFDAKGTPFIAGKRIVPVIENHR
FTGRYRDLYPANELIALLEEKGIVFRDGSNILPKLLENDDSHAIDTMVALIRSVLQMRNSNA
ATGEDYINSPVRDLNGVCFDSRFQNPEWPMDADANGAYHIALKGQLLLNHLKESKDLKLQNG
ISNQDWLAYIQELRNGRSSDDEATADSQHAAPPKKKRKV
Cpf1 variant 6 amino acid sequence
(SEQ ID NO: 1108)
MTQFEGFTNLYQVSKTLRFELIPQGKTLKHIQEQGFIEEDKARNDHYKELKP
IIDRIYKTYADQCLQLVQLDWENLSAAIDSYRKEKTEETRNALIEEQATYRNAIHDYFIGRT
DNLTDAINKRHAEIYKGLFKAELFNGKVLKQLGTVTTTEHENALLRSFDKFTTYFSGFYENR
KNVFSAEDISTAIPHRIVQDNFPKFKENCHIFTRLITAVPSLREHFENVKKAIGIFVSTSIE
EVFSFPFYNQLLTQTQIDLYNQLLGGISREAGTEKIKGLNEVLNLAIQKNDETAHIIASLPH
RFIPLFKQILSDRNTLSFILEEFKSDEEVIQSFCKYKTLLRNENVLETAEALFNELNSIDLT
HIFISHKKLETISSALCDHWDTLRNALYERRISELTGKITKSAKEKVQRSLKHEDINLQEII
SAAGKELSEAFKQKTSEILSHAHAALDQPLPTTLKKQEEKEILKSQLDSLLGLYHLLDWFAV
DESNEVDPEFSARLTGIKLEMEPSLSFYNKARNYATKKPYSVEKFKLNFQRPTLASGWDVNK
EKNNGAILFVKNGLYYLGIMPKQKGRYKALSFEPTEKTSEGFDKMYYDYFPDAAKMIPKCST
QLKAVTAHFQTHTTPILLSNNFIEPLEITKEIYDLNNPEKEPKKFQTAYAKKTGDQKGYREA
LCKWIDFTRDFLSKYTKTTSIDLSSLRPSSQYKDLGEYYAELNPLLYHISFQRIAEKEIMDA
VETGKLYLFQIYNKDFAKGHHGKPNLHTLYWTGLFSPENLAKTSIKLNGQAELFYRPKSRMK
RMAHRLGEKMLNKKLKDQKTPIPDTLYQELYDYVNHRLSHDLSDEARALLPNVITKEVSHEI
IKDRRFTSDKFLFHVPITLNYQAANSPSKFNQRVNAYLKEHPETPIIGIDRGERNLIYITVI
DSTGKILEQRSLNTIQQFDYQKKLDNREKERVAARQAWSVVGTIKDLKQGYLSQVIHEIVDL
MIHYQAVVVLENLNFGFKSKRTGIAEKAVYQQFEKMLIDKLNCLVLKDYPAEKVGGVLNPYQ
LTDQFTSFAKMGTQSGFLFYVPAPYTSKIDPLTGFVDPFVWKTIKNHESRKHFLEGFDFLHY
DVKTGDFILHFKMNRNLSFQRGLPGFMPAWDIVFEKNETQFDAKGTPFIAGKRIVPVIENHR
FTGRYRDLYPANELIALLEEKGIVFRDGSNILPKLLENDDSHAIDTMVALIRSVLQMRNSNA
ATGEDYINSPVRDLNGVCFDSRFQNPEWPMDADANGAYHIALKGQLLLNHLKESKDLKLQNG
ISNQDWLAYIQELRNGRSSDDEATADSQHAAPPKKKRKVGGSGGSGGSGGSGGSGGSGGSGG
SLEHHHHHH
Cpf1 variant 7 amino acid sequence
(SEQ ID NO: [[XX]])
MGRDPGKPIPNPLLGLDSTAPKKKRKVGIHGVPAATQFEGFTNLYQVSKTLR
FELIPQGKTLKHIQEQGFIEEDKARNDHYKELKPIIDRIYKTYADQCLQLVQLDWENLSAAI
DSYRKEKTEETRNALIEEQATYRNAIHDYFIGRTDNLTDAINKRHAEIYKGLFKAELFNGKV
LKQLGTVTTTEHENALLRSFDKFTTYFSGFYENRKNVFSAEDISTAIPHRIVQDNFPKFKEN
CHIFTRLITAVPSLREHFENVKKAIGIFVSTSIEEVFSFPFYNQLLTQTQIDLYNQLLGGIS
REAGTEKIKGLNEVLNLAIQKNDETAHIIASLPHRFIPLFKQILSDRNTLSFILEEFKSDEE
VIQSFCKYKTLLRNENVLETAEALFNELNSIDLTHIFISHKKLETISSALCDHWDTLRNALY
ERRISELTGKITKSAKEKVQRSLKHEDINLQEIISAAGKELSEAFKQKTSEILSHAHAALDQ
PLPTTLKKQEEKEILKSQLDSLLGLYHLLDWFAVDESNEVDPEFSARLTGIKLEMEPSLSFY
NKARNYATKKPYSVEKFKLNFQMPTLASGWDVNKEKNNGAILFVKNGLYYLGIMPKQKGRYK
ALSFEPTEKTSEGFDKMYYDYFPDAAKMIPKCSTQLKAVTAHFQTHTTPILLSNNFIEPLEI
TKEIYDLNNPEKEPKKFQTAYAKKTGDQKGYREALCKWIDFTRDFLSKYTKTTSIDLSSLRP
SSQYKDLGEYYAELNPLLYHISFQRIAEKEIMDAVETGKLYLFQIYNKDFAKGHHGKPNLHT
LYWTGLFSPENLAKTSIKLNGQAELFYRPKSRMKRMAHRLGEKMLNKKLKDQKTPIPDTLYQ
ELYDYVNHRLSHDLSDEARALLPNVITKEVSHEIIKDRRFTSDKFFFHVPITLNYQAANSPS
KFNQRVNAYLKEHPETPIIGIDRGERNLIYITVIDSTGKILEQRSLNTIQQFDYQKKLDNRE
KERVAARQAWSVVGTIKDLKQGYLSQVIHEIVDLMIHYQAVVVLENLNFGFKSKRTGIAEKA
VYQQFEKMLIDKLNCLVLKDYPAEKVGGVLNPYQLTDQFTSFAKMGTQSGFLFYVPAPYTSK
IDPLTGFVDPFVWKTIKNHESRKHFLEGFDFLHYDVKTGDFILHFKMNRNLSFQRGLPGFMP
AWDIVFEKNETQFDAKGTPFIAGKRIVPVIENHRFTGRYRDLYPANELIALLEEKGIVFRDG
SNILPKLLENDDSHAIDTMVALIRSVLQMRNSNAATGEDYINSPVRDLNGVCFDSRFQNPEW
PMDADANGAYHIALKGQLLLNHLKESKDLKLQNGISNQDWLAYIQELRNPKKKRKVKLAAAL
EHHHHHH
Exemplary AsCpf1 wild-type amino acid sequence
(SEQ ID NO: [[XX]]):
MTQFEGFTNLYQVSKTLRFELIPQGKTLKHIQEQGFIEEDKARNDHYKELKP
IIDRIYKTYADQCLQLVQLDWENLSAAIDSYRKEKTEETRNALIEEQATYRNAIHDYFIGRT
DNLTDAINKRHAEIYKGLFKAELFNGKVLKQLGTVTTTEHENALLRSFDKFTTYFSGFYENR
KNVFSAEDISTAIPHRIVQDNFPKFKENCHIFTRLITAVPSLREHFENVKKAIGIFVSTSIE
EVFSFPFYNQLLTQTQIDLYNQLLGGISREAGTEKIKGLNEVLNLAIQKNDETAHIIASLPH
RFIPLFKQILSDRNTLSFILEEFKSDEEVIQSFCKYKTLLRNENVLETAEALFNELNSIDLT
HIFISHKKLETISSALCDHWDTLRNALYERRISELTGKITKSAKEKVQRSLKHEDINLQEII
SAAGKELSEAFKQKTSEILSHAHAALDQPLPTTLKKQEEKEILKSQLDSLLGLYHLLDWFAV
DESNEVDPEFSARLTGIKLEMEPSLSFYNKARNYATKKPYSVEKFKLNFQMPTLASGWDVNK
EKNNGAILFVKNGLYYLGIMPKQKGRYKALSFEPTEKTSEGFDKMYYDYFPDAAKMIPKCST
QLKAVTAHFQTHTTPILLSNNFIEPLEITKEIYDLNNPEKEPKKFQTAYAKKTGDQKGYREA
LCKWIDFTRDFLSKYTKTTSIDLSSLRPSSQYKDLGEYYAELNPLLYHISFQRIAEKEIMDA
VETGKLYLFQIYNKDFAKGHHGKPNLHTLYWTGLFSPENLAKTSIKLNGQAELFYRPKSRMK
RMAHRLGEKMLNKKLKDQKTPIPDTLYQELYDYVNHRLSHDLSDEARALLPNVITKEVSHEI
IKDRRFTSDKFFFHVPITLNYQAANSPSKFNQRVNAYLKEHPETPIIGIDRGERNLIYITVI
DSTGKILEQRSLNTIQQFDYQKKLDNREKERVAARQAWSVVGTIKDLKQGYLSQVIHEIVDL
MIHYQAVVVLENLNFGFKSKRTGIAEKAVYQQFEKMLIDKLNCLVLKDYPAEKVGGVLNPYQ
LTDQFTSFAKMGTQSGFLFYVPAPYTSKIDPLTGFVDPFVWKTIKNHESRKHFLEGFDFLHY
DVKTGDFILHFKMNRNLSFQRGLPGFMPAWDIVFEKNETQFDAKGTPFIAGKRIVPVIENHR
FTGRYRDLYPANELIALLEEKGIVFRDGSNILPKLLENDDSHAIDTMVALIRSVLQMRNSNA
ATGEDYINSPVRDLNGVCFDSRFQNPEWPMDADANGAYHIALKGQLLLNHLKESKDLKLQNG
ISNQDWLAYIQELRN

Nucleic Acids Encoding RNA-Guided Nucleases

Nucleic acids encoding RNA-guided nucleases, e.g., Cas9, Cpf1 or functional fragments thereof, are provided herein. Exemplary nucleic acids encoding RNA-guided nucleases have been described previously (see, e.g., Cong 2013; Wang 2013; Mali 2013; Jinek 2012).

In some cases, a nucleic acid encoding an RNA-guided nuclease can be a synthetic nucleic acid sequence. For example, the synthetic nucleic acid molecule can be chemically modified. In certain embodiments, an mRNA encoding an RNA-guided nuclease will have one or more (e.g., all) of the following properties: it can be capped; polyadenylated; and substituted with 5-methylcytidine and/or pseudouridine.

Synthetic nucleic acid sequences can also be codon optimized, e.g., at least one non-common codon or less-common codon has been replaced by a common codon. For example, the synthetic nucleic acid can direct the synthesis of an optimized messenger mRNA, e.g., optimized for expression in a mammalian expression system, e.g., described herein. Examples of codon optimized Cas9 coding sequences are presented in Cotta-Ramusino.

In addition, or alternatively, a nucleic acid encoding an RNA-guided nuclease may comprise a nuclear localization sequence (NLS). Nuclear localization sequences are known in the art.

Functional Analysis of Candidate Molecules

Candidate RNA-guided nucleases, gRNAs, and complexes thereof, can be evaluated by standard methods known in the art. See, e.g. Cotta-Ramusino. The stability of RNP complexes may be evaluated by differential scanning fluorimetry, as described below.

Differential Scanning Fluorimetry (DSF)

The thermostability of ribonucleoprotein (RNP) complexes comprising gRNAs and RNA-guided nucleases can be measured via DSF. The DSF technique measures the thermostability of a protein, which can increase under favorable conditions such as the addition of a binding RNA molecule, e.g., a gRNA.

A DSF assay can be performed according to any suitable protocol, and can be employed in any suitable setting, including without limitation (a) testing different conditions (e.g. different stoichiometric ratios of gRNA: RNA-guided nuclease protein, different buffer solutions, etc.) to identify optimal conditions for RNP formation; and (b) testing modifications (e.g. chemical modifications, alterations of sequence, etc.) of an RNA-guided nuclease and/or a gRNA to identify those modifications that improve RNP formation or stability. One readout of a DSF assay is a shift in melting temperature of the RNP complex; a relatively high shift suggests that the RNP complex is more stable (and may thus have greater activity or more favorable kinetics of formation, kinetics of degradation, or another functional characteristic) relative to a reference RNP complex characterized by a lower shift. When the DSF assay is deployed as a screening tool, a threshold melting temperature shift may be specified, so that the output is one or more RNPs having a melting temperature shift at or above the threshold. For instance, the threshold can be 5-10° C. (e.g. 5°, 6°, 7°, 8°, 9°, 10°) or more, and the output may be one or more RNPs characterized by a melting temperature shift greater than or equal to the threshold.

Two non-limiting examples of DSF assay conditions are set forth below:

To determine the best solution to form RNP complexes, a fixed concentration (e.g. 2 μM) of Cas9 in water+10×SYPRO Orange® (Life Technologies cat #S-6650) is dispensed into a 384 well plate. An equimolar amount of gRNA diluted in solutions with varied pH and salt is then added. After incubating at room temperature for 10′ and brief centrifugation to remove any bubbles, a Bio-Rad CFX384™ Real-Time System C1000 Touch™ Thermal Cycler with the Bio-Rad CFX Manager software is used to run a gradient from 20° C. to 90° C. with a 1° C. increase in temperature every 10 seconds.

The second assay consists of mixing various concentrations of gRNA with fixed concentration (e.g. 2 μM) Cas9 in optimal buffer from assay 1 above and incubating (e.g. at RT for 10′) in a 384 well plate. An equal volume of optimal buffer+10×SYPRO Orange® (Life Technologies cat #S-6650) is added and the plate sealed with Microseal® B adhesive (MSB-1001). Following brief centrifugation to remove any bubbles, a Bio-Rad CFX384™ Real-Time System C1000 Touch™ Thermal Cycler with the Bio-Rad CFX Manager software is used to run a gradient from 20° C. to 90° C. with a 1° C. increase in temperature every 10 seconds.

### Genome Editing Strategies

The genome editing systems described above are used, in various embodiments of the present disclosure, to generate edits in (i.e. to alter) targeted regions of DNA within or obtained from a cell. Various strategies are described herein to generate particular edits, and these strategies are generally described in terms of the desired repair outcome, the number and positioning of individual edits (e.g. SSBs or DSBs), and the target sites of such edits.

Genome editing strategies that involve the formation of SSBs or DSBs are characterized by repair outcomes including: (a) deletion of all or part of a targeted region; (b) insertion into or replacement of all or part of a targeted region; or (c) interruption of all or part of a targeted region. This grouping is not intended to be limiting, or to be binding to any particular theory or model, and is offered solely for economy of presentation. Skilled artisans will appreciate that the listed outcomes are not mutually exclusive and that some repairs may result in other outcomes. The description of a particular editing strategy or method should not be understood to require a particular repair outcome unless otherwise specified.

Replacement of a targeted region generally involves the replacement of all or part of the existing sequence within the targeted region with a homologous sequence, for instance through gene correction or gene conversion, two repair outcomes that are mediated by HDR pathways. HDR is promoted by the use of a donor template, which can be single-stranded or double stranded, as described in greater detail below. Single or double stranded templates can be exogenous, in which case they will promote gene correction, or they can be endogenous (e.g. a homologous sequence within the cellular genome), to promote gene conversion. Exogenous templates can have asymmetric overhangs (i.e. the portion of the template that is complementary to the site of the DSB may be offset in a 3′ or 5′ direction, rather than being centered within the donor template), for instance as described by Richardson et al. (Nature Biotechnology 34, 339-344 (2016), (Richardson), incorporated by reference). In instances where the template is single stranded, it can correspond to either the complementary (top) or non-complementary (bottom) strand of the targeted region.

### Gene Constructs

In some aspects, the present disclosure provides complex editing strategies, and resulting modified cells having complex genomic alterations, that allow for the generation of advanced NK cell products for clinical applications, e.g., for immunooncology therapeutic approaches.

In some embodiments, the genomic alterations are introduced by use of one or more HDR expression constructs. In some embodiments, the genomic alterations are introduced by use of one or more HDR expression constructs. In some embodiments, the one or more HDR expression constructs comprise one or more donor HDR templates. In some embodiments, the one or more donor HDR templates comprise one or more expression cassettes encoding one or more cDNAs. In some embodiments, the donor HDR template comprises one expression cassette. In some embodiments, the donor HDR template comprises two expression cassettes. In some embodiments, the donor HDR template comprises three expression cassettes. In some embodiments, the donor HDR template comprises four expression cassettes. In some embodiments, the donor HDR template comprises five expression cassettes. In some embodiments, the donor HDR template comprises six expression cassettes. In some embodiments, the donor HDR template comprises seven expression cassettes. In some embodiments, the donor HDR template comprises eight expression cassettes. In some embodiments, the donor HDR template comprises nine expression cassettes. In some embodiments, the donor HDR template comprises ten expression cassettes. In some embodiments, the one or more expression cassette is monocistronic. In some embodiments, the one or more expression cassette is bicistronic.

In some embodiments, the one or more expression cassettes comprise one cDNA. In some embodiments, the one or more expression cassettes comprise two cDNAs. In some embodiments, the one or more expression cassettes comprise three cDNAs. In some embodiments, the one or more expression cassettes comprise four cDNAs. In some embodiments, the one or more expression cassettes comprise five cDNAs. In some embodiments, the one or more expression cassettes comprise six cDNAs. In some embodiments, the one or more expression cassettes comprise seven cDNAs. In some embodiments, the one or more expression cassettes comprise eight cDNAs. In some embodiments, the one or more expression cassettes comprise nine cDNAs. In some embodiments, the one or more expression cassettes comprise ten cDNAs. In some embodiments, the one or more expression cassettes comprise one or more cDNAs separated by a 2A sequence. In some embodiments, the one or more expression cassettes comprise two cDNAs separated by a 2A sequence. In some embodiments, the one or more expression cassettes comprise three cDNAs separated by a 2A sequence.

In some embodiments, the HDR expression construct comprises one or more cDNAs driven by a heterologous promoter.

In some embodiments, the one or more expression cassettes comprise a cDNA for the expression of one or more genes listed in Table 10.

In some embodiments, the HDR expression construct comprises one or more donor templates for inserting an inactivating mutation in a target gene, wherein the gene product has less, or no, function (being partially or wholly inactivated). In some embodiments, the HDR expression construct comprises one or more donor templates for inserting an inactivating mutation in a target gene, wherein the gene product has no function (wholly inactivated).

In some embodiments, the modified cell of the disclosure comprises at least one exogenous nucleic acid construct encoding a cDNA of one or more genes listed in Table 10. In some embodiments, the modified cell of the disclosure, comprise any combination of two or more exogenous nucleic acid constructs encoding cDNAs of one or more genes listed in Table 10. In some embodiments, the modified cell of the disclosure, comprise any combination of three or more exogenous nucleic acid constructs encoding cDNAs of one or more genes listed in Table 10. In some embodiments, the modified cell of the disclosure, comprise any combination of four or more exogenous nucleic acid constructs encoding cDNAs of one or more genes listed in Table 10. In some embodiments, the modified cell of the disclosure, comprise any combination of five or more exogenous nucleic acid constructs encoding cDNAs of one or more genes listed in Table 10. In some embodiments, the modified cell of the disclosure, comprise any combination of six or more exogenous nucleic acid constructs encoding cDNAs of one or more genes listed in Table 10. In some embodiments, the modified cell of the disclosure, comprise any combination of seven or more exogenous nucleic acid constructs encoding cDNAs of one or more genes listed in Table 10. In some embodiments, the modified cell of the disclosure, comprise any combination of eight or more exogenous nucleic acid constructs encoding cDNAs of one or more genes listed in Table 10. In some embodiments, the modified cell of the disclosure, comprise any combination of nine or more exogenous nucleic acid constructs encoding cDNAs of one or more genes listed in Table 10. In some embodiments, the modified cell of the disclosure, comprise any combination of ten or more exogenous nucleic acid constructs encoding cDNAs of one or more genes listed in Table 10.

In some embodiments, the modified NK cell of the disclosure, comprises at least one exogenous nucleic acid construct encoding a cDNA of one or more genes listed in Table 10. In some embodiments, the modified cell of the disclosure, comprise any combination of two or more exogenous nucleic acid constructs encoding cDNAs of one or more genes listed in Table 10. In some embodiments, the modified cell of the disclosure, comprise any combination of three or more exogenous nucleic acid constructs encoding cDNAs of one or more genes listed in Table 10. In some embodiments, the modified cell of the disclosure, comprise any combination of four or more exogenous nucleic acid constructs encoding cDNAs of one or more genes listed in Table 10. In some embodiments, the modified cell of the disclosure, comprise any combination of five or more exogenous nucleic acid constructs encoding cDNAs of one or more genes listed in Table 10. In some embodiments, the modified cell of the disclosure, comprise any combination of six or more exogenous nucleic acid constructs encoding cDNAs of one or more genes listed in Table 10. In some embodiments, the modified cell of the disclosure, comprise any combination of seven or more exogenous nucleic acid constructs encoding cDNAs of one or more genes listed in Table 10. In some embodiments, the modified cell of the disclosure, comprise any combination of eight or more exogenous nucleic acid constructs encoding cDNAs of one or more genes listed in Table 10. In some embodiments, the modified cell of the disclosure, comprise any combination of nine or more exogenous nucleic acid constructs encoding cDNAs of one or more genes listed in Table 10. In some embodiments, the modified cell of the disclosure, comprise any combination of ten or more exogenous nucleic acid constructs encoding cDNAs of one or more genes listed in Table 10.

In some embodiments, the modified cell of the disclosure exhibits a loss of function of at least one or more genes listed in Table 11, or any combination of two or more thereof. In some embodiments, the modified cell of the disclosure exhibits a loss of function of at least two or more genes listed in Table 11. In some embodiments, the modified cell of the disclosure exhibits a loss of function of at least three or more genes listed in Table 11. In some embodiments, the modified cell of the disclosure exhibits a loss of function of at least four or more genes listed in Table 11. In some embodiments, the modified cell of the disclosure exhibits a loss of function of at least five or more genes listed in Table 11. In some embodiments, the modified cell of the disclosure exhibits a loss of function of at least six or more genes listed in Table 11. In some embodiments, the modified cell of the disclosure exhibits a loss of function of at least seven or more genes listed in Table 11. In some embodiments, the modified cell of the disclosure exhibits a loss of function of at least eight or more genes listed in Table 11. In some embodiments, the modified cell of the disclosure exhibits a loss of function of at least nine or more genes listed in Table 11. In some embodiments, the modified cell of the disclosure exhibits a loss of function of at least ten or more genes listed in Table 11.

In some embodiments, the modified NK cell of the disclosure, exhibits a loss of function of at least one or more genes listed in Table 11, or any combination of two or more thereof. In some embodiments, the modified cell of the disclosure exhibits a loss of function of at least two or more genes listed in Table 11. In some embodiments, the modified cell of the disclosure exhibits a loss of function of at least three or more genes listed in Table 11. In some embodiments, the modified cell of the disclosure exhibits a loss of function of at least four or more genes listed in Table 11. In some embodiments, the modified cell of the disclosure exhibits a loss of function of at least five or more genes listed in Table 11. In some embodiments, the modified cell of the disclosure exhibits a loss of function of at least six or more genes listed in Table 11. In some embodiments, the modified cell of the disclosure exhibits a loss of function of at least seven or more genes listed in Table 11. In some embodiments, the modified cell of the disclosure exhibits a loss of function of at least eight or more genes listed in Table 11. In some embodiments, the modified cell of the disclosure exhibits a loss of function of at least nine or more genes listed in Table 11. In some embodiments, the modified cell of the disclosure exhibits a loss of function of at least ten or more genes listed in Table 11.

In some embodiments, the modified cell of the disclosure comprises at least one exogenous nucleic acid construct encoding a cDNA of one or more genes listed in Table 10 and exhibits a loss of function of at least one gene listed in Table 11. In some embodiments, the modified cell of the disclosure, comprise any combination of two or more exogenous nucleic acid constructs encoding cDNAs of one or more genes listed in Table 10 and at least one gene listed in Table 11. In some embodiments, the modified cell of the disclosure comprises at least one exogenous nucleic acid construct encoding cDNAs of one or more genes listed in Table 10 and a loss of function of two or more genes listed in Table 11. In some embodiments, the modified cell of the disclosure comprises two or more exogenous nucleic acid constructs encoding cDNAs of one or more genes listed in Table 10 and a loss of function of two or more genes listed in Table 11.

Gene conversion and gene correction are facilitated, in some cases, by the formation of one or more nicks in or around the targeted region, as described in Ran and Cotta-Ramusino. In some cases, a dual-nickase strategy is used to form two offset SSBs that, in turn, form a single DSB having an overhang (e.g. a 5′ overhang).

Interruption and/or deletion of all or part of a targeted sequence can be achieved by a variety of repair outcomes. As one example, a sequence can be deleted by simultaneously generating two or more DSBs that flank a targeted region, which is then excised when the DSBs are repaired, as is described in Maeder for the LCA10 mutation. As another example, a sequence can be interrupted by a deletion generated by formation of a double strand break with single-stranded overhangs, followed by exonucleolytic processing of the overhangs prior to repair.

One specific subset of target sequence interruptions is mediated by the formation of an indel within the targeted sequence, where the repair outcome is typically mediated by NHEJ pathways (including Alt-NHEJ). NHEJ is referred to as an “error prone” repair pathway because of its association with indel mutations. In some cases, however, a DSB is repaired by NHEJ without alteration of the sequence around it (a so-called “perfect” or “scarless” repair); this generally requires the two ends of the DSB to be perfectly ligated. Indels, meanwhile, are thought to arise from enzymatic processing of free DNA ends before they are ligated that adds and/or removes nucleotides from either or both strands of either or both free ends.

Because the enzymatic processing of free DSB ends may be stochastic in nature, indel mutations tend to be variable, occurring along a distribution, and can be influenced by a variety of factors, including the specific target site, the cell type used, the genome editing strategy used, etc. Even so, it is possible to draw limited generalizations about indel formation: deletions formed by repair of a single DSB are most commonly in the 1-50 bp range, but can reach greater than 100-200 bp. Insertions formed by repair of a single DSB tend to be shorter and often include short duplications of the sequence immediately surrounding the break site. However, it is possible to obtain large insertions, and in these cases, the inserted sequence has often been traced to other regions of the genome or to plasmid DNA present in the cells.

Indel mutations—and genome editing systems configured to produce indels—are useful for interrupting target sequences, for example, when the generation of a specific final sequence is not required and/or where a frameshift mutation would be tolerated. They can also be useful in settings where particular sequences are preferred, insofar as the certain sequences desired tend to occur preferentially from the repair of an SSB or DSB at a given site. Indel mutations are also a useful tool for evaluating or screening the activity of particular genome editing systems and their components. In these and other settings, indels can be characterized by (a) their relative and absolute frequencies in the genomes of cells contacted with genome editing systems and (b) the distribution of numerical differences relative to the unedited sequence, e.g. ±1, ±2, ±3, etc. As one example, in a lead-finding setting, multiple gRNAs can be screened to identify those gRNAs that most efficiently drive cutting at a target site based on an indel readout under controlled conditions. Guides that produce indels at or above a threshold frequency, or that produce a particular distribution of indels, can be selected for further study and development. Indel frequency and distribution can also be useful as a readout for evaluating different genome editing system implementations or formulations and delivery methods, for instance by keeping the gRNA constant and varying certain other reaction conditions or delivery methods.

Multiplex Strategies

While exemplary strategies discussed above have focused on repair outcomes mediated by single DSBs, genome editing systems according to this disclosure may also be employed to generate two or more DSBs, either in the same locus or in different loci. Strategies for editing that involve the formation of multiple DSBs, or SSBs, are described in, for instance, Cotta-Ramusino. In some embodiments, where multiple edits are made in the genome of an NK cell, or a cell that an NK cell is derived from, the edits are made at the same time or in close temporal proximity. In some such embodiments, two or more genomic edits are effected by two or more different RNA-guided nucleases. For example, one of the genomic edits may be effected by saCas9 (in connection with the respective saCas9 guide RNA), and a different genomic edit may be effected by Cpf 1 (in connection with the respective Cpf1 guide RNA). In some embodiments, using different RNA-guided nucleases in the context of multiplex genomic editing approaches is advantageous as compared to using the same RNA-guided nuclease for two or more edits, e.g., in that it allows to decrease the likelihood or frequency of undesirable effects, such as, for example, off-target cutting, and the occurrence of genomic translocations.

Donor Template Design

Donor template design is described in detail in the literature, for instance in Cotta-Ramusino. DNA oligomer donor templates (oligodeoxynucleotides or ODNs), which can be single stranded (ssODNs) or double-stranded (dsODNs), can be used to facilitate HDR-based repair of DSBs, and are particularly useful for introducing alterations into a target DNA sequence, inserting a new sequence into the target sequence, or replacing the target sequence altogether.

Whether single-stranded or double stranded, donor templates generally include regions that are homologous to regions of DNA within or near (e.g. flanking or adjoining) a target sequence to be cleaved. These homologous regions are referred to here as “homology arms,” and are illustrated schematically below:

[5′ homology arm]-[replacement sequence]-[3′ homology arm].

The homology arms can have any suitable length (including 0 nucleotides if only one homology arm is used), and 3′ and 5′ homology arms can have the same length, or can differ in length. The selection of appropriate homology arm lengths can be influenced by a variety of factors, such as the desire to avoid homologies or microhomologies with certain sequences such as Alu repeats or other very common elements. For example, a 5′ homology arm can be shortened to avoid a sequence repeat element. In other embodiments, a 3′ homology arm can be shortened to avoid a sequence repeat element. In some embodiments, both the 5′ and the 3′ homology arms can be shortened to avoid including certain sequence repeat elements. In addition, some homology arm designs can improve the efficiency of editing or increase the frequency of a desired repair outcome. For example, Richardson et al. Nature Biotechnology 34, 339-344 (2016) (Richardson), which is incorporated by reference, found that the relative asymmetry of 3′ and 5′ homology arms of single stranded donor templates influenced repair rates and/or outcomes.

Replacement sequences in donor templates have been described elsewhere, including in Cotta-Ramusino et al. A replacement sequence can be any suitable length (including zero nucleotides, where the desired repair outcome is a deletion), and typically includes one, two, three or more sequence modifications relative to the naturally-occurring sequence within a cell in which editing is desired. One common sequence modification involves the alteration of the naturally-occurring sequence to repair a mutation that is related to a disease or condition of which treatment is desired. Another common sequence modification involves the alteration of one or more sequences that are complementary to, or code for, the PAM sequence of the RNA-guided nuclease or the targeting domain of the gRNA(s) being used to generate an SSB or DSB, to reduce or eliminate repeated cleavage of the target site after the replacement sequence has been incorporated into the target site.

Where a linear ssODN is used, it can be configured to (i) anneal to the nicked strand of the target nucleic acid, (ii) anneal to the intact strand of the target nucleic acid, (iii) anneal to the plus strand of the target nucleic acid, and/or (iv) anneal to the minus strand of the target nucleic acid. An ssODN may have any suitable length, e.g., about, at least, or no more than 150-200 nucleotides (e.g., 150, 160, 170, 180, 190, or 200 nucleotides).

It should be noted that a template nucleic acid can also be a nucleic acid vector, such as a viral genome or circular double stranded DNA, e.g., a plasmid. Nucleic acid vectors comprising donor templates can include other coding or non-coding elements. For example, a template nucleic acid can be delivered as part of a viral genome (e.g. in an AAV or lentiviral genome) that includes certain genomic backbone elements (e.g. inverted terminal repeats, in the case of an AAV genome) and optionally includes additional sequences coding for a gRNA and/or an RNA-guided nuclease. In certain embodiments, the donor template can be adjacent to, or flanked by, target sites recognized by one or more gRNAs, to facilitate the formation of free DSBs on one or both ends of the donor template that can participate in repair of corresponding SSBs or DSBs formed in cellular DNA using the same gRNAs. Exemplary nucleic acid vectors suitable for use as donor templates are described in Cotta-Ramusino.

Whatever format is used, a template nucleic acid can be designed to avoid undesirable sequences. In certain embodiments, one or both homology arms can be shortened to avoid overlap with certain sequence repeat elements, e.g., Alu repeats, LINE elements, etc.

Quantitative Measurement of On-Target Gene Editing

It should be noted that the genome editing systems of the present disclosure allow for the detection and quantitative measurement of on-target gene editing outcomes, including targeted integration. The compositions and methods described herein can rely on the use of donor templates comprising a 5′ homology arm, a cargo, a one or more priming sites, a 3′ homology arm, and optionally stuffer sequence. For example, International Patent Publication No. WO2019/014564 by Ramusino et al. (Ramusino), which is incorporated by reference herein in its entirety, describes compositions and methods which allow for the quantitative analysis of on-target gene editing outcomes, including targeted integration events, by embedding one or more primer binding sites (i.e., priming sites) into a donor template that are substantially identical to a priming site present at the targeted genomic DNA locus (i.e., the target nucleic acid). The priming sites are embedded into the donor template such that, when homologous recombination of the donor template with a target nucleic acid occurs, successful targeted integration of the donor template integrates the priming sites from the donor template into the target nucleic acid such that at least one amplicon can be generated in order to quantitatively determine the on-target editing outcomes.

In some embodiments, the target nucleic acid comprises a first priming site (P1) and a second priming site (P2), and the donor template comprises a cargo sequence, a first priming site (P1′), and a second priming site (P2′), wherein P2′ is located 5′ from the cargo sequence, wherein P1′ is located 3′ from the cargo sequence (i.e., A1-P2′-N-P1′-A2), wherein P1′ is substantially identical to P1, and wherein P2′ is substantially identical to P2. After accurate homology-driven targeted integration, three amplicons are produced using a single PCR reaction with two oligonucleotide primers. The first amplicon, Amplicon X, is generated from the primer binding sites originally present in the genomic DNA (P1 and P2), and may be sequenced to analyze on-target editing events that do not result in targeted integration (e.g., insertions, deletions, gene conversion). The remaining two amplicons are mapped to the 5′ and 3′ junctions after homology-driven targeted integration. The second amplicon, Amplicon Y, results from the amplification of the nucleic acid sequence between P1 and P2′ following a targeted integration event at the target nucleic acid, thereby amplifying the 5′ junction. The third amplicon, Amplicon Z, results from the amplification of the nucleic acid sequence between P1′ and P2 following a targeted integration event at the target nucleic acid, thereby amplifying the 3′ junction. Sequencing of these amplicons provides a quantitative assessment of targeted integration at the target nucleic acid, in addition to information about the fidelity of the targeted integration. To avoid any biases inherent to amplicon size, stuffer sequence may optionally be included in the donor template to keep all three expected amplicons the same length.

Implementation of Genome Editing Systems: Delivery, Formulations, and Routes of Administration

As discussed above, the genome editing systems of this disclosure can be implemented in any suitable manner, meaning that the components of such systems, including without limitation the RNA-guided nuclease, gRNA, and optional donor template nucleic acid, can be delivered, formulated, or administered in any suitable form or combination of forms that results in the transduction, expression or introduction of a genome editing system and/or causes a desired repair outcome in a cell, tissue or subject. The genome editing systems according to this disclosure can incorporate multiple gRNAs, multiple RNA-guided nucleases, and other components such as proteins, and a variety of implementations will be evident to the skilled artisan based on the principles illustrated in systems of the disclosure. In some embodiments the genome editing system of the disclosure are delivered into cells as an ribonucleoprotein (RNP) complex. In some embodiments, one or more RNP complexes are delivered to the cell sequentially in any order, or simultaneously.

Nucleic acids encoding the various elements of a genome editing system according to the present disclosure can be administered to subjects or delivered into cells by art-known methods or as described herein. For example, RNA-guided nuclease-encoding and/or gRNA-encoding DNA, as well as donor template nucleic acids can be delivered by, e.g., vectors (e.g., viral or non-viral vectors), non-vector based methods (e.g., using naked DNA or DNA complexes), or a combination thereof. In some embodiments the genome editing system of the disclosure are delivered by AAV.

Nucleic acids encoding genome editing systems or components thereof can be delivered directly to cells as naked DNA or RNA, for instance by means of transfection or electroporation, or can be conjugated to molecules (e.g., N-acetylgalactosamine) promoting uptake by the target cells (e.g., erythrocytes, HSCs). In some embodiments the genome editing system of the disclosure are delivered into cells by electroporation.

One promising solution to improve cell therapy processes consists on the direct delivery of active proteins into human cells. A protein delivery agent, the Feldan Shuttle, is a protein-based delivery agent, which is designed for cell therapy (Del′Guidice et al., PLoS One. 2018 Apr. 4; 13(4):e0195558; incorporated in its entirety herein by reference). In some embodiments the genome editing system of the disclosure are delivered into cells by the Feldan Shuttle.

The modified cells of the disclosure can be administered by any known routes of administration known to a person of kill in the art, at the time of filing this application. In some embodiments the modified cells of the disclosure are administered intravenously (IV). In some embodiments the modified NK cells of the disclosure are administered intravenously (IV).

As used herein, “dose” refers to a specific quantity of a pharmacologically active material for administration to a subject for a given time. Unless otherwise specified, the doses recited refer to NK cells having complex genomic alterations, that allow for the generation of advanced NK cell products for clinical applications. In some embodiments, a dose of modified NK cells refers to an effective amount of modified NK cells. For example, in some embodiments a dose or effective amount of modified NK cells refers to about 1×10⁹−5×10⁹ modified NK cells, or about 2×10⁹−5×10⁹ modified NK cells per dose. In some embodiments a dose or effective amount of modified NK cells refers to about 3×10⁹−5×10⁹ modified NK cells, or about 4×10⁹−5×10⁹ modified NK cells per dose.

### Generation of Modified iNK Cells

Some aspects of this disclosure relate to the generation of genetically modified NK cells that are derived from stem cells, e.g., from multipotent cells, such as, e.g., HSCs, or from pluripotent stem cells, such as, e.g., ES cells or iPS cells. In some embodiments, where genetically modified iNK cells are derived from iPS cells, the iPS cells are derived from a somatic donor cell. In some embodiments, where genetically modified iNK cells are derived from iPS cells, the iPS cells are derived from a multipotent donor cell, e.g., from an HSC.

The genomic edits present in the final iNK cell can be made at any stage of the process of reprogramming the donor cell to the iPS cell state, during the iPS cell state, and/or at any stage of the process of differentiating the iPS cell to an iNK state, e.g., at an intermediary state, such as, for example, an iPS cell-derived HSC state, or even up to or at the final iNK cell state. In some embodiments, one or more genomic edits present in a modified iNK cell provided herein is made before reprogramming the donor cell to the iPS cell state. In some embodiments, all edits present in a modified iNK cell provided herein are made at the same time, in close temporal proximity, and/or at the same cell stage of the reprogramming/differentiation process, e.g., at the donor cell stage, during the reprogramming process, at the iPS cell stage, or during the differentiation process. In some embodiments, two or more edits present in a modified iNK cell provided herein are made at different times and/or at different cell stages of the reprogramming/differentiation process. For example, in some embodiments, an edit is made at the donor cell stage and an different edit is made at the iPS cell stage; in some embodiments, an edit is made at the reprogramming stage and a different edit is made at the iPS cell stage. These examples are provided to illustrate some of the strategies provided herein, and are not meant to be limiting.

A variety of cell types can be used as a donor cell that can be subjected to the reprogramming, differentiation, and genomic editing strategies provided herein for the derivation of modified iNK cells. The donor cell to be subjected to the reprogramming, differentiation, and genomic editing strategies provided herein can be any suitable cell type. For example, the donor cell can be a pluripotent stem cell or a differentiated cell, e.g., a somatic cell, such as, for example, a fibroblast or a T lymphocyte.

In some embodiments, the donor cell is a human cell. In some embodiments, the donor cell is a non-human primate cell. In some embodiments, the donor cell is a mammalian cell. In some embodiments, the donor cell is a somatic cell. In some embodiments, the donor cell is a stem or progenitor cell. In certain embodiments, the donor cell is not part of a human embryo and its derivation does not involve the destruction of a human embryo.

In some embodiments, iNK cells, and methods of deriving such iNK cells, having one or more genomic alterations (e.g., a knock-out of a gene undesirable for immunooncology therapeutic approaches, and/or a knock-in of an exogenous nucleic acid, e.g. an expression construct encoding a gene product desirable for immunooncology therapeutic approaches) are provided herein. In some embodiments, the iNK cells are derived from an iPS cell, which in turn is derived from a somatic donor cell. Any suitable somatic cell can be used in the generation of iPS cells, and in turn, the generation of iNK cells. Suitable strategies for deriving iPS cells from various somatic donor cell types have been described and are known in the art. In some embodiments, the somatic donor cell is a fibroblast cell. In some embodiments, the somatic donor cell is a mature T cell.

For example, in some embodiments, the somatic donor cell, from which an iPS cell, and subsequently an iNK cell is derived, is a developmentally mature T cell (a T cell that has undergone thymic selection). One hallmark of developmentally mature T cells is a rearranged T cell receptor locus. During T cell maturation, the TCR locus undergoes V(D)J rearrangements to generate complete V-domain exons. These rearrangements are retained throughout reprogramming of a T cells to an induced pluripotent stem (iPS) cell, and throughout differentiation of the resulting iPS cell to a somatic cell.

In certain embodiments, the somatic donor cell is a CD8⁺ T cell, a CD8⁺ naïve T cell, a CD4⁺ central memory T cell, a CD8⁺ central memory T cell, a CD4⁺ effector memory T cell, a CD4⁺ effector memory T cell, a CD4⁺ T cell, a CD4⁺ stem cell memory T cell, a CD8⁺ stem cell memory T cell, a CD4⁺ helper T cell, a regulatory T cell, a cytotoxic T cell, a natural killer T cell, a CD4+ naïve T cell, a TH17 CD4⁺ T cell, a TH1 CD4⁺ T cell, a TH2 CD4⁺ T cell, a TH9 CD4⁺ T cell, a CD4⁺ Foxp3⁺ T cell, a CD4⁺ CD25⁺ CD127⁻ T cell, or a CD4⁺ CD25⁺ CD127⁻ Foxp3⁺ T cell.

One advantage of using T cells for the generation of iPS cells is that T cells can be edited with relative ease, e.g., by CRISPR-based methods or other gene-editing methods. Another advantage of using T cells for the generation of iPS cells is that the rearranged TCR locus allows for genetic tracking of individual cells and their daughter cells. If the reprogramming, expansion, culture, and/or differentiation strategies involved in the generation of NK cells a clonal expansion of a single cell, the rearranged TCR locus can be used as a genetic marker unambiguously identifying a cell and its daughter cells. This, in turn, allows for the characterization of a cell population as truly clonal, or for the identification of mixed populations, or contaminating cells in a clonal population.

A third advantage of using T cells in generating iNK cells carrying multiple edits is that certain karyotypic aberrations associated with chromosomal translocations are selected against in T cell culture. Such aberrations pose a concern when editing cells by CRISPR technology, and in particular when generating cells carrying multiple edits.

A fourth advantage of using T cell derived iPS cells as a starting point for the derivation of therapeutic lymphocytes is that it allows for the expression of a pre-screened TCR in the lymphocytes, e.g., via selecting the T cells for binding activity against a specific antigen, e.g., a tumor antigen, reprogramming the selected T cells to iPS cells, and then deriving lymphocytes from these iPS cells that express the TCR (e.g., T cells). This strategy would also allow for activating the TCR in other cell types, e.g., by genetic or epigenetic strategies.

A fifth advantage of using T cell derived iPS cells as a starting point for iNK differentiation is that the T cells retain at least part of their “epigenetic memory” throughout the reprogramming process, and thus subsequent differentiation of the same or a closely related cell type, such as iNK cells will be more efficient and/or result in higher quality cell populations as compared to approaches using non-related cells, such as fibroblasts, as a starting point for iNK derivation.

In certain embodiments, the donor cell being manipulated, e.g., the cell being reprogrammed and/or the cell, the genome of which is being edited, is a long term hematopoietic stem cell, a short term hematopoietic stem cell, a multipotent progenitor cell, a lineage restricted progenitor cell, a lymphoid progenitor cell, a myeloid progenitor cell, a common myeloid progenitor cell, an erythroid progenitor cell, a megakaryocyte erythroid progenitor cell, a retinal cell, a photoreceptor cell, a rod cell, a cone cell, a retinal pigmented epithelium cell, a trabecular meshwork cell, a cochlear hair cell, an outer hair cell, an inner hair cell, a pulmonary epithelial cell, a bronchial epithelial cell, an alveolar epithelial cell, a pulmonary epithelial progenitor cell, a striated muscle cell, a cardiac muscle cell, a muscle satellite cell, a neuron, a neuronal stem cell, a mesenchymal stem cell, an induced pluripotent stem (iPS) cell, an embryonic stem cell, a fibroblast, a monocyte-derived macrophage or dendritic cell, a megakaryocyte, a neutrophil, an eosinophil, a basophil, a mast cell, a reticulocyte, a B cell, e.g., a progenitor B cell, a Pre B cell, a Pro B cell, a memory B cell, a plasma B cell, a gastrointestinal epithelial cell, a biliary epithelial cell, a pancreatic ductal epithelial cell, an intestinal stem cell, a hepatocyte, a liver stellate cell, a Kupffer cell, an osteoblast, an osteoclast, an adipocyte, a preadipocyte, a pancreatic islet cell (e.g., a beta cell, an alpha cell, a delta cell), a pancreatic exocrine cell, a Schwann cell, or an oligodendrocyte.

In certain embodiments, the donor cell is a circulating blood cell, e.g., a reticulocyte, megakaryocyte erythroid progenitor (MEP) cell, myeloid progenitor cell (CMP/GMP), lymphoid progenitor (LP) cell, hematopoietic stem/progenitor cell (HSC), or endothelial cell (EC). In certain embodiments, the donor cell is a bone marrow cell (e.g., a reticulocyte, an erythroid cell (e.g., erythroblast), an MEP cell, myeloid progenitor cell (CMP/GMP), LP cell, erythroid progenitor (EP) cell, HSC, multipotent progenitor (MPP) cell, endothelial cell (EC), hemogenic endothelial (HE) cell, or mesenchymal stem cell). In certain embodiments, the donor cell is a myeloid progenitor cell (e.g., a common myeloid progenitor (CMP) cell or granulocyte macrophage progenitor (GMP) cell). In certain embodiments, the donor cell is a lymphoid progenitor cell, e.g., a common lymphoid progenitor (CLP) cell. In certain embodiments, the donor cell is an erythroid progenitor cell (e.g., an MEP cell). In certain embodiments, the donor cell is a hematopoietic stem/progenitor cell (e.g., a long term HSC (LT-HSC), short term HSC (ST-HSC), MPP cell, or lineage restricted progenitor (LRP) cell). In certain embodiments, the donor cell is a CD34⁺ cell, CD34⁺ CD90⁺ cell, CD34⁺ CD38⁻ cell, CD34⁺CD90⁺CD49f⁺CD38⁻ CD45RA⁻ cell, CD105⁺ cell, CD31⁺, or CD133⁺ cell, or a CD34⁺CD90⁺ CD133⁺ cell. In certain embodiments, the donor cell is an umbilical cord blood CD34⁺ HSPC, umbilical cord venous endothelial cell, umbilical cord arterial endothelial cell, amniotic fluid CD34⁺ cell, amniotic fluid endothelial cell, placental endothelial cell, or placental hematopoietic CD34⁺ cell. In certain embodiments, the donor cell is a mobilized peripheral blood hematopoietic CD34⁺ cell (after the patient is treated with a mobilization agent, e.g., G-CSF or Plerixafor). In certain embodiments, the donor cell is a peripheral blood endothelial cell.

In some embodiments, the donor cell is a dividing cell. In other embodiments, the donor cell is a non-dividing cell.

In some embodiments, the modified iNK cells resulting from the methods and strategies of reprogramming, differentiating, and editing provided herein, are administered to a subject in need thereof, e.g., in the context of an immunooncology therapeutic approach. In some embodiments, donor cells, or any cells of any stage of the reprogramming, differentiating, and editing strategies provided herein can be maintained in culture or stored (e.g., frozen in liquid nitrogen) using any suitable method known in the art, e.g., for subsequent characterization or administration to a subject in need thereof.

### Cell Reprogramming

A cell that has an increased cell potency has more developmental plasticity (i.e., can differentiate into more cell types) compared to the same cell in the non-reprogrammed state. In other words, a reprogrammed cell is one that is in a less differentiated state than the same cell in a non-reprogrammed state.

The reprogramming of the cells of the disclosure can be performed by utilizing several methods. Examples of some methods for reprogramming somatic cells of the disclosure are described in, but are not limited to, Valamehr et al. WO2017/078807 (“Valamehr”) and Mendlein et al. WO2010/108126 (“Mendlein”), which are hereby incorporated by reference in their entireties.

Briefly, a method for directing differentiation of pluripotent stem cells into cells of a definitive hematopoietic lineage, may comprise: (i) contacting pluripotent stem cells with a composition comprising a BMP activator, and optionally bFGF, to initiate differentiation and expansion of mesodermal cells from the pluripotent stem cells; (ii) contacting the mesodermal cells with a composition comprising a BMP activator, bFGF, and a GSK3 inhibitor, wherein the composition is optionally free of TGFβ receptor/ALK inhibitor, to initiate differentiation and expansion of mesodermal cells having definitive HE potential from the mesodermal cells; (iii) contacting the mesodermal cells having definitive HE potential with a composition comprising a ROCK inhibitor; one or more growth factors and cytokines selected from the group consisting of bFGF, VEGF, SCF, IGF, EPO, IL6, and IL11; and optionally, a Wnt pathway activator, wherein the composition is optionally free of TGFβ receptor/ALK inhibitor, to initiate differentiation and expansion of definitive hemogenic endothelium from pluripotent stem cell-derived mesodermal cells having definitive hemogenic endothelium potential; and optionally, subjecting pluripotent stem cells, pluripotent stem cell-derived mesodermal cells, mesodermal cells having hemogenic endothelium, and/or definitive hemogenic endothelium under low oxygen tension between about 2% to about 10%.

In some embodiments of the method for directing differentiation of pluripotent stem cells into cells of a hematopoietic lineage, the method further comprises contacting pluripotent stem cells with a composition comprising a MEK inhibitor, a GSK3 inhibitor, and a ROCK inhibitor, wherein the composition is free of TGFβ receptor/ALK inhibitors, to seed and expand the pluripotent stem cells. In some embodiments, the pluripotent stem cells are iPSCs. In some embodiments, the iPSCs are naïve iPSCs. In some embodiments, the iPSC comprises one or more genetic imprints, and wherein the one or more genetic imprints comprised in the iPSC are retained in the pluripotent stem cell derived hematopoietic cells differentiated therefrom.

In some embodiments of the method for directing differentiation of pluripotent stem cells into cells of a hematopoietic lineage, the differentiation of the pluripotent stem cells into cells of hematopoietic lineage is void of generation of embryoid bodies, and is in a monolayer culturing form.

In some embodiments of the above method, the obtained pluripotent stem cell-derived definitive hemogenic endothelium cells are CD34+. In some embodiments, the obtained definitive hemogenic endothelium cells are CD34+CD43−. In some embodiments, the definitive hemogenic endothelium cells are CD34+CD43−CXCR4−CD73−. In some embodiments, the definitive hemogenic endothelium cells are CD34+CXCR4−CD73−. In some embodiments, the definitive hemogenic endothelium cells are CD34+CD43−CD93−. In some embodiments, the definitive hemogenic endothelium cells are CD34+CD93−.

In some embodiments of the above method, the method further comprises (i) contacting pluripotent stem cell-derived definitive hemogenic endothelium with a composition comprising a ROCK inhibitor; one or more growth factors and cytokines selected from the group consisting of VEGF, bFGF, SCF, Flt3L, TPO, and IL7; and optionally a BMP activator; to initiate the differentiation of the definitive hemogenic endothelium to pre-T cell progenitors; and optionally, (ii) contacting the pre-T cell progenitors with a composition comprising one or more growth factors and cytokines selected from the group consisting of SCF, Flt3L, and IL7, but free of one or more of VEGF, bFGF, TPO, BMP activators and ROCK inhibitors, to initiate the differentiation of the pre-T cell progenitors to T cell progenitors or T cells. In some embodiments of the method, the pluripotent stem cell-derived T cell progenitors are CD34+CD45+CD7+. In some embodiments of the method, the pluripotent stem cell-derived T cell progenitors are CD45+CD7+.

In yet some embodiments of the above method for directing differentiation of pluripotent stem cells into cells of a hematopoietic lineage, the method further comprises: (i) contacting pluripotent stem cell-derived definitive hemogenic endothelium with a composition comprising a ROCK inhibitor; one or more growth factors and cytokines selected from the group consisting of VEGF, bFGF, SCF, Flt3L, TPO, IL3, IL7, and IL15; and optionally, a BMP activator, to initiate differentiation of the definitive hemogenic endothelium to pre-NK cell progenitor; and optionally, (ii) contacting pluripotent stem cells-derived pre-NK cell progenitors with a composition comprising one or more growth factors and cytokines selected from the group consisting of SCF, Flt3L, IL3, IL7, and IL15, wherein the medium is free of one or more of VEGF, bFGF, TPO, BMP activators and ROCK inhibitors, to initiate differentiation of the pre-NK cell progenitors to NK cell progenitors or NK cells. In some embodiments, the pluripotent stem cell-derived NK progenitors are CD3−CD45+CD56+CD7+. In some embodiments, the pluripotent stem cell-derived NK cells are CD3−CD45+CD56+, and optionally further defined by NKp46+, CD57+ and CD16+.

In yet some embodiments of the above method for directing differentiation of pluripotent stem cells into NK cells, the method further comprises knocking out the gene Nrg1 in the pluripotent stem cells.

In some embodiments, the disclosure provides a method for generating pluripotent stem cell-derived T lineage cells, which comprises: (i) contacting pluripotent stem cells with a composition comprising a BMP activator, and optionally bFGF, to initiate differentiation and expansion of mesodermal cells from pluripotent stem cells; (ii) contacting the mesodermal cells with a composition comprising a BMP activator, bFGF, and a GSK3 inhibitor, but free of TGFβ receptor/ALK inhibitor, to initiate differentiation and expansion of the mesodermal cells having definitive HE potential from the mesodermal cells; (iii) contacting mesodermal cells having definitive HE potential with a composition comprising a ROCK inhibitor; one or more growth factors and cytokines selected from the group consisting of bFGF, VEGF, SCF, IGF, EPO, IL6, and IL11; and optionally, a Wnt pathway activator; wherein the composition is free of TGFβ receptor/ALK inhibitor, to initiate differentiation and expansion of definitive hemogenic endothelium from mesodermal cells having definitive HE potential; (iv) contacting definitive hemogenic endothelium with a composition comprising a ROCK inhibitor; one or more growth factors and cytokines selected from the group consisting of VEGF, bFGF, SCF, Flt3L, TPO, and IL7; and optionally a BMP activator; to initiate differentiation of the definitive hemogenic endothelium to pre-T cell progenitors; and (v) contacting the pre-T cell progenitors with a composition comprising one or more growth factors and cytokines selected from the group consisting of SCF, Flt3L, and IL7, wherein the composition is free of one or more of VEGF, bFGF, TPO, BMP activators and ROCK inhibitors; to initiate differentiation of the pre-T cell progenitors to T cell progenitors or T cells; and optionally, the seeded pluripotent stem cells, mesodermal cells, mesodermal cells having definitive HE potential, and/or definitive hemogenic endothelium may be subject to low oxygen tension between about 2% to about 10%. In some embodiments, group II of the above method further comprises: contacting iPSCs with a composition comprising a MEK inhibitor, a GSK3 inhibitor, and a ROCK inhibitor, but free of TGFβ receptor/ALK inhibitors, to seed and expand pluripotent stem cells; and/or wherein the pluripotent stem cells. In some embodiments, the pluripotent stem cells are iPSCs. In some embodiments, the iPSCs are naïve iPSC. In some embodiments of the method, the differentiation of the pluripotent stem cells into T cell lineages is void of generation of embryoid bodies, and is in a monolayer culturing format.

In some embodiments, the disclosure provides a method for generating pluripotent stem cell-derived NK lineage cells, which comprises: (i) contacting pluripotent stem cells with a composition comprising a BMP activator, and optionally bFGF, to initiate differentiation and expansion of mesodermal cells from the pluripotent stem cells; (ii) contacting mesodermal cells with a composition comprising a BMP activator, bFGF, and a GSK3 inhibitor, and optionally free of TGFβ receptor/ALK inhibitor, to initiate differentiation and expansion of mesodermal cells having definitive HE potential from mesodermal cells; (iii) contacting mesodermal cells having definitive HE potential with a composition comprising one or more growth factors and cytokines selected from the group consisting of bFGF, VEGF, SCF, IGF, EPO, IL6, and IL11; a ROCK inhibitor; optionally a Wnt pathway activator; and optionally free of TGFβ receptor/ALK inhibitor, to initiate differentiation and expansion of pluripotent stem cell-derived definitive hemogenic endothelium from the pluripotent stem cell-derived mesodermal cells having definitive HE potential; (iv) contacting pluripotent stem cell-derived definitive hemogenic endothelium with a composition comprising a ROCK inhibitor; one or more growth factors and cytokines selected from the group consisting of VEGF, bFGF, SCF, Flt3L, TPO, IL3, IL7, and IL15, and optionally, a BMP activator, to initiate differentiation of the pluripotent stem cell-derived definitive hemogenic endothelium to pre-NK cell progenitors; and (v) contacting pluripotent stem cell-derived pre-NK cell progenitors with a composition comprising one or more growth factors and cytokines selected from the group consisting of SCF, Flt3L, IL3, IL7, and IL15, but free of one or more of VEGF, bFGF, TPO, BMP activators and ROCK inhibitors, to initiate differentiation of the pluripotent stem cell-derived pre-NK cell progenitors to pluripotent stem cell-derived NK cell progenitors or NK cells; and optionally, subjecting seeded pluripotent stem cells, pluripotent stem cell-derived-mesodermal cells, and/or definitive hemogenic endothelium under low oxygen tension between about 2% to about 10%. In some embodiments, the method for generating pluripotent stem cell-derived NK lineage cells of group II further comprises contacting iPSCs with a composition comprising a MEK inhibitor, a GSK3 inhibitor, and a ROCK inhibitor, but free of TGFβ receptor/ALK inhibitors, to seed and expand the iPSCs. In some embodiments, the iPSCs are naïve iPSCs. In some embodiments, the method for generating pluripotent stem cell-derived NK lineage cells is void of generation of embryoid bodies, and is in a monolayer culturing format.

In some embodiments, the disclosure provides a method for generating pluripotent stem cell-derived definitive hemogenic endothelium, the method comprises: (i) contacting iPSCs with a composition comprising a BMP activator, and optionally bFGF, to initiate differentiation and expansion of pluripotent stem cell-derived mesodermal cells from pluripotent stem cells; (ii) contacting pluripotent stem cell-derived mesodermal cells with a composition comprising a BMP activator, bFGF, and a GSK3 inhibitor, and optionally free of TGFβ receptor/ALK inhibitor, to initiate differentiation and expansion of pluripotent stem cell-derived mesodermal cells having definitive HE potential from pluripotent stem cell-derived mesodermal cells; (iii) contacting pluripotent stem cell-derived mesodermal cells having definitive HE potential with a composition comprising one or more growth factors and cytokines selected from the group consisting of bFGF, VEGF, SCF, IGF, EPO, IL6, and IL11; a ROCK inhibitor; and optionally a Wnt pathway activator, and optionally free of TGFβ receptor/ALK inhibitor, to initiate differentiation and expansion of pluripotent stem cell-derived definitive hemogenic endothelium from the pluripotent stem cell-derived mesodermal cells having definitive HE potential; and optionally, subjecting seeded pluripotent stem cells, pluripotent stem cell-derived mesodermal cells, and/or definitive hemogenic endothelium under low oxygen tension between about 2% to about 10%. In some embodiments, the above method for generating pluripotent stem cell-derived definitive hemogenic endothelium, further comprises: contacting iPSCs with a composition comprising a MEK inhibitor, a GSK3 inhibitor, and a ROCK inhibitor, but free of TGFβ receptor/ALK inhibitors, to seed and expand the iPSCs; and/or wherein the iPSCs are naïve iPSCs. In some embodiments, the iPSC comprises one or more genetic imprints, and wherein the one or more genetic imprints comprised in the iPSC are retained in the pluripotent stem cell derived definitive hemogenic endothelium cells differentiated therefrom. In some embodiments, the above method of differentiating iPSCs into cells of a definitive hemogenic endothelium is void of generation of embryoid bodies, and is in monolayer culturing format.

In some embodiments, the disclosure provides a method for generating pluripotent stem cell-derived multipotent progenitors of hematopoietic lineage, comprising: (i) contacting iPSCs with a composition comprising a BMP activator, and optionally bFGF, to initiate differentiation and expansion of pluripotent stem cell-derived mesodermal cells from iPSCs; (ii) contacting pluripotent stem cell-derived mesodermal cells with a composition comprising a BMP activator, bFGF, and a GSK3 inhibitor, but free of TGFβ receptor/ALK inhibitor, to initiate differentiation and expansion of the mesodermal cells having definitive HE potential from the mesodermal cells; (iii) contacting mesodermal cells having definitive HE potential with a composition comprising a ROCK inhibitor; one or more growth factors and cytokines selected from the group consisting of bFGF, VEGF, SCF, IGF, EPO, IL6, and IL11; and optionally, a Wnt pathway activator, wherein the composition is free of TGFβ receptor/ALK inhibitor, to initiate differentiation and expansion of definitive hemogenic endothelium from mesodermal cells having definitive HE potential; (iv) contacting definitive hemogenic endothelium with a composition comprising a BMP activator, a ROCK inhibitor, one or more growth factors and cytokines selected from the group consisting of TPO, IL3, GMCSF, EPO, bFGF, VEGF, SCF, IL6, Flt3L and IL11, to initiate differentiation of definitive hemogenic endothelium to pre-HSC; and (v) contacting pre-HSC with a composition comprising a BMP activator, one or more growth factors and cytokines selected from the group consisting of TPO, IL3, GMCSF, EPO, bFGF, VEGF, SCF, IL6, and IL11, but free of ROCK inhibitor, to initiate differentiation of the pre-HSC to hematopoietic multipotent progenitors; and optionally, subjecting seeded pluripotent stem cells, mesodermal cells, and/or definitive hemogenic endothelium under low oxygen tension between about 2% to about 10%. In some embodiments, the above method for generating pluripotent stem cell-derived hematopoiesis multipotent progenitors further comprises contacting pluripotent stem cells with a composition comprising a MEK inhibitor, a GSK3 inhibitor, and a ROCK inhibitor, but free of TGFβ receptor/ALK inhibitors, to seed and expand the pluripotent stem cells. In some embodiments, the pluripotent stem cells are iPSCs. In some embodiments, the iPSCs are naïve iPSCs. In some embodiments, the iPSC comprises one or more genetic imprints, and wherein the one or more genetic imprints comprised in the iPSC are retained in the pluripotent stem cell derived hematopoietic multipotent progenitor cells differentiated therefrom. In some embodiments, the differentiation of the pluripotent stem cells into hematopoiesis multipotent progenitors using the above method is void of generation of embryoid bodies, and is in monolayer culturing format.

In some embodiments, the disclosure provides a composition comprising: one or more cell populations generated from the culture platform disclosed herein: pluripotent stem cells-derived (i) CD34+ definitive hemogenic endothelium (iCD34), wherein the iCD34 cells have capacity to differentiate into multipotent progenitor cells, T cell progenitors, NK cell progenitors, T cells, NK cells, NKT cells and B cells, and wherein the iCD34 cells are CD34+CD43−; (ii) definitive hemogenic endothelium (iHE), wherein the iHE cells are CD34+, and at least one of CD43−, CD93−, CXCR4−, CD73−, and CXCR4−CD73−; (iii) pluripotent stem cell-derived definitive HSCs, wherein the iHSC is CD34+CD45+; (iv) hematopoietic multipotent progenitor cells, wherein the iMPP cells are CD34+CD45+; (v) T cell progenitors, wherein the T cell progenitors are CD34+CD45+CD7+ or CD34−CD45+CD7+; (vi) T cells, wherein the T cells are CD45+CD3+CD4+ or CD45+CD3+CD8+; (vii) NK cell progenitors, wherein the NK cell progenitors are CD45+CD56+CD7+; (viii) NK cells, wherein the NK cells are CD3−CD45+CD56+, and optionally further defined by NKp46+, CD57+, and CD16+; (ix) NKT cells, wherein the NKT cells are CD45+Vα24Jα18+CD3+; and (x) B cells, wherein the B cells are CD45+CD19+.

In some embodiments, the disclosure provides one or more cell lines, or clonal cells generated using the methods disclosed herein: pluripotent stem cell-derived (i) CD34+ definitive hemogenic endothelium (iCD34), wherein the iCD34 cells have capacity to differentiate into multipotent progenitor cells, T cell progenitors, NK cell progenitors, T cells, NK cells, and NKT cells, and wherein the iCD34 cells are CD34+CD43−; (ii) definitive hemogenic endothelium (iHE), wherein the iHE cell line or clonal cells are CD34+, and at least one of CD43−, CD93−, CXCR4−, CD73−, and CXCR4−CD73−; (iii) definitive HSCs, wherein the iHSCs is CD34+CD45+; (iv) hematopoietic multipotent progenitor cells (iMPP), wherein the iMPP cells are CD34+CD45+; (v) T cell progenitors, wherein the T cell progenitors are CD34+CD45+CD7+ or CD34−CD45+CD7+; (vi) T cells, wherein the T cells are CD45+CD3+CD4+ or CD45+CD3+CD8+; (vii) NK cell progenitors, wherein the NK cell progenitors are CD45+CD56+CD7+; (viii) NK cells, wherein the NK cells are CD3−CD45+CD56+, and optionally further defined by NKp46+, CD57+, and CD16+; (ix) NKT cells, wherein the NKT cells are CD45+Vα24Jα18+CD3+; and (x) B cells, wherein the B cells are CD45+CD19+.

In some embodiments, the present disclosure provides a method of promoting hematopoietic self-renewal, reconstitution or engraftment using one or more of cell populations, cell lines or clonal cells generated using methods as disclosed: pluripotent stem cell-derived (i) CD34+ definitive hemogenic endothelium (iCD34), wherein the iCD34 cells have capacity to differentiate into multipotent progenitor cells, T cell progenitors, NK cell progenitors, T cells NK cells and NKT cells, and wherein the iCD34 cells are CD34+CD43−; (ii) definitive hemogenic endothelium (iHE), wherein the iHE cell line or clonal cells are CD34+, and at least one of CD43−, CD93−, CXCR4−, CD73−, and CXCR4−CD73−; (iii) definitive HSCs, wherein the iHSCs are CD34+CD45+; (iv) hematopoietic multipotent progenitor cells, wherein the iMPP cells are CD34+CD45+; (v) T cell progenitors, wherein the T cell progenitors are CD34+CD45+CD7+ or CD34−CD45+CD7+; (vi) T cells, wherein the T cells are CD45+CD3+CD4+ or CD45+CD3+CD8+; (vii) NK cell progenitors, wherein the NK cell progenitors are CD45+CD56+CD7+; (viii) NK cells, wherein the NK cells are CD3−CD45+CD56+, and optionally further defined by NKp46+, CD57+, and CD16+; (ix) NKT cells, wherein the NKT cells are CD45+Vα24Jα18+CD3+; and (x) B cells, wherein the B cells are CD45+CD19+.

In some embodiments, the present disclosure provides a method of generating hematopoietic lineage cells with enhanced therapeutic properties, and the method comprises: obtaining iPSCs comprising one or more genetic imprints; and directing differentiation of iPSCs to hematopoietic lineage cells. The step of directed differentiation further comprises: (i) contacting the pluripotent stem cells with a composition comprising a BMP pathway activator, and optionally bFGF, to obtain mesodermal cells; and (ii) contacting the mesodermal cells with a composition comprising a BMP pathway activator, bFGF, and a WNT pathway activator, to obtain mesodermal cells having definitive hemogenic endothelium (HE) potential, wherein the mesodermal cells having definitive hemogenic endothelium (HE) potential are capable of providing hematopoietic lineage cells. Preferably, the mesodermal cells and mesodermal cells having definitive HE potential are obtained in steps (i) and (ii) without the step of forming embryoid bodies, and the obtained hematopoietic lineage cells comprise definitive hemogenic endothelium cells, hematopoietic stem and progenitor cells (HSC), hematopoietic multipotent progenitor cell (MPP), pre-T cell progenitor cells, pre-NK cell progenitor cells, T cell progenitor cells, NK cell progenitor cells, T cells, NK cells, NKT cells, or B cells. Moreover, the hematopoietic lineage cells retain the genetic imprints comprised in the iPSCs for directed differentiation.

In some embodiments, the step of directed differentiation of the above method further comprises: (i) contacting the mesodermal cells having definitive HE potential with a composition comprising bFGF and a ROCK inhibitor to obtain definitive HE cells; (ii) contacting the definitive HE cells with a composition comprising a BMP activator, and optionally a ROCK inhibitor, and one or more growth factors and cytokines selected from the group consisting of TPO, IL3, GMCSF, EPO, bFGF, VEGF, SCF, IL6, Flt3L and IL11 to obtain hematopoietic multipotent progenitor cells (MPP); (iii) contacting the definitive HE cells with a composition comprising one or more growth factors and cytokines selected from the group consisting of SCF, Flt3L, and IL7; and optionally one or more of a BMP activator, a ROCK inhibitor, TPO, VEGF and bFGF to obtain pre-T cell progenitors, T cell progenitors, and/or T cells; or (iv) contacting the definitive HE cells with a composition comprising one or more growth factors and cytokines selected from the group consisting of SCF, Flt3L, TPO, IL7 and IL15, and optionally one or more of a BMP activator, a ROCK inhibitor, VEGF and bFGF to obtain pre-NK cell progenitors, NK cell progenitors, and/or NK cells.

Briefly, the method may comprise reprogramming a mature source T or B cell to obtain induced pluripotent stem cells (iPSCs); and detecting the presence, in the iPSCs or the hematopoietic lineage cells derived therefrom, of a specific V(D)J recombination that is same as the one comprised in the mature T or B cell for generating the iPSC. In some embodiments, the above method further comprises isolating iPSCs or hematopoietic lineage cells comprising the same V(D)J recombination as that of the mature source T or B cell. In some embodiments, the above method comprises, prior to reprogramming the source cells, obtaining a mature source T or B cell for reprogramming; and determining V(D)J recombination comprised in immunoglobulins (Ig) or T cell receptors (TCR) that is specific to the mature source T or B cell.

A “pluripotency factor,” or “reprogramming factor,” refers to an agent capable of increasing the developmental potency of a cell, either alone or in combination with other agents. Pluripotency factors include, without limitation, polynucleotides, polypeptides, and small molecules capable of increasing the developmental potency of a cell. Exemplary pluripotency factors include, for example, transcription factors and small molecule reprogramming agents.

A number of various cell types from all three germ layers have been shown to be suitable for somatic cell reprogramming, including, but not limited to liver and stomach (Aoi et al., 2008); pancreatic β cells (Stadtfeld et al., 2008); mature B lymphocytes (Hanna et al., 2008); human dermal fibroblasts (Takahashi et al., 2007; Yu et al., 2007; Lowry et al., 2008; Aasen et al., 2008); meningiocytes (Qin et al., 2008); neural stem cells (DiSteffano et al., 2008); and neural progenitor cells (Eminli et al., 2008). Thus, the present disclosure contemplates, in part, methods to reprogram and/or program cells from any cell lineage.

The present disclosure contemplates, in part, to alter the potency of a cell by contacting the cell with one or more repressors and/or activators to modulate the epigenetic state, chromatin structure, transcription, mRNA splicing, post-transcriptional modification, mRNA stability and/or half-life, translation, post-translational modification, protein stability and/or half-life and/or protein activity of a component of a cellular pathway associated with determining or influencing cell potency.

Thus, in various embodiments, the present disclosure uses predictable and highly controlled methods for gene expression, as discussed elsewhere herein, that enable the reprogramming or de-differentiation and programming or differentiation of somatic cells ex vivo or in vivo. As, noted above, the intentional genetic engineering of cells, however, is not preferred, since it alters the cellular genome and would likely result in genetic or epigenetic abnormalities. In contrast, the compositions and methods of the present disclosure provide repressors and/or activators that non-genetically alter the potency of a cell by mimicking the cell's endogenous developmental potency pathways to achieve reprogramming and/or programming of the cell.

Small Molecules in Reprogramming

Reprogramming of somatic cells into induced pluripotent stem cells has also been achieved by retroviral infection of defined genes (e.g., Oct-3/4, Sox-2, Klf-4, c-Myc, and Lin28, and the like) in combination with small molecules.

In some embodiments, the present disclosure provides a method of altering the potency of a cell that comprises contacting the cell with one or more repressors and/or activators or a composition comprising the same, wherein said one or more repressors and/or activators modulates at least one component of a cellular pathway associated with the potency of the cell, thereby altering the potency of the cell. In particular embodiments, the one or more repressors and/or activators modulate one or more components of a cellular pathway associated with the potency of the cell and thereby alter the potency of the cell. In certain embodiments, the one or more repressors and/or activators modulate one or more components of one or more cellular pathways associated with the potency of the cell and thereby alter the potency of the cell. In certain related embodiments, the modulation of the component(s) is synergistic and increases the overall efficacy of altering the potency of a cell. The potency of the cell can be altered, compared to the ground potency state, to a more potent state (e.g., from a differentiated cell to a multipotent, pluripotent, or totipotent cell) or a less potent state (e.g., from a totipotent, pluripotent, or multipotent cell to a differentiated somatic cell). In still yet other embodiments, the potency of a cell may be altered more than once. For example, a cell may first be reprogrammed to a more potent state, then programmed to a particular somatic cell.

In another embodiment, the methods of the present disclosure provide for increasing the potency a cell, wherein the cell is reprogrammed or dedifferentiated to a totipotent state, comprising contacting the cell with a composition comprising one or more repressors and/or activators, wherein the one or more repressors and/or activators modulates at least one component of a cellular pathway associated with the totipotency of the cell, thereby increasing the potency of the cell to a totipotent state.

In a particular embodiment, a method of increasing the potency a cell to a pluripotent state comprises contacting the cell with one or more repressors and/or activators, wherein the one or more repressors and/or activators modulates at least one component of a cellular pathway associated with the potency of the cell, thereby increasing the potency of the cell to a pluripotent state.

In another particular embodiment, a method of increasing the potency a cell to a multipotent state comprises contacting the cell with one or more repressors and/or activators, wherein the one or more repressors and/or activators modulates at least one component of a cellular pathway associated with the potency of the cell, thereby increasing the potency of the cell to a multipotent state.

In certain embodiments, a method of increasing the potency of a cell further comprises a step of contacting the totipotent cell, the pluripotent cell or the multipotent cell with a second composition, wherein the second composition modulates the at least one component of a cellular potency pathway to decrease the totipotency, pluripotency or multipotency of the cell and differentiate the cell to a mature somatic cell.

In another related embodiment, the present disclosure provides a method of reprogramming a cell that comprises contacting the cell with a composition comprising one or more repressors and/or activators, wherein the one or more repressors and/or activators modulates at least one component of a cellular pathway or pathways associated with the reprogramming of a cell, thereby reprogramming the cell.

In other embodiments, the present disclosure provides a method of dedifferentiating a cell to a more potent state, comprising contacting the cell with the composition comprising one/or more activators, wherein the one or more repressors and/or activators modulates at least one component of a cellular pathway or pathways associated with the dedifferentiation of the cell to the more potent state, thereby dedifferentiating the cell to an impotent state.

According to various embodiments of the present disclosure a repressor can be an antibody or an antibody fragment, an intrabody, a transbody, a DNAzyme, an ssRNA, a dsRNA, an mRNA, an antisense RNA, a ribozyme, an antisense oligonucleotide, a pri-miRNA, an shRNA, an antagomir, an aptamer, an siRNA, a dsDNA, a ssDNA; a polypeptide or an active fragment thereof, a peptidomimetic, a peptoid, or a small organic molecule. Polypeptide-based repressors include, but are not limited to fusion polypeptides. Polypeptide-based repressors also include transcriptional repressors, which can further be fusion polypeptides and/or artificially designed transcriptional repressors as described elsewhere herein.

According to other various embodiments, an activator can be an antibody or an antibody fragment, an mRNA, a bifunctional antisense oligonucleotide, a dsDNA, a polypeptide or an active fragment thereof, a peptidomimetic, a peptoid, or a small organic molecule.

In some embodiments, repressors modulate at least one component of a cellular potency pathway by a) repressing the at least one component; b) de-repressing a repressor of the at least one component; or c) repressing an activator of the at least one component. In related embodiments, one or more repressors can modulate at least one component of a pathway associated with the potency of a cell by a) de-repressing the at least one component; b) repressing a repressor of the at least one component; or c) de-repressing an activator of the at least one component.

In certain embodiments, one or more repressors modulates at least one component of a cellular pathway associated with the potency of a cell by a) repressing a histone methyltransferase or repressing the at least one component's epigenetic state, chromatin structure, transcription, mRNA splicing, post-transcriptional modification, mRNA stability and/or half-life, translation, post-translational modification, protein stability and/or half-life and/or protein activity; or b) de-repressing a demethylase or activating the at least one component's epigenetic state, chromatin structure, transcription, mRNA splicing, post-transcriptional modification, mRNA stability and/or half-life, translation, post-translational modification, protein stability and/or half-life and/or protein activity.

In related embodiments, activators modulate at least one component of a cellular pathway associated with the potency of a cell by a) activating the at least one component; b) activating a repressor of a repressor of the at least one component; or c) activating an activator of the at least one component.

In certain embodiments, one or more activators modulates at least one component by a) activating a histone demethylase or activating the at least one component's epigenetic state, chromatin structure, transcription, mRNA splicing, post-transcriptional modification, mRNA stability and/or half-life, translation, post-translational modification, protein stability and/or half-life and/or protein activity; or b) activating a repressor of a histone methyltransferase or activating a repressor of the at least one component's epigenetic state, chromatin structure, transcription, mRNA splicing, post-transcriptional modification, mRNA stability and/or half-life, translation, post-translational modification, protein stability and/or half-life and/or protein activity.

In various other embodiments, the present disclosure contemplates, in part, a method of reprogramming a cell, comprising contacting the cell with one or more repressors, wherein the one or more repressors modulates at least one component of a cellular pathway associated with the reprogramming of a cell, thereby reprogramming the cell.

In various other embodiments, the present disclosure contemplates, in part, a method of reprogramming a cell, comprising contacting the cell with a composition comprising one or more activators, wherein the one or more activators modulates at least one component of a cellular pathway associated with the reprogramming of a cell, thereby re-programming the cell.

While some exemplary methods for reprogramming/NK cell differentiation are provided herein, these are exemplary and not meant to limit the scope of the present disclosure. Additional suitable methods for reprogramming/NK cell differentiation will be apparent to those of skill in the art based on the present disclosure in view of the knowledge in the art.

Methods for culturing NK cells on feeder layers or with feeder cells are described in detail in, for e.g., EP3184109 by Valamehr et al. (“Valamehr”) incorporated in its entirety herein by reference.

In general, any type of NK cell population can be cultured using a variety of methods and devices. Selection of culture apparatus is usually based on the scale and purpose of the culture. Scaling up of cell culture preferably involves the use of dedicated devices. Apparatus for large scale, clinical grade NK cell production is detailed, for example, in Spanholtz et al. (PLoS ONE 2010; 5:e9221) and Sutlu et al. (Cytotherapy 2010, Early Online 1-12).

The methods described hereinabove for ex vivo culturing NK cells populations can result, inter alia, in a cultured population of NK cells.

### Types of Edits

Some aspects of the present disclosure provide complex editing strategies, and resulting NK cells having complex genomic alterations, that allow for the generation of advanced NK cell products for clinical applications, e.g., for immunooncology therapeutic approaches. In some embodiments, the modified NK cells provided herein can serve as an off-the-shelf clinical solution for patients having, or having been diagnosed with, a hyperproliferative disease, such as, for example, a cancer. In some embodiments, the modified NK cells exhibit an enhanced survival, proliferation, NK cell response level, NK cell response duration, resistance against NK cell exhaustion, and/or target recognition as compared to non-modified NK cells. For example, the modified NK cells provided herein may comprise genomic edits that result in: expression of a chimeric antigen receptor (CAR) of interest, e.g., a CAR targeting mesothelin, EGFR, HER2 and/or MICA/B; may express a CD16 variant, e.g., hnCD16; expression of an IL15/IL15RA fusion; a loss-of-function in TGF beta receptor 2 (TGFbetaR2); and/or expression of a dominant-negative variant of TGFbetaR2; a loss-of-function of ADORA2A; a loss-of-function of B2M; expression of HLA-G: a loss-of-function of a CIITA; a loss-of-function of a PD1; a loss-of-function of TIGIT; and/or a loss-of-function of CISH; or any combination of two or more thereof in the modified NK cell.

In some embodiments, the modified NK cells provided herein may comprise genomic edits that result in: expression of an exogenous a CD16 variant, e.g., hnCD16, expression of an exogenous IL15/IL15RA fusion, expression of an exogenous HLA-G, expression of an exogenous DN-TGFbetaR2, a loss of function in TGFbetaR2, a loss of function in B2M, a loss of function of PD1, a loss of function of TIGIT, and/or a loss of function of ADORA2A.

In some embodiments, the modified NK cells provided herein may comprise genomic edits that result in: expression of an exogenous a CD16 variant, e.g., hnCD16, expression of an exogenous IL15/IL15RA fusion, expression of an exogenous HLA-G, expression of an exogenous DN-TGFbetaR2, expression of a soluble MICA and/or MICB, a loss of function in TGFbetaR2, a loss of function in B2M, a loss of function of PD1, a loss of function of TIGIT, and/or a loss of function of ADORA2A.

In some embodiments, the modified NK cells provided herein may comprise genomic edits that result in: expression of an exogenous a CD16 variant, e.g., hnCD16, expression of an exogenous IL15/IL15RA fusion, expression of an exogenous HLA-G, expression of an exogenous DN-TGFbetaR2, expression of a soluble MICA and/or MICB, expression of an exogenous IL-12, expression of an exogenous IL-18, a loss of function in TGFbetaR2, a loss of function in B2M, a loss of function of PD1, a loss of function of TIGIT, and/or a loss of function of ADORA2A.

In some embodiments, the modified NK cells provided herein may comprise genomic edits that result in: expression of an exogenous a CD16 variant, e.g., hnCD16, expression of an exogenous IL15/IL15RA fusion, expression of an exogenous HLA-G, expression of an exogenous DN-TGFbetaR2, expression of an exogenous IL-12, expression of an exogenous IL-18, a loss of function in TGFbetaR2, a loss of function in B2M, a loss of function of PD1, a loss of function of TIGIT, and/or a loss of function of ADORA2A.

The modified NK cells may exhibit one or more edits in their genome that results in a loss-of-function in a target gene, and/or one or more modifications that results in a gain-of-function, or an overexpression, of a gene product, e.g., of a protein, from an exogenous nucleic acid construct, e.g., from an expression construct comprising a cDNA encoding for the gene product that is integrated into the genome of the modified NK cell or provided in an extrachromosomal manner, e.g., in the form of an episomal expression construct.

A loss-of-function of a target gene is characterized by a decrease in the expression of a target gene based on a genomic modification, e.g., an RNA-guided nuclease-mediated cut in the target gene that results in an inactivation, or in diminished expression or function, of the encoded gene product.

A gain-of-function of a gene product is characterized by an increased expression (also referred to herein as overexpression) of a gene product, e.g., of a protein, in a cell, which can include, for example, an increased expression level of the gene product, or expression of the gene product in a cell that does not express the gene product endogenously, e.g., from an endogenous gene.

In some embodiments, increased expression of a gene product is effected by introducing an exogenous nucleic acid construct that encodes the gene product into a cell, e.g., an exogenous nucleic acid construct that comprises a cDNA encoding the gene product under the control of a heterologous promoter. In some embodiments, the exogenous nucleic acid construct is integrated into a specific locus, e.g., via HDR-mediated gene editing, as described in more detail elsewhere herein. Methods for effecting loss-of-function edits as well as methods for effecting increased expression of gene products, e.g., via RNA-guided nuclease technology are well known to those of ordinary skill in the art.

Some exemplary gene products, one or more of which may be overexpressed in a modified NK cells provided in some embodiments of this disclosure are provided in Table 10 below:

TABLE 10
CAR Chimeric antigen receptor (e.g., binding to Her2, EGFR, alpha folate
receptor, CEA, cMET, MUC1, Mesothelin, ROR1, or other targets.)
CD16 or CD16 variant (e.g., hnCD16)
IL-15/IL-15R/IL-15RA
IL-12/IL-12R/IL-12RA
IL-2/IL-2R/IL-2RA
HLA-G
HLA-E
CD47
CXCR1
CX3CR1
mTRAIL
TOSO

Some exemplary target genes, one or more of which are modified to exhibit a loss-of-function in modified NK cells provided in some embodiments of this disclosure are provided in Table 11 below.

TABLE 11
TGFβR2
ADORA2A
TIGIT
B2M
PD-1
CISH
CIITA
HLA class II histocompatibility antigen alpha chain genes, e.g., HLA-
DQA1, HLA-DRA, HLA-DPA1, HLA-DMA, HLA-DQA2, and/or HLA-
DOA
HLA class II histocompatibility antigen beta chain genes, e.g., HLA-
DMB, HLA-DOB, HLA-DPB1, HLA-DQB1, HLA-DQB2, HLA-DQB3,
HLA-DRB1, HLA-DRB3, HLA-DRB4, and/or HLA-DRB5
CD32B (FCGR2B)
CTLA4
NKG2A
BIM
CBLB
CCR5
CCR7
CD96
CDK8
CXCR3
EP4 (PGE2 receptor)
Fas
GITR
IL1R8
KIRDL1
KIR2DL1-3
LAG3
SOCS genes
Sortilin
TIM3
TRAC
NLRC5

The present disclosure embraces modified NK cells exhibiting any of the edits and/or increased expression of gene products listed in TABLES 7 and TABLES 8 combined, as well as any combination of such edits and/or increased expression of gene products listed in these tables. For example, it is to be understood that the present disclosure embraces embodiments in which modified NK cells are provided that comprise a single edit listed in TABLE 10 or TABLE 11, e.g., loss of function of ADORA2A, or loss of function of B2M, or increased expression of HLA-G, etc. It is to be understood that the present disclosure embraces embodiments in which modified NK cells are provided that comprise a single edit listed in TABLE 11 and increased expression of a gene product listed in TABLE 10, e.g., loss of function of ADORA2A or loss of function of B2M; and increased expression of HLA-G. It is further to be understood that the present disclosure embraces embodiments in which modified NK cells are provided that comprise two or more edits listed in TABLE 11, and increased expression of a single gene product listed in TABLE 10. It is further to be understood that the present disclosure embraces embodiments in which modified NK cells are provided that comprise a single edit listed in TABLE 11, and increased expression of two or more gene products listed in TABLE 10. It is further to be understood that the present disclosure embraces embodiments in which modified NK cells are provided that comprise two or more edits listed in TABLE 11, and increased expression of two or more gene products listed in TABLE 10.

In order to illustrate some of the configurations of modified NK cells embraced by the present disclosure, some exemplary, non-limiting embodiments are provided below and elsewhere herein. In some embodiments, modified NK cells are provided that exhibit a loss-of-function of ADORA2A. In some embodiments, modified NK cells are provided that exhibit a loss-of-function of B2M. In some embodiments, modified NK cells are provided that exhibit a loss-of-function of TGFbRII. In some embodiments, modified NK cells are provided that exhibit a loss-of-function of ADORA2A and B2M. In some embodiments, modified NK cells are provided that exhibit a gain-of-function of hnCD16. In some embodiments, modified NK cells are provided that exhibit a gain-of-function of a CAR, e.g., a CAR binding Her2, EGFR, alpha folate receptor, CEA, cMET, MUC1, Mesothelin, ROR1, or a different target, e.g., as disclosed herein or otherwise known in the art. In some embodiments, modified NK cells are provided that exhibit a gain-of-function of HLA-G. In some embodiments, modified NK cells are provided that exhibit a gain-of-function of a single-chain IL-15/IL-15R fusion protein. In some embodiments, modified NK cells are provided that exhibit a loss-of-function of ADORA2A and B2M, and a gain-of-function of hnCD16. In some embodiments, modified NK cells are provided that exhibit a loss-of-function of ADORA2A and B2M, and a gain-of-function of a CAR, e.g., a CAR binding Her2, EGFR, alpha folate receptor, CEA, cMET, MUC1, Mesothelin, ROR1, or a different target, e.g., as disclosed herein or otherwise known in the art. In some embodiments, modified NK cells are provided that exhibit a loss-of-function of ADORA2A and B2M, and a gain-of-function of HLA-G. In some embodiments, modified NK cells are provided that exhibit a loss-of-function of ADORA2A and B2M, and a gain-of-function of a single-chain IL-15/IL-15R fusion protein. In some embodiments, modified NK cells are provided that exhibit a loss-of-function of ADORA2A and B2M, and a gain-of-function of hnCD16 and a dominant-negative TGFbRII variant. In some embodiments, modified NK cells are provided that exhibit a loss-of-function of ADORA2A and B2M, and a gain-of-function of a CAR, e.g., a CAR binding Her2, EGFR, alpha folate receptor, CEA, cMET, MUC1, Mesothelin, ROR1, or a different target, e.g., as disclosed herein or otherwise known in the art, and a dominant-negative TGFbRII variant. In some embodiments, modified NK cells are provided that exhibit a loss-of-function of ADORA2A and B2M, and a gain-of-function of HLA-G and a dominant-negative TGFbRII variant. In some embodiments, modified NK cells are provided that exhibit a loss-of-function of ADORA2A and B2M, and a gain-of-function of a single-chain IL-15/IL-15R fusion protein, and a dominant-negative TGFbRII variant. In some embodiments, modified NK cells are provided that exhibit a loss-of-function of ADORA2A, CISH, and B2M, and a gain-of-function of hnCD16 and HLA-G. In some embodiments, modified NK cells are provided that exhibit a loss-of-function of ADORA2A and B2M, and a gain-of-function of a single-chain IL-15/IL-15R fusion protein, HLA-G, and a dominant-negative TGFbRII variant. In some embodiments, modified NK cells are provided that exhibit a loss-of-function of TIGIT and B2M, and a gain-of-function of hnCD16 and a dominant-negative TGFbRII variant. In some embodiments, modified NK cells are provided that exhibit a loss-of-function of TIGIT and B2M, and a gain-of-function of a CAR, e.g., a CAR binding Her2, EGFR, alpha folate receptor, CEA, cMET, MUC1, Mesothelin, ROR1, or a different target, e.g., as disclosed herein or otherwise known in the art, and a dominant-negative TGFbRII variant. In some embodiments, modified NK cells are provided that exhibit a loss-of-function of TIGIT and B2M, and a gain-of-function of HLA-G and a dominant-negative TGFbRII variant. In some embodiments, modified NK cells are provided that exhibit a loss-of-function of TIGIT and B2M, and a gain-of-function of a single-chain IL-15/IL-15R fusion protein, and a dominant-negative TGFbRII variant. In some embodiments, modified NK cells are provided that exhibit a loss-of-function of TIGIT, CISH, and B2M, and a gain-of-function of hnCD16 and HLA-G. In some embodiments, modified NK cells are provided that exhibit a loss-of-function of TIGIT and B2M, and a gain-of-function of a single-chain IL-15/IL-15R fusion protein, HLA-G, and a dominant-negative TGFbRII variant. In some embodiments, modified NK cells are provided that exhibit a loss-of-function of ADORA2A, TIGIT, PD-1, and B2M, and a gain-of-function of a single-chain IL-15/IL-15R fusion protein, HLA-G, and a dominant-negative TGFbRII variant.

It is to be understood that the exemplary embodiments provided herein are meant to illustrate some examples of NK cells embraced by the present disclosure. Additional configurations are embraced that are not described here in detail for the sake of brevity, but such embodiments will be immediately apparent to those of skill in the art based on the present disclosure.

Chimeric Antigen Receptors (CARs)

As used herein, the term “chimeric antigen receptor” or (CAR″ refers to a receptor protein that has been modified to give cells expressing the CAR the new ability to target a specific protein. Within the context of the disclosure, an NK cell modified to comprise a CAR may be used for immunotherapy to target and destroy cells associated with a disease or disorder, e.g., cancer cells.

CARs of interest include, but are not limited to, a CAR targeting mesothelin, EGFR, HER2 and/or MICA/B. To date, mesothelin-targeted CAR T-cell therapy has shown early evidence of efficacy in a phase I clinical trial of subjects having mesothelioma, non-small cell lung cancer, and breast cancer (NCT02414269). Similarly, CARs targeting EGFR, HER2 and MICA/B have shown promise in early studies (see, e.g., Li et al. (2018), Cell Death & Disease, 9(177); Han et al. (2018) Am. J. Cancer Res., 8(1):106-119; and Demoulin 2017) Future Oncology, 13(8); the entire contents of each of which are expressly incorporated herein by reference in their entireties).

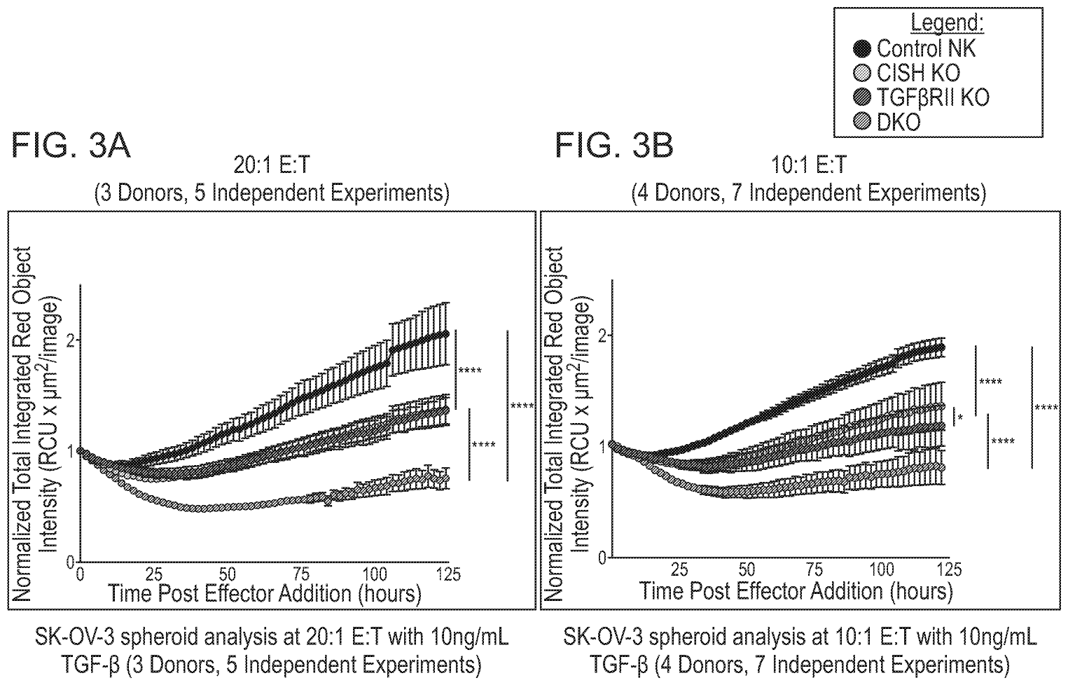

CARs are well-known to those of ordinary skill in the art and include those described in, for example: WO13/063419 (mesothelin), WO15/164594 (EGFR), WO13/063419 (HER2), WO16/154585 (MICA and MICB), the entire contents of each of which are expressly incorporated herein by reference in their entireties. Any suitable CAR, NK-CAR, or other binder that targets a cell, e.g., an NK cell, to a target cell, e.g., a cell associated with a disease or disorder, may be expressed in the modified NK cells provided herein. Exemplary CARs, and binders, include, but are not limited to, CARs and binders that bind BCMA, CD19, CD22, CD20, CD33, CD123, androgen receptor, PSMA, PSCA, Muc1, HPV viral peptides (ie. E7), EBV viral peptides, CD70, WT1, CEA, EGFRvIII, IL13Ra2, and GD2, CA125, CD7, EpCAM, Muc16, CD30. Additional suitable CARs and binders for use in the modified NK cells provided herein will be apparent to those of skill in the art based on the present disclosure and the general knowledge in the art. Such additional suitable CARs include those described in FIG. 3 of Davies and Maher, Adoptive T-cell Immunotherapy of Cancer Using Chimeric Antigen Receptor-Grafted T Cells, Archivum Immunologiae et Therapiae Experimentalis 58(3):165-78 (2010), the entire contents of which are incorporated herein by reference.

Modified NK cells provided herein may, in some embodiments, comprise a CAR and a CD16 variant, e.g., hnCD16, or a CAR and no CD16 variant. Any cell expressing CD16, or a variant thereof, would be suitable for combination therapy with a monoclonal antibody, e.g., a monoclonal antibody used in cancer therapy, or with an Fc fusion protein targeting pathological cells.

Knock-Ins and Knock-Outs

In some embodiments, a modified cell may express one or more of an exogenous hnCD16, an exogenous IL-15, an exogenous IL-15RA, a loss of function in TGFbetaR2, an exogenous DN-TGFbetaR2, and/or a loss of function in ADORA2A. In yet another embodiment, the modified cell may comprise a loss of function in B2M, an exogenous HLA-G, a loss of function in CIITA, a loss of function in PD1, a loss of function in TIGIT, or a loss of function in CISH.

In some embodiments, a modified cell may express one or more of an exogenous hnCD16, an exogenous IL-15, an exogenous IL-15RA, an exogenous HLA-G, an exogenous DN-TGFbetaR2, a loss of function in TGFbetaR2, a loss of function in B2M, a loss of function in PD1, a loss of function in TIGIT, and/or a loss of function in ADORA2A.

In some embodiments, a modified cell may express one or more of an exogenous hnCD16, an exogenous IL-15, an exogenous IL-15RA, an exogenous HLA-G, an exogenous DN-TGFbetaR2, a soluble MICA and/or MICB, a loss of function in TGFbetaR2, a loss of function in B2M, a loss of function in PD1, a loss of function in TIGIT, and/or a loss of function in ADORA2A.

In some embodiments, a modified cell may express one or more of an exogenous hnCD16, an exogenous IL-15, an exogenous IL-15RA, an exogenous HLA-G, an exogenous DN-TGFbetaR2, an exogenous IL-12, an exogenous IL-18, a loss of function in TGFbetaR2, a loss of function in B2M, a loss of function in PD1, a loss of function in TIGIT, and/or a loss of function in ADORA2A.

In some embodiments, a modified cell may express one or more of an exogenous hnCD16, an exogenous IL-15, an exogenous IL-15RA, an exogenous HLA-G, an exogenous DN-TGFbetaR2, an exogenous IL-12, an exogenous IL-18, a soluble MICA and/or MICB, a loss of function in TGFbetaR2, a loss of function in B2M, a loss of function in PD1, a loss of function in TIGIT, and/or a loss of function in ADORA2A.

As used herein, the term “express” or “expression” refers to the process to produce a polypeptide, including transcription and translation. Expression may be, e.g., increased by a number of approaches, including: increasing the number of genes encoding the polypeptide, increasing the transcription of the gene (such as by placing the gene under the control of a constitutive promoter), increasing the translation of the gene, knocking out of a competitive gene, or a combination of these and/or other approaches.

As used herein, the term “knock-in” refers to the addition of a target gene into a genetic locus of a cell.

As used herein, the term “knock-out” refers to an inactivating mutation in a target gene, wherein the product of the target gene comprises a loss of function.

As used herein, the term “loss of function” refers to an inactivating mutation in a target gene, wherein the gene product has less, or no, function (being partially or wholly inactivated). As used herein the term “complete loss of function” refers to an inactivating mutation in a target gene, wherein the gene product has no function (wholly inactivated).

As used herein, the term “hnCD16a” refers to a high affinity, non-cleavable variant of CD16 (a low-affinity Fcγ receptor involved in antibody-dependent cellular cytotoxicity (ADCC). Typically, CD16 is cleaved during ADCC—the hnCD16 CAR does not undergo this cleavage and thus sustains an ADCC signal longer. In some embodiments, the hnCD16a is disclosed in Blood 2016 128:3363, the entire contents of which are expressly incorporated herein by reference.

As used herein, the term “MICA/B” refers to MHC class I chain-related protein A (MICA) and B (MICB) are polymorphic proteins induced upon stress, damage or (malignant) transformation of cells, and act as a ‘kill me’ signal through the natural-killer group 2, member D receptor expressed on cytotoxic lymphocytes. MICA/B are not thought to be constitutively expressed by healthy normal cells, but expression has been reported for most tumor types. Exemplary sequences for MICA are provided in NG_034139.1, and exemplary sequences for MICB are provided in NG_021405.1.

As used herein, the term “AAVSI” refers to Adeno associated integration site 1.

As used herein, the term “2A” refers to self-cleaving 2A peptide.

As used herein, the term “TGFβRII” or “TGFbetaR2” refers to a transmembrane protein that has a protein kinase domain, forms a heterodimeric complex with TGF-beta receptor type-1, and binds TGF-beta. This receptor/ligand complex phosphorylates proteins, which then enter the nucleus and regulate the transcription of genes related to cell proliferation, cell cycle arrest, wound healing, immunosuppression, and tumorigenesis. Exemplary sequences of TGFβRII are set forth in KR710923.1, NM_001024847.2, and NM_003242.5.

As used herein, the term “DN-TGFβRII” refers to dominant negative TGF beta receptor II (could be expressed from an NK-specific promoter) TGFβRII plays an important role in T-cell differentiation, and KO in iPSCs would prevent CD34+ differentiation; KO would have to be performed later, but DN could be expressed from NK specific promoter (would turn on after CD34+ diff). In some embodiments, DN-TGFβRII is disclosed in Immunity. 2000 February; 12(2):171-81, the entire contents of which are expressly incorporated herein by reference.

The strategy used by tumor cells to protect themselves against the effects of TGF-β can be manipulated to shield tumor-specific cytotoxic T lymphocytes (CTLs) from the inhibitory effects of tumor-secreted TGF-β. Tumor-specific CTLs expressing a dominant negative TGF beta receptor II (for e.g., a TGFβRIIDNR sequence) have a selective functional and survival advantage over unmodified CTLs in the presence of TGF-β-secreting tumors (Bollard et al., 2002 Blood. 2002 May 1; 99(9):3179-87; incorporated in its entirety herein by reference). Accordingly, in some embodiments, the modified cell of the disclosure expresses a DN-TGFβRII construct. In some embodiments, the DN-TGFβRII construct is driven by an EF1a long promoter. In some embodiments, the DN-TGFβRII construct is knocked into an ADORA2A locus by using an S. pyogenes gRNA. In some embodiments, the DN-TGFβRII construct comprises a TGFβRIIDNR sequence, immediately followed by a 2A sequence, and further followed by a truncated EGFR sequence (EGFRt), to enable tracking of cells that efficiently express the construct. In some embodiments, the DN-TGFβRII construct is produced as a long single stranded DNA molecule. In some embodiments, the DN-TGFβRII construct is delivered to cells in an RNP. In some embodiments, the DN-TGFβRII construct is delivered to cells by AAV delivery (for e.g., via AAV6).

As used herein, the term “Neural cell adhesion molecule” (NCAM), also called CD56, refers to a homophilic binding glycoprotein expressed on the surface of neurons, glia and skeletal muscle and certain cells of the hematopoietic system. Expression of CD56 is associated with, but not limited to, natural killer cells. Exemplary sequences for NCAM are provided in NM_000615.6, NM_181351.4, NM_001076682.3, NM_001242608.1, and NM_001242607.1.

As used herein, the term “CISH” refers to the Cytokine Inducible SH2 Containing Protein, for e.g., see Delconte et al., Nat Immunol. 2016 July; 17(7):816-24; incorporated in its entirety herein by reference. Exemplary sequences for CISH are set forth as NG_023194.1.

As used herein, the term “IL-15/IL15RA” or “Interleukin-15” (IL-15) refers to a cytokine with structural similarity to Interleukin-2 (IL-2). Like IL-2, IL-15 binds to and signals through a complex composed of IL-2/IL-15 receptor beta chain (CD122) and the common gamma chain (gamma-C, CD132). IL-15 is secreted by mononuclear phagocytes (and some other cells) following infection by virus(es). This cytokine induces cell proliferation of natural killer cells; cells of the innate immune system whose principal role is to kill virally infected cells. IL-15 Receptor alpha (IL15RA) specifically binds IL15 with very high affinity, and is capable of binding IL-15 independently of other subunits. It is suggested that this property allows IL-15 to be produced by one cell, endocytosed by another cell, and then presented to a third party cell. IL15RA is reported to enhance cell proliferation and expression of apoptosis inhibitor BCL2L1/BCL2-XL and BCL2. Exemplary sequences of IL-15 are provided in NG_029605.2, and exemplary sequences of IL-15RA are provided in NM_002189.4.

IL-15 is a key cytokine in promoting NK cell growth and homeostatic maintenance of memory T cells. IL-15 and its receptor chain, IL-15Ra, are essential for NK survival and do not stimulate regulatory T cells. IL-15/IL-15Ra binds to the beta and gamma subunits of IL-2 receptor and thereby activates JAK1/3 and STATS. In some embodiments, the modified cell of the disclosure (for e.g., an NK cell) expresses an exogenous IL-15/IL-15Ra. In some embodiments, the exogenous IL-15/IL-15Ra is expressed as a membrane-bound IL15.IL15Ra complex, as described in Imamura et al., Blood. 2014 Aug. 14; 124(7):1081-8 and Hurton L V et al., PNAS, 2016; incorporated in their entirety herein by reference. In some embodiments, the exogenous IL-15/IL-15Ra is expressed as a soluble IL15Ra.IL15 complex, as described in Mortier E et al, JBC 2006; Bessard A, Mol Cancer Ther 2009; and Desbois M, JI 2016; incorporated in their entirety herein by reference. In some embodiments, the modified cell of the disclosure (for e.g., an NK cell) expresses a membrane-bound IL15.IL15Ra complex and a soluble IL15Ra.IL15 complex. In some embodiments, the modified cell of the disclosure (for e.g., an NK cell) express a membrane-bound form of IL15.IL15Ra complex with a cleavable linker. A knockout of CISH is associated with further promoting the IL-15 signaling, as described in Delconte P, Nat Immunol 2016; incorporated in its entirety herein by reference. In some embodiments, the modified cell of the disclosure (for e.g., an NK cell) expresses a loss of function in CISH. In some embodiments, the modified cell of the disclosure (for e.g., an NK cell) express exogenous IL-15/IL-15Ra and a loss of function in CISH.

As used herein, the term “ADORA2A” refers to the adenosine A2A receptor encodes a member of the guanine nucleotide-binding protein (G protein)-coupled receptor (GPCR) superfamily, which is subdivided into classes and subtypes. The receptors are seven-pass transmembrane proteins that respond to extracellular cues and activate intracellular signal transduction pathways. This protein, an adenosine receptor of A2A subtype, uses adenosine as the preferred endogenous agonist and preferentially interacts with the G(s) and G(olf) family of G proteins to increase intracellular cAMP levels. It plays an important role in many biological functions, such as cardiac rhythm and circulation, cerebral and renal blood flow, immune function, pain regulation, and sleep. It has been implicated in pathophysiological conditions such as inflammatory diseases and neurodegenerative disorders. Exemplary sequences of ADORA2a are provided in NG_052804.1.

As used herein, the term “B2M” (β2 microglobulin) refers to a serum protein found in association with the major histocompatibility complex (MHC) class I heavy chain on the surface of nearly all nucleated cells. The protein has a predominantly beta-pleated sheet structure that can form amyloid fibrils in some pathological conditions. The encoded antimicrobial protein displays antibacterial activity in amniotic fluid. Exemplary sequences for B2M are set forth as NG_012920.2.

As used herein, the term “CD32B” refers to a low affinity immunoglobulin gamma Fc region receptor II-b protein that, in humans, is encoded by the FCGR2B gene. See, e.g., Rankin C T et al., CD32B, the human inhibitory Fc-gamma receptor IIB, as a target for monoclonal antibody therapy of B-cell lymphoma. Blood 2006 108(7):2384-91, the entire contents of which are incorporated herein by reference.

As used herein, the term “CD47,” also sometimes referred to as “integrin associated protein” (IAP), refers to a transmembrane protein that in humans is encoded by the CD47 gene. CD47 belongs to the immunoglobulin superfamily, partners with membrane integrins, and also binds the ligands thrombospondin-1 (TSP-1) and signal-regulatory protein alpha (SIRPα). CD47 acts as a signal to macrophages that allows CD47-expressing cells to escape macrophage attack. See, e.g., Deuse-T, et al., Nature Biotechnology 2019 37: 252-258, the entire contents of which are incorporated herein by reference.

As used herein, the term “HLA-E” refers to the HLA class I histocompatibility antigen, alpha chain E, also sometimes referred to as MHC class I antigen E. The HLA-E protein in humans is encoded by the HLA-E gene. The human HLA-E is a non-classical MHC class I molecule that is characterized by a limited polymorphism and a lower cell surface expression than its classical paralogues. This class I molecule is a heterodimer consisting of a heavy chain and a light chain (beta-2 microglobulin). The heavy chain is anchored in the membrane. HLA-E binds a restricted subset of peptides derived from the leader peptides of other class I molecules. HLA-E expressing cells escape allogeneic responses and lysis by NK cells. See e.g., Geornalusse-G et al., Nature Biotechnology 2017 35(8), the entire contents of which are incorporated herein by reference. Exemplary sequences of the HLA-E protein are provided in NM_005516.6.

In some embodiments, two or more HLA class II histocompatibility antigen alpha chain genes and/or two or more HLA class II histocompatibility antigen alpha chain genes are knocked out, e.g., by genomic editing, in the modified lymphocytes provided herein. For example, in some embodiments, two or more HLA class II histocompatibility antigen alpha chain genes selected from HLA-DQA1, HLA-DRA, HLA-DPA1, HLA-DMA, HLA-DQA2, and HLA-DOA are knocked out. For another example, in some embodiments, the two or more HLA class II histocompatibility antigen beta chain genes selected from HLA-DMB, HLA-DOB, HLA-DPB1, HLA-DQB1, HLA-DQB3, HLA-DQB2, HLA-DRB1, HLA-DRB3, HLA-DRB4, and HLA-DRB5 are knocked out. See, e.g., Crivello et al., J Immunol January 2019, ji1800257; DOI: https://doi.org/10.4049/jimmunol.1800257, the entire contents of which are incorporated herein by reference.

As used herein, the term “HLA-G” refers to the HLA non-classical class I heavy chain paralogues. This class I molecule is a heterodimer consisting of a heavy chain and a light chain (beta-2 microglobulin). The heavy chain is anchored in the membrane. HLA-G is expressed on fetal derived placental cells. HLA-G is a ligand for NK cell inhibitory receptor KIR2DL4, and therefore expression of this HLA by the trophoblast defends it against NK cell-mediated death. See e.g., Favier et al., Tolerogenic Function of Dimeric Forms of HLA-G Recombinant Proteins: A Comparative Study In Vivo PLOS One 2011, the entire contents of which are incorporated herein by reference. An exemplary sequence of HLA-G is set forth as NG_029039.1.

As used herein, the term “CITTA” refers to the protein located in the nucleus that acts as a positive regulator of class II major histocompatibility complex gene transcription, and is referred to as the “master control factor” for the expression of these genes. The protein also binds GTP and uses GTP binding to facilitate its own transport into the nucleus. Once in the nucleus it does not bind DNA but rather uses an intrinsic acetyltransferase (AT) activity to act in a coactivator-like fashion. Mutations in this gene have been associated with bare lymphocyte syndrome type II (also known as hereditary MHC class II deficiency or HLA class II-deficient combined immunodeficiency), increased susceptibility to rheumatoid arthritis, multiple sclerosis, and possibly myocardial infarction. See, e.g., Chang et al., J Exp Med 180:1367-1374; and Chang et al., Immunity. 1996 February; 4(2):167-78, the entire contents of each of which are incorporated by reference herein. An exemplary sequence of CIITA is set forth as NG_009628.1.

As used herein, the term “PD1” Programmed cell death protein 1, also known CD279 (cluster of differentiation 279), refers to a protein found on the surface of cells that has a role in regulating the immune system's response to the cells of the human body by down-regulating the immune system and promoting self-tolerance by suppressing T cell inflammatory activity. This prevents autoimmune diseases, but it can also prevent the immune system from killing cancer cells. PD-1 is an immune checkpoint and guards against autoimmunity through two mechanisms. First, it promotes apoptosis (programmed cell death) of antigen-specific T-cells in lymph nodes. Second, it reduces apoptosis in regulatory T cells (anti-inflammatory, suppressive T cells). Exemplary sequences for PD1 are set forth as NM_005018.3.

As used herein, the term “TIGIT” refers to a member of the PVR (poliovirus receptor) family of immunoglobin proteins. The product of this gene is expressed on several classes of T cells including follicular B helper T cells (TFH). The protein has been shown to bind PVR with high affinity; this binding is thought to assist interactions between TFH and dendritic cells to regulate T cell dependent B cell responses. Exemplary sequences for TIGIT are set forth in NM_173799.4.

As used herein, the term “NLRC5” refers to a NOD-like receptor family CARD domain containing 5 intracellular protein that plays a role in the immune system. NLRC5 is a pattern recognition receptor implicated in innate immunity to viruses potentially by regulating interferon activity. Exemplary sequences forNLRC5 are set forth as NM_032206.4.

As used herein, the term “CTLA4” refers to a member of the immunoglobulin superfamily which transmits an inhibitory signal to T cells. The protein contains a V domain, a transmembrane domain, and a cytoplasmic tail. Exemplary sequences forCTLA4 are set forth as AF414120.1.

As used herein, the term “LAG3” refers to the lymphocyte-activation protein 3, which belongs to the Ig superfamily and contains 4 extracellular Ig-like domains. Exemplary sequences for LAG3 are set forth as NM_002286.6.

As used herein, the term “CBLB” refers to a E3 ubiquitin-protein ligase which promotes proteosome-mediated protein degradation by transferring ubiquitin from an E2 ubiquitin-conjugating enzyme to a substrate. The encoded protein is involved in the regulation of immune response by limiting T-cell receptor, B-cell receptor, and high affinity immunoglobulin epsilon receptor activation. Exemplary sequences for CBLB are set forth as KR709533.1.

As used herein, the term “NKG2A” refers to a protein belonging to the killer cell lectin-like receptor family, also called NKG2 family, which is a group of transmembrane proteins preferentially expressed in NK cells. This family of proteins is characterized by the type II membrane orientation and the presence of a C-type lectin domain. This protein forms a complex with another family member, KLRD1/CD94, and has been implicated in the recognition of the MHC class I HLA-E molecules in NK cells. See, e.g., Kamiya-T et al., J Clin Invest 2019 https://doi.org/10.1172/JCI123955, the entire contents of which are incorporated herein by reference. Exemplary sequences forNKG2A are set forth as AF461812.1.

As used herein, the term “CCR5” refers to a member of the beta chemokine receptor family, which is predicted to be a seven transmembrane protein similar to G protein-coupled receptors. This protein is expressed by T cells and macrophages, and is known to be an important co-receptor for macrophage-tropic virus, including HIV, to enter host cells. Exemplary sequences for CCR5 are set forth as U54994.1.

As used herein, the term “SOCS” refers to a family of genes involved in inhibiting the JAK-STAT signaling pathway.

As used herein, the term “BIM” refers to a pro-apoptotic member of the BCL-2 protein family, which interacts with other members of the BCL-2 protein family, including BCL2, BCL2L1/BCL-X(L), and MCL1, and act as an apoptotic activator.

As used herein, the term “FAS” refers to a member of the TNF-receptor superfamily. This receptor contains a death domain. It has been shown to play a central role in the physiological regulation of programmed cell death.

As used herein, the term “GITR” refers to a Tumor necrosis factor receptor superfamily member 18 (TNFRSF18) also known as activation-inducible TNFR family receptor (AITR) or glucocorticoid-induced TNFR-related protein. It involved in interactions between activated T-lymphocytes and endothelial cells and in the regulation of T-cell receptor-mediated cell death.

As used herein, the term “sortilin” refers to the VPS10-related sortilin family of proteins.

As used herein, the term “TIM3” refers to a T-cell immunoglobulin and mucin-domain containing-3 (TIM-3) protein that in humans is encoded by the HAVCR2 gene.

As used herein, the term “CD96” or “TACTILE” refers to a type I membrane protein that plays a role in the adhesive interactions of activated T and NK cells during the late phase of the immune response.

As used herein, the term “IL1R8” refers to a member of the interleukin 1 receptor family and is similar to the interleukin 1 accessory proteins.

As used herein, the term, “KIR2DL1”, “KIR2DL2” and “KIR2DL3” refer to killer cell immunoglobulin-like receptors (KIRs), which are transmembrane glycoproteins expressed by natural killer cells and subsets of T cells.

As used herein, the term “CDK8” refers to a member of the cyclin-dependent protein kinase (CDK) family, that functions as a regulator of cell cycle progression.

As used herein, the term “CXCR3” refers to a G protein-coupled receptor with selectivity for three chemokines, termed CXCL9/Mig (monokine induced by interferon-g), CXCL10/IP10 (interferon-g-inducible 10 kDa protein) and CXCL11/I-TAC (interferon-inducible T cell a-chemoattractant).

As used herein, the term “CCR7” refers to a member of the G protein-coupled receptor family. This receptor is expressed in various lymphoid tissues and activates B and T lymphocytes.

As used herein, the term “EP4” refers to a member of the G-protein coupled receptor family. This protein is one of four receptors identified for prostaglandin E2 (PGE2). This receptor can activate T-cell factor signaling.

As used herein, the term “IL-2” refers to interleukin-2, a secreted cytokine that is important for the proliferation of T and B lymphocytes.

As used herein, the term “IL-12” refers to interleukin-12, a cytokine that acts on T and natural killer cells.

As used herein, the term “IL-18” refers to interleukin-18, a proinflammatory cytokine primarily involved in polarized T-helper 1 (Th1) cell and natural killer (NK) cell immune responses.

As used herein, the term “CXCR1” refers to a member of the G-protein-coupled receptor family. This protein is a receptor for interleukin 8 (IL8).

As used herein, the term “CX3CR1” refers to a transmembrane protein and chemokine involved in the adhesion and migration of leukocytes.

As used herein, the term “mTRAIL” refers to a cytokine that belongs to the tumor necrosis factor (TNF) ligand family. This protein preferentially induces apoptosis in transformed and tumor cells.

As used herein, the term “TOSO” refers to an Fc Fragment of the IgM Receptor

As used herein, the term “CD16” refers to a receptor for the Fc portion of immunoglobulin G, and it is involved in the removal of antigen-antibody complexes from the circulation, as well as other antibody-dependent responses.

In some embodiments, modified cells are provided herein that exhibit a loss of function of TRAC. The term “TRAC” refers to the T-cell receptor alpha subunit (constant), encoded by the TRAC locus. Cells exhibiting a loss-of-function of TRAC do not express a T-cell receptor (TCR). In some embodiments, modified cells, e.g., pluripotent or multipotent stem cells or differentiated daughter cells thereof (e.g., iNK cells), are provided herein that are derived from a cell expressing a TCR or from a cell having a rearranged endogenous TCR locus, e.g., from a T-cell. In some embodiments, such cells comprise a modification that effects a loss-of-function of TRAC and thus do not express a functional TCR. Suitable methods and compositions for effecting a loss-of-function of TRAC will be apparent to those of ordinary skill in the art based on the present disclosure. Such methods and compositions include, without limitation, those disclosed in PCT Application PCT/US2015/026504, entitled “CRISPR-CAS-related methods, compositions and components for cancer immunotherapy”; PCT Application PCT/US2016/024353, entitled “CRISPR-CAS-related methods, compositions and components”; and PCT Application PCT/US2017/020598, entitled “CRISPR-CPF1-related methods, compositions and components for cancer immunotherapy”; the entire contents of each of which are incorporated herein by reference.

The disclosure specifically encompasses variants of the above genes and CARs, including variants having at least 75%, 80%, 85%, 90%, 91%, 92%, 93%, 94%, 95%, 96%, 97%, 98%, or 99% percent identity to the above-identified gene sequences. As used herein, the term “percent (%) sequence identity” or “percent (%) identity,” also including “homology,” is defined as the percentage of amino acid residues or nucleotides in a candidate sequence that are identical with the amino acid residues or nucleotides in the reference sequences after aligning the sequences and introducing gaps, if necessary, to achieve the maximum percent sequence identity, and not considering any conservative substitutions as part of the sequence identity. Optimal alignment of the sequences for comparison may be produced, besides manually, by means of the local homology algorithm of Smith and Waterman, 1981, Ads App. Math. 2, 482, by means of the local homology algorithm of Neddleman and Wunsch, 1970, J. Mol. Biol. 48, 443, by means of the similarity search method of Pearson and Lipman, 1988, Proc. Natl. Acad. Sci. USA 85, 2444, or by means of computer programs which use these algorithms (GAP, BESTFIT, FASTA, BLAST P, BLAST N and TFASTA in Wisconsin Genetics Software Package, Genetics Computer Group, 575 Science Drive, Madison, Wis.).

Knock-ins and knock-outs can be effected by genome editing technologies known to those of skill in the art and include CRISPR/Cas technologies. Single-cut as well as multiplex editing strategies are suitable to achieve the desired product configurations provided herein, and such strategies are described herein or otherwise known to those of ordinary skill in the art.

In some embodiments, exemplary modified cells, e.g., modified pluripotent cells or differentiated progeny thereof, e.g., iNK cells or other modified lymphocyte types, are evaluated for their ability to escape the immune system of a non-autologous host, e.g., a patient in need of immunotherapy. In some embodiments, such an evaluation includes an in vitro assay. Suitable in vitro assays for such evaluations are known to those of ordinary skill in the relevant art, and include, without limitation, mixed lymphocyte reactivity (MLR) assays. This assay and other suitable assays are described, e.g., in Abbas et al., Cellular and Molecular Immunology, 7^{th} edition, ISBN 9781437735734, the entire contents of which are incorporated herein by reference. Other suitable assays will be apparent to the skilled artisan in view of the present disclosure.

### Methods of Use

A variety of diseases may be ameliorated by introducing the modified cells of the invention to a subject. Examples of diseases are, including but not limited to, cancer, including but not limited to solid tumors, including but not limited to, tumor of the brain, prostate, breast, lung, colon, uterus, skin, liver, bone, pancreas, ovary, testes, bladder, kidney, head, neck, stomach, cervix, rectum, larynx, or esophagus; and hematological malignancies, including but not limited to, acute and chronic leukemias, lymphomas, multiple myeloma and myelodysplastic syndromes.

Particular embodiments of the present invention are directed to methods of treating a subject in need thereof by administering to the subject a composition comprising any of the cells described herein. In particular embodiments, the terms “treating,” “treatment,” and the like are used herein to generally mean obtaining a desired pharmacologic and/or physiologic effect. The effect may be prophylactic in terms of completely or partially preventing a disease and/or may be therapeutic in terms of a partial or complete cure for a disease and/or adverse effect attributable to the disease. “Treatment” as used herein covers any treatment of a disease in a mammal, and includes: preventing the disease from occurring in a subject which may be predisposed to the disease but has not yet been diagnosed as having it; inhibiting the disease, i.e., arresting its development; or relieving the disease, i.e., causing regression of the disease. The therapeutic agent or composition may be administered before, during or after the onset of disease or injury. The treatment of ongoing disease, where the treatment stabilizes or reduces the undesirable clinical symptoms of the patient, is of particular interest.

In particular embodiments, the subject has a disease, condition, and/or an injury that can be treated, ameliorated, and/or improved by a cell therapy. Some embodiments contemplate that a subject in need of cell therapy is a subject with an injury, disease, or condition, whereby a cell therapy, e.g., a therapy in which a cellular material is administered to the subject, can treat, ameliorate, improve, and/or reduce the severity of at least one symptom associated with the injury, disease, or condition. Certain embodiments contemplate that a subject in need of cell therapy, includes, but is not limited to, a candidate for bone marrow or stem cell transplantation, a subject who has received chemotherapy or irradiation therapy, a subject who has or is at risk of having a hyperproliferative disorder or a cancer, e.g. a hyperproliferative disorder or a cancer of hematopoietic system, a subject having or at risk of developing a tumor, e.g., a solid tumor, a subject who has or is at risk of having a viral infection or a disease associated with a viral infection.

According, the present invention further provides pharmaceutical compositions comprising the pluripotent cell derived hematopoietic lineage cells made by the methods and composition disclosed herein, wherein the pharmaceutical compositions further comprise a pharmaceutically acceptable medium. In some embodiments, the pharmaceutical composition comprises the pluripotent cell derived T cells made by the methods and composition disclosed herein. In some embodiments, the pharmaceutical composition comprises the pluripotent cell derived NK cells made by the methods and composition disclosed herein. In some embodiments, the pharmaceutical composition comprises the pluripotent cell derived CD34 HE cells made by the methods and composition disclosed herein. In some embodiments, the pharmaceutical composition comprises the pluripotent cell derived HSCs made by the methods and composition disclosed herein.

Additionally, the present invention provides therapeutic use of the above pharmaceutical compositions by introducing the composition to a subject suitable for adoptive cell therapy, wherein the subject has an autoimmune disorder; a hematological malignancy; a solid tumor; or an infection associated with HIV, RSV, EBV, CMV, adenovirus, or BK polyomavirus.

The isolated pluripotent stem cell derived hematopoietic lineage cells can have at least 50%, 60%, 70%, 80%, 90%, 95%, 98%, or 99% T cells, NK cells, NKT cells, CD34+ HE cells or HSCs. In some embodiments, the isolated pluripotent stem cell derived hematopoietic lineage cells has about 95% to about 100% T cells, NK cells, NKT cells, CD34+ HE cells or HSCs. In some embodiments, the present invention provides pharmaceutical compositions having purified T cells, NK cells, NKT cells, CD34+ HE cells or HSCs, such as a composition having an isolated population of about 95% T cells, NK cells, NKT cells, CD34+ HE cells or HSCs to treat a subject in need of the cell therapy.

In some embodiments, the pharmaceutical composition includes an isolated population of pluripotent stem cell derived hematopoietic lineage cells, wherein population has less than about 0.1%, 0.5%, 1%, 2%, 5%, 10%, 15%, 20%, 25%, or 30% iPSC derived T cells, NK cells, NKT cells, CD34+ HE cells or HSCs. The isolated population of derived hematopoietic lineage cells in some embodiments can have more than about 0.1%, 0.5%, 1%, 2%, 5%, 10%, 15%, 20%, 25%, or 30% T cells, NK cells, NKT cells, CD34+ HE cells or HSCs. In other embodiments, the isolated population of derived hematopoietic lineage cells can have about 0.1% to about 1%, about 1% to about 3%, about 3% to about 5%, about 10%-about 15%, about 15%-20%, about 20%-25%, about 25%-30%, about 30%-35%, about 35%-40%, about 40%-45%, about 45%-50%, about 60%-70%, about 70%-80%, about 80%-90%, about 90%-95%, or about 95% to about 100% T cells, NK cells, NKT cells, CD34+ HE cells or HSCs.

In particular embodiments, the derived hematopoietic lineage cells can have about 0.1%, about 1%, about 3%, about 5%, about 10%, about 15%, about 20%, about 25%, about 30%, about 35%, about 40%, about 45%, about 50%, about 60%, about 70%, about 80%, about 90%, about 95%, about 98%, about 99%, or about 100% T cells, NK cells, NKT cells, CD34+ HE cells or HSCs.

As a person of ordinary skill in the art would understand, both autologous and allogeneic immune cells can be used in cell therapies. Autologous cell therapies can have reduced infection, low probability for GvHD, and rapid immune reconstitution. Allogeneic cell therapies can have an immune mediated graft-versus-malignancy (GVM) effect, and low rate of relapse. Based on the specific conditions of the patients or subject in need of the cell therapy, a person of ordinary skill in the art would be able to determine which specific type of therapy to administer.

In particular embodiments, the derived hematopoietic lineage cells of the pharmaceutical composition of the invention are allogeneic to a subject. In particular embodiments, the derived hematopoietic lineage cells of the pharmaceutical formulation of the invention are autologous to a subject. For autologous transplantation, the isolated population of derived hematopoietic lineage cells are either complete or partial HLA-match with the patient. In another embodiment, the derived hematopoietic lineage cells are not HLA-matched to the subject.

The derived hematopoietic lineage cells provided by the invention can be administration to a subject without being expanded ex vivo or in vitro prior to administration. In particular embodiments, an isolated population of derived hematopoietic lineage cells is modulated and treated ex vivo using one or more agent to obtain immune cells with improved therapeutic potential. The modulated population of derived hematopoietic lineage cells can be washed to remove the treatment agent(s), and the improved population is administered to a patient without further expansion of the population in vitro.

In other embodiments, the invention provides an isolated population of derived hematopoietic lineage cells that are expanded prior to modulating the isolated population or subpopulation of T lymphocytes with one or more agents. The isolated population of derived hematopoietic lineage cells can be recombinantly produced to express TCR, CAR or other proteins.

For genetically engineered derived hematopoietic lineage cells that express recombinant TCR or CAR, whether prior to or after genetic modification of the cells, the cells can be activated and expanded using methods as described, for example, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; 6,867,041; and U.S. Patent Application Publication No. 20060121005.

Cancers

Cancers that are suitable therapeutic targets of the present disclosure include cancer cells from the bladder, blood, bone, bone marrow, brain, breast, colon, esophagus, eye, gastrointestine, gum, head, kidney, liver, lung, nasopharynx, neck, ovary, prostate, skin, stomach, testis, tongue, or uterus. In addition, the cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; androblastoma, malignant; sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malig melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; Hodgkin's disease; Hodgkin's lymphoma; paragranuloma; malignant lymphoma, small lymphocytic; malignant lymphoma, large cell, diffuse; malignant lymphoma, follicular; mycosis fungoides; other specified non-Hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia.

In some embodiments, the cancer is a breast cancer. In another embodiment, the cancer is colon cancer. In another embodiment, the cancer is gastric cancer. In another embodiment, the cancer is RCC. In another embodiment, the cancer is non-small cell lung cancer (NSCLC).

In some embodiments, solid cancer indications that can be treated with the modified NK cells provided herein, either alone or in combination with one or more additional cancer treatment modality, include: bladder cancer, hepatocellular carcinoma, prostate cancer, ovarian/uterine cancer, pancreatic cancer, mesothelioma, melanoma, glioblastoma, HPV-associated and/or HPV-positive cancers such as cervical and HPV+ head and neck cancer, oral cavity cancer, cancer of the pharynx, thyroid cancer, gallbladder cancer, and soft tissue sarcomas;

In some embodiments, hematological cancer indications that can be treated with the modified NK cells provided herein, either alone or in combination with one or more additional cancer treatment modality, include: ALL, CLL, NHL, DLBCL, AML, CML, multiple myeloma (MM).

As used herein, the term “cancer” (also used interchangeably with the terms, “hyperproliferative” and “neoplastic”) refers to cells having the capacity for autonomous growth, i.e., an abnormal state or condition characterized by rapidly proliferating cell growth. Cancerous disease states may be categorized as pathologic, i.e., characterizing or constituting a disease state, e.g., malignant tumor growth, or may be categorized as non-pathologic, i.e., a deviation from normal but not associated with a disease state, e.g., cell proliferation associated with wound repair. The term is meant to include all types of cancerous growths or oncogenic processes, metastatic tissues or malignantly transformed cells, tissues, or organs, irrespective of histopathologic type or stage of invasiveness. The term “cancer” includes malignancies of the various organ systems, such as those affecting lung, breast, thyroid, lymphoid, gastrointestinal, and genito-urinary tract, as well as adenocarcinomas which include malignancies such as most colon cancers, renal-cell carcinoma, prostate cancer and/or testicular tumors, non-small cell carcinoma of the lung, cancer of the small intestine and cancer of the esophagus. The term “carcinoma” is art recognized and refers to malignancies of epithelial or endocrine tissues including respiratory system carcinomas, gastrointestinal system carcinomas, genitourinary system carcinomas, testicular carcinomas, breast carcinomas, prostatic carcinomas, endocrine system carcinomas, and melanomas. Exemplary carcinomas include those forming from tissue of the cervix, lung, prostate, breast, head and neck, colon and ovary. The term “carcinoma” also includes carcinosarcomas, e.g., which include malignant tumors composed of carcinomatous and sarcomatous tissues. An “adenocarcinoma” refers to a carcinoma derived from glandular tissue or in which the tumor cells form recognizable glandular structures. The term “sarcoma” is art recognized and refers to malignant tumors of mesenchymal derivation.

Examples of cellular proliferative and/or differentiative disorders of the lung include, but are not limited to, tumors such as bronchogenic carcinoma, including paraneoplastic syndromes, bronchioloalveolar carcinoma, neuroendocrine tumors, such as bronchial carcinoid, miscellaneous tumors, metastatic tumors, and pleural tumors, including solitary fibrous tumors (pleural fibroma) and malignant mesothelioma.

Examples of cellular proliferative and/or differentiative disorders of the breast include, but are not limited to, proliferative breast disease including, e.g., epithelial hyperplasia, sclerosing adenosis, and small duct papillomas; tumors, e.g., stromal tumors such as fibroadenoma, phyllodes tumor, and sarcomas, and epithelial tumors such as large duct papilloma; carcinoma of the breast including in situ (noninvasive) carcinoma that includes ductal carcinoma in situ (including Paget's disease) and lobular carcinoma in situ, and invasive (infiltrating) carcinoma including, but not limited to, invasive ductal carcinoma, invasive lobular carcinoma, medullary carcinoma, colloid (mucinous) carcinoma, tubular carcinoma, and invasive papillary carcinoma, and miscellaneous malignant neoplasms. Disorders in the male breast include, but are not limited to, gynecomastia and carcinoma.

Examples of cellular proliferative and/or differentiative disorders involving the colon include, but are not limited to, tumors of the colon, such as non-neoplastic polyps, adenomas, familial syndromes, colorectal carcinogenesis, colorectal carcinoma, and carcinoid tumors.

Examples of cancers or neoplastic conditions, in addition to the ones described above, include, but are not limited to, a fibrosarcoma, myosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, gastric cancer, esophageal cancer, rectal cancer, pancreatic cancer, ovarian cancer, prostate cancer, uterine cancer, cancer of the head and neck, skin cancer, brain cancer, squamous cell carcinoma, sebaceous gland carcinoma, papillary carcinoma, papillary adenocarcinoma, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, testicular cancer, small cell lung carcinoma, non-small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma, leukemia, lymphoma, or Kaposi sarcoma.

Contemplated useful secondary or adjunctive therapeutic agents in this context include, but are not limited to: chemotherapeutic agents include alkylating agents such as thiotepa and CYTOXAN® cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); delta-9-tetrahydrocannabinol (dronabinol, MARINOL®); beta-lapachone; lapachol; colchicines; betulinic acid; a camptothecin (including the synthetic analogue topotecan (HYCAMTIN®), CPT-11 (irinotecan, CAMPTOSAR®)), acetylcamptothecin, scopolectin, and 9-aminocamptothecin); bryostatin; callystatin; CC-1065 (including its adozelesin, carzelesin and bizelesin synthetic analogues); podophyllotoxin; podophyllinic acid; teniposide; cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfanide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gamma1I and calicheamicin omegal1 (see, e.g., Agnew, Chem. Intl. Ed. Engl., 33: 183-186 (1994)); dynemicin, including dynemicin A; an esperamicin; as well as neocarzinostatin chromophore and related chromoprotein enediyne antiobiotic chromophores), aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, doxorubicin (including ADRIAMYCIN®, morpholino-doxorubicin, cyanomorpholino-doxorubicin, 2-pyrrolino-doxorubicin, doxorubicin HCl liposome injection (DOXIL®) and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate, gemcitabine (GEMZAR®), tegafur (UFTORAL®), capecitabine (XELODA®), an epothilone, and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; 2-ethylhydrazide; procarbazine; PSK® polysaccharide complex (JHS Natural Products, Eugene, Oreg.); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine (ELDISINE®, FILDESIN®); dacarbazine; mannomustine; mitobronitol; mitolactol; pipobroman; gacytosine; arabinoside (“Ara-C”); thiotepa; taxoids, e.g., paclitaxel (TAXOL®), albumin-engineered nanoparticle formulation of paclitaxel (ABRAXANET™), and doxetaxel (TAXOTERE®); chloranbucil; 6-thioguanine; mercaptopurine; methotrexate; platinum analogs such as cisplatin and carboplatin; vinblastine (VELBAN®); platinum; etoposide (VP-16); ifosfamide; mitoxantrone; vincristine (ONCOVIN®); oxaliplatin; leucovovin; vinorelbine (NAVELBINE®); novantrone; edatrexate; daunomycin; aminopterin; cyclosporine, sirolimus, rapamycin, rapalogs, ibandronate; topoisomerase inhibitor RFS 2000; difluoromethylornithine (DMFO); retinoids such as retinoic acid; CHOP, an abbreviation for a combined therapy of cyclophosphamide, doxorubicin, vincristine, and prednisolone, and FOLFOX, an abbreviation for a treatment regimen with oxaliplatin (ELOXATINTM) combined with 5-FU, leucovovin; anti-estrogens and selective estrogen receptor modulators (SERMs), including, for example, tamoxifen (including NOLVADEX® tamoxifen), raloxifene (EVISTA®), droloxifene, 4-hydroxytamoxifen, trioxifene, keoxifene, LY117018, onapristone, and toremifene (FARESTON®); anti-progesterones; estrogen receptor down-regulators (ERDs); estrogen receptor antagonists such as fulvestrant (FASLODEX®); agents that function to suppress or shut down the ovaries, for example, leutinizing hormone-releasing hormone (LHRH) agonists such as leuprolide acetate (LUPRON® and ELIGARD®), goserelin acetate, buserelin acetate and tripterelin; other anti-androgens such as flutamide, nilutamide and bicalutamide; and aromatase inhibitors that inhibit the enzyme aromatase, which regulates estrogen production in the adrenal glands, such as, for example, 4(5)-imidazoles, aminoglutethimide, megestrol acetate (MEGASE®), exemestane (AROMASIN®), formestanie, fadrozole, vorozole (RIVISOR®), letrozole (FEMARA®), and anastrozole (ARIMIDEX®); bisphosphonates such as clodronate (for example, BONEFOS® or OSTAC®), etidronate (DIDROCAL®), NE-58095, zoledronic acid/zoledronate (ZOMETA®), alendronate (FOSAMAX®), pamidronate (AREDIA®), tiludronate (SKELID®), or risedronate (ACTONEL®); troxacitabine (a 1,3-dioxolane nucleoside cytosine analog); aptamers, described for example in U.S. Pat. No. 6,344,321, which is herein incorporated by reference in its entirety; anti HGF monoclonal antibodies (e.g., AV299 from Aveo, AMG102, from Amgen); truncated mTOR variants (e.g., CGEN241 from Compugen); protein kinase inhibitors that block mTOR induced pathways (e.g., ARQ197 from Arqule, XL880 from Exelexis, SGX523 from SGX Pharmaceuticals, MP470 from Supergen, PF2341066 from Pfizer); vaccines such as THERATOPE® vaccine and gene therapy vaccines, for example, ALLOVECTIN® vaccine, LEUVECTIN® vaccine, and VAXID® vaccine; topoisomerase 1 inhibitor (e.g., LURTOTECAN®); rmRH (e.g., ABARELIX®); lapatinib ditosylate (an ErbB-2 and EGFR dual tyrosine kinase small-molecule inhibitor also known as GW572016); COX-2 inhibitors such as celecoxib (CELEBREX®; 4-(5-(4-methylphenyl)-3-(trifluoromethyl)-1H-pyrazol-1-yl) benzenesulfonamide; and pharmaceutically acceptable salts, acids or derivatives of any of the above.

Other compounds that are effective in treating cancer are known in the art and described herein that are suitable for use with the compositions and methods of the present disclosure are described, for example, in the “Physicians Desk Reference, 62nd edition. Oradell, N.J.: Medical Economics Co., 2008”, Goodman & Gilman's “The Pharmacological Basis of Therapeutics, Eleventh Edition. McGraw-Hill, 2005”, “Remington: The Science and Practice of Pharmacy, 20th Edition. Baltimore, Md.: Lippincott Williams & Wilkins, 2000.”, and “The Merck Index, Fourteenth Edition. Whitehouse Station, N.J.: Merck Research Laboratories, 2006”, incorporated herein by reference in relevant parts

All publications, patents and patent applications cited herein, whether supra or infra, are hereby incorporated by reference in their entirety.

Throughout this specification, unless the context requires otherwise, the words “comprise”, “comprises” and “comprising” will be understood to imply the inclusion of a stated step or element or group of steps or elements but not the exclusion of any other step or element or group of steps or elements. By “consisting of is meant including, and limited to, whatever follows the phrase “consisting of:” Thus, the phrase “consisting of indicates that the listed elements are required or mandatory, and that no other elements may be present. By “consisting essentially of is meant including any elements listed after the phrase, and limited to other elements that do not interfere with or contribute to the activity or action specified in the disclosure for the listed elements. Thus, the phrase “consisting essentially of indicates that the listed elements are required or mandatory, but that no other elements are optional and may or may not be present depending upon whether or not they affect the activity or action of the listed elements.

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet are incorporated herein by reference, in their entirety. The contents of database entries, e.g., NCBI nucleotide or protein database entries provided herein, are incorporated herein in their entirety. Where database entries are subject to change over time, the contents as of the filing date of the present application are incorporated herein by reference. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

### EXAMPLES

The following Examples are merely illustrative and are not intended to limit the scope or content of the disclosure in any way.

### Example 1: Generation of Modified iNK Cells from iPS Cells

The use of iPS cell technology for implementing a complex editing strategy and subsequent derivation of iNK cells or other lymphocytes, for example, enable the generation of an iNK cell that express a CAR of interest such as mesothelin, EGFR, HER2, and MICA/B, and/or having one or more edits from List A and/or Table 10, and one or more edits from List B and/or Table 11.

List A:
Exogenous expression of an enhanced variant of CD16, e.g., of hnCD16a (high affinity, non-cleavable variant of CD 16—a low-affinity Fcy receptor involved in antibody-dependent cellular cytotoxicity (ADCC)). Typically, CD16 is cleaved during ADCC by proteases, the hnCD16 CAR does not undergo this cleavage and thus sustains an ADCC signal longer.
Exogenous expression of IL-15/IL 15RA
Loss-of-function of TGFbR2, or exogenous expression of a dominant-negative variant of TGFbR2 (dominant negative TGF beta receptor II is expressed from an NK-specific promoter, in order to not interfere with TGFbRII's role in the differentiation of CD34 cells, which can be derived from iPS cells, and typically serve as a cell type from which heme lineages (like NK cells) are differentiated)
Loss-of-function of ADORA2A

List B:
Loss-of-function of B2M (e.g., eliminate MHC class I expression by targeting B2M expression) Exogenous expression of HLA-G
Loss-of-function of CIITA (e.g., eliminate MHC class II expression by targeting CIITA)
Loss-of-function of PD1
Loss-of-function of TIGIT
Loss-of-function CISH (cytokine inducible SH2 containing protein)

Loss-of-function preferably includes complete elimination of surface expression of the respective protein.

For example, iNK cells with exogenous expression of a CAR and a CD16 variant (e.g., hnCD16), or a CAR and no CD16 variant can be generated. Cells expressing no CAR but a CD16 variant can also be generated. Any cell expressing CD16, or an enhanced variant thereof (e.g., hnCD16), would be suitable for combination therapy with a monoclonal antibody (e.g., used in cancer therapy), or with an Fc fusion protein targeting pathological cells.

If more than two transgenes are knocked-in, a multicistronic expression construct, or a 2A construct may be advantageous in order to avoid having to insert an individual construct for each transgene.

Such iNK cells are useful for a wide range of immunotherapy applications, including, but not limited to the treatment of proliferative diseases, e.g., certain forms of cancers. When using the CARs outlined above, applications in breast cancer, colon cancer, gastric cancer, renal cell carcinoma, and NSCLC are envisioned. The altered surface molecule repertoire of such cells would also enable the successful treatment of solid tumors, which has been proven difficult with current NK-cell based strategies.

Exemplary iNK cells obtained from reprogrammed somatic cells (or their daughter cells) comprise one or more (e.g., one or more, two or more, three or more, four or more, five or more, or six or more) of the following characteristics:
They comprise a rearranged endogenous TCR locus (e.g., TCRα VJ and/or TCRf3 V(D)J section rearrangement & complete V-domain exons);
They do not express an endogenous T cell co-receptor, e.g., CD3, CD4, and/or CD8;
They express an NK-cell biomarker, for example:
CD56 (NCAM), CD49, and/or CD45;
NK cell receptor immunoglobulin gamma Fc region receptor III (FcγRIII, cluster of differentiation 16 (CD16));
natural killer group-2 member 0 (NKG2D, MICAIB stress ligand receptor);
CD69;
a natural cytotoxicity receptor (e.g., NKp30; NKp44; NKp46; and/or CD158b); or any combination of two or more of these;

They may express:
a chimeric antigen receptor (CAR),
a non-naturally occurring variant of immunoglobulin gamma Fc region receptor III (FcγRIII, CD16)
an interleukin 15 (IL-15) pathway agonist, e.g., interleukin-15 (IL-15), interleukin 15 receptor (IL-15R) or a variant thereof (e.g., a constitutively active variant of IL-15R, e.g., an IL-15R fused to an IL-15R agonist (IL-15RA); other interleukin pathway agonists are also contemplated, either in the alternative or in combination with the IL-15 pathway agonist, e.g., an interleukin 2 (IL-2) pathway agonist, e.g., IL-2, interleukin 2 receptor (IL-2R) or a variant thereof (e.g., a constitutively active variant of IL-2R, e.g., an IL-2R fused to an IL-2R agonist (IL-2RA); and/or an interleukin 12 (IL12) pathway agonist, e.g., IL-12, interleukin 12 receptor (IL-12R) or a variant thereof (e.g., a constitutively active variant of IL-12R, e.g., an IL-12R fused to an IL-12R agonist (IL-12RA); a combination of two or more interleukins is also contemplated, e.g., a combination of an IL-15 pathway agonist or an IL-2 agonist and an IL-12 agonist, e.g., an IL-15R fused to an IL-15R agonist (IL-15RA) in combination with an IL-12R fused to an IL-12R agonist (IL-15RA).
human leukocyte antigen G (HLA-G); or any combination of two or more thereof;
human leukocyte antigen E (HLA-E)
leukocyte surface antigen cluster of differentiation CD47 (CD47)
and

They may exhibit a loss of function of:
transforming growth factor beta receptor 2 (TGFbetaR2, e.g., either by modification of the encoding sequence or by expression of a dominant negative variant);
adenosine A2a receptor (ADORA2A);
T cell immunoreceptor with Ig and ITIM domains (TIGIT);
β-2 microgobulin (B2M);
class II, major histocompatibility complex, transactivator (CIITA);
programmed cell death protein 1 (PD-1, CD279), or express a PD-1 antagonist;
cytokine inducible SH2 containing protein (CISH);
natural killer cell receptor NKG2A (natural killer group 2A);
two or more HLA class II histocompatibility antigen alpha chain genes, and/or two or more HLA class II histocompatibility antigen beta chain genes;
cluster of differentiation 32B (CD32B, FCGR2B);
or any combination of two or more thereof.

It is desirable to achieve specific combinations of these characteristics, e.g., an iNK cell expressing a CAR, IL-15, and HLA-G, and exhibiting a loss-of-function in B2M and PD-1 by minimizing the number of edits. For example, an expression construct encoding the CAR could be inserted into the B2M locus and an expression construct encoding IL-15 and HLA-G could be inserted into the B2M locus. Similar strategies would apply to other combinations.

The iNK cells could be used as a monotherapy, and those expressing a CAR (e.g., a CAR binding mesothelin, EGFR, or HER2) would be particularly suitable for therapeutic approaches specifically targeting cells expressing a surface antigen the CAR binds. Some iNK cells envisioned may also be suitable for combination therapy approaches, e.g., in combination with a monoclonal antibody targeting cancer cells.

In some embodiments, the generation of iPS cells would include obtaining a donor cell, e.g., a somatic cell from a healthy donor individual. In some embodiments, a donor cell or cell population is confirmed to be karyotypically normal, and not to exhibit expression of a gene or a combination of genes known to be associated with a pathological state, e.g., a malignant state. In some embodiments, the somatic cell is edited and then reprogrammed to a pluripotent state. In some embodiments, the somatic cell is reprogrammed and at the same time edited. In some embodiments, the somatic cell is reprogrammed and a resulting pluripotent cell is edited. In some embodiments, the generation of iPS cells comprises clonal expansion of reprogrammed cell lines, characterization of a number of such clonal iPS cell lines, and selection of a line that includes all the desired edits while being karyotypically normal.

The end product for clinical use is a population of iNK cells carrying the respective edits. The number of cells would be sufficient to elicit a desired immune response after administration to a subject. The precise number would depend on the specific desired clinical outcome, the patient, and the disease to be treated, amongst other factors, and may vary greatly. It is anticipated that a suitable cell population for administration may range from about 1,000 cells to about 100,000,000 cells. The iNK cell population for clinical use should be free of remaining stem cells, e.g., of iPS cells expressing Oct-4 and/or Sox2, should ideally be free of or contain only a minimal amount of cells harboring episomal expression constructs, e.g., episomal expression constructs used during reprogramming of T cells; should be free of, or not contain more than 1%, 5%, or 10%, of cells not expressing the desired combination of cell markers and overexpressed surface molecules.

### Example 2: Use of T Cells as Cells of Origin for a Complex Editing Strategy and Subsequent Derivation of iNK Cells

The use of T cells as cells of origin for a complex editing strategy and subsequent derivation of iNK cells or other lymphocytes, for example, enable the generation of an iNK cell that express a CAR of interest such as mesothelin, EGFR, HER2, and MICA/B, and/or having one or more edits from List A and/or Table 10, and one or more edits from List B and/or Table 11.

List A:
Exogenous expression of an enhanced variant of CD16, e.g., of hnCD16a (high affinity, non-cleavable variant of CD 16—a low-affinity Fcy receptor involved in antibody-dependent cellular cytotoxicity (ADCC)). Typically, CD16 is cleaved during ADCC by proteases, the hnCD16 CAR does not undergo this cleavage and thus sustains an ADCC signal longer.
Exogenous expression of IL-15/IL 15RA
Loss-of-function of TGFbR2, or exogenous expression of a dominant-negative variant of TGFbR2 (dominant negative TGF beta receptor II is expressed from an NK-specific promoter, in order to not interfere with TGFbRII's role in the differentiation of CD34 cells, which can be derived from iPS cells, and typically serve as a cell type from which heme lineages (like NK cells) are differentiated)
Loss-of-function of ADORA2A

List B:
Loss-of-function of B2M (e.g., eliminate MHC class I expression by targeting B2M expression) Exogenous expression of HLA-G
Loss-of-function of CIITA (e.g., eliminate MHC class II expression by targeting CIITA)
Loss-of-function of PD1
Loss-of-function of TIGIT
Loss-of-function CISH (cytokine inducible SH2 containing protein)

Loss-of-function preferably includes complete elimination of surface expression of the respective protein.

For example, iNK cells with exogenous expression of a CAR and a CD16 variant (e.g., hnCD16), or a CAR and no CD16 variant can be generated. Cells expressing no CAR but a CD16 variant can also be generated. Any cell expressing CD16, or an enhanced variant thereof (e.g., hnCD16), would be suitable for combination therapy with a monoclonal antibody (e.g., used in cancer therapy), or with an Fc fusion protein targeting pathological cells.

If more than two transgenes are knocked-in, a multicistronic expression construct, or a 2A construct may be advantageous in order to avoid having to insert an individual construct for each transgene.

Such iNK cells are useful for a wide range of immunotherapy applications, including, but not limited to the treatment of proliferative diseases, e.g., certain forms of cancers. When using the CARs outlined above, applications in breast cancer, colon cancer, gastric cancer, renal cell carcinoma, and NSCLC are envisioned. The altered surface molecule repertoire of such cells would also enable the successful treatment of solid tumors, which has been proven difficult with current NK-cell based strategies.

Exemplary iNK cells obtained from reprogrammed/edited T cells (or their daughter cells) comprise one or more (e.g., one or more, two or more, three or more, four or more, five or more, or six or more) of the following characteristics:
They comprise a rearranged endogenous TCR locus (e.g., TCRa VJ and/or TCRf3 V(D)J section rearrangement & complete V-domain exons);
They do not express an endogenous T cell co-receptor, e.g., CD3, CD4, and/or CD8;
They express an NK-cell biomarker, for example:
   CD56 (NCAM), CD49, and/or CD45;
   NK cell receptor immunoglobulin gamma Fc region receptor III (FcγRIII, cluster of differentiation 16 (CD16));
   natural killer group-2 member 0 (NKG2D, MICAIB stress ligand receptor);
   CD69;
   a natural cytotoxicity receptor (e.g., NKp30; NKp44; NKp46; and/or CD158b); or any combination of two or more of these;
They may express:
   a chimeric antigen receptor (CAR),
   a non-naturally occurring variant of immunoglobulin gamma Fc region receptor III (FcγRIII, CD16)
   an interleukin 15 (IL-15) pathway agonist, e.g., interleukin-15 (IL-15), interleukin 15 receptor (IL-15R) or a variant thereof (e.g., a constitutively active variant of IL-15R, e.g., an IL-15R fused to an IL-15R agonist (IL-15RA); other interleukin pathway agonists are also contemplated, either in the alternative or in combination with the IL-15 pathway agonist, e.g., an interleukin 2 (IL-2) pathway agonist, e.g., IL-2, interleukin 2 receptor (IL-2R) or a variant thereof (e.g., a constitutively active variant of IL-2R, e.g., an IL-2R fused to an IL-2R agonist (IL-2RA); and/or an interleukin 12 (IL12) pathway agonist, e.g., IL-12, interleukin 12 receptor (IL-12R) or a variant thereof (e.g., a constitutively active variant of IL-12R, e.g., an IL-12R fused to an IL-12R agonist (IL-12RA); a combination of two or more interleukins is also contemplated, e.g., a combination of an IL-15 pathway agonist or an IL-2 agonist and an IL-12 agonist, e.g., an IL-15R fused to an IL-15R agonist (IL-15RA) in combination with an IL-12R fused to an IL-12R agonist (IL-15RA).
   human leukocyte antigen G (HLA-G); or any combination of two or more thereof;
   human leukocyte antigen E (HLA-E)
   leukocyte surface antigen cluster of differentiation CD47 (CD47)
   and
They may exhibit a loss of function of:
   transforming growth factor beta receptor 2 (TGFf3R2, e.g., either by modification of the encoding sequence or by expression of a dominant negative variant);
   adenosine A2a receptor (ADORA2A);
   T cell immunoreceptor with Ig and ITIM domains (TIGIT);
   β-2 microgobulin (B2M);
   class II, major histocompatibility complex, transactivator (CIITA);
   programmed cell death protein 1 (PD-1, CD279), or express a PD-1 antagonist;
   cytokine inducible SH2 containing protein (CISH);
   natural killer cell receptor NKG2A (natural killer group 2A);
   two or more HLA class II histocompatibility antigen alpha chain genes, and/or two or more HLA class II histocompatibility antigen beta chain genes;
   cluster of differentiation 32B (CD32B, FCGR2B);
   T cell receptor alpha constant (TRAC);
   or any combination of two or more thereof.

It is desirable to achieve specific combinations of these characteristics, e.g., an iNK cell expressing a CAR, IL-15, and HLA-G, and exhibiting a loss-of-function in B2M and PD-1 by minimizing the number of edits. For example, an expression construct encoding the CAR could be inserted into the B2M locus and an expression construct encoding IL-15 and HLA-G could be inserted into the B2M locus. Similar strategies would apply to other combinations.

The iNK cells could be used as a monotherapy, and those expressing a CAR (e.g., a CAR binding mesothelin, EGFR, or HER2) would be particularly suitable for therapeutic approaches specifically targeting cells expressing a surface antigen the CAR binds. Some the iNK cells envisioned may also be suitable for combination therapy approaches, e.g., in combination with a monoclonal antibody targeting cancer cells.

The generation of iPS cells would include the clonal expansion of reprogrammed cell lines, the characterization of a number of such clonal iPS cell lines, and the selection of a line that includes all the desired edits while being karyotypically normal.

The end product for clinical use is a population of iNK cells carrying the respective edits. The number of cells would be sufficient to elicit a desired immune response after administration to a subject. The precise number would depend on the specific desired clinical outcome, the patient, and the disease to be treated, amongst other factors, and may vary greatly. It is anticipated that a suitable cell population for administration may range from about 1,000 cells to about 100,000,000 cells. The iNK cell population for clinical use should be free of remaining stem cells, e.g., of iPS cells expressing Oct-4 and/or Sox2, should ideally be free of or contain only a minimal amount of cells harboring episomal expression constructs, e.g., episomal expression constructs used during reprogramming of T cells; should be free of, or not contain more than 1%, 5%, or 10%, of cells not expressing the desired combination of cell markers and overexpressed surface molecules.

### Example 3: iPS/iNK Cells for Clinical Applications

For clinical use as an immunotherapeutic, e.g., in the context of immunooncology applications, modified lymphocytes, here iNK cells, are generated that comprise a loss-of-function of B2M; a loss-of-function of CIITA; and an exogenous nucleic acid expression construct comprising a nucleic acid sequence encoding HLA-G. These edits allow the edited cells, and/or differentiated iNK cells derived therefrom, to escape the immune system of a non-autologous host. Additional edits may be made to enhance the clinical properties of the iNK cells. These iNK cells are obtained by reprogramming a somatic donor cell from a healthy donor, reprogramming the donor cell into a pluripotent state and effecting the desired edits. Once edited, the pluripotent cells are differentiated into NK cells, resulting in a population of modified iNK cells for clinical application.

### Example 4: iPS/iNK Cells for Clinical Applications

For clinical use as an immunotherapeutic, e.g., in the context of immunooncology applications, modified lymphocytes, here iNK cells, are generated that comprise a loss-of-function of B2M; a loss-of-function of CIITA; and an exogenous nucleic acid expression construct comprising a nucleic acid sequence encoding HLA-E. In some embodiments, the cells further comprise a loss-of function of NKG2A. These edits allow the edited cells, and/or differentiated iNK cells derived therefrom, to escape the immune system of a non-autologous host. Additional edits may be made to enhance the clinical properties of the iNK cells. These iNK cells are obtained by reprogramming a somatic donor cell from a healthy donor, reprogramming the donor cell into a pluripotent state and effecting the desired edits. Once edited, the pluripotent cells are differentiated into NK cells, resulting in a population of modified iNK cells for clinical application.

### Example 5: iPS/iNK Cells for Clinical Applications

For clinical use as an immunotherapeutic, e.g., in the context of immunooncology applications, modified lymphocytes, here iNK cells, are generated that comprise a loss-of-function of B2M; a loss-of-function of CIITA; and an exogenous nucleic acid expression construct comprising a nucleic acid sequence encoding CD47. These edits allow the edited cells, and/or differentiated iNK cells derived therefrom, to escape the immune system of a non-autologous host. Additional edits may be made to enhance the clinical properties of the iNK cells. These iNK cells are obtained by reprogramming a somatic donor cell from a healthy donor, reprogramming the donor cell into a pluripotent state and effecting the desired edits. Once edited, the pluripotent cells are differentiated into NK cells, resulting in a population of modified iNK cells for clinical application.

### Example 6: iPS/iNK Cells for Clinical Applications

For clinical use as an immunotherapeutic, e.g., in the context of immunooncology applications, modified lymphocytes, here iNK cells, are generated that comprise a loss-of-function of B2M; a loss-of-function of HLA-DRB1, HLA-DRB3, HLA-DRB4, HLA-DRB5, HLA-DQB1, and HLA-DPB1; and an exogenous nucleic acid expression construct comprising a nucleic acid sequence encoding HLA-G. These edits allow the edited cells, and/or differentiated iNK cells derived therefrom, to escape the immune system of a non-autologous host. Additional edits may be made to enhance the clinical properties of the iNK cells. These iNK cells are obtained by reprogramming a somatic donor cell from a healthy donor, reprogramming the donor cell into a pluripotent state and effecting the desired edits. Once edited, the pluripotent cells are differentiated into NK cells, resulting in a population of modified iNK cells for clinical application.

### Example 7: iPS/iNK Cells for Clinical Applications

For clinical use as an immunotherapeutic, e.g., in the context of immunooncology applications, modified lymphocytes, here iNK cells, are generated that comprise a loss-of-function of B2M; a loss-of-function of HLA-DRB1, HLA-DRB3, HLA-DRB4, HLA-DRB5, HLA-DQB1, and HLA-DPB1; and an exogenous nucleic acid expression construct comprising a nucleic acid sequence encoding HLA-E. In some embodiments, the cells further comprise a loss-of function of NKG2A. These edits allow the edited cells, and/or differentiated iNK cells derived therefrom, to escape the immune system of a non-autologous host. Additional edits may be made to enhance the clinical properties of the iNK cells. These iNK cells are obtained by reprogramming a somatic donor cell from a healthy donor, reprogramming the donor cell into a pluripotent state and effecting the desired edits. Once edited, the pluripotent cells are differentiated into NK cells, resulting in a population of modified iNK cells for clinical application.

### Example 8: iPS/iNK Cells for Clinical Applications

For clinical use as an immunotherapeutic, e.g., in the context of immunooncology applications, modified lymphocytes, here iNK cells, are generated that comprise a loss-of-function of B2M; a loss-of-function of HLA-DRB1, HLA-DRB3, HLA-DRB4, HLA-DRB5, HLA-DQB1, and HLA-DPB1; and an exogenous nucleic acid expression construct comprising a nucleic acid sequence encoding CD47. These edits allow the edited cells, and/or differentiated iNK cells derived therefrom, to escape the immune system of a non-autologous host. Additional edits may be made to enhance the clinical properties of the iNK cells. These iNK cells are obtained by reprogramming a somatic donor cell from a healthy donor, reprogramming the donor cell into a pluripotent state and effecting the desired edits. Once edited, the pluripotent cells are differentiated into NK cells, resulting in a population of modified iNK cells for clinical application.

### Example 9: iPS/iNK Cells for Clinical Applications

Additional edits are made to the cells provided in examples 3-8 that enhance the effectiveness of the iNK cell as a therapeutic.

These edits include, in some embodiments, knock-in of an exogenous nucleic acid expression construct comprising a nucleic acid sequence encoding a variant of IL-15R, here a fusion of IL-15R with its ligand (IL-15, or an IL-15-binding fragment thereof), resulting in a constitutively active IL-15 pathway in the iNK cells.

These edits further include, in some embodiments, knock-in of an exogenous nucleic acid expression construct comprising a nucleic acid sequence encoding transforming growth factor beta receptor 2 (TGFβR2) under the control of an NK-cell specific promoter, e.g., a CD45 promoter.

These edits further include, in some embodiments, a loss-of-function of CD32B(FCGR2).

### Example 10: Gene Edited NK Cells, Exhibiting Loss-of-Function of CISH and/or TGFBR2, Demonstrate Improved Effector Function in Response to Tumor Cells

A next-generation allogeneic NK cell therapy was developed using CRISPR-Cpf1 gene editing to enhance NK cell function through knockout of the CISH and TGFBR2 genes.

NK cells were expanded from CD3⁻PBMC cultures in 20 ng/mL IL-15. Gene editing was performed at different NK cell expansion stages (between day 8-21). For editing of CISH and TGFBR2, guides for either targets were complexed with Cpf1 nuclease at a 2:1 ratio to form ribonucleoproteins (RNPs). Where cells were edited with both targets, RNP complexation for each target was done separately and then mixed at a 1:1 ratio prior to electroporation.

For electroporation, NK cells were suspended in HyClone buffer at a density of 80×10⁶ cells/mL. Ninety microliters of NK cells were mixed with 10 microliters of the appropriate RNPs. The cell and RNP mixtures were then transferred to a MaxCyte OC-100 or OC-400 cassette for electroporation. Immediately post-electroporation, NK cells were recovered in 100 microliters of culture media for 10 minutes at 37° C., before transferring to a 24-well Grex plate for post editing recovery and functional analyses.

The following guide RNA sequences were used for editing of CISH and TGFBR2: Both guides were generated with a targeting domain consisting of RNA, an AsCpf1 scaffold of the sequence UAAUUUCUACUCUUGUAGAU 5′ of the targeting domain, and a 25-mer DNA extension of the sequence ATGTGTTTTTGTCAAAAGACCTTTT at the 5′ terminus of the scaffold sequence.

TABLE 12

| | gRNA targeting domain | |
|---|---|---|
| Target | Sequence (DNA) | gRNA sequence |
| CISH 7050 | GGTGTACAGCAGTGGCTGGT | ATGTGTTTTTGTCAAAAGACCTTTTrUrA |
| | | rArUrUrUrCrUrArCrUrCrUrUrGrUr |
| | | ArGrArUrGrGrUrGrUrArCrArGrCrA |
| | | rGrUrGrGrCrUrGrGrU |
| TGFBR2 | TGATGTGAGATTTTCCACCT | ATGTGTTTTTGTCAAAAGACCTTTTrUrA |
| 24026 | | rArUrUrUrCrUrArCrUrCrUrUrGrUr |
| | | ArGrArUrUrGrArUrGrUrGrArGrArU |
| | | rUrUrUrCrCrArCrCrU |

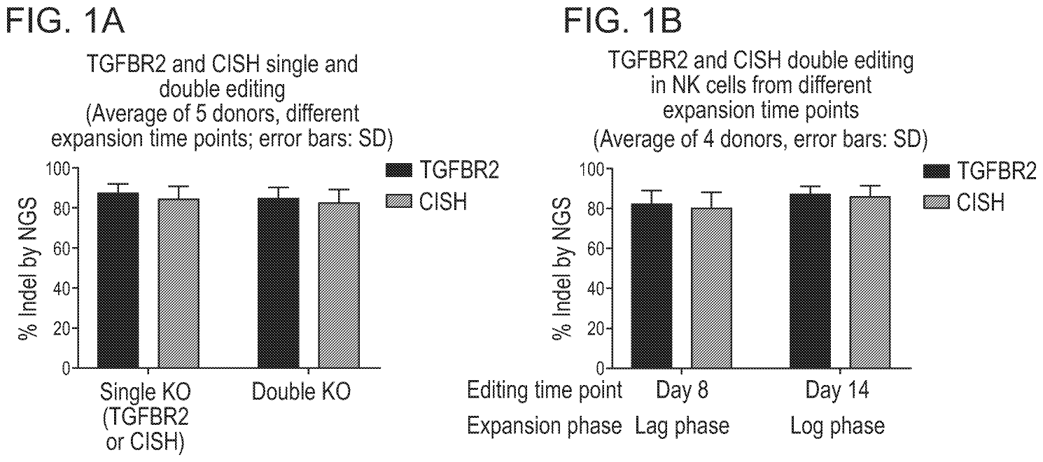

As demonstrated in FIGS. 1A-1B, robust single and double-gene editing of TGFBR2 and CISH was achieved in NK cells. Both single and simultaneous targeting of TGFBR2 and CISH in NK cells using CRISPR-Cpf1 produced indels at both targets in greater than 80% of NK cells, with greater than 90% of edited NK cells viable at 72 hours post-editing.

The efficacy of the effector cells was assessed in vitro by 3D tumor spheroid assays

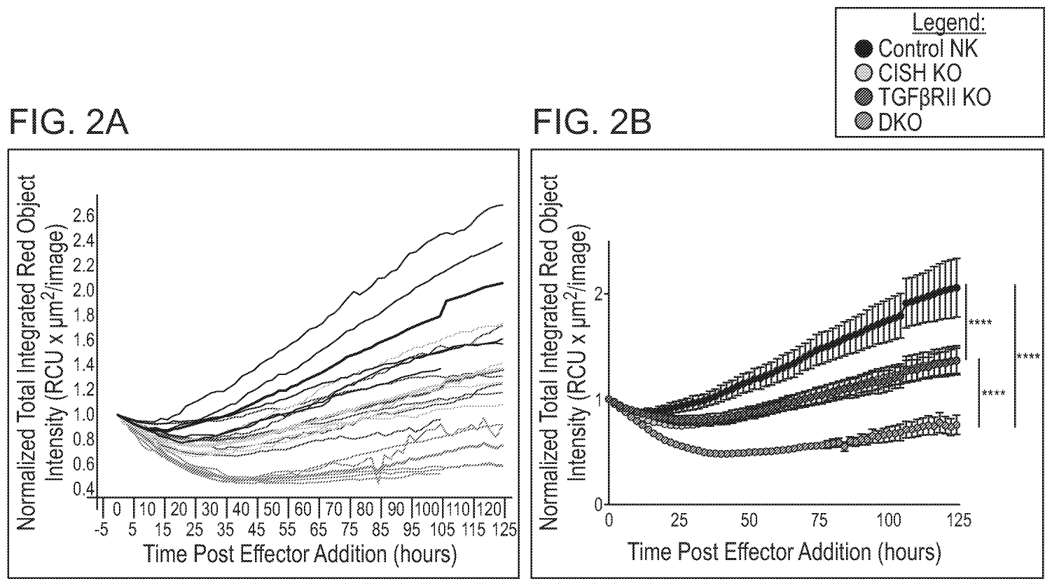

To form spheroids, 5,000 NucLight Red labeled PC-3 or SK-OV-3 tumor cells were plated in a single well of ultra-low attachment 96 well plates, centrifuged at 1,000 rpm for 10 minutes, and incubated for 96 hours at 37° C. At 96 hours, effector cells (primary human NK cells treated with different RNPs) were added to spheroids at multiple effector to target cell ratios with or without 10 ng/mL of TGF-beta. Red object intensity was measured every two hours for 6 days on an Incucyte imaging system. Data shown are normalized to the red object intensity at time of effector addition. Normalization of spheroid curves maintains the same efficacy patterns observed in non-normalized data (FIGS. 2A-2B).

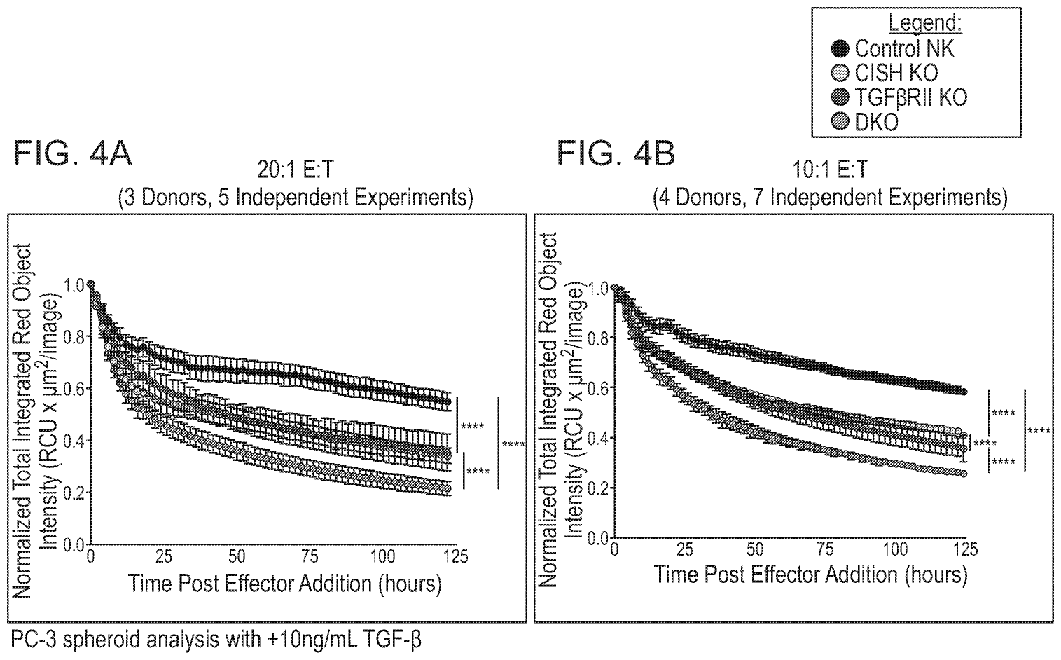

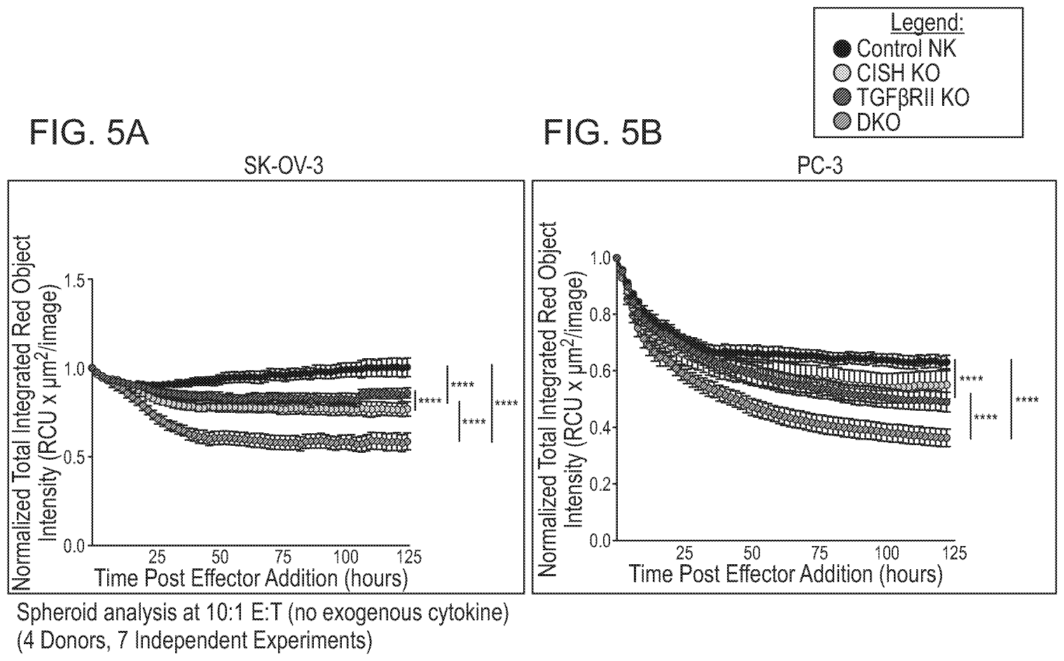

Moreover, CISH KO NK cells reduced the growth of SK-OV-3 ovarian (FIGS. 3A-3B and FIG. 5A) and PC-3 prostate tumor spheroids (FIGS. 4A-4B and FIG. 5B) by an average of 23% and 12% (p<0.0001 in both cases), respectively, in comparison to unedited controls. However, the activity of CISH KO NK cells were dampened by addition of exogenous TGF-β.

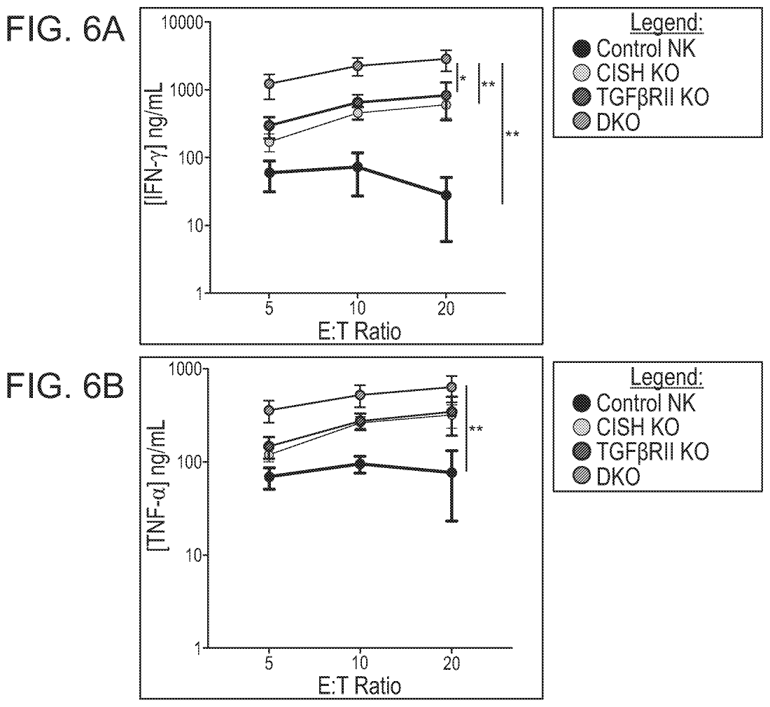

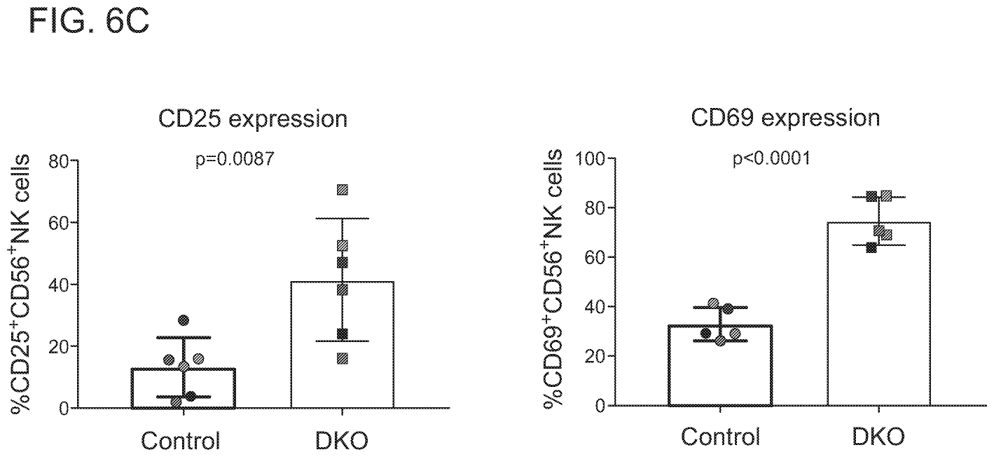

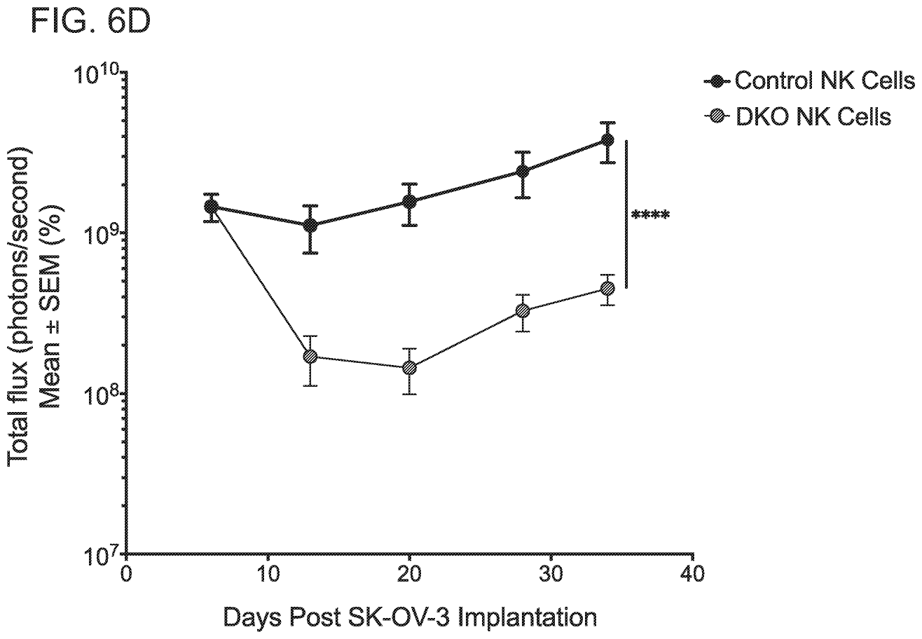

Given this observation, a knockout of the TGF-β receptor gene, TGFBR2, with the CISH KO was generated. Single knockout of TGFBR2 rendered NK cells resistant to TGFβ inhibition (p<0.0001). Importantly, across 4 unique donors and 7 independent experiments, TGFBR2/CISH double knockout (DKO) NK cells demonstrated superior effector function and attenuated SK-OV-3 and PC-3 tumor spheroid growth by greater than 60% for both tumor types, with supplement of exogenous TGF-β (FIGS. 3A-3B and FIGS. 4A-4B) and without supplement of exogenous TGF-β (FIG. 5A-5B). These effector functions were statistically greater than control NK cells or TGFBR2 and CISH single knockout NK cells (p<0.0001 in all cases). In addition, TGFBR2/CISH DKO NK cells produced higher concentrations of TNF-α (FIG. 6A) and IFN-γ (FIG. 6B) p<0.01 in both cases as assessed by ELISA.

Double KO NK cells expressed significantly higher levels of activation markers CD25 and CD69 as compared to control NK cells (FIG. 6C).

Anti-tumor activity of edited NK cells was measured in an in vivo model. NSG mice received an intraperitoneal injection of 500,000 SKOV3 tumor cells labeled with luciferase. Seven days post-tumor implantation, 10 million edited (CISH/TGFBR2 double-knockout) or unedited (control) NK cells were injected into the peritoneal cavity of the tumor-bearing mice. Tumor burden was monitored weekly by IP administration of luciferin and IVIS imaging. Two-way ANOVA analysis was performed at day 34 to determine statistical significance between control and DKO NK cell groups (****, P<0.0001) (FIG. 6.D).

These results demonstrate efficient editing of primary human NK cells at two unique targets simultaneously with CRISPR-Cpf1. Together, the increased effector function of CISH/TGFBR2 DKO primary human NK cells in vitro and in vivo relative to either single knockout or unedited NK cells indicates an enhanced and synergistic effect of the CISH/TGFBR2 DKO.

### Example 11: Gene Edited NK Cells, Exhibiting Loss-of-Function of TIGIT, NKG2A or ADORA2A, Demonstrate Improved Effector Function in Response to Tumor Cells

A next-generation allogeneic NK cell therapy was developed using CRISPR-Cpf1 gene editing to enhance NK cell function through knockout of the TIGIT, NKG2A, or ADORA2A genes.

NK cells were expanded as previously described in Example 10. Briefly, NK cells were expanded ex-vivo for 14 days in IL15, and then edited with the respective targeting RNP complex. Gene editing was performed at different NK cell expansion stages (between day 8-21). For editing of TIGIT, NKG2A, or ADORA2A, guides for the corresponding targets were complexed with Cpf1 nuclease at a 2:1 ratio to form ribonucleoproteins (RNPs). Where cells were edited with both targets, RNP complexation for each target was done separately and then mixed at a 1:1 ratio prior to electroporation. NK cells were electroporated as previously described in Example 10.

The following guide RNA sequences were used for editing of TIGIT, NKG2A, or ADORA2A:

TABLE 13

| | gRNA targeting domain | |
|---|---|---|
| Target | Sequence (DNA) | gRNA sequence |
| TIGIT | TGCAGAGAAAGGTGGCTCT | ATGTGTTTTTGTCAAAAGACCT |
| | A | TTTrUrArArUrUrUrCrUrArCrUrCr |
| | | UrUrGrUrArGrArUrUrGrCrArGrAr |
| | | GrArArArGrGrUrGrGrCrUrCrUrA |
| NKG2A | GCAACTGAACAGGAAATAA | UAAUUUCUACUCUUGUAGAUG |
| | CC | CAACUGAACAGGAAAUAACC |
| ADORA2A | CCTGTGTGCTGGTGCCCCTG | ATGTGTTTTTGTCAAAAGACCT |
| | | TTTrUrArArUrUrUrCrUrArCrUrCr |
| | | UrUrGrUrArGrArUrCrCrUrGrUrGr |
| | | UrGrCrUrGrGrUrGrCrCrCrCrUrG |

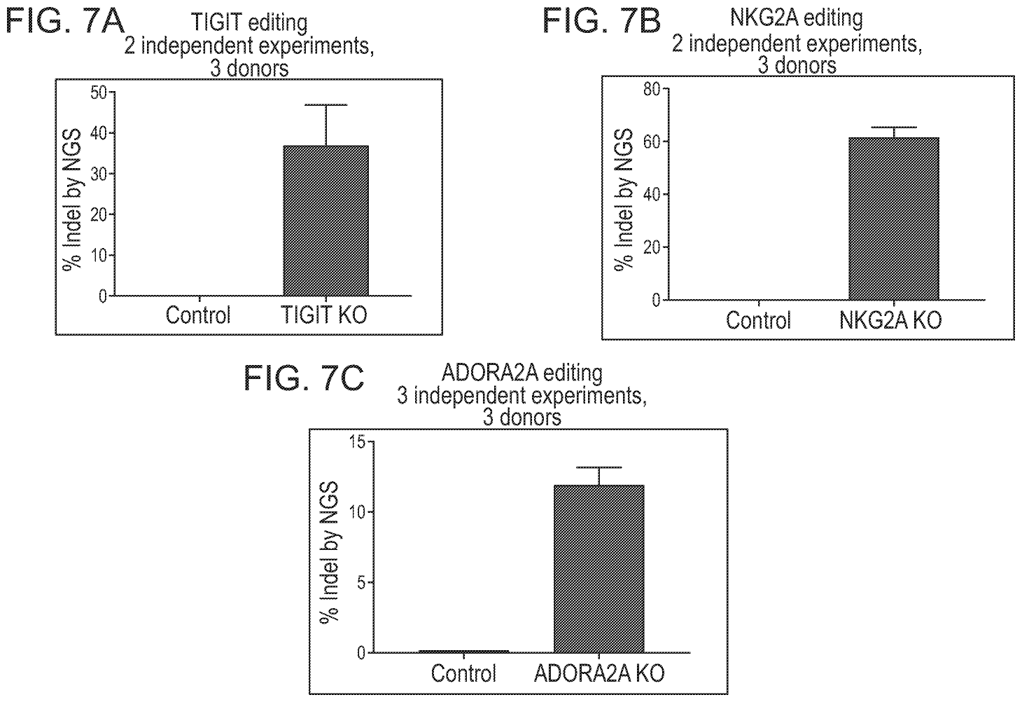

As demonstrated in FIGS. 7A-7C, robust single gene editing of TIGIT, NKG2A, and ADORA2A was achieved in NK cells.

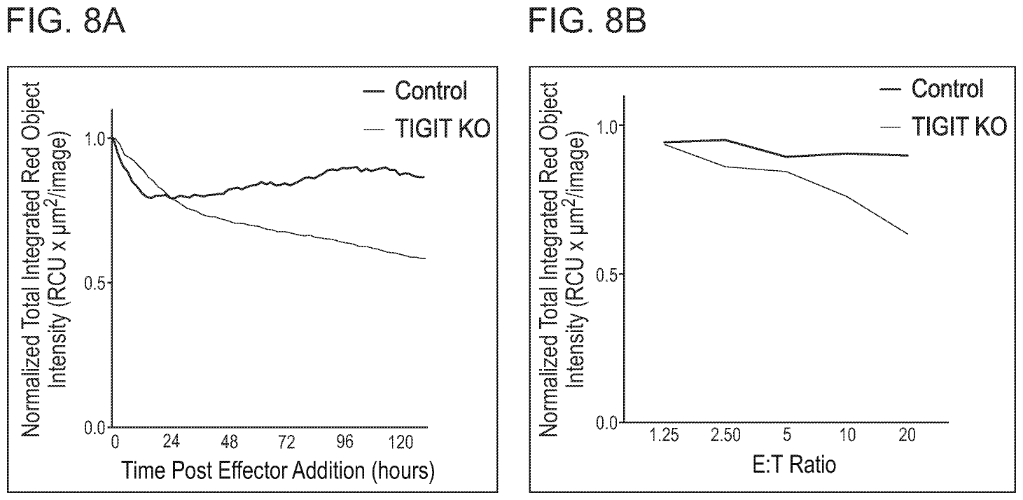

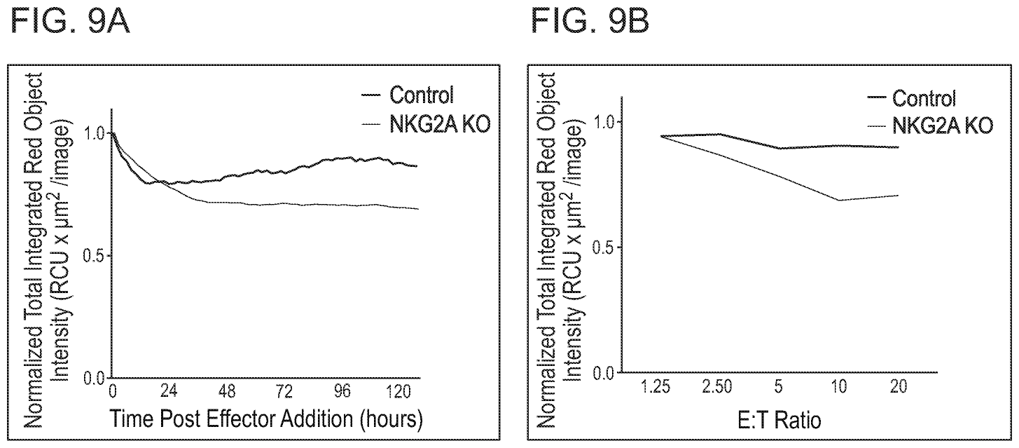

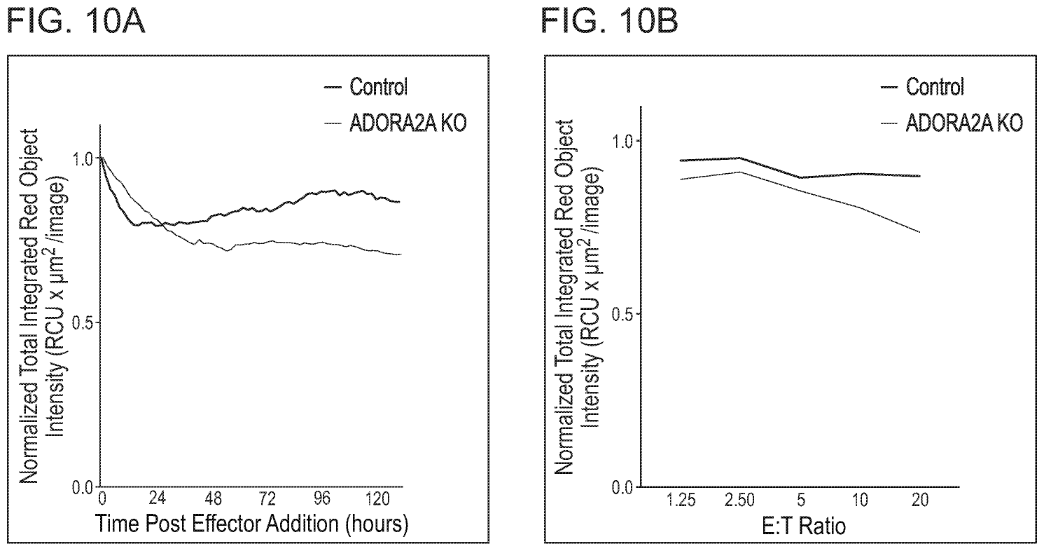

The efficacy of the effector cells (primary human NK cells treated with different RNPs), was assessed in vitro to determine the function of TIGIT single KO (FIGS. 8A-8B), NKG2A single KO (FIGS. 9A-9B) and ADORA2A single KO (FIGS. 10A-10B) by 3D tumor spheroid assays

To form spheroids, 5,000 NucLight Red labeled PC-3 or SK-OV-3 tumor cells were plated in a single well of ultra-low attachment 96 well plates, centrifuged at 1,000 rpm for 10 minutes, and incubated for 96 hours at 37° C. At 96 hours, effector cells (primary human NK cells treated with different RNPs) were added to spheroids at multiple effector to target cell ratios with or without 10 ng/mL of TGF-beta. Red object intensity was measured every two hours for 6 days on an Incucyte imaging system. Data shown are normalized to the red object intensity at time of effector addition.

Across 2 unique donors and 2 independent experiments, TIGIT single KO (FIGS. 8A-8B), NKG2A single KO (FIGS. 9A-9B) and ADORA2A single KO (FIGS. 10A-10B) NK cells demonstrated superior effector function and attenuated SK-OV-3 and PC-3 tumor spheroid growth. These data demonstrate efficient editing of primary human NK cells at three independent unique targets with CRISPR-Cpf1, resulting in increased effector function of TIGIT single KO, NKG2A single KO and ADORA2A single KO primary human NK cells in vitro relative to unedited NK cells.

### Example 12: Gene Edited NK Cells, Exhibiting Loss-of-Function of CISH, TGFBR2 and TIGIT, Demonstrate Improved Effector Function in Response to Tumor Cells

A next-generation allogeneic NK cell therapy was developed using CRISPR-Cpf1 gene editing to enhance NK cell function through knockout of the CISH, TGFBR2 and TIGIT genes.

NK cells were expanded as previously described in Example 10. Briefly, NK cells were expanded ex-vivo for 14 days in IL15, and then edited with the respective targeting RNP complex. Gene editing was performed at different NK cell expansion stages (between day 8-21). For editing of CISH, TGFBR2 and TIGIT, guides for the targets were complexed with Cpf1 nuclease to form ribonucleoproteins (RNPs), RNP complexation for each target was done separately and then mixed at a 1:1 ratio prior to electroporation. NK cells were electroporated as previously described in Example 10.

The guide RNA sequences that were used for editing of CISH, TGFBR2 and TIGIT are indicated in Table 14 below:

TABLE 14

| | gRNA targeting domain | |
|---|---|---|
| Target | Sequence (DNA) | gRNA sequence |
| CISH | ACTGACAGCGTGAACAGGTAG | ATGTGTTTTTGTCAAAAGACCTTTTrUrA |
| | | rArUrUrUrCrUrArCrUrCrUrUrGrUr |
| | | ArGrArUrArCrUrGrArCrArGrCrGrU |
| | | rGrArArCrArGrGrUrArG |
| TGFBR2 | TGATGTGAGATTTTCCACCT | ATGTGTTTTTGTCAAAAGACCTTTTrUrA |
| | | rArUrUrUrCrUrArCrUrCrUrUrGrUr |
| | | ArGrArUrUrGrArUrGrUrGrArGrArU |
| | | rUrUrUrCrCrArCrCrU |
| TIGIT | TGCAGAGAAAGGTGGCTCTA | ATGTGTTTTTGTCAAAAGACCTTTTr |
| | | UrArArUrUrUrCrUrArCrUrCrUrUrGrUrA |
| | | rGrArUrUrGrCrArGrArGrArArArGrGrUr |
| | | GrGrCrUrCrUrA |

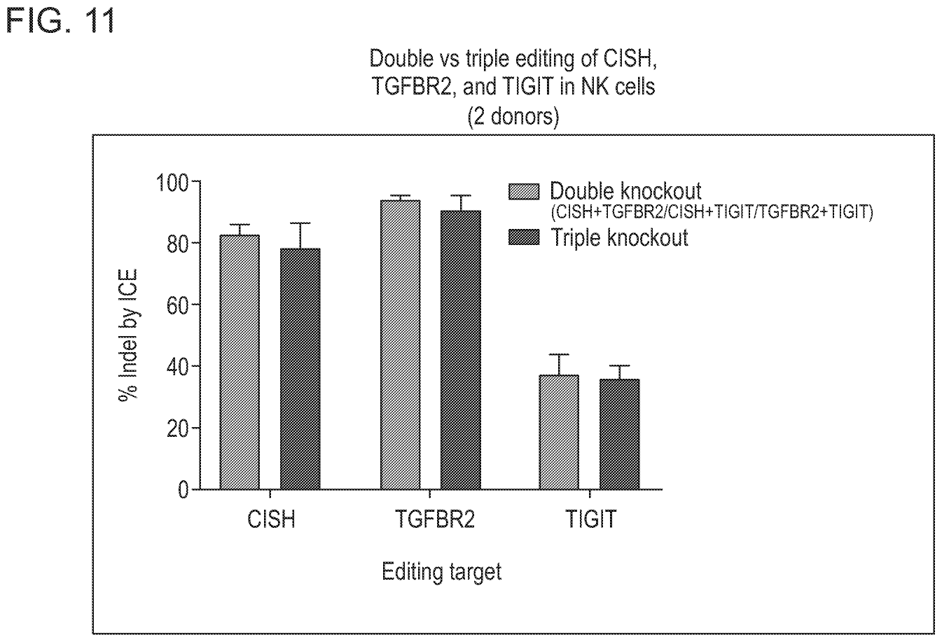

As demonstrated in FIG. 11, robust triple-gene editing of TGFBR2, CISH and TIGIT was achieved in NK cells.

The efficacy of the effector cells was assessed in vitro by 3D tumor spheroid assays.

To form spheroids, 5,000 NucLight Red labeled PC-3 or SK-OV-3 tumor cells were plated in a single well of ultra-low attachment 96 well plates, centrifuged at 1,000 rpm for 10 minutes, and incubated for 96 hours at 37° C. At 96 hours, effector cells (primary human NK cells treated with different RNPs) were added to spheroids at multiple effector to target cell ratios with or without 10 ng/mL of TGF-beta. Red object intensity was measured every two hours for 6 days on an Incucyte imaging system. Data shown are normalized to the red object intensity at time of effector addition. Normalization of spheroid curves maintains the same efficacy patterns observed in non-normalized data.

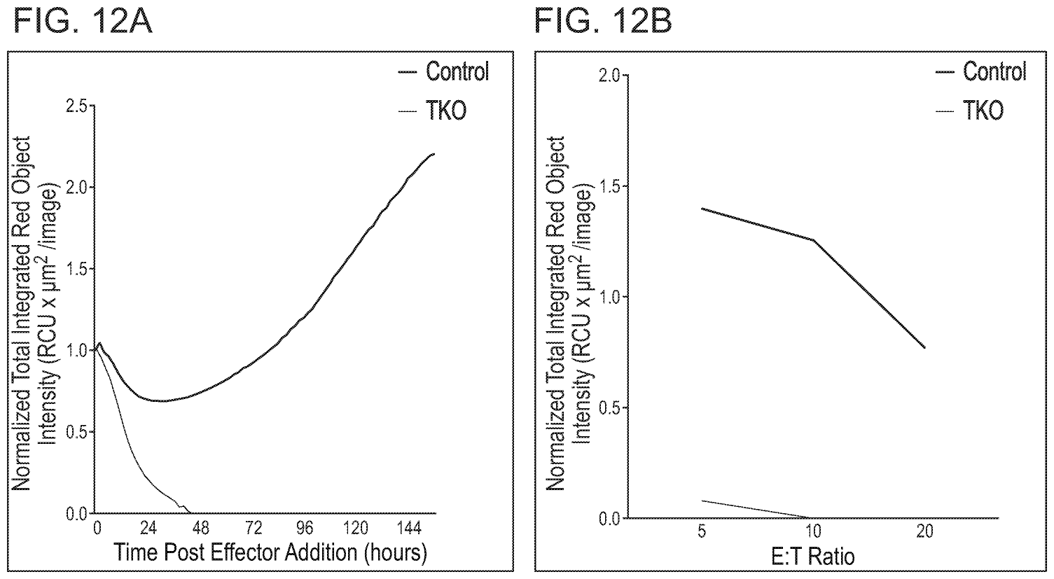

TGFBR2/CISH/TIGIT triple knockout (TKO) NK cells demonstrated superior effector function and attenuated SK-OV-3 and PC-3 tumor spheroid growth (FIGS. 12A-12B). These effector functions were statistically greater than control NK cells. These results demonstrate efficient editing of primary human NK cells at three unique targets simultaneously with CRISPR-Cpf1. Together, the increased effector function of CISH/TGFBR2/TIGIT TKO primary human NK cells in vitro and relative to unedited NK cells indicates an enhanced effect of the CISH/TGFBR2/TIGIT TKO.

1. A modified lymphocyte, wherein the modified lymphocyte:
(a) does not express endogenous CD3, CD4, and/or CD8; and
(b) expresses at least one endogenous gene encoding:
(i) CD56 (NCAM), CD49, and/or CD45;
(ii) NK cell receptor immunoglobulin gamma Fc region receptor III (FcγRIII, cluster of differentiation 16 (CD16));
(iii) natural killer group-2 member D (NKG2D);
(iv) CD69;
(v) a natural cytotoxicity receptor;
or any combination of two or more thereof;
wherein the modified lymphocyte further:
(1) comprises at least one exogenous nucleic acid expression construct comprising a nucleic acid sequence encoding:
(i) a chimeric antigen receptor (CAR);
(ii) a non-naturally occurring variant of FcγRIII (CD16);
(iii) interleukin 15 (IL-15);
(iv) IL-15 receptor (IL-15R), or a variant thereof;
(v) interleukin 12 (IL-12);
(vi) IL-12 receptor (IL-12R), or a variant thereof;
(vii) human leukocyte antigen G (HLA-G);
(viii) human leukocyte antigen E (HLA-E);
(ix) leukocyte surface antigen cluster of differentiation CD47 (CD47);
or any combination of two or more thereof;
and/or
(2) exhibits a loss of function of at least one of:
(i) transforming growth factor beta receptor 2 (TGFβR2);
(ii) adenosine A2a receptor (ADORA2A);
(iii) T cell immunoreceptor with Ig and ITIM domains (TIGIT);
(iv) β-2 microgobulin (B2M);
(v) programmed cell death protein 1 (PD-1);
(vi) cytokine inducible SH2 containing protein (CISH);
(vii) class II, major histocompatibility complex, transactivator (CIITA);
(viii) natural killer cell receptor NKG2A (natural killer group 2A);
(ix) two or more HLA class II histocompatibility antigen alpha chain genes, and/or two or more HLA class II histocompatibility antigen beta chain genes;
(x) cluster of differentiation 32B (CD32B, FCGR2B);
(xi) T cell receptor alpha constant (TRAC);
or any combination of two or more thereof.

2. The modified lymphocyte of claim 1, wherein
(a) the lymphocyte exhibits a loss of function of:
(i) TGFβR2, CISH, TIGIT, ADORA2A, or NKG2A;
(ii) TGFβR2 and CISH, TGFβR2 and TIGIT, TGFβR2 and ADORA2A, TGFβR2 and NKG2A, CISH and TIGIT, CISH and ADORA2A, CISH and NKG2A, TIGIT and ADORA2A, TIGIT and NKG2A, or ADORA2A and NKG2A; or
(iii) TGFβR2, CISH and TIGIT; TGFβR2, CISH and ADORA2A; TGFβR2, CISH and NKG2A; TGFβR2, TIGIT and ADORA2A; TGFβR2, TIGIT and NKG2A; TGFβR2, ADORA2A and NKG2A; CISH, TIGIT and ADORA2A; CISH, TIGIT and NKG2A; CISH, ADORA2A and NKG2A; or TIGIT, ADORA2A and NKG2A;
(b) the lymphocyte comprises a rearranged endogenous T-cell receptor (TCR) locus;
(c) the natural cytotoxicity receptor is NKp30, NKp44, NKp46, and/or CD158b;
(d) the IL-15R variant is a constitutively active IL-15R variant and/or wherein the IL12-R variant is a constitutively active IL12-R variant;
(e) the loss of TGFβR2 is associated with exogenous expression of a dominant-negative variant of TGFβ receptor II (DN-TGFβR2);
(f) the CAR is capable of binding mesothelin, EGFR, HER2, MICA/B, BCMA, CD19, CD22, CD20, CD33, CD123, androgen receptor, PSMA, PSCA, Muc1, HPV viral peptides (i.e. E7), EBV viral peptides, CD70, WT1, CEA, EGFRvIII, IL13Ra2, and GD2, CA125, CD7, EpCAM, Muc16, or CD30;
(g) the lymphocyte exhibits a loss-of-function in two or more of the genes/proteins listed under (2);
(h) the lymphocyte comprises an indel or an insertion of an exogenous nucleotide construct into a genomic locus harboring a gene or encoding a protein under (2)
(i) the lymphocyte comprises an indel or an insertion of an exogenous nucleotide construct into two or more genomic loci harboring a gene or encoding a protein under (2)
(j) the modified lymphocyte expresses endogenous CD56, CD49, and CD45; and/or
(k) the lymphocyte is a natural killer (NK) cell.

3-10. (canceled)

11. The modified lymphocyte of claim 1, wherein the lymphocyte is derived from a pluripotent or multipotent stem cell.

12. The modified lymphocyte of claim 11, wherein the multipotent stem cell is a hematopoietic stem cell (HSC); and/or the pluripotent stem cell is an induced pluripotent stem cell (iPSC) or an embryonic stem cell (ESC).

13-14. (canceled)

15. The modified lymphocyte of claim 1, wherein the lymphocyte is derived from a pluripotent or multipotent stem cell that comprises at least one or more exogenous nucleic acid constructs encoding any of (1)(i)-(1)(xi), or any combination thereof; and/or at least one genomic alteration that effects the loss-of-function of any of (2)(i)-(2) (iii), or any combination thereof, in the lymphocyte.

16. The modified lymphocyte of claim 15, wherein
the lymphocyte is derived from a pluripotent or multipotent stem cell that comprises at least one genomic alteration that effects the loss-of-function of any of (2)(i)-(2) (iii), or any combination thereof, in the lymphocyte; or
the at least one genomic alteration that effects the loss-of-function of one or more (2)(i)-(2)(iii) in the lymphocyte comprises an insertion of an exogenous nucleic acid construct.

17-21. (canceled)

22. The modified lymphocyte of claim 1, wherein the lymphocyte is obtained by editing a genomic locus with an RNA-guided nuclease.

23. The modified lymphocyte of claim 22, wherein
the RNA-guided nuclease is a CRISPR/Cas nuclease; or
the RNA-guided nuclease is selected from the group consisting of SpCas9, SaCas9, (KKH) SaCas9, AsCpf1 (AsCas12a), LbCpf1, (LbCas12a), CasX, CasY, Cas12h1, Cast2i1, Cas12c1, Cas12c2, eSpCas9, Cas9-HF1, HypaCas9, dCas9-Fokl, Sniper-Cas9, xCas9, AaCas12b, evoCas9, SpCas9-NG, VRQR, VRER, NmeCas9, CjCas9, BhCas12b, and BhCas12b V4.

24. (canceled)

25. The modified lymphocyte of claim 1, wherein the lymphocyte is obtained by editing two or more genomic loci harboring genes encoding any of the proteins under (2).

26. The modified lymphocyte of claim 25, wherein
at least two of the two or more genomic loci harboring genes encoding any of the proteins under (2) have been edited by a different RNA-guided nuclease; or
at least one of the two or more genomic loci harboring genes encoding any of the proteins under (2) has been edited by Cas9, and wherein at least one of the loci has been edited by Cpf1.

27-29. (canceled)

30. A modified cell, wherein the modified cell
(1) comprises at least one exogenous nucleic acid expression construct comprising a nucleic acid sequence encoding:
(i) a chimeric antigen receptor (CAR);
(ii) a non-naturally occurring variant of FcγRIII (CD16);
(iii) interleukin 15 (IL-15);
(iv) IL-15 receptor (IL-15R), or a variant thereof;
(v) interleukin 12 (IL-12);
(vi) IL-12 receptor (IL-12R), or a variant thereof;
(vii) human leukocyte antigen G (HLA-G);
(viii)human leukocyte antigen E (HLA-E);
(ix) leukocyte surface antigen cluster of differentiation CD47 (CD47);
or any combination of two or more thereof;
and/or
(2) exhibits a loss of function of at least one of:
(i) transforming growth factor beta receptor 2 (TGFβR2);
(ii) adenosine A2a receptor (ADORA2A);
(iii) T cell immunoreceptor with Ig and ITIM domains (TIGIT);
(iv) β-2 microgobulin (B2M);
(v) programmed cell death protein 1 (PD-1);
(vi) cytokine inducible SH2 containing protein (CISH);
(vii) class II, major histocompatibility complex, transactivator (CIITA);
(viii) natural killer cell receptor NKG2A (natural killer group 2A);
(ix) two or more HLA class II histocompatibility antigen alpha chain genes, and/or two or more HLA class II histocompatibility antigen beta chain genes;
(x) cluster of differentiation 32B (CD32B, FCGR2B);
(xi) T cell receptor alpha constant (TRAC);
or any combination of two or more thereof.

31. The modified cell of claim 30, wherein
(a) the modified cell exhibits a loss of function of:
(i) TGFβR2, CISH, TIGIT, ADORA2A, or NKG2A;
(ii) TGFβR2 and CISH, TGFβR2 and TIGIT, TGFβR2 and ADORA2A, TGFβR2 and NKG2A, CISH and TIGIT, CISH and ADORA2A, CISH and NKG2A, TIGIT and ADORA2A, TIGIT and NKG2A, or ADORA2A and NKG2A; or
(iii) TGFβR2, CISH and TIGIT; TGFβR2, CISH and ADORA2A; TGFβR2, CISH and NKG2A; TGFβR2, TIGIT and ADORA2A; TGFβR2, TIGIT and NKG2A; TGFβR2, ADORA2A and NKG2A; CISH, TIGIT and ADORA2A; CISH, TIGIT and NKG2A; CISH, ADORA2A and NKG2A; or TIGIT, ADORA2A and NKG2A;
(b) the modified cell is an immune cell;
(c) the cell is a pluripotent stem cell, or a differentiated daughter cell derived therefrom;
(d) the cell comprises a rearranged endogenous TCR locus, wherein the rearranged TCR comprises TCRα VJ and/or TCRβ V(D)J section rearrangements and complete V-domain exons:
(e) the modified cell expresses at least one endogenous gene encoding:
(i) CD56 (NCAM), CD49, and/or CD45;
(ii) FcγRIII (CD16);
(iii) natural killer group-2 member D (NKG2D);
(iv) CD69;
(v) a natural cytotoxicity receptor;
or any combination of two or more thereof; or
(f) the cell expresses at least one NK cell biomarker.

32-42. (canceled)

43. A population of cells comprising the modified lymphocyte of claim 1.

44. A pharmaceutical composition comprising the population of cells of claim 43.

45. An isolated population of lymphocytes, wherein the population comprises at least 1×10³, at least 1×10⁴, at least 1×10⁵, at least 2×10⁵, at least 3×10⁵, at least 4×10⁵, at least 5×10⁵, at least 1×10⁶, at least 2×10⁶, at least 3×10⁶, at least 4×10⁶, at least 5×10⁶, at least 1×10⁷, at least 1×10⁷, at least 2×10⁷, at least 3×10⁷, at least 4×10⁷, at least 5×10⁷, at least 1×10⁸, at least 2×10⁸, at least 3×10⁸, at least 4×10⁸, at least 5×10⁸, at least 1×10⁹, at least 1×10⁹, at least 2×10⁹, at least 3×10⁹, at least 4×10⁹, at least 5×10⁹, at least 1×10¹⁰, at least 2×10¹⁰, at least 3×10¹⁰, at least 4×10¹⁰, at least 5×10¹⁰, at least 1×10¹¹, or at least 1×10¹² cells, and wherein at least 50%, at least 60%, at least 70%, at least 80%, at least 90%, at least 95%, at least 98%, at least 99%, at least 99.9%, at least 99.99%, at least 99.999%, or virtually 100% of the lymphocytes in the population:
(a) comprise a rearranged T-cell receptor (TCR) locus;
(b) do not express endogenous CD3;
(c) express endogenous CD56 (NCAM), CD49, and/or CD45; and
(d) expresses at least one endogenous gene encoding:
(i) FcγRIII (CD16);
(ii) natural killer group-2 member D (NKG2D);
(iii) CD69;
(iv) a natural cytotoxicity receptor;
or any combination of two or more thereof; and
wherein the modified lymphocyte further:
(1) comprises at least one exogenous nucleic acid expression construct comprising a nucleic acid sequence encoding:
(i) chimeric antigen receptor (CAR);
(ii) non-naturally occurring variant of immunoglobulin gamma Fc region receptor III (FcγRIII, CD16);
(iii) interleukin 15 (IL-15);
(iv) IL-15 receptor (IL-15R), or a variant thereof;
(v) interleukin 12 (IL-12);
(vi) IL-12 receptor (IL-12R), or a variant thereof;
(vii) human leukocyte antigen G (HLA-G);
(viii) human leukocyte antigen E (HLA-E);
(ix) leukocyte surface antigen cluster of differentiation CD47 (CD47);
or any combination of two or more thereof;
and/or
(2) exhibits a loss of function of at least one of:
(i) transforming growth factor beta receptor 2 (TGFβR2);
(ii) adenosine A2a receptor (ADORA2A);
(iii) T cell immunoreceptor with Ig and ITIM domains (TIGIT);
(iv) β-2 microgobulin (B2M);
(v) programmed cell death protein 1 (PD-1);
(vi) cytokine inducible SH2 containing protein (CISH);
(vii) class II, major histocompatibility complex, transactivator (CIITA);
(viii) natural killer cell receptor NKG2A (natural killer group 2A);
(ix) two or more HLA class II histocompatibility antigen alpha chain genes, and/or two or more HLA class II histocompatibility antigen beta chain genes;
(x) cluster of differentiation 32B (CD32B, FCGR2B);
(xi) T cell receptor alpha constant (TRAC);
or any combination of two or more thereof.

46. The isolated population of lymphocytes of claim 45, wherein
(a) the modified lymphocyte exhibits a loss of function of:
(i) TGFβR2, CISH, TIGIT, ADORA2A, or NKG2A;
(ii) TGFβR2 and CISH, TGFβR2 and TIGIT, TGFβR2 and ADORA2A, TGFβR2 and NKG2A, CISH and TIGIT, CISH and ADORA2A, CISH and NKG2A, TIGIT and ADORA2A, TIGIT and NKG2A, or ADORA2A and NKG2A; or
(iii) TGFβR2, CISH and TIGIT; TGFβR2, CISH and ADORA2A; TGFβR2, CISH and NKG2A; TGFβR2, TIGIT and ADORA2A; TGFβR2, TIGIT and NKG2A; TGFβR2, ADORA2A and NKG2A; CISH, TIGIT and ADORA2A; CISH, TIGIT and NKG2A; CISH, ADORA2A and NKG2A; or TIGIT, ADORA2A and NKG2A;
(b) the rearranged TCR locus comprises of TCRα VJ and/or TCRβ V(D)J section rearrangements and complete V-domain exons;
(c) the natural cytotoxicity receptor is NKp30, NKp44, NKp46, and/or CD158b;
(d) the population does not comprise cells harboring episomal expression constructs encoding a reprogramming factor;
(e) each cell in the population of cells comprises the same combination of (1) and (2); or
(f) the population comprises less than 0.001%, less than 0.002%, less than 0.003%, less than 0.004%, less than 0.005%, less than 0.006%, less than 0.007%, less than 0.008%, less than 0.009%, less than 0.01%, less than 0.02%, less than 0.03%, less than 0.04%, less than 0.05%, less than 0.06%, less than 0.07%, less than 0.08%, less than 0.09%, less than 0.1%, less than 0.2%, less than 0.3%, less than 0.4%, less than 0.5%, less than 0.6%, less than 0.7%, less than 0.8%, less than 0.9%, less than 1%, less than 2%, less than 3%, less than 4%, less than 5%, less than 6%, less than 7%, less than 8%, less than 9%, or less than 10% cell that harbor a chromosomal translocation.

47-49. (canceled)

50. The isolated in vitro population of lymphocytes of claim 45, wherein the population does not comprise more than 1%, more than 0.1%, more than 0.001%, more than 0.0001%, more than 0.00001%, more than 0.000001%, more than 0.0000001%, more than 0.00000001%, more than 0.000000001%, more than 0.0000000001%, or more than more than 0.00000000001% of cells expressing a reprogramming factor from an exogenous nucleic acid construct.

51. The isolated population of lymphocytes of claim 50, wherein
the population does not comprise a cell expressing a reprogramming factor from an exogenous nucleic acid construct; or
the reprogramming factor is Oct-4 and/or Sox-2.

52-55. (canceled)

56. A method of treating a subject, the method comprising administering the pharmaceutical composition of claim 44 to a subject in need thereof, thereby treating the subject.

57. The method of claim 56, wherein the subject has, or is diagnosed with, a proliferative disease.

58. The method of claim 57, wherein the proliferative disease is cancer.

59. The method of claim 58, wherein the cancer is selected from the group consisting of breast cancer, colorectal cancer, gastric cancer, renal cell carcinoma (RCC), or non-small cell lung cancer (NSCLC), solid tumors, bladder cancer, hepatocellular carcinoma, prostate cancer, ovarian/uterine cancer, pancreatic cancer, mesothelioma, melanoma, glioblastoma, HPV-associated and/or HPV-positive cancers such as cervical and HPV+ head and neck cancer, oral cavity cancer, cancer of the pharynx, thyroid cancer, gallbladder cancer, soft tissue sarcomas, and hematological cancers like ALL, CLL, NHL, DLBCL, AML, CML, multiple myeloma (MM).

60. A method of generating the lymphocyte of claim 1, the modified cell of claim 30 or the isolated population of lymphocytes of claim 45, the method comprising:
(a) obtaining an induced pluripotent stem cell (iPSC);
(b) modifying the iPSC, or an undifferentiated or differentiated daughter cell thereof, to comprise express at least one exogenous nucleic acid expression construct of (1) and/or to comprise a loss of function in at least one gene of (2);
(c) directing differentiation of the iPSC to hematopoietic lineage cells,
wherein the hematopoietic lineage cells retain the edited genetic loci comprised in the iPSCs.

61. The method of claim 60, wherein
(a) step (c) comprises:
(i) contacting iPSCs with a composition comprising a BMP pathway activator, and optionally bFGF, to obtain mesodermal cells; and
(ii) contacting the mesodermal cells with a composition comprising a BMP pathway activator, bFGF, and a WNT pathway activator, to obtain mesodermal cells having definitive hemogenic endothelium (HE) potential, wherein the mesodermal cells having definitive hemogenic endothelium (HE) potential are capable of providing hematopoietic lineage cells;
wherein mesodermal cells and mesodermal cells having definitive HE potential are obtained in steps (i) and (ii) without the step of forming embryoid bodies;
wherein the hematopoietic lineage cells comprise definitive hemogenic endothelium cells, hematopoietic stem and progenitor cells (HSC), hematopoietic multipotent progenitor cell (MPP), pre-T cell progenitor cells, pre-NK cell progenitor cells, T cell progenitor cells, NK cell progenitor cells, T cells, NK cells, NKT cells, or B cells;
(b) the method further comprises contacting the definitive HE cells with a composition comprising a BMP activator, and optionally a ROCK inhibitor, and one or more growth factors and cytokines selected from the group consisting of TPO, IL3, GMCSF, EPO, bFGF, VEGF, SCF, IL6, Flt3L and IL11 to obtain hematopoietic multipotent progenitor cells (MPP);
(c) the method further comprises contacting the definitive HE cells with a composition comprising one or more growth factors and cytokines selected from the group consisting of SCF, Flt3L, and IL7; and optionally one or more of a BMP activator, a ROCK inhibitor, TPO, VEGF and bFGF to obtain pre-T cell progenitors, T cell progenitors, and/or T cells:
(d) the method further comprises contacting the definitive HE cells with a composition comprising one or more growth factors and cytokines selected from the group consisting of SCF, Flt3L, TPO, IL7 and IL15, and optionally one or more of a BMP activator, a ROCK inhibitor, VEGF and bFGF to obtain pre-NK cell progenitors, NK cell progenitors, and/or NK cells;
(e) the method further comprises prior to step (c), contacting the pluripotent stem cells with a composition comprising a MEK inhibitor, a GSK3 inhibitor, and a ROCK inhibitor, to seed and expand the cells; or
(f) the method further comprises detecting a rearranged T-cell receptor (TCR) locus in the hematopoietic lineage cells.

62-69. (canceled)

70. A method, the method comprising:
reprogramming a donor cell to a pluripotent state;
editing a target locus in the donor cell genome; and
differentiating the reprogrammed donor cell into a lymphocyte.

71. The method of claim 70, wherein
the editing is performed before or during the step of reprogramming of the donor cell to a pluripotent state; or
the donor cell is a fibroblast, a peripheral blood cell, a lymphocyte, or a T cell.

72. (canceled)

73. A method, the method comprising:
differentiating a genetically modified pluripotent stem cell into a lymphocyte, wherein the genetically modified pluripotent stem cell comprises:
(1) an exogenous nucleic acid expression construct comprising:
(i) a nucleic acid sequence encoding a chimeric antigen receptor (CAR);
(ii) a nucleic acid sequence encoding a non-naturally occurring variant of FcγRIII (CD16);
(iii) a nucleic acid sequence encoding interleukin 15 (IL-15);
(iv) a nucleic acid sequence encoding interleukin 15 receptor (IL-15R) or a variant thereof;
(v) a nucleic acid sequence encoding interleukin 12 (IL12);
(vi) a nucleic acid sequence encoding interleukin-12 receptor (IL-12R) or a variant thereof;
(vii) a nucleic acid sequence encoding human leukocyte antigen G (HLA-G);
(viii) a nucleic acid sequence encoding human leukocyte antigen E (HLA-E);
(ix) a nucleic acid sequence encoding leukocyte surface antigen cluster of differentiation CD47 (CD47);
or any combination of two or more thereof; and
(2) an indel, or an insertion of an exogenous nucleic acid in one or more of the following genetic loci:
(i) transforming growth factor beta receptor 2 (TGFβR2);
(ii) adenosine A2a receptor (ADORA2A);
(iii) T cell immunoreceptor with Ig and ITIM domains (TIGIT);
(iv) β-2 microgobulin (B2M);
(v) programmed cell death protein 1 (PD-1, CD279);
(vi) cytokine inducible SH2 containing protein (CISH);
(vii) class II, major histocompatibility complex, transactivator (CIITA);
(viii) natural killer cell receptor NKG2A (natural killer group 2A);
(ix) two or more HLA class II histocompatibility antigen alpha chain genes, and/or two or more HLA class II histocompatibility antigen beta chain genes;
(x) cluster of differentiation 32B (CD32B, FCGR2B);
(xi) T cell receptor alpha constant (TRAC);
or any combination of two or more thereof,
wherein the indel or insertion results in a loss-of-function of a gene product encoded by the respective genetic locus or loci.

74. The method of claim 73, wherein
(a) the indel, or the insertion of an exogenous nucleic acid is in the following genetic loci:
(i) TGFβR2, CISH, TIGIT, ADORA2A, or NKG2A;
(ii) TGFβR2 and CISH, TGFβR2 and TIGIT, TGFβR2 and ADORA2A, TGFβR2 and NKG2A, CISH and TIGIT, CISH and ADORA2A, CISH and NKG2A, TIGIT and ADORA2A, TIGIT and NKG2A, or ADORA2A and NKG2A; or
(iii) TGFβR2, CISH and TIGIT; TGFβR2, CISH and ADORA2A; TGFβR2, CISH and NKG2A; TGFβR2, TIGIT and ADORA2A; TGFβR2, TIGIT and NKG2A;
TGFβR2, ADORA2A and NKG2A; CISH, TIGIT and ADORA2A; CISH, TIGIT and NKG2A; CISH, ADORA2A and NKG2A; or TIGIT, ADORA2A and NKG2A,
wherein the indel or insertion results in a loss-of-function of a gene product encoded by the respective genetic locus or loci;
(b) the exogenous nucleic acid of (2) is the exogenous nucleic acid of (1);
(c) the pluripotent stem cell is an iPS cell; or
(d) the differentiating comprises contacting the pluripotent stem cell with a differentiation medium or a sequence of differentiation media.

75-77. (canceled)

78. The modified lymphocyte of claim 1, wherein
the two or more HLA class II histocompatibility antigen alpha chain genes are selected from HLA-DQA1, HLA-DRA, HLA-DPA1, HLA-DMA, HLA-DQA2, and HLA-DOA; or
the two or more HLA class II histocompatibility antigen beta chain genes are selected from HLA-DMB, HLA-DOB, HLA-DPB1, HLA-DQB1, HLA-DQB3, HLA-DQB2, HLA-DRB1, HLA-DRB3, HLA-DRB4, and HLA-DRB5.

79. (canceled)

80. The modified lymphocyte of claim 1, the modified cell of claim 30, or the isolated population of lymphocytes of claim 45, wherein the exogenous nucleic acid expression construct comprises the encoding nucleic acid sequence listed under (1) under the control of a heterologous promoter.

81-88. (canceled)


SEQUENCE LISTING (1168 sequences)

SEQ ID NO: 1 - RNA, 10 nt, Artificial Sequence
  source: /note=“Description of Artificial Sequence Synthetic oligonucleotide”
aagaccuuuu

SEQ ID NO: 2 - RNA, 25 nt, Artificial Sequence
  source: /note=“Description of Artificial Sequence Synthetic oligonucleotide”
auguguuuuugucaaaagaccuuuu

SEQ ID NO: 3 - RNA, 60 nt, Artificial Sequence
  source: /note=“Description of Artificial Sequence Synthetic oligonucleotide”
aggccagcuugccgguuuuuuagucgugcugcuucauguguuuuugucaaaagaccuuuu

SEQ ID NO: 4 - DNA, 10 bp, Artificial Sequence
  source: /note=“Description of Artificial Sequence Synthetic oligonucleotide”
aagacctttt

SEQ ID NO: 5 - DNA, 25 bp, Artificial Sequence
  source: /note=“Description of Artificial Sequence Synthetic oligonucleotide”
atgtgtttttgtcaaaagacctttt

SEQ ID NO: 6 - DNA, 60 bp, Artificial Sequence
  source: /note=“Description of Artificial Sequence Synthetic oligonucleotide”
aggccagcttgccggttttttagtcgtgctgcttcatgtgtttttgtcaaaagacctttt

SEQ ID NO: 7 - DNA, 20 bp, Artificial Sequence
  source: /note=“Description of Artificial Sequence Synthetic oligonucleotide”
tttttgtcaaaagacctttt

SEQ ID NO: 8 - DNA, 30 bp, Artificial Sequence
  source: /note=“Description of Artificial Sequence Synthetic oligonucleotide”
gcttcatgtgtttttgtcaaaagacctttt

SEQ ID NO: 9 - DNA, 50 bp, Artificial Sequence
  source: /note=“Description of Artificial Sequence Synthetic oligonucleotide”
gccggttttttagtcgtgctgcttcatgtgtttttgtcaaaagacctttt

SEQ ID NO: 10 - DNA, 40 bp, Artificial Sequence
  source: /note=“Description of Artificial Sequence Synthetic oligonucleotide”
tagtcgtgctgcttcatgtgtttttgtcaaaagacctttt

SEQ ID NO: 11 - DNA, 20 bp, Artificial Sequence
  source: /note=“Description of Artificial Sequence Synthetic oligonucleotide”
ccgaagttttcttcggtttt

SEQ ID NO: 12 - DNA, 25 bp, Artificial Sequence
  source: /note=“Description of Artificial Sequence Synthetic oligonucleotide”
tttttccgaagttttcttcggtttt

SEQ ID NO: 13 - DNA, 30 bp, Artificial Sequence
  source: /note=“Description of Artificial Sequence Synthetic oligonucleotide”
aacgctttttccgaagttttcttcggtttt

SEQ ID NO: 14 - DNA, 41 bp, Artificial Sequence
  source: /note=“Description of Artificial Sequence Synthetic oligonucleotide”
gcgttgttttcaacgctttttccgaagttttcttcggtttt

SEQ ID NO: 15 - DNA, 62 bp, Artificial Sequence
  source: /note=“Description of Artificial Sequence Synthetic oligonucleotide”
ggcttcttttgaagcctttttgcgttgttttcaacgctttttccgaagttttcttcggtttt

SEQ ID NO: 16 - DNA, 25 bp, Artificial Sequence
  source: /note=“Description of Artificial Sequence Synthetic oligonucleotide”
atgtgtttttgtcaaaagacctttt

SEQ ID NO: 17 - RNA, 25 nt, Artificial Sequence
  source: /note=“Description of Artificial Sequence Synthetic oligonucleotide”
aaaaaaaaaaaaaaaaaaaaaaaaa

SEQ ID NO: 18 - DNA, 25 bp, Artificial Sequence
  source: /note=“Description of Artificial Sequence Synthetic oligonucleotide”
ttttttttttttttttttttttttt

SEQ ID NO: 19 - RNA, 25 nt, Artificial Sequence
  source: /note=“Description of Artificial Sequence Synthetic oligonucleotide”
auguguuuuugucaaaagaccuuuu

SEQ ID NO: 20 - RNA, 25 nt, Artificial Sequence
  source: /note=“Description of Artificial Sequence Synthetic oligonucleotide”
aaaaaaaaaaaaaaaaaaaaaaaaa

SEQ ID NO: 21 - RNA, 25 nt, Artificial Sequence
  source: /note=“Description of Artificial Sequence Synthetic oligonucleotide”
uuuuuuuuuuuuuuuuuuuuuuuuu

SEQ ID NO: 22 - DNA, 20 bp, Artificial Sequence
  source: /note=“Description of Artificial Sequence Synthetic oligonucleotide”
tctgcagaaatgttccccgt

SEQ ID NO: 23 - RNA, 20 nt, Artificial Sequence
  source: /note=“Description of Artificial Sequence Synthetic oligonucleotide”
ucugcagaaauguuccccgu

SEQ ID NO: 24 - RNA, 20 nt, Artificial Sequence
  source: /note=“Description of Artificial Sequence Synthetic oligonucleotide”
uaauuucuacucuuguagau

SEQ ID NO: 25 - RNA, 40 nt, Artificial Sequence
  source: /note=“Description of Artificial Sequence Synthetic oligonucleotide”
uaauuucuacucuuguagauucugcagaaauguuccccgu

SEQ ID NO: 26 - DNA, 65 bp, Artificial Sequence
  source: /note=“Description of Artificial Sequence Synthetic oligonucleotide”
  source: /note=“Description of Combined DNA/RNA Molecule Synthetic oligonucleotide”
atgtgtttttgtcaaaagaccttttuaauuucuacucuuguagauucugcagaaauguuccccgu

SEQ ID NO: 27 - DNA, 20 bp, Artificial Sequence
  source: /note=“Description of Artificial Sequence Synthetic oligonucleotide”
tctgcagaaatgttccccgt

SEQ ID NO: 28 - DNA, 20 bp, Artificial Sequence
  source: /note=“Description of Artificial Sequence Synthetic oligonucleotide”
tgcagagaaaggtggctcta

SEQ ID NO: 29 - DNA, 20 bp, Artificial Sequence
  source: /note=“Description of Artificial Sequence Synthetic oligonucleotide”
taatgctgacttggggtggc

SEQ ID NO: 30 - DNA, 20 bp, Artificial Sequence
  source: /note=“Description of Artificial Sequence Synthetic oligonucleotide”
taggacctccaggaagattc

SEQ ID NO: 31 - DNA, 20 bp, Artificial Sequence
  source: /note=“Description of Artificial Sequence Synthetic oligonucleotide”
tagtcaacgcgaccaccacg

SEQ ID NO: 32 - DNA, 20 bp, Artificial Sequence
  source: /note=“Description of Artificial Sequence Synthetic oligonucleotide”
tcctgaggtcaccttccaca

SEQ ID NO: 33 - DNA, 20 bp, Artificial Sequence
  source: /note=“Description of Artificial Sequence Synthetic oligonucleotide”
tattgtgcctgtcatcattc

SEQ ID NO: 34 - DNA, 21 bp, Artificial Sequence
  source: /note=“Description of Artificial Sequence Synthetic oligonucleotide”
tgacaggcacaatagaaacaa

SEQ ID NO: 35 - DNA, 21 bp, Artificial Sequence
  source: /note=“Description of Artificial Sequence Synthetic oligonucleotide”
gacaggcacaatagaaacaac

SEQ ID NO: 36 - DNA, 21 bp, Artificial Sequence
  source: /note=“Description of Artificial Sequence Synthetic oligonucleotide”
aaacaacggggaacatttctg

SEQ ID NO: 37 - DNA, 21 bp, Artificial Sequence
  source: /note=“Description of Artificial Sequence Synthetic oligonucleotide”
acaacggggaacatttctgca

SEQ ID NO: 38 - DNA, 21 bp, Artificial Sequence
  source: /note=“Description of Artificial Sequence Synthetic oligonucleotide”
tgatagagccacctttctctg

SEQ ID NO: 39 - DNA, 21 bp, Artificial Sequence
  source: /note=“Description of Artificial Sequence Synthetic oligonucleotide”
gggtcacttgtgccgtggtgg

SEQ ID NO: 40 - DNA, 21 bp, Artificial Sequence
  source: /note=“Description of Artificial Sequence Synthetic oligonucleotide”
ggcacaagtgacccaggtcaa

SEQ ID NO: 41 - DNA, 21 bp, Artificial Sequence
  source: /note=“Description of Artificial Sequence Synthetic oligonucleotide”
gtcctgctgctcccagttgac

SEQ ID NO: 42 - DNA, 21 bp, Artificial Sequence
  source: /note=“Description of Artificial Sequence Synthetic oligonucleotide”
tggccatttgtaatgctgact

SEQ ID NO: 43 - DNA, 21 bp, Artificial Sequence
  source: /note=“Description of Artificial Sequence Synthetic oligonucleotide”
tggcacatctccccatccttc

SEQ ID NO: 44 - DNA, 21 bp, Artificial Sequence
  source: /note=“Description of Artificial Sequence Synthetic oligonucleotide”
catctccccatccttcaagga

SEQ ID NO: 45 - DNA, 21 bp, Artificial Sequence
  source: /note=“Description of Artificial Sequence Synthetic oligonucleotide”
ccactcgatccttgaaggatg

SEQ ID NO: 46 - DNA, 21 bp, Artificial Sequence
  source: /note=“Description of Artificial Sequence Synthetic oligonucleotide”
ggccactcgatccttgaagga

SEQ ID NO: 47 - DNA, 21 bp, Artificial Sequence
  source: /note=“Description of Artificial Sequence Synthetic oligonucleotide”
cctggggccactcgatccttg

SEQ ID NO: 48 - DNA, 21 bp, Artificial Sequence
  source: /note=“Description of Artificial Sequence Synthetic oligonucleotide”
gactggagggtgaggcccagg

SEQ ID NO: 49 - DNA, 21 bp, Artificial Sequence
  source: /note=“Description of Artificial Sequence Synthetic oligonucleotide”
atcgttcacggtcagcgactg

SEQ ID NO: 50 - DNA, 21 bp, Artificial Sequence
  source: /note=“Description of Artificial Sequence Synthetic oligonucleotide”
gtcgctgaccgtgaacgatac

SEQ ID NO: 51 - DNA, 21 bp, Artificial Sequence
  source: /note=“Description of Artificial Sequence Synthetic oligonucleotide”
cgctgaccgtgaacgatacag

SEQ ID NO: 52 - DNA, 21 bp, Artificial Sequence
  source: /note=“Description of Artificial Sequence Synthetic oligonucleotide”
gcatctatcacacctaccctg

SEQ ID NO: 53 - DNA, 21 bp, Artificial Sequence
  source: /note=“Description of Artificial Sequence Synthetic oligonucleotide”
cctaccctgatgggacgtaca

SEQ ID NO: 54 - DNA, 21 bp, Artificial Sequence
  source: /note=“Description of Artificial Sequence Synthetic oligonucleotide”
taccctgatgggacgtacact

SEQ ID NO: 55 - DNA, 21 bp, Artificial Sequence
  source: /note=“Description of Artificial Sequence Synthetic oligonucleotide”
ccctgatgggacgtacactgg

SEQ ID NO: 56 - DNA, 21 bp, Artificial Sequence
  source: /note=“Description of Artificial Sequence Synthetic oligonucleotide”
ttctcccagtgtacgtcccat

SEQ ID NO: 57 - DNA, 21 bp, Artificial Sequence
  source: /note=“Description of Artificial Sequence Synthetic oligonucleotide”
ggagaatcttcctggaggtcc

SEQ ID NO: 58 - DNA, 21 bp, Artificial Sequence
  source: /note=“Description of Artificial Sequence Synthetic oligonucleotide”
catggctccaagcaatggaat

SEQ ID NO: 59 - DNA, 21 bp, Artificial Sequence
  source: /note=“Description of Artificial Sequence Synthetic oligonucleotide”
cgcggccatggctccaagcaa

SEQ ID NO: 60 - DNA, 21 bp, Artificial Sequence
  source: /note=“Description of Artificial Sequence Synthetic oligonucleotide”
tcgcggccatggctccaagca

SEQ ID NO: 61 - DNA, 21 bp, Artificial Sequence
  source: /note=“Description of Artificial Sequence Synthetic oligonucleotide”
catcgtggtggtcgcgttgac

SEQ ID NO: 62 - DNA, 21 bp, Artificial Sequence
  source: /note=“Description of Artificial Sequence Synthetic oligonucleotide”
aaagccctcagaatccattct

SEQ ID NO: 63 - DNA, 21 bp, Artificial Sequence
  source: /note=“Description of Artificial Sequence Synthetic oligonucleotide”
cattctgtggaaggtgacctc

SEQ ID NO: 64 - DNA, 21 bp, Artificial Sequence
  source: /note=“Description of Artificial Sequence Synthetic oligonucleotide”
ttctgtggaaggtgacctcag

SEQ ID NO: 65 - DNA, 21 bp, Artificial Sequence
  source: /note=“Description of Artificial Sequence Synthetic oligonucleotide”
cctgaggtcaccttccacaga

SEQ ID NO: 66 - DNA, 21 bp, Artificial Sequence
  source: /note=“Description of Artificial Sequence Synthetic oligonucleotide”
ttctcctgaggtcaccttcca

SEQ ID NO: 67 - DNA, 21 bp, Artificial Sequence
  source: /note=“Description of Artificial Sequence Synthetic oligonucleotide”
aggagaaaatcagctggacag

SEQ ID NO: 68 - DNA, 21 bp, Artificial Sequence
  source: /note=“Description of Artificial Sequence Synthetic oligonucleotide”
ggagaaaatcagctggacagg

SEQ ID NO: 69 - DNA, 21 bp, Artificial Sequence
  source: /note=“Description of Artificial Sequence Synthetic oligonucleotide”
gccccagtgctccctcacccc

SEQ ID NO: 70 - DNA, 21 bp, Artificial Sequence
  source: /note=“Description of Artificial Sequence Synthetic oligonucleotide”
tggacacagcttcctgggggt

SEQ ID NO: 71 - DNA, 21 bp, Artificial Sequence
  source: /note=“Description of Artificial Sequence Synthetic oligonucleotide”
tctgcctggacacagcttcct

SEQ ID NO: 72 - DNA, 21 bp, Artificial Sequence
  source: /note=“Description of Artificial Sequence Synthetic oligonucleotide”
agctgcacctgctgggctctg

SEQ ID NO: 73 - DNA, 21 bp, Artificial Sequence
  source: /note=“Description of Artificial Sequence Synthetic oligonucleotide”
gctgggctctgtggagagcag

SEQ ID NO: 74 - DNA, 21 bp, Artificial Sequence
  source: /note=“Description of Artificial Sequence Synthetic oligonucleotide”
tgggctctgtggagagcagcg

SEQ ID NO: 75 - DNA, 21 bp, Artificial Sequence
  source: /note=“Description of Artificial Sequence Synthetic oligonucleotide”
ctgcatgactacttcaatgtc

SEQ ID NO: 76 - DNA, 21 bp, Artificial Sequence
  source: /note=“Description of Artificial Sequence Synthetic oligonucleotide”
aatgtcctgagttacagaagc

SEQ ID NO: 77 - DNA, 21 bp, Artificial Sequence
  source: /note=“Description of Artificial Sequence Synthetic oligonucleotide”
tgggtaactgcagcttcttca

SEQ ID NO: 78 - DNA, 20 bp, Artificial Sequence
  source: /note=“Description of Artificial Sequence Synthetic oligonucleotide”
gacaggcacaatagaaacaa

SEQ ID NO: 79 - DNA, 20 bp, Artificial Sequence
  source: /note=“Description of Artificial Sequence Synthetic oligonucleotide”
acaggcacaatagaaacaac

SEQ ID NO: 80 - DNA, 20 bp, Artificial Sequence
  source: /note=“Description of Artificial Sequence Synthetic oligonucleotide”
caggcacaatagaaacaacg

SEQ ID NO: 81 - DNA, 20 bp, Artificial Sequence
  source: /note=“Description of Artificial Sequence Synthetic oligonucleotide”
gggaacatttctgcagagaa

SEQ ID NO: 82 - DNA, 20 bp, Artificial Sequence
  source: /note=“Description of Artificial Sequence Synthetic oligonucleotide”
aacatttctgcagagaaagg

SEQ ID NO: 83 - DNA, 20 bp, Artificial Sequence
  source: /note=“Description of Artificial Sequence Synthetic oligonucleotide”
atgtcacctctcctccacca

SEQ ID NO: 84 - DNA, 20 bp, Artificial Sequence
  source: /note=“Description of Artificial Sequence Synthetic oligonucleotide”
cttgtgccgtggtggaggag

SEQ ID NO: 85 - DNA, 20 bp, Artificial Sequence
  source: /note=“Description of Artificial Sequence Synthetic oligonucleotide”
ggtcacttgtgccgtggtgg

SEQ ID NO: 86 - DNA, 20 bp, Artificial Sequence
  source: /note=“Description of Artificial Sequence Synthetic oligonucleotide”
caccacggcacaagtgaccc

SEQ ID NO: 87 - DNA, 20 bp, Artificial Sequence
  source: /note=“Description of Artificial Sequence Synthetic oligonucleotide”
ctgggtcacttgtgccgtgg

SEQ ID NO: 88 - DNA, 20 bp, Artificial Sequence
  source: /note=“Description of Artificial Sequence Synthetic oligonucleotide”
gacctgggtcacttgtgccg

SEQ ID NO: 89 - DNA, 20 bp, Artificial Sequence
  source: /note=“Description of Artificial Sequence Synthetic oligonucleotide”
cacaagtgacccaggtcaac

SEQ ID NO: 90 - DNA, 20 bp, Artificial Sequence
  source: /note=“Description of Artificial Sequence Synthetic oligonucleotide”
acaagtgacccaggtcaact

SEQ ID NO: 91 - DNA, 20 bp, Artificial Sequence
  source: /note=“Description of Artificial Sequence Synthetic oligonucleotide”
ccaggtcaactgggagcagc

SEQ ID NO: 92 - DNA, 20 bp, Artificial Sequence
  source: /note=“Description of Artificial Sequence Synthetic oligonucleotide”
ctgctgctcccagttgacct

SEQ ID NO: 93 - DNA, 20 bp, Artificial Sequence
  source: /note=“Description of Artificial Sequence Synthetic oligonucleotide”
cctgctgctcccagttgacc

SEQ ID NO: 94 - DNA, 20 bp, Artificial Sequence
  source: /note=“Description of Artificial Sequence Synthetic oligonucleotide”
ggagcagcaggaccagcttc

SEQ ID NO: 95 - DNA, 20 bp, Artificial Sequence
  source: /note=“Description of Artificial Sequence Synthetic oligonucleotide”
cattacaaatggccagaagc

SEQ ID NO: 96 - DNA, 20 bp, Artificial Sequence
  source: /note=“Description of Artificial Sequence Synthetic oligonucleotide”
ggccatttgtaatgctgact

SEQ ID NO: 97 - DNA, 20 bp, Artificial Sequence
  source: /note=“Description of Artificial Sequence Synthetic oligonucleotide”
gccatttgtaatgctgactt

SEQ ID NO: 98 - DNA, 20 bp, Artificial Sequence
  source: /note=“Description of Artificial Sequence Synthetic oligonucleotide”
ccatttgtaatgctgacttg

SEQ ID NO: 99 - DNA, 20 bp, Artificial Sequence
  source: /note=“Description of Artificial Sequence Synthetic oligonucleotide”
tttgtaatgctgacttgggg

SEQ ID NO: 100 - DNA, 20 bp, Artificial Sequence
  source: /note=“Description of Artificial Sequence Synthetic oligonucleotide”
ccccaagtcagcattacaaa

SEQ ID NO: 101 - DNA, 20 bp, Artificial Sequence
  source: /note=“Description of Artificial Sequence Synthetic oligonucleotide”
gcacatctccccatccttca

SEQ ID NO: 102 - DNA, 20 bp, Artificial Sequence
  source: /note=“Description of Artificial Sequence Synthetic oligonucleotide”
cccatccttcaaggatcgag

SEQ ID NO: 103 - DNA, 20 bp, Artificial Sequence
  source: /note=“Description of Artificial Sequence Synthetic oligonucleotide”
cactcgatccttgaaggatg

SEQ ID NO: 104 - DNA, 20 bp, Artificial Sequence
  source: /note=“Description of Artificial Sequence Synthetic oligonucleotide”
ccactcgatccttgaaggat

SEQ ID NO: 105 - DNA, 20 bp, Artificial Sequence
  source: /note=“Description of Artificial Sequence Synthetic oligonucleotide”
gccactcgatccttgaagga

SEQ ID NO: 106 - DNA, 20 bp, Artificial Sequence
  source: /note=“Description of Artificial Sequence Synthetic oligonucleotide”
ttcaaggatcgagtggcccc

SEQ ID NO: 107 - DNA, 20 bp, Artificial Sequence
  source: /note=“Description of Artificial Sequence Synthetic oligonucleotide”
tggggccactcgatccttga

SEQ ID NO: 108 - DNA, 20 bp, Artificial Sequence
  source: /note=“Description of Artificial Sequence Synthetic oligonucleotide”
gatcgagtggccccaggtcc

SEQ ID NO: 109 - DNA, 20 bp, Artificial Sequence
  source: /note=“Description of Artificial Sequence Synthetic oligonucleotide”
agtggccccaggtcccggcc

SEQ ID NO: 110 - DNA, 20 bp, Artificial Sequence
  source: /note=“Description of Artificial Sequence Synthetic oligonucleotide”
gtggccccaggtcccggcct

SEQ ID NO: 111 - DNA, 20 bp, Artificial Sequence
  source: /note=“Description of Artificial Sequence Synthetic oligonucleotide”
gaggcccaggccgggacctg

SEQ ID NO: 112 - DNA, 20 bp, Artificial Sequence
  source: /note=“Description of Artificial Sequence Synthetic oligonucleotide”
tgaggcccaggccgggacct

SEQ ID NO: 113 - DNA, 20 bp, Artificial Sequence
  source: /note=“Description of Artificial Sequence Synthetic oligonucleotide”
gtgaggcccaggccgggacc

SEQ ID NO: 114 - DNA, 20 bp, Artificial Sequence
  source: /note=“Description of Artificial Sequence Synthetic oligonucleotide”
tggagggtgaggcccaggcc

SEQ ID NO: 115 - DNA, 20 bp, Artificial Sequence
  source: /note=“Description of Artificial Sequence Synthetic oligonucleotide”
ctggagggtgaggcccaggc

SEQ ID NO: 116 - DNA, 20 bp, Artificial Sequence
  source: /note=“Description of Artificial Sequence Synthetic oligonucleotide”
gcgactggagggtgaggccc

SEQ ID NO: 117 - DNA, 20 bp, Artificial Sequence
  source: /note=“Description of Artificial Sequence Synthetic oligonucleotide”
cggtcagcgactggagggtg

SEQ ID NO: 118 - DNA, 20 bp, Artificial Sequence
  source: /note=“Description of Artificial Sequence Synthetic oligonucleotide”
gttcacggtcagcgactgga

SEQ ID NO: 119 - DNA, 20 bp, Artificial Sequence
  source: /note=“Description of Artificial Sequence Synthetic oligonucleotide”
cgttcacggtcagcgactgg

SEQ ID NO: 120 - DNA, 20 bp, Artificial Sequence
  source: /note=“Description of Artificial Sequence Synthetic oligonucleotide”
tatcgttcacggtcagcgac

SEQ ID NO: 121 - DNA, 20 bp, Artificial Sequence
  source: /note=“Description of Artificial Sequence Synthetic oligonucleotide”
tcgctgaccgtgaacgatac

SEQ ID NO: 122 - DNA, 20 bp, Artificial Sequence
  source: /note=“Description of Artificial Sequence Synthetic oligonucleotide”
cgctgaccgtgaacgataca

SEQ ID NO: 123 - DNA, 20 bp, Artificial Sequence
  source: /note=“Description of Artificial Sequence Synthetic oligonucleotide”
gctgaccgtgaacgatacag

SEQ ID NO: 124 - DNA, 20 bp, Artificial Sequence
  source: /note=“Description of Artificial Sequence Synthetic oligonucleotide”
gtactcccctgtatcgttca

SEQ ID NO: 125 - DNA, 20 bp, Artificial Sequence
  source: /note=“Description of Artificial Sequence Synthetic oligonucleotide”
atctatcacacctaccctga

SEQ ID NO: 126 - DNA, 20 bp, Artificial Sequence
  source: /note=“Description of Artificial Sequence Synthetic oligonucleotide”
tctatcacacctaccctgat

SEQ ID NO: 127 - DNA, 20 bp, Artificial Sequence
  source: /note=“Description of Artificial Sequence Synthetic oligonucleotide”
taccctgatgggacgtacac

SEQ ID NO: 128 - DNA, 20 bp, Artificial Sequence
  source: /note=“Description of Artificial Sequence Synthetic oligonucleotide”
accctgatgggacgtacact

SEQ ID NO: 129 - DNA, 20 bp, Artificial Sequence
  source: /note=“Description of Artificial Sequence Synthetic oligonucleotide”
agtgtacgtcccatcagggt

SEQ ID NO: 130 - DNA, 20 bp, Artificial Sequence
  source: /note=“Description of Artificial Sequence Synthetic oligonucleotide”
tcccagtgtacgtcccatca

SEQ ID NO: 131 - DNA, 20 bp, Artificial Sequence
  source: /note=“Description of Artificial Sequence Synthetic oligonucleotide”
ctcccagtgtacgtcccatc

SEQ ID NO: 132 - DNA, 20 bp, Artificial Sequence
  source: /note=“Description of Artificial Sequence Synthetic oligonucleotide”
gtacactgggagaatcttcc

SEQ ID NO: 133 - DNA, 20 bp, Artificial Sequence
  source: /note=“Description of Artificial Sequence Synthetic oligonucleotide”
cactgggagaatcttcctgg

SEQ ID NO: 134 - DNA, 20 bp, Artificial Sequence
  source: /note=“Description of Artificial Sequence Synthetic oligonucleotide”
ctgagctttctaggacctcc

SEQ ID NO: 135 - DNA, 20 bp, Artificial Sequence
  source: /note=“Description of Artificial Sequence Synthetic oligonucleotide”
aggttccagattccattgct

SEQ ID NO: 136 - DNA, 20 bp, Artificial Sequence
  source: /note=“Description of Artificial Sequence Synthetic oligonucleotide”
aagcaatggaatctggaacc

SEQ ID NO: 137 - DNA, 20 bp, Artificial Sequence
  source: /note=“Description of Artificial Sequence Synthetic oligonucleotide”
gattccattgcttggagcca

SEQ ID NO: 138 - DNA, 20 bp, Artificial Sequence
  source: /note=“Description of Artificial Sequence Synthetic oligonucleotide”
tggctccaagcaatggaatc

SEQ ID NO: 139 - DNA, 20 bp, Artificial Sequence
  source: /note=“Description of Artificial Sequence Synthetic oligonucleotide”
gcggccatggctccaagcaa

SEQ ID NO: 140 - DNA, 20 bp, Artificial Sequence
  source: /note=“Description of Artificial Sequence Synthetic oligonucleotide”
tggagccatggccgcgacgc

SEQ ID NO: 141 - DNA, 20 bp, Artificial Sequence
  source: /note=“Description of Artificial Sequence Synthetic oligonucleotide”
agccatggccgcgacgctgg

SEQ ID NO: 142 - DNA, 20 bp, Artificial Sequence
  source: /note=“Description of Artificial Sequence Synthetic oligonucleotide”
gaccaccagcgtcgcggcca

SEQ ID NO: 143 - DNA, 20 bp, Artificial Sequence
  source: /note=“Description of Artificial Sequence Synthetic oligonucleotide”
gcagatgaccaccagcgtcg

SEQ ID NO: 144 - DNA, 20 bp, Artificial Sequence
  source: /note=“Description of Artificial Sequence Synthetic oligonucleotide”
catctgcacagcagtcatcg

SEQ ID NO: 145 - DNA, 20 bp, Artificial Sequence
  source: /note=“Description of Artificial Sequence Synthetic oligonucleotide”
ctgcacagcagtcatcgtgg

SEQ ID NO: 146 - DNA, 20 bp, Artificial Sequence
  source: /note=“Description of Artificial Sequence Synthetic oligonucleotide”
agccctcagaatccattctg

SEQ ID NO: 147 - DNA, 20 bp, Artificial Sequence
  source: /note=“Description of Artificial Sequence Synthetic oligonucleotide”
ctcagaatccattctgtgga

SEQ ID NO: 148 - DNA, 20 bp, Artificial Sequence
  source: /note=“Description of Artificial Sequence Synthetic oligonucleotide”
ttccacagaatggattctga

SEQ ID NO: 149 - DNA, 20 bp, Artificial Sequence
  source: /note=“Description of Artificial Sequence Synthetic oligonucleotide”
cttccacagaatggattctg

SEQ ID NO: 150 - DNA, 20 bp, Artificial Sequence
  source: /note=“Description of Artificial Sequence Synthetic oligonucleotide”
attctgtggaaggtgacctc

SEQ ID NO: 151 - DNA, 20 bp, Artificial Sequence
  source: /note=“Description of Artificial Sequence Synthetic oligonucleotide”
tgaggtcaccttccacagaa

SEQ ID NO: 152 - DNA, 20 bp, Artificial Sequence
  source: /note=“Description of Artificial Sequence Synthetic oligonucleotide”
gacctcaggagaaaatcagc

SEQ ID NO: 153 - DNA, 20 bp, Artificial Sequence
  source: /note=“Description of Artificial Sequence Synthetic oligonucleotide”
caggagaaaatcagctggac

SEQ ID NO: 154 - DNA, 20 bp, Artificial Sequence
  source: /note=“Description of Artificial Sequence Synthetic oligonucleotide”
gtccagctgattttctcctg

SEQ ID NO: 155 - DNA, 20 bp, Artificial Sequence
  source: /note=“Description of Artificial Sequence Synthetic oligonucleotide”
gagaaaatcagctggacagg

SEQ ID NO: 156 - DNA, 20 bp, Artificial Sequence
  source: /note=“Description of Artificial Sequence Synthetic oligonucleotide”
aatcagctggacaggaggaa

SEQ ID NO: 157 - DNA, 20 bp, Artificial Sequence
  source: /note=“Description of Artificial Sequence Synthetic oligonucleotide”
cccagtgctccctcaccccc

SEQ ID NO: 158 - DNA, 20 bp, Artificial Sequence
  source: /note=“Description of Artificial Sequence Synthetic oligonucleotide”
ctgggggtgagggagcactg

SEQ ID NO: 159 - DNA, 20 bp, Artificial Sequence
  source: /note=“Description of Artificial Sequence Synthetic oligonucleotide”
cctgggggtgagggagcact

SEQ ID NO: 160 - DNA, 20 bp, Artificial Sequence
  source: /note=“Description of Artificial Sequence Synthetic oligonucleotide”
tcctgggggtgagggagcac

SEQ ID NO: 161 - DNA, 20 bp, Artificial Sequence
  source: /note=“Description of Artificial Sequence Synthetic oligonucleotide”
acacagcttcctgggggtga

SEQ ID NO: 162 - DNA, 20 bp, Artificial Sequence
  source: /note=“Description of Artificial Sequence Synthetic oligonucleotide”
gacacagcttcctgggggtg

SEQ ID NO: 163 - DNA, 20 bp, Artificial Sequence
  source: /note=“Description of Artificial Sequence Synthetic oligonucleotide”
acccccaggaagctgtgtcc

SEQ ID NO: 164 - DNA, 20 bp, Artificial Sequence
  source: /note=“Description of Artificial Sequence Synthetic oligonucleotide”
gcctggacacagcttcctgg

SEQ ID NO: 165 - DNA, 20 bp, Artificial Sequence
  source: /note=“Description of Artificial Sequence Synthetic oligonucleotide”
tgcctggacacagcttcctg

SEQ ID NO: 166 - DNA, 20 bp, Artificial Sequence
  source: /note=“Description of Artificial Sequence Synthetic oligonucleotide”
ctgcctggacacagcttcct

SEQ ID NO: 167 - DNA, 20 bp, Artificial Sequence
  source: /note=“Description of Artificial Sequence Synthetic oligonucleotide”
tctgcctggacacagcttcc

SEQ ID NO: 168 - DNA, 20 bp, Artificial Sequence
  source: /note=“Description of Artificial Sequence Synthetic oligonucleotide”
caggcagaagctgcacctgc

SEQ ID NO: 169 - DNA, 20 bp, Artificial Sequence
  source: /note=“Description of Artificial Sequence Synthetic oligonucleotide”
aggcagaagctgcacctgct

SEQ ID NO: 170 - DNA, 20 bp, Artificial Sequence
  source: /note=“Description of Artificial Sequence Synthetic oligonucleotide”
cagcaggtgcagcttctgcc

SEQ ID NO: 171 - DNA, 20 bp, Artificial Sequence
  source: /note=“Description of Artificial Sequence Synthetic oligonucleotide”
gctgcacctgctgggctctg

SEQ ID NO: 172 - DNA, 20 bp, Artificial Sequence
  source: /note=“Description of Artificial Sequence Synthetic oligonucleotide”
tgctctccacagagcccagc

SEQ ID NO: 173 - DNA, 20 bp, Artificial Sequence
  source: /note=“Description of Artificial Sequence Synthetic oligonucleotide”
ctgggctctgtggagagcag

SEQ ID NO: 174 - DNA, 20 bp, Artificial Sequence
  source: /note=“Description of Artificial Sequence Synthetic oligonucleotide”
tgggctctgtggagagcagc

SEQ ID NO: 175 - DNA, 20 bp, Artificial Sequence
  source: /note=“Description of Artificial Sequence Synthetic oligonucleotide”
gggctctgtggagagcagcg

SEQ ID NO: 176 - DNA, 20 bp, Artificial Sequence
  source: /note=“Description of Artificial Sequence Synthetic oligonucleotide”
ctgtggagagcagcggggag

SEQ ID NO: 177 - DNA, 20 bp, Artificial Sequence
  source: /note=“Description of Artificial Sequence Synthetic oligonucleotide”
attgaagtagtcatgcagct

SEQ ID NO: 178 - DNA, 20 bp, Artificial Sequence
  source: /note=“Description of Artificial Sequence Synthetic oligonucleotide”
tgtcctgagttacagaagcc

SEQ ID NO: 179 - DNA, 20 bp, Artificial Sequence
  source: /note=“Description of Artificial Sequence Synthetic oligonucleotide”
gtcctgagttacagaagcct

SEQ ID NO: 180 - DNA, 20 bp, Artificial Sequence
  source: /note=“Description of Artificial Sequence Synthetic oligonucleotide”
tacccaggcttctgtaactc

SEQ ID NO: 181 - DNA, 20 bp, Artificial Sequence
  source: /note=“Description of Artificial Sequence Synthetic oligonucleotide”
tgaagaagctgcagttaccc

SEQ ID NO: 182 - DNA, 20 bp, Artificial Sequence
  source: /note=“Description of Artificial Sequence Synthetic oligonucleotide”
tgcagcttcttcacagagac

SEQ ID NO: 183 - DNA, 20 bp, Artificial Sequence
  source: /note=“Description of Artificial Sequence Synthetic oligonucleotide”
gttgtttctattgtgcctgt

SEQ ID NO: 184 - DNA, 20 bp, Artificial Sequence
  source: /note=“Description of Artificial Sequence Synthetic oligonucleotide”
cgttgtttctattgtgcctg

SEQ ID NO: 185 - DNA, 20 bp, Artificial Sequence
  source: /note=“Description of Artificial Sequence Synthetic oligonucleotide”
ccgttgtttctattgtgcct

SEQ ID NO: 186 - DNA, 20 bp, Artificial Sequence
  source: /note=“Description of Artificial Sequence Synthetic oligonucleotide”
ccacggcacaagtgacccag

SEQ ID NO: 187 - DNA, 20 bp, Artificial Sequence
  source: /note=“Description of Artificial Sequence Synthetic oligonucleotide”
agttgacctgggtcacttgt

SEQ ID NO: 188 - DNA, 20 bp, Artificial Sequence
  source: /note=“Description of Artificial Sequence Synthetic oligonucleotide”
aagtcagcattacaaatggc

SEQ ID NO: 189 - DNA, 20 bp, Artificial Sequence
  source: /note=“Description of Artificial Sequence Synthetic oligonucleotide”
catccttcaaggatcgagtg

SEQ ID NO: 190 - DNA, 20 bp, Artificial Sequence
  source: /note=“Description of Artificial Sequence Synthetic oligonucleotide”
atccttcaaggatcgagtgg

SEQ ID NO: 191 - DNA, 20 bp, Artificial Sequence
  source: /note=“Description of Artificial Sequence Synthetic oligonucleotide”
aggatcgagtggccccaggt

SEQ ID NO: 192 - DNA, 20 bp, Artificial Sequence
  source: /note=“Description of Artificial Sequence Synthetic oligonucleotide”
aggtcccggcctgggcctca

SEQ ID NO: 193 - DNA, 20 bp, Artificial Sequence
  source: /note=“Description of Artificial Sequence Synthetic oligonucleotide”
ggcctgggcctcaccctcca

SEQ ID NO: 194 - DNA, 20 bp, Artificial Sequence
  source: /note=“Description of Artificial Sequence Synthetic oligonucleotide”
cggtcagcgactggagggtg

SEQ ID NO: 195 - DNA, 20 bp, Artificial Sequence
  source: /note=“Description of Artificial Sequence Synthetic oligonucleotide”
gtcgctgaccgtgaacgata

SEQ ID NO: 196 - DNA, 20 bp, Artificial Sequence
  source: /note=“Description of Artificial Sequence Synthetic oligonucleotide”
tgtatcgttcacggtcagcg

SEQ ID NO: 197 - DNA, 20 bp, Artificial Sequence
  source: /note=“Description of Artificial Sequence Synthetic oligonucleotide”
ctgtatcgttcacggtcagc

SEQ ID NO: 198 - DNA, 20 bp, Artificial Sequence
  source: /note=“Description of Artificial Sequence Synthetic oligonucleotide”
atcagggtaggtgtgataga

SEQ ID NO: 199 - DNA, 20 bp, Artificial Sequence
  source: /note=“Description of Artificial Sequence Synthetic oligonucleotide”
agtgtacgtcccatcagggt

SEQ ID NO: 200 - DNA, 20 bp, Artificial Sequence
  source: /note=“Description of Artificial Sequence Synthetic oligonucleotide”
ggaagattctcccagtgtac

SEQ ID NO: 201 - DNA, 20 bp, Artificial Sequence
  source: /note=“Description of Artificial Sequence Synthetic oligonucleotide”
tggaggtcctagaaagctca

SEQ ID NO: 202 - DNA, 20 bp, Artificial Sequence
  source: /note=“Description of Artificial Sequence Synthetic oligonucleotide”
agcaatggaatctggaacct

SEQ ID NO: 203 - DNA, 20 bp, Artificial Sequence
  source: /note=“Description of Artificial Sequence Synthetic oligonucleotide”
agattccattgcttggagcc

SEQ ID NO: 204 - DNA, 20 bp, Artificial Sequence
  source: /note=“Description of Artificial Sequence Synthetic oligonucleotide”
gattccattgcttggagcca

SEQ ID NO: 205 - DNA, 20 bp, Artificial Sequence
  source: /note=“Description of Artificial Sequence Synthetic oligonucleotide”
attgcttggagccatggccg

SEQ ID NO: 206 - DNA, 20 bp, Artificial Sequence
  source: /note=“Description of Artificial Sequence Synthetic oligonucleotide”
ttgcttggagccatggccgc

SEQ ID NO: 207 - DNA, 20 bp, Artificial Sequence
  source: /note=“Description of Artificial Sequence Synthetic oligonucleotide”
cagaatggattctgagggct

SEQ ID NO: 208 - DNA, 20 bp, Artificial Sequence
  source: /note=“Description of Artificial Sequence Synthetic oligonucleotide”
acagaatggattctgagggc

SEQ ID NO: 209 - DNA, 20 bp, Artificial Sequence
  source: /note=“Description of Artificial Sequence Synthetic oligonucleotide”
ttctgtggaaggtgacctca

SEQ ID NO: 210 - DNA, 20 bp, Artificial Sequence
  source: /note=“Description of Artificial Sequence Synthetic oligonucleotide”
gctgattttctcctgaggtc

SEQ ID NO: 211 - DNA, 20 bp, Artificial Sequence
  source: /note=“Description of Artificial Sequence Synthetic oligonucleotide”
tcctgtccagctgattttct

SEQ ID NO: 212 - DNA, 20 bp, Artificial Sequence
  source: /note=“Description of Artificial Sequence Synthetic oligonucleotide”
ttcctcctgtccagctgatt

SEQ ID NO: 213 - DNA, 20 bp, Artificial Sequence
  source: /note=“Description of Artificial Sequence Synthetic oligonucleotide”
tgggggtgagggagcactgg

SEQ ID NO: 214 - DNA, 20 bp, Artificial Sequence
  source: /note=“Description of Artificial Sequence Synthetic oligonucleotide”
agtgctccctcacccccagg

SEQ ID NO: 215 - DNA, 20 bp, Artificial Sequence
  source: /note=“Description of Artificial Sequence Synthetic oligonucleotide”
tcacccccaggaagctgtgt

SEQ ID NO: 216 - DNA, 20 bp, Artificial Sequence
  source: /note=“Description of Artificial Sequence Synthetic oligonucleotide”
caggaagctgtgtccaggca

SEQ ID NO: 217 - DNA, 20 bp, Artificial Sequence
  source: /note=“Description of Artificial Sequence Synthetic oligonucleotide”
aggaagctgtgtccaggcag

SEQ ID NO: 218 - DNA, 20 bp, Artificial Sequence
  source: /note=“Description of Artificial Sequence Synthetic oligonucleotide”
ggcagaagctgcacctgctg

SEQ ID NO: 219 - DNA, 20 bp, Artificial Sequence
  source: /note=“Description of Artificial Sequence Synthetic oligonucleotide”
cagagcccagcaggtgcagc

SEQ ID NO: 220 - DNA, 20 bp, Artificial Sequence
  source: /note=“Description of Artificial Sequence Synthetic oligonucleotide”
gctgctctccacagagccca

SEQ ID NO: 221 - DNA, 20 bp, Artificial Sequence
  source: /note=“Description of Artificial Sequence Synthetic oligonucleotide”
cgctgctctccacagagccc

SEQ ID NO: 222 - DNA, 20 bp, Artificial Sequence
  source: /note=“Description of Artificial Sequence Synthetic oligonucleotide”
atgtcctgagttacagaagc

SEQ ID NO: 223 - DNA, 20 bp, Artificial Sequence
  source: /note=“Description of Artificial Sequence Synthetic oligonucleotide”
gagcacacccactgcgatgt

SEQ ID NO: 224 - DNA, 20 bp, Artificial Sequence
  source: /note=“Description of Artificial Sequence Synthetic oligonucleotide”
gatggccaggagactgaaga

SEQ ID NO: 225 - DNA, 20 bp, Artificial Sequence
  source: /note=“Description of Artificial Sequence Synthetic oligonucleotide”
ctgctcaccggagcgggatg

SEQ ID NO: 226 - DNA, 20 bp, Artificial Sequence
  source: /note=“Description of Artificial Sequence Synthetic oligonucleotide”
gtctgtggccatgcccatca

SEQ ID NO: 227 - DNA, 20 bp, Artificial Sequence
  source: /note=“Description of Artificial Sequence Synthetic oligonucleotide”
tcaccggagcgggatgcgga

SEQ ID NO: 228 - DNA, 20 bp, Artificial Sequence
  source: /note=“Description of Artificial Sequence Synthetic oligonucleotide”
gtggcaggcagcgcagaacc

SEQ ID NO: 229 - DNA, 20 bp, Artificial Sequence
  source: /note=“Description of Artificial Sequence Synthetic oligonucleotide”
agcacaccagcacattgccc

SEQ ID NO: 230 - DNA, 20 bp, Artificial Sequence
  source: /note=“Description of Artificial Sequence Synthetic oligonucleotide”
caggttgctgttgagccaca

SEQ ID NO: 231 - DNA, 20 bp, Artificial Sequence
  source: /note=“Description of Artificial Sequence Synthetic oligonucleotide”
cttcattgcctgcttcgtcc

SEQ ID NO: 232 - DNA, 20 bp, Artificial Sequence
  source: /note=“Description of Artificial Sequence Synthetic oligonucleotide”
gtacaccgaggagcccatga

SEQ ID NO: 233 - DNA, 20 bp, Artificial Sequence
  source: /note=“Description of Artificial Sequence Synthetic oligonucleotide”
gatggcaatgtagcggtcaa

SEQ ID NO: 234 - DNA, 20 bp, Artificial Sequence
  source: /note=“Description of Artificial Sequence Synthetic oligonucleotide”
ctcctcggtgtacatcacgg

SEQ ID NO: 235 - DNA, 20 bp, Artificial Sequence
  source: /note=“Description of Artificial Sequence Synthetic oligonucleotide”
cgaggagcccatgatgggca

SEQ ID NO: 236 - DNA, 20 bp, Artificial Sequence
  source: /note=“Description of Artificial Sequence Synthetic oligonucleotide”
gggctcctcggtgtacatca

SEQ ID NO: 237 - DNA, 20 bp, Artificial Sequence
  source: /note=“Description of Artificial Sequence Synthetic oligonucleotide”
ctttgtggtgtcactggcgg

SEQ ID NO: 238 - DNA, 20 bp, Artificial Sequence
  source: /note=“Description of Artificial Sequence Synthetic oligonucleotide”
ccgctccggtgagcagggcc

SEQ ID NO: 239 - DNA, 20 bp, Artificial Sequence
  source: /note=“Description of Artificial Sequence Synthetic oligonucleotide”
gggttctgcgctgcctgcca

SEQ ID NO: 240 - DNA, 20 bp, Artificial Sequence
  source: /note=“Description of Artificial Sequence Synthetic oligonucleotide”
ggacgaagcaggcaatgaag

SEQ ID NO: 241 - DNA, 20 bp, Artificial Sequence
  source: /note=“Description of Artificial Sequence Synthetic oligonucleotide”
gtgctgatggtgatggcaaa

SEQ ID NO: 242 - DNA, 20 bp, Artificial Sequence
  source: /note=“Description of Artificial Sequence Synthetic oligonucleotide”
agcgcagaacccggtgctga

SEQ ID NO: 243 - DNA, 20 bp, Artificial Sequence
  source: /note=“Description of Artificial Sequence Synthetic oligonucleotide”
gagctccatcttcagtctcc

SEQ ID NO: 244 - DNA, 20 bp, Artificial Sequence
  source: /note=“Description of Artificial Sequence Synthetic oligonucleotide”
tgctgatggtgatggcaaag

SEQ ID NO: 245 - DNA, 20 bp, Artificial Sequence
  source: /note=“Description of Artificial Sequence Synthetic oligonucleotide”
ggcggcggccgacatcgcag

SEQ ID NO: 246 - DNA, 20 bp, Artificial Sequence
  source: /note=“Description of Artificial Sequence Synthetic oligonucleotide”
aatgaagaggcagccgtggc

SEQ ID NO: 247 - DNA, 20 bp, Artificial Sequence
  source: /note=“Description of Artificial Sequence Synthetic oligonucleotide”
gggcaatgtgctggtgtgct

SEQ ID NO: 248 - DNA, 20 bp, Artificial Sequence
  source: /note=“Description of Artificial Sequence Synthetic oligonucleotide”
catgcccatcatgggctcct

SEQ ID NO: 249 - DNA, 20 bp, Artificial Sequence
  source: /note=“Description of Artificial Sequence Synthetic oligonucleotide”
aatgtagcggtcaatggcga

SEQ ID NO: 250 - DNA, 20 bp, Artificial Sequence
  source: /note=“Description of Artificial Sequence Synthetic oligonucleotide”
agtagttggtgacgttctgc

SEQ ID NO: 251 - DNA, 20 bp, Artificial Sequence
  source: /note=“Description of Artificial Sequence Synthetic oligonucleotide”
agcggtcaatggcgatggcc

SEQ ID NO: 252 - DNA, 20 bp, Artificial Sequence
  source: /note=“Description of Artificial Sequence Synthetic oligonucleotide”
cgcatcccgctccggtgagc

SEQ ID NO: 253 - DNA, 20 bp, Artificial Sequence
  source: /note=“Description of Artificial Sequence Synthetic oligonucleotide”
gcatcccgctccggtgagca

SEQ ID NO: 254 - DNA, 20 bp, Artificial Sequence
  source: /note=“Description of Artificial Sequence Synthetic oligonucleotide”
tgggcaatgtgctggtgtgc

SEQ ID NO: 255 - DNA, 20 bp, Artificial Sequence
  source: /note=“Description of Artificial Sequence Synthetic oligonucleotide”
caactactttgtggtgtcac

SEQ ID NO: 256 - DNA, 20 bp, Artificial Sequence
  source: /note=“Description of Artificial Sequence Synthetic oligonucleotide”
cgctccggtgagcagggccg

SEQ ID NO: 257 - DNA, 20 bp, Artificial Sequence
  source: /note=“Description of Artificial Sequence Synthetic oligonucleotide”
gatggtgatggcaaagggga

SEQ ID NO: 258 - DNA, 20 bp, Artificial Sequence
  source: /note=“Description of Artificial Sequence Synthetic oligonucleotide”
ggtgtacatcacggtggagc

SEQ ID NO: 259 - DNA, 20 bp, Artificial Sequence
  source: /note=“Description of Artificial Sequence Synthetic oligonucleotide”
gaacgtcaccaactactttg

SEQ ID NO: 260 - DNA, 20 bp, Artificial Sequence
  source: /note=“Description of Artificial Sequence Synthetic oligonucleotide”
cagtgacaccacaaagtagt

SEQ ID NO: 261 - DNA, 20 bp, Artificial Sequence
  source: /note=“Description of Artificial Sequence Synthetic oligonucleotide”
ggccatcctgggcaatgtgc

SEQ ID NO: 262 - DNA, 20 bp, Artificial Sequence
  source: /note=“Description of Artificial Sequence Synthetic oligonucleotide”
cccggccctgctcaccggag

SEQ ID NO: 263 - DNA, 20 bp, Artificial Sequence
  source: /note=“Description of Artificial Sequence Synthetic oligonucleotide”
caccagcacattgcccagga

SEQ ID NO: 264 - DNA, 20 bp, Artificial Sequence
  source: /note=“Description of Artificial Sequence Synthetic oligonucleotide”
tttgccatcaccatcagcac

SEQ ID NO: 265 - DNA, 20 bp, Artificial Sequence
  source: /note=“Description of Artificial Sequence Synthetic oligonucleotide”
ctccaccgtgatgtacaccg

SEQ ID NO: 266 - DNA, 20 bp, Artificial Sequence
  source: /note=“Description of Artificial Sequence Synthetic oligonucleotide”
ggagctggccattgctgtgc

SEQ ID NO: 267 - DNA, 20 bp, Artificial Sequence
  source: /note=“Description of Artificial Sequence Synthetic oligonucleotide”
caggatggccagcacagcaa

SEQ ID NO: 268 - DNA, 20 bp, Artificial Sequence
  source: /note=“Description of Artificial Sequence Synthetic oligonucleotide”
gaacccggtgctgatggtga

SEQ ID NO: 269 - DNA, 20 bp, Artificial Sequence
  source: /note=“Description of Artificial Sequence Synthetic oligonucleotide”
tggagctctgcgtgaggacc

SEQ ID NO: 270 - DNA, 20 bp, Artificial Sequence
  source: /note=“Description of Artificial Sequence Synthetic oligonucleotide”
cccgctccggtgagcagggc

SEQ ID NO: 271 - DNA, 20 bp, Artificial Sequence
  source: /note=“Description of Artificial Sequence Synthetic oligonucleotide”
aggcaatgaagaggcagccg

SEQ ID NO: 272 - DNA, 20 bp, Artificial Sequence
  source: /note=“Description of Artificial Sequence Synthetic oligonucleotide”
ccggccctgctcaccggagc

SEQ ID NO: 273 - DNA, 20 bp, Artificial Sequence
  source: /note=“Description of Artificial Sequence Synthetic oligonucleotide”
gcggcggccgacatcgcagt

SEQ ID NO: 274 - DNA, 20 bp, Artificial Sequence
  source: /note=“Description of Artificial Sequence Synthetic oligonucleotide”
ggtgctgatggtgatggcaa

SEQ ID NO: 275 - DNA, 20 bp, Artificial Sequence
  source: /note=“Description of Artificial Sequence Synthetic oligonucleotide”
ctactttgtggtgtcactgg

SEQ ID NO: 276 - DNA, 20 bp, Artificial Sequence
  source: /note=“Description of Artificial Sequence Synthetic oligonucleotide”
tacaccgaggagcccatgat

SEQ ID NO: 277 - DNA, 20 bp, Artificial Sequence
  source: /note=“Description of Artificial Sequence Synthetic oligonucleotide”
tctgtggccatgcccatcat

SEQ ID NO: 278 - DNA, 20 bp, Artificial Sequence
  source: /note=“Description of Artificial Sequence Synthetic oligonucleotide”
attgctgtgctggccatcct

SEQ ID NO: 279 - DNA, 20 bp, Artificial Sequence
  source: /note=“Description of Artificial Sequence Synthetic oligonucleotide”
cgtgaggaccaggacgaagc

SEQ ID NO: 280 - DNA, 20 bp, Artificial Sequence
  source: /note=“Description of Artificial Sequence Synthetic oligonucleotide”
ttgccatcaccatcagcacc

SEQ ID NO: 281 - DNA, 20 bp, Artificial Sequence
  source: /note=“Description of Artificial Sequence Synthetic oligonucleotide”
ggatgcggatggcaatgtag

SEQ ID NO: 282 - DNA, 20 bp, Artificial Sequence
  source: /note=“Description of Artificial Sequence Synthetic oligonucleotide”
ttgccatccgcatcccgctc

SEQ ID NO: 283 - DNA, 20 bp, Artificial Sequence
  source: /note=“Description of Artificial Sequence Synthetic oligonucleotide”
tgaagatggagctctgcgtg

SEQ ID NO: 284 - DNA, 20 bp, Artificial Sequence
  source: /note=“Description of Artificial Sequence Synthetic oligonucleotide”
cattgctgtgctggccatcc

SEQ ID NO: 285 - DNA, 20 bp, Artificial Sequence
  source: /note=“Description of Artificial Sequence Synthetic oligonucleotide”
tgctggtgtgctgggccgtg

SEQ ID NO: 286 - DNA, 21 bp, Artificial Sequence
  source: /note=“Description of Artificial Sequence Synthetic oligonucleotide”
ggctcctcggtgtacatcacg

SEQ ID NO: 287 - DNA, 21 bp, Artificial Sequence
  source: /note=“Description of Artificial Sequence Synthetic oligonucleotide”
gagctctgcgtgaggaccagg

SEQ ID NO: 288 - DNA, 21 bp, Artificial Sequence
  source: /note=“Description of Artificial Sequence Synthetic oligonucleotide”
gatggagctctgcgtgaggac

SEQ ID NO: 289 - DNA, 21 bp, Artificial Sequence
  source: /note=“Description of Artificial Sequence Synthetic oligonucleotide”
ccagcacaccagcacattgcc

SEQ ID NO: 290 - DNA, 21 bp, Artificial Sequence
  source: /note=“Description of Artificial Sequence Synthetic oligonucleotide”
aggaccaggacgaagcaggca

SEQ ID NO: 291 - DNA, 21 bp, Artificial Sequence
  source: /note=“Description of Artificial Sequence Synthetic oligonucleotide”
tgccatccgcatcccgctccg

SEQ ID NO: 292 - DNA, 21 bp, Artificial Sequence
  source: /note=“Description of Artificial Sequence Synthetic oligonucleotide”
gtgtggctcaacagcaacctg

SEQ ID NO: 293 - DNA, 21 bp, Artificial Sequence
  source: /note=“Description of Artificial Sequence Synthetic oligonucleotide”
agctccaccgtgatgtacacc

SEQ ID NO: 294 - DNA, 21 bp, Artificial Sequence
  source: /note=“Description of Artificial Sequence Synthetic oligonucleotide”
gtagcggtcaatggcgatggc

SEQ ID NO: 295 - DNA, 21 bp, Artificial Sequence
  source: /note=“Description of Artificial Sequence Synthetic oligonucleotide”
cggtgctgatggtgatggcaa

SEQ ID NO: 296 - DNA, 21 bp, Artificial Sequence
  source: /note=“Description of Artificial Sequence Synthetic oligonucleotide”
ccctgctcaccggagcgggat

SEQ ID NO: 297 - DNA, 21 bp, Artificial Sequence
  source: /note=“Description of Artificial Sequence Synthetic oligonucleotide”
gtgacgttctgcaggttgctg

SEQ ID NO: 298 - DNA, 21 bp, Artificial Sequence
  source: /note=“Description of Artificial Sequence Synthetic oligonucleotide”
gctccaccgtgatgtacaccg

SEQ ID NO: 299 - DNA, 21 bp, Artificial Sequence
  source: /note=“Description of Artificial Sequence Synthetic oligonucleotide”
actgaagatggagctctgcgt

SEQ ID NO: 300 - DNA, 21 bp, Artificial Sequence
  source: /note=“Description of Artificial Sequence Synthetic oligonucleotide”
ccagctccaccgtgatgtaca

SEQ ID NO: 301 - DNA, 21 bp, Artificial Sequence
  source: /note=“Description of Artificial Sequence Synthetic oligonucleotide”
cctttgccatcaccatcagca

SEQ ID NO: 302 - DNA, 21 bp, Artificial Sequence
  source: /note=“Description of Artificial Sequence Synthetic oligonucleotide”
ccggtgctgatggtgatggca

SEQ ID NO: 303 - DNA, 21 bp, Artificial Sequence
  source: /note=“Description of Artificial Sequence Synthetic oligonucleotide”
cctgggcaatgtgctggtgtg

SEQ ID NO: 304 - DNA, 21 bp, Artificial Sequence
  source: /note=“Description of Artificial Sequence Synthetic oligonucleotide”
aggcagccgtggcaggcagcg

SEQ ID NO: 305 - DNA, 21 bp, Artificial Sequence
  source: /note=“Description of Artificial Sequence Synthetic oligonucleotide”
gcgatggccaggagactgaag

SEQ ID NO: 306 - DNA, 21 bp, Artificial Sequence
  source: /note=“Description of Artificial Sequence Synthetic oligonucleotide”
cgatggccaggagactgaaga

SEQ ID NO: 307 - DNA, 21 bp, Artificial Sequence
  source: /note=“Description of Artificial Sequence Synthetic oligonucleotide”
tcccgctccggtgagcagggc

SEQ ID NO: 308 - DNA, 21 bp, Artificial Sequence
  source: /note=“Description of Artificial Sequence Synthetic oligonucleotide”
tgcttcgtcctggtcctcacg

SEQ ID NO: 309 - DNA, 21 bp, Artificial Sequence
  source: /note=“Description of Artificial Sequence Synthetic oligonucleotide”
accaggacgaagcaggcaatg

SEQ ID NO: 310 - DNA, 21 bp, Artificial Sequence
  source: /note=“Description of Artificial Sequence Synthetic oligonucleotide”
atgtacaccgaggagcccatg

SEQ ID NO: 311 - DNA, 21 bp, Artificial Sequence
  source: /note=“Description of Artificial Sequence Synthetic oligonucleotide”
tcgtctgtggccatgcccatc

SEQ ID NO: 312 - DNA, 21 bp, Artificial Sequence
  source: /note=“Description of Artificial Sequence Synthetic oligonucleotide”
tcaatggcgatggccaggaga

SEQ ID NO: 313 - DNA, 21 bp, Artificial Sequence
  source: /note=“Description of Artificial Sequence Synthetic oligonucleotide”
ggtgctgatggtgatggcaaa

SEQ ID NO: 314 - DNA, 21 bp, Artificial Sequence
  source: /note=“Description of Artificial Sequence Synthetic oligonucleotide”
tagcggtcaatggcgatggcc

SEQ ID NO: 315 - DNA, 21 bp, Artificial Sequence
  source: /note=“Description of Artificial Sequence Synthetic oligonucleotide”
tccgcatcccgctccggtgag

SEQ ID NO: 316 - DNA, 21 bp, Artificial Sequence
  source: /note=“Description of Artificial Sequence Synthetic oligonucleotide”
ctggcggcggccgacatcgca

SEQ ID NO: 317 - DNA, 21 bp, Artificial Sequence
  source: /note=“Description of Artificial Sequence Synthetic oligonucleotide”
gccattgctgtgctggccatc

SEQ ID NO: 318 - DNA, 21 bp, Artificial Sequence
  source: /note=“Description of Artificial Sequence Synthetic oligonucleotide”
atcccgctccggtgagcaggg

SEQ ID NO: 319 - DNA, 21 bp, Artificial Sequence
  source: /note=“Description of Artificial Sequence Synthetic oligonucleotide”
agactgaagatggagctctgc

SEQ ID NO: 320 - DNA, 21 bp, Artificial Sequence
  source: /note=“Description of Artificial Sequence Synthetic oligonucleotide”
ccccggccctgctcaccggag

SEQ ID NO: 321 - DNA, 21 bp, Artificial Sequence
  source: /note=“Description of Artificial Sequence Synthetic oligonucleotide”
atggtgatggcaaaggggatg

SEQ ID NO: 322 - DNA, 21 bp, Artificial Sequence
  source: /note=“Description of Artificial Sequence Synthetic oligonucleotide”
gctcctcggtgtacatcacgg

SEQ ID NO: 323 - DNA, 20 bp, Artificial Sequence
  source: /note=“Description of Artificial Sequence Synthetic oligonucleotide”
tgtcgatggcaatagccaag

SEQ ID NO: 324 - DNA, 20 bp, Artificial Sequence
  source: /note=“Description of Artificial Sequence Synthetic oligonucleotide”
agaagttggtgacgttctgc

SEQ ID NO: 325 - DNA, 20 bp, Artificial Sequence
  source: /note=“Description of Artificial Sequence Synthetic oligonucleotide”
ttcgccatcaccatcagcac

SEQ ID NO: 326 - DNA, 20 bp, Artificial Sequence
  source: /note=“Description of Artificial Sequence Synthetic oligonucleotide”
gaagaagaggcagccatggc

SEQ ID NO: 327 - DNA, 20 bp, Artificial Sequence
  source: /note=“Description of Artificial Sequence Synthetic oligonucleotide”
cacaagcacgttacccagga

SEQ ID NO: 328 - DNA, 20 bp, Artificial Sequence
  source: /note=“Description of Artificial Sequence Synthetic oligonucleotide”
caacttcttcgtggtatctc

SEQ ID NO: 329 - DNA, 20 bp, Artificial Sequence
  source: /note=“Description of Artificial Sequence Synthetic oligonucleotide”
caggatggccagcacagcaa

SEQ ID NO: 330 - DNA, 20 bp, Artificial Sequence
  source: /note=“Description of Artificial Sequence Synthetic oligonucleotide”
aattccactccggtgagcca

SEQ ID NO: 331 - DNA, 20 bp, Artificial Sequence
  source: /note=“Description of Artificial Sequence Synthetic oligonucleotide”
agcgcagaagccagtgctga

SEQ ID NO: 332 - DNA, 20 bp, Artificial Sequence
  source: /note=“Description of Artificial Sequence Synthetic oligonucleotide”
gtgctgatggtgatggcgaa

SEQ ID NO: 333 - DNA, 20 bp, Artificial Sequence
  source: /note=“Description of Artificial Sequence Synthetic oligonucleotide”
ggagctggccattgctgtgc

SEQ ID NO: 334 - DNA, 20 bp, Artificial Sequence
  source: /note=“Description of Artificial Sequence Synthetic oligonucleotide”
aatagccaagaggctgaaga

SEQ ID NO: 335 - DNA, 20 bp, Artificial Sequence
  source: /note=“Description of Artificial Sequence Synthetic oligonucleotide”
ctcctcggtgtacatcatgg

SEQ ID NO: 336 - DNA, 20 bp, Artificial Sequence
  source: /note=“Description of Artificial Sequence Synthetic oligonucleotide”
ggacaaagcaggcgaagaag

SEQ ID NO: 337 - DNA, 20 bp, Artificial Sequence
  source: /note=“Description of Artificial Sequence Synthetic oligonucleotide”
tctggcggcggctgacatcg

SEQ ID NO: 338 - DNA, 20 bp, Artificial Sequence
  source: /note=“Description of Artificial Sequence Synthetic oligonucleotide”
tgggtaacgtgcttgtgtgc

SEQ ID NO: 339 - DNA, 20 bp, Artificial Sequence
  source: /note=“Description of Artificial Sequence Synthetic oligonucleotide”
gatgtacaccgaggagccca

SEQ ID NO: 340 - DNA, 20 bp, Artificial Sequence
  source: /note=“Description of Artificial Sequence Synthetic oligonucleotide”
taacccctggctcaccggag

SEQ ID NO: 341 - DNA, 20 bp, Artificial Sequence
  source: /note=“Description of Artificial Sequence Synthetic oligonucleotide”
tcaccggagtggaattcgga

SEQ ID NO: 342 - DNA, 20 bp, Artificial Sequence
  source: /note=“Description of Artificial Sequence Synthetic oligonucleotide”
gcggcggctgacatcgcggt

SEQ ID NO: 343 - DNA, 20 bp, Artificial Sequence
  source: /note=“Description of Artificial Sequence Synthetic oligonucleotide”
gatggtgatggcgaatggga

SEQ ID NO: 344 - DNA, 20 bp, Artificial Sequence
  source: /note=“Description of Artificial Sequence Synthetic oligonucleotide”
ggcttctgcgctgcctgcca

SEQ ID NO: 345 - DNA, 20 bp, Artificial Sequence
  source: /note=“Description of Artificial Sequence Synthetic oligonucleotide”
attccactccggtgagccag

SEQ ID NO: 346 - DNA, 20 bp, Artificial Sequence
  source: /note=“Description of Artificial Sequence Synthetic oligonucleotide”
ggtgtacatcatggtggagc

SEQ ID NO: 347 - DNA, 20 bp, Artificial Sequence
  source: /note=“Description of Artificial Sequence Synthetic oligonucleotide”
attgctgtgctggccatcct

SEQ ID NO: 348 - DNA, 20 bp, Artificial Sequence
  source: /note=“Description of Artificial Sequence Synthetic oligonucleotide”
ctccaccatgatgtacaccg

SEQ ID NO: 349 - DNA, 20 bp, Artificial Sequence
  source: /note=“Description of Artificial Sequence Synthetic oligonucleotide”
ggcggcggctgacatcgcgg

SEQ ID NO: 350 - DNA, 20 bp, Artificial Sequence
  source: /note=“Description of Artificial Sequence Synthetic oligonucleotide”
tacaccgaggagcccatggc

SEQ ID NO: 351 - DNA, 20 bp, Artificial Sequence
  source: /note=“Description of Artificial Sequence Synthetic oligonucleotide”
gggtaacgtgcttgtgtgct

SEQ ID NO: 352 - DNA, 20 bp, Artificial Sequence
  source: /note=“Description of Artificial Sequence Synthetic oligonucleotide”
caggttgctgttgatccaca

SEQ ID NO: 353 - DNA, 20 bp, Artificial Sequence
  source: /note=“Description of Artificial Sequence Synthetic oligonucleotide”
tgaagatggaactctgcgtg

SEQ ID NO: 354 - DNA, 20 bp, Artificial Sequence
  source: /note=“Description of Artificial Sequence Synthetic oligonucleotide”
gatggcgatgtatctgtcga

SEQ ID NO: 355 - DNA, 20 bp, Artificial Sequence
  source: /note=“Description of Artificial Sequence Synthetic oligonucleotide”
cttcttcgcctgctttgtcc

SEQ ID NO: 356 - DNA, 20 bp, Artificial Sequence
  source: /note=“Description of Artificial Sequence Synthetic oligonucleotide”
aggcgaagaagaggcagcca

SEQ ID NO: 357 - DNA, 20 bp, Artificial Sequence
  source: /note=“Description of Artificial Sequence Synthetic oligonucleotide”
tgcttgtgtgctgggccgtg

SEQ ID NO: 358 - DNA, 20 bp, Artificial Sequence
  source: /note=“Description of Artificial Sequence Synthetic oligonucleotide”
gaagccagtgctgatggtga

SEQ ID NO: 359 - DNA, 20 bp, Artificial Sequence
  source: /note=“Description of Artificial Sequence Synthetic oligonucleotide”
cgtgaggaccaggacaaagc

SEQ ID NO: 360 - DNA, 20 bp, Artificial Sequence
  source: /note=“Description of Artificial Sequence Synthetic oligonucleotide”
tggaactctgcgtgaggacc

SEQ ID NO: 361 - DNA, 20 bp, Artificial Sequence
  source: /note=“Description of Artificial Sequence Synthetic oligonucleotide”
cattgctgtgctggccatcc

SEQ ID NO: 362 - DNA, 20 bp, Artificial Sequence
  source: /note=“Description of Artificial Sequence Synthetic oligonucleotide”
ttctcccgccatgggctcct

SEQ ID NO: 363 - DNA, 20 bp, Artificial Sequence
  source: /note=“Description of Artificial Sequence Synthetic oligonucleotide”
tggctcaccggagtggaatt

SEQ ID NO: 364 - DNA, 20 bp, Artificial Sequence
  source: /note=“Description of Artificial Sequence Synthetic oligonucleotide”
tgctgatggtgatggcgaat

SEQ ID NO: 365 - DNA, 20 bp, Artificial Sequence
  source: /note=“Description of Artificial Sequence Synthetic oligonucleotide”
cttcgtggtatctctggcgg

SEQ ID NO: 366 - DNA, 20 bp, Artificial Sequence
  source: /note=“Description of Artificial Sequence Synthetic oligonucleotide”
agcacacaagcacgttaccc

SEQ ID NO: 367 - DNA, 20 bp, Artificial Sequence
  source: /note=“Description of Artificial Sequence Synthetic oligonucleotide”
gggctcctcggtgtacatca

SEQ ID NO: 368 - DNA, 20 bp, Artificial Sequence
  source: /note=“Description of Artificial Sequence Synthetic oligonucleotide”
gtacaccgaggagcccatgg

SEQ ID NO: 369 - DNA, 20 bp, Artificial Sequence
  source: /note=“Description of Artificial Sequence Synthetic oligonucleotide”
gaacgtcaccaacttcttcg

SEQ ID NO: 370 - DNA, 20 bp, Artificial Sequence
  source: /note=“Description of Artificial Sequence Synthetic oligonucleotide”
tcgccatccgaattccactc

SEQ ID NO: 371 - DNA, 20 bp, Artificial Sequence
  source: /note=“Description of Artificial Sequence Synthetic oligonucleotide”
gagttccatcttcagcctct

SEQ ID NO: 372 - DNA, 20 bp, Artificial Sequence
  source: /note=“Description of Artificial Sequence Synthetic oligonucleotide”
gaattccactccggtgagcc

SEQ ID NO: 373 - DNA, 20 bp, Artificial Sequence
  source: /note=“Description of Artificial Sequence Synthetic oligonucleotide”
cagagataccacgaagaagt

SEQ ID NO: 374 - DNA, 20 bp, Artificial Sequence
  source: /note=“Description of Artificial Sequence Synthetic oligonucleotide”
cttcttcgtggtatctctgg

SEQ ID NO: 375 - DNA, 21 bp, Artificial Sequence
  source: /note=“Description of Artificial Sequence Synthetic oligonucleotide”
cagtgctgatggtgatggcga

SEQ ID NO: 376 - DNA, 21 bp, Artificial Sequence
  source: /note=“Description of Artificial Sequence Synthetic oligonucleotide”
cgaattccactccggtgagcc

SEQ ID NO: 377 - DNA, 21 bp, Artificial Sequence
  source: /note=“Description of Artificial Sequence Synthetic oligonucleotide”
ccgaattccactccggtgagc

SEQ ID NO: 378 - DNA, 21 bp, Artificial Sequence
  source: /note=“Description of Artificial Sequence Synthetic oligonucleotide”
gctgaagatggaactctgcgt

SEQ ID NO: 379 - DNA, 21 bp, Artificial Sequence
  source: /note=“Description of Artificial Sequence Synthetic oligonucleotide”
cgtgcttgtgtgctgggccgt

SEQ ID NO: 380 - DNA, 21 bp, Artificial Sequence
  source: /note=“Description of Artificial Sequence Synthetic oligonucleotide”
gtgaggaccaggacaaagcag

SEQ ID NO: 381 - DNA, 21 bp, Artificial Sequence
  source: /note=“Description of Artificial Sequence Synthetic oligonucleotide”
tcgatggcaatagccaagagg

SEQ ID NO: 382 - DNA, 21 bp, Artificial Sequence
  source: /note=“Description of Artificial Sequence Synthetic oligonucleotide”
catcgacagatacatcgccat

SEQ ID NO: 383 - DNA, 21 bp, Artificial Sequence
  source: /note=“Description of Artificial Sequence Synthetic oligonucleotide”
gtacaccgaggagcccatggc

SEQ ID NO: 384 - DNA, 21 bp, Artificial Sequence
  source: /note=“Description of Artificial Sequence Synthetic oligonucleotide”
gctccaccatgatgtacaccg

SEQ ID NO: 385 - DNA, 21 bp, Artificial Sequence
  source: /note=“Description of Artificial Sequence Synthetic oligonucleotide”
aagccagtgctgatggtgatg

SEQ ID NO: 386 - DNA, 21 bp, Artificial Sequence
  source: /note=“Description of Artificial Sequence Synthetic oligonucleotide”
caccgcgatgtcagccgccgc

SEQ ID NO: 387 - DNA, 21 bp, Artificial Sequence
  source: /note=“Description of Artificial Sequence Synthetic oligonucleotide”
aggctgaagatggaactctgc

SEQ ID NO: 388 - DNA, 21 bp, Artificial Sequence
  source: /note=“Description of Artificial Sequence Synthetic oligonucleotide”
gccgccgccagagataccacg

SEQ ID NO: 389 - DNA, 21 bp, Artificial Sequence
  source: /note=“Description of Artificial Sequence Synthetic oligonucleotide”
agctccaccatgatgtacacc

SEQ ID NO: 390 - DNA, 21 bp, Artificial Sequence
  source: /note=“Description of Artificial Sequence Synthetic oligonucleotide”
aggcagccatggcaggcagcg

SEQ ID NO: 391 - DNA, 21 bp, Artificial Sequence
  source: /note=“Description of Artificial Sequence Synthetic oligonucleotide”
cctggctcaccggagtggaat

SEQ ID NO: 392 - DNA, 21 bp, Artificial Sequence
  source: /note=“Description of Artificial Sequence Synthetic oligonucleotide”
ccagctccaccatgatgtaca

SEQ ID NO: 393 - DNA, 21 bp, Artificial Sequence
  source: /note=“Description of Artificial Sequence Synthetic oligonucleotide”
accaggacaaagcaggcgaag

SEQ ID NO: 394 - DNA, 21 bp, Artificial Sequence
  source: /note=“Description of Artificial Sequence Synthetic oligonucleotide”
cctgggtaacgtgcttgtgtg

SEQ ID NO: 395 - DNA, 21 bp, Artificial Sequence
  source: /note=“Description of Artificial Sequence Synthetic oligonucleotide”
aggaccaggacaaagcaggcg

SEQ ID NO: 396 - DNA, 21 bp, Artificial Sequence
  source: /note=“Description of Artificial Sequence Synthetic oligonucleotide”
tcagccgccgccagagatacc

SEQ ID NO: 397 - DNA, 21 bp, Artificial Sequence
  source: /note=“Description of Artificial Sequence Synthetic oligonucleotide”
ggctcctcggtgtacatcatg

SEQ ID NO: 398 - DNA, 21 bp, Artificial Sequence
  source: /note=“Description of Artificial Sequence Synthetic oligonucleotide”
ctggcggcggctgacatcgcg

SEQ ID NO: 399 - DNA, 21 bp, Artificial Sequence
  source: /note=“Description of Artificial Sequence Synthetic oligonucleotide”
gatggaactctgcgtgaggac

SEQ ID NO: 400 - DNA, 21 bp, Artificial Sequence
  source: /note=“Description of Artificial Sequence Synthetic oligonucleotide”
gctcctcggtgtacatcatgg

SEQ ID NO: 401 - DNA, 21 bp, Artificial Sequence
  source: /note=“Description of Artificial Sequence Synthetic oligonucleotide”
tgtacaccgaggagcccatgg

SEQ ID NO: 402 - DNA, 21 bp, Artificial Sequence
  source: /note=“Description of Artificial Sequence Synthetic oligonucleotide”
gccattgctgtgctggccatc

SEQ ID NO: 403 - DNA, 21 bp, Artificial Sequence
  source: /note=“Description of Artificial Sequence Synthetic oligonucleotide”
caatagccaagaggctgaaga

SEQ ID NO: 404 - DNA, 21 bp, Artificial Sequence
  source: /note=“Description of Artificial Sequence Synthetic oligonucleotide”
atggtgatggcgaatgggatg

SEQ ID NO: 405 - DNA, 21 bp, Artificial Sequence
  source: /note=“Description of Artificial Sequence Synthetic oligonucleotide”
atgtacaccgaggagcccatg

SEQ ID NO: 406 - DNA, 21 bp, Artificial Sequence
  source: /note=“Description of Artificial Sequence Synthetic oligonucleotide”
gtgtggatcaacagcaacctg

SEQ ID NO: 407 - DNA, 21 bp, Artificial Sequence
  source: /note=“Description of Artificial Sequence Synthetic oligonucleotide”
tgctttgtcctggtcctcacg

SEQ ID NO: 408 - DNA, 21 bp, Artificial Sequence
  source: /note=“Description of Artificial Sequence Synthetic oligonucleotide”
gtaacccctggctcaccggag

SEQ ID NO: 409 - DNA, 21 bp, Artificial Sequence
  source: /note=“Description of Artificial Sequence Synthetic oligonucleotide”
ccagcacacaagcacgttacc

SEQ ID NO: 410 - DNA, 21 bp, Artificial Sequence
  source: /note=“Description of Artificial Sequence Synthetic oligonucleotide”
tatctgtcgatggcaatagcc

SEQ ID NO: 411 - DNA, 21 bp, Artificial Sequence
  source: /note=“Description of Artificial Sequence Synthetic oligonucleotide”
gcaatagccaagaggctgaag

SEQ ID NO: 412 - DNA, 21 bp, Artificial Sequence
  source: /note=“Description of Artificial Sequence Synthetic oligonucleotide”
agtgctgatggtgatggcgaa

SEQ ID NO: 413 - DNA, 21 bp, Artificial Sequence
  source: /note=“Description of Artificial Sequence Synthetic oligonucleotide”
acaccgaggagcccatggcgg

SEQ ID NO: 414 - DNA, 21 bp, Artificial Sequence
  source: /note=“Description of Artificial Sequence Synthetic oligonucleotide”
cgccatccgaattccactccg

SEQ ID NO: 415 - DNA, 20 bp, Artificial Sequence
  source: /note=“Description of Artificial Sequence Synthetic oligonucleotide”
tggtgtcactggcggcggcc

SEQ ID NO: 416 - DNA, 20 bp, Artificial Sequence
  source: /note=“Description of Artificial Sequence Synthetic oligonucleotide”
ccatcaccatcagcaccggg

SEQ ID NO: 417 - DNA, 20 bp, Artificial Sequence
  source: /note=“Description of Artificial Sequence Synthetic oligonucleotide”
ccatcggcctgactcccatg

SEQ ID NO: 418 - DNA, 20 bp, Artificial Sequence
  source: /note=“Description of Artificial Sequence Synthetic oligonucleotide”
gctgaccgcagttgttccaa

SEQ ID NO: 419 - DNA, 20 bp, Artificial Sequence
  source: /note=“Description of Artificial Sequence Synthetic oligonucleotide”
aggatgtggtccccatgaac

SEQ ID NO: 420 - DNA, 20 bp, Artificial Sequence
  source: /note=“Description of Artificial Sequence Synthetic oligonucleotide”
cctgtgtgctggtgcccctg

SEQ ID NO: 421 - DNA, 20 bp, Artificial Sequence
  source: /note=“Description of Artificial Sequence Synthetic oligonucleotide”
cggatcttcctggcggcgcg

SEQ ID NO: 422 - DNA, 20 bp, Artificial Sequence
  source: /note=“Description of Artificial Sequence Synthetic oligonucleotide”
ccctctgctggctgccccta

SEQ ID NO: 423 - DNA, 20 bp, Artificial Sequence
  source: /note=“Description of Artificial Sequence Synthetic oligonucleotide”
ttctgccccgactgcagcca

SEQ ID NO: 424 - DNA, 20 bp, Artificial Sequence
  source: /note=“Description of Artificial Sequence Synthetic oligonucleotide”
aaggcagctggcaccagtgc

SEQ ID NO: 425 - DNA, 21 bp, Artificial Sequence
  source: /note=“Description of Artificial Sequence Synthetic oligonucleotide”
taagggcatcattgccatctg

SEQ ID NO: 426 - DNA, 21 bp, Artificial Sequence
  source: /note=“Description of Artificial Sequence Synthetic oligonucleotide”
cggcctgactcccatgctagg

SEQ ID NO: 427 - DNA, 21 bp, Artificial Sequence
  source: /note=“Description of Artificial Sequence Synthetic oligonucleotide”
gcagttgttccaacctagcat

SEQ ID NO: 428 - DNA, 21 bp, Artificial Sequence
  source: /note=“Description of Artificial Sequence Synthetic oligonucleotide”
ccgcagttgttccaacctagc

SEQ ID NO: 429 - DNA, 21 bp, Artificial Sequence
  source: /note=“Description of Artificial Sequence Synthetic oligonucleotide”
caagaaccactcccagggctg

SEQ ID NO: 430 - DNA, 21 bp, Artificial Sequence
  source: /note=“Description of Artificial Sequence Synthetic oligonucleotide”
cttggccctccccgcagccct

SEQ ID NO: 431 - DNA, 21 bp, Artificial Sequence
  source: /note=“Description of Artificial Sequence Synthetic oligonucleotide”
cacttggccctccccgcagcc

SEQ ID NO: 432 - DNA, 21 bp, Artificial Sequence
  source: /note=“Description of Artificial Sequence Synthetic oligonucleotide”
ggccaagtggcctgtctcttt

SEQ ID NO: 433 - DNA, 21 bp, Artificial Sequence
  source: /note=“Description of Artificial Sequence Synthetic oligonucleotide”
ttcatggggaccacatcctca

SEQ ID NO: 434 - DNA, 21 bp, Artificial Sequence
  source: /note=“Description of Artificial Sequence Synthetic oligonucleotide”
tgaagtacaccatgtagttca

SEQ ID NO: 435 - DNA, 21 bp, Artificial Sequence
  source: /note=“Description of Artificial Sequence Synthetic oligonucleotide”
ctggtgcccctgctgctcatg

SEQ ID NO: 436 - DNA, 21 bp, Artificial Sequence
  source: /note=“Description of Artificial Sequence Synthetic oligonucleotide”
gctcatgctgggtgtctattt

SEQ ID NO: 437 - DNA, 21 bp, Artificial Sequence
  source: /note=“Description of Artificial Sequence Synthetic oligonucleotide”
cttcagctgtcgtcgcgccgc

SEQ ID NO: 438 - DNA, 21 bp, Artificial Sequence
  source: /note=“Description of Artificial Sequence Synthetic oligonucleotide”
cgcgacgacagctgaagcaga

SEQ ID NO: 439 - DNA, 21 bp, Artificial Sequence
  source: /note=“Description of Artificial Sequence Synthetic oligonucleotide”
gatggagagccagcctctgcc

SEQ ID NO: 440 - DNA, 21 bp, Artificial Sequence
  source: /note=“Description of Artificial Sequence Synthetic oligonucleotide”
gcgtggctgcagtcggggcag

SEQ ID NO: 441 - DNA, 21 bp, Artificial Sequence
  source: /note=“Description of Artificial Sequence Synthetic oligonucleotide”
acgatggccaggtacatgagc

SEQ ID NO: 442 - DNA, 21 bp, Artificial Sequence
  source: /note=“Description of Artificial Sequence Synthetic oligonucleotide”
ctctcccacaccaattcggtt

SEQ ID NO: 443 - DNA, 21 bp, Artificial Sequence
  source: /note=“Description of Artificial Sequence Synthetic oligonucleotide”
gattcacaaccgaattggtgt

SEQ ID NO: 444 - DNA, 21 bp, Artificial Sequence
  source: /note=“Description of Artificial Sequence Synthetic oligonucleotide”
gggattcacaaccgaattggt

SEQ ID NO: 445 - DNA, 21 bp, Artificial Sequence
  source: /note=“Description of Artificial Sequence Synthetic oligonucleotide”
cgtagatgaagggattcacaa

SEQ ID NO: 446 - DNA, 21 bp, Artificial Sequence
  source: /note=“Description of Artificial Sequence Synthetic oligonucleotide”
ggatacggtaggcgtagatga

SEQ ID NO: 447 - DNA, 21 bp, Artificial Sequence
  source: /note=“Description of Artificial Sequence Synthetic oligonucleotide”
tcatctacgcctaccgtatcc

SEQ ID NO: 448 - DNA, 21 bp, Artificial Sequence
  source: /note=“Description of Artificial Sequence Synthetic oligonucleotide”
cggatacggtaggcgtagatg

SEQ ID NO: 449 - DNA, 21 bp, Artificial Sequence
  source: /note=“Description of Artificial Sequence Synthetic oligonucleotide”
gcggaaggtctggcggaactc

SEQ ID NO: 450 - DNA, 21 bp, Artificial Sequence
  source: /note=“Description of Artificial Sequence Synthetic oligonucleotide”
aatgatcttgcggaaggtctg

SEQ ID NO: 451 - DNA, 21 bp, Artificial Sequence
  source: /note=“Description of Artificial Sequence Synthetic oligonucleotide”
gacgtggctgcgaatgatctt

SEQ ID NO: 452 - DNA, 21 bp, Artificial Sequence
  source: /note=“Description of Artificial Sequence Synthetic oligonucleotide”
ttgctgcctcaggacgtggct

SEQ ID NO: 453 - DNA, 21 bp, Artificial Sequence
  source: /note=“Description of Artificial Sequence Synthetic oligonucleotide”
caaggcagctggcaccagtgc

SEQ ID NO: 454 - DNA, 21 bp, Artificial Sequence
  source: /note=“Description of Artificial Sequence Synthetic oligonucleotide”
cgggcactggtgccagctgcc

SEQ ID NO: 455 - DNA, 21 bp, Artificial Sequence
  source: /note=“Description of Artificial Sequence Synthetic oligonucleotide”
cttggcagctcatggcagtga

SEQ ID NO: 456 - DNA, 21 bp, Artificial Sequence
  source: /note=“Description of Artificial Sequence Synthetic oligonucleotide”
ccgtctcaacggccacccgcc

SEQ ID NO: 457 - DNA, 21 bp, Artificial Sequence
  source: /note=“Description of Artificial Sequence Synthetic oligonucleotide”
cacactcctggcgggtggccg

SEQ ID NO: 458 - DNA, 21 bp, Artificial Sequence
  source: /note=“Description of Artificial Sequence Synthetic oligonucleotide”
tgccgttggcccacactcctg

SEQ ID NO: 459 - DNA, 21 bp, Artificial Sequence
  source: /note=“Description of Artificial Sequence Synthetic oligonucleotide”
ccattgggcctccgctcaggg

SEQ ID NO: 460 - DNA, 21 bp, Artificial Sequence
  source: /note=“Description of Artificial Sequence Synthetic oligonucleotide”
catagccattgggcctccgct

SEQ ID NO: 461 - DNA, 21 bp, Artificial Sequence
  source: /note=“Description of Artificial Sequence Synthetic oligonucleotide”
aatggctatgccctggggctg

SEQ ID NO: 462 - DNA, 21 bp, Artificial Sequence
  source: /note=“Description of Artificial Sequence Synthetic oligonucleotide”
atgccctggggctggtgagtg

SEQ ID NO: 463 - DNA, 21 bp, Artificial Sequence
  source: /note=“Description of Artificial Sequence Synthetic oligonucleotide”
gccctggggctggtgagtgga

SEQ ID NO: 464 - DNA, 21 bp, Artificial Sequence
  source: /note=“Description of Artificial Sequence Synthetic oligonucleotide”
tggtgagtggagggagtgccc

SEQ ID NO: 465 - DNA, 21 bp, Artificial Sequence
  source: /note=“Description of Artificial Sequence Synthetic oligonucleotide”
gagggagtgcccaagagtccc

SEQ ID NO: 466 - DNA, 21 bp, Artificial Sequence
  source: /note=“Description of Artificial Sequence Synthetic oligonucleotide”
agggagtgcccaagagtccca

SEQ ID NO: 467 - DNA, 21 bp, Artificial Sequence
  source: /note=“Description of Artificial Sequence Synthetic oligonucleotide”
gtctgggaggcccgtgttccc

SEQ ID NO: 468 - DNA, 21 bp, Artificial Sequence
  source: /note=“Description of Artificial Sequence Synthetic oligonucleotide”
catggctaaggagctccacgt

SEQ ID NO: 469 - DNA, 21 bp, Artificial Sequence
  source: /note=“Description of Artificial Sequence Synthetic oligonucleotide”
gagctccttagccatgagctc

SEQ ID NO: 470 - DNA, 21 bp, Artificial Sequence
  source: /note=“Description of Artificial Sequence Synthetic oligonucleotide”
gctccttagccatgagctcaa

SEQ ID NO: 471 - DNA, 21 bp, Artificial Sequence
  source: /note=“Description of Artificial Sequence Synthetic oligonucleotide”
ggcctagatgaccccctggcc

SEQ ID NO: 472 - DNA, 21 bp, Artificial Sequence
  source: /note=“Description of Artificial Sequence Synthetic oligonucleotide”
ccccctggcccaggatggagc

SEQ ID NO: 473 - DNA, 21 bp, Artificial Sequence
  source: /note=“Description of Artificial Sequence Synthetic oligonucleotide”
ctcctgctccatcctgggcca

SEQ ID NO: 474 - DNA, 20 bp, Artificial Sequence
  source: /note=“Description of Artificial Sequence Synthetic oligonucleotide”
ccgtgatgtacaccgaggag

SEQ ID NO: 475 - DNA, 20 bp, Artificial Sequence
  source: /note=“Description of Artificial Sequence Synthetic oligonucleotide”
ctttgccatcaccatcagca

SEQ ID NO: 476 - DNA, 20 bp, Artificial Sequence
  source: /note=“Description of Artificial Sequence Synthetic oligonucleotide”
tttgccatcaccatcagcac

SEQ ID NO: 477 - DNA, 20 bp, Artificial Sequence
  source: /note=“Description of Artificial Sequence Synthetic oligonucleotide”
ttgcctgcttcgtcctggtc

SEQ ID NO: 478 - DNA, 20 bp, Artificial Sequence
  source: /note=“Description of Artificial Sequence Synthetic oligonucleotide”
tcctggtcctcacgcagagc

SEQ ID NO: 479 - DNA, 20 bp, Artificial Sequence
  source: /note=“Description of Artificial Sequence Synthetic oligonucleotide”
tcttcagtctcctggccatc

SEQ ID NO: 480 - DNA, 20 bp, Artificial Sequence
  source: /note=“Description of Artificial Sequence Synthetic oligonucleotide”
gtctcctggccatcgccatt

SEQ ID NO: 481 - DNA, 20 bp, Artificial Sequence
  source: /note=“Description of Artificial Sequence Synthetic oligonucleotide”
acctagcatgggagtcaggc

SEQ ID NO: 482 - DNA, 20 bp, Artificial Sequence
  source: /note=“Description of Artificial Sequence Synthetic oligonucleotide”
aacctagcatgggagtcagg

SEQ ID NO: 483 - DNA, 20 bp, Artificial Sequence
  source: /note=“Description of Artificial Sequence Synthetic oligonucleotide”
atgctaggttggaacaactg

SEQ ID NO: 484 - DNA, 20 bp, Artificial Sequence
  source: /note=“Description of Artificial Sequence Synthetic oligonucleotide”
gcagccctgggagtggttct

SEQ ID NO: 485 - DNA, 20 bp, Artificial Sequence
  source: /note=“Description of Artificial Sequence Synthetic oligonucleotide”
cgcagccctgggagtggttc

SEQ ID NO: 486 - DNA, 20 bp, Artificial Sequence
  source: /note=“Description of Artificial Sequence Synthetic oligonucleotide”
agggctgcggggagggccaa

SEQ ID NO: 487 - DNA, 20 bp, Artificial Sequence
  source: /note=“Description of Artificial Sequence Synthetic oligonucleotide”
tggggaccacatcctcaaag

SEQ ID NO: 488 - DNA, 20 bp, Artificial Sequence
  source: /note=“Description of Artificial Sequence Synthetic oligonucleotide”
catgaactacatggtgtact

SEQ ID NO: 489 - DNA, 20 bp, Artificial Sequence
  source: /note=“Description of Artificial Sequence Synthetic oligonucleotide”
atgaactacatggtgtactt

SEQ ID NO: 490 - DNA, 20 bp, Artificial Sequence
  source: /note=“Description of Artificial Sequence Synthetic oligonucleotide”
acttctttgcctgtgtgctg

SEQ ID NO: 491 - DNA, 20 bp, Artificial Sequence
  source: /note=“Description of Artificial Sequence Synthetic oligonucleotide”
tgctgctcatgctgggtgtc

SEQ ID NO: 492 - DNA, 20 bp, Artificial Sequence
  source: /note=“Description of Artificial Sequence Synthetic oligonucleotide”
caaatagacacccagcatga

SEQ ID NO: 493 - DNA, 20 bp, Artificial Sequence
  source: /note=“Description of Artificial Sequence Synthetic oligonucleotide”
gctgtcgtcgcgccgccagg

SEQ ID NO: 494 - DNA, 20 bp, Artificial Sequence
  source: /note=“Description of Artificial Sequence Synthetic oligonucleotide”
tggcggcgcgacgacagctg

SEQ ID NO: 495 - DNA, 20 bp, Artificial Sequence
  source: /note=“Description of Artificial Sequence Synthetic oligonucleotide”
tctgcttcagctgtcgtcgc

SEQ ID NO: 496 - DNA, 20 bp, Artificial Sequence
  source: /note=“Description of Artificial Sequence Synthetic oligonucleotide”
ggcagaggctggctctccat

SEQ ID NO: 497 - DNA, 20 bp, Artificial Sequence
  source: /note=“Description of Artificial Sequence Synthetic oligonucleotide”
cggcagaggctggctctcca

SEQ ID NO: 498 - DNA, 20 bp, Artificial Sequence
  source: /note=“Description of Artificial Sequence Synthetic oligonucleotide”
ccggcagaggctggctctcc

SEQ ID NO: 499 - DNA, 20 bp, Artificial Sequence
  source: /note=“Description of Artificial Sequence Synthetic oligonucleotide”
cactgcagaaggaggtccat

SEQ ID NO: 500 - DNA, 20 bp, Artificial Sequence
  source: /note=“Description of Artificial Sequence Synthetic oligonucleotide”
tgctgccaagtcactggcca

SEQ ID NO: 501 - DNA, 20 bp, Artificial Sequence
  source: /note=“Description of Artificial Sequence Synthetic oligonucleotide”
acaatgatggccagtgactt

SEQ ID NO: 502 - DNA, 20 bp, Artificial Sequence
  source: /note=“Description of Artificial Sequence Synthetic oligonucleotide”
tacacatcatcaactgcttc

SEQ ID NO: 503 - DNA, 20 bp, Artificial Sequence
  source: /note=“Description of Artificial Sequence Synthetic oligonucleotide”
ctttcttctgccccgactgc

SEQ ID NO: 504 - DNA, 20 bp, Artificial Sequence
  source: /note=“Description of Artificial Sequence Synthetic oligonucleotide”
gactgcagccacgcccctct

SEQ ID NO: 505 - DNA, 20 bp, Artificial Sequence
  source: /note=“Description of Artificial Sequence Synthetic oligonucleotide”
tctctggctcatgtacctgg

SEQ ID NO: 506 - DNA, 20 bp, Artificial Sequence
  source: /note=“Description of Artificial Sequence Synthetic oligonucleotide”
caaccgaattggtgtgggag

SEQ ID NO: 507 - DNA, 20 bp, Artificial Sequence
  source: /note=“Description of Artificial Sequence Synthetic oligonucleotide”
acaccaattcggttgtgaat

SEQ ID NO: 508 - DNA, 20 bp, Artificial Sequence
  source: /note=“Description of Artificial Sequence Synthetic oligonucleotide”
gttgtgaatcccttcatcta

SEQ ID NO: 509 - DNA, 20 bp, Artificial Sequence
  source: /note=“Description of Artificial Sequence Synthetic oligonucleotide”
ttcatctacgcctaccgtat

SEQ ID NO: 510 - DNA, 20 bp, Artificial Sequence
  source: /note=“Description of Artificial Sequence Synthetic oligonucleotide”
tctacgcctaccgtatccgc

SEQ ID NO: 511 - DNA, 20 bp, Artificial Sequence
  source: /note=“Description of Artificial Sequence Synthetic oligonucleotide”
cgagttccgccagaccttcc

SEQ ID NO: 512 - DNA, 20 bp, Artificial Sequence
  source: /note=“Description of Artificial Sequence Synthetic oligonucleotide”
gccagaccttccgcaagatc

SEQ ID NO: 513 - DNA, 20 bp, Artificial Sequence
  source: /note=“Description of Artificial Sequence Synthetic oligonucleotide”
ccagaccttccgcaagatca

SEQ ID NO: 514 - DNA, 20 bp, Artificial Sequence
  source: /note=“Description of Artificial Sequence Synthetic oligonucleotide”
gcaagatcattcgcagccac

SEQ ID NO: 515 - DNA, 20 bp, Artificial Sequence
  source: /note=“Description of Artificial Sequence Synthetic oligonucleotide”
caagatcattcgcagccacg

SEQ ID NO: 516 - DNA, 20 bp, Artificial Sequence
  source: /note=“Description of Artificial Sequence Synthetic oligonucleotide”
cagccacgtcctgaggcagc

SEQ ID NO: 517 - DNA, 20 bp, Artificial Sequence
  source: /note=“Description of Artificial Sequence Synthetic oligonucleotide”
aggcagctggcaccagtgcc

SEQ ID NO: 518 - DNA, 20 bp, Artificial Sequence
  source: /note=“Description of Artificial Sequence Synthetic oligonucleotide”
tcactgccatgagctgccaa

SEQ ID NO: 519 - DNA, 20 bp, Artificial Sequence
  source: /note=“Description of Artificial Sequence Synthetic oligonucleotide”
tctcaacggccacccgccag

SEQ ID NO: 520 - DNA, 20 bp, Artificial Sequence
  source: /note=“Description of Artificial Sequence Synthetic oligonucleotide”
ctcagggtggggagcactgc

SEQ ID NO: 521 - DNA, 20 bp, Artificial Sequence
  source: /note=“Description of Artificial Sequence Synthetic oligonucleotide”
caccctgagcggaggcccaa

SEQ ID NO: 522 - DNA, 20 bp, Artificial Sequence
  source: /note=“Description of Artificial Sequence Synthetic oligonucleotide”
accctgagcggaggcccaat

SEQ ID NO: 523 - DNA, 20 bp, Artificial Sequence
  source: /note=“Description of Artificial Sequence Synthetic oligonucleotide”
agggcatagccattgggcct

SEQ ID NO: 524 - DNA, 20 bp, Artificial Sequence
  source: /note=“Description of Artificial Sequence Synthetic oligonucleotide”
ctcaccagccccagggcata

SEQ ID NO: 525 - DNA, 20 bp, Artificial Sequence
  source: /note=“Description of Artificial Sequence Synthetic oligonucleotide”
tccactcaccagccccaggg

SEQ ID NO: 526 - DNA, 20 bp, Artificial Sequence
  source: /note=“Description of Artificial Sequence Synthetic oligonucleotide”
tgggactcttgggcactccc

SEQ ID NO: 527 - DNA, 20 bp, Artificial Sequence
  source: /note=“Description of Artificial Sequence Synthetic oligonucleotide”
ctgggactcttgggcactcc

SEQ ID NO: 528 - DNA, 20 bp, Artificial Sequence
  source: /note=“Description of Artificial Sequence Synthetic oligonucleotide”
cctgggactcttgggcactc

SEQ ID NO: 529 - DNA, 20 bp, Artificial Sequence
  source: /note=“Description of Artificial Sequence Synthetic oligonucleotide”
aggggaacacgggcctccca

SEQ ID NO: 530 - DNA, 20 bp, Artificial Sequence
  source: /note=“Description of Artificial Sequence Synthetic oligonucleotide”
cgtctgggaggcccgtgttc

SEQ ID NO: 531 - DNA, 20 bp, Artificial Sequence
  source: /note=“Description of Artificial Sequence Synthetic oligonucleotide”
agacgtggagctccttagcc

SEQ ID NO: 532 - DNA, 20 bp, Artificial Sequence
  source: /note=“Description of Artificial Sequence Synthetic oligonucleotide”
ttgagctcatggctaaggag

SEQ ID NO: 533 - DNA, 20 bp, Artificial Sequence
  source: /note=“Description of Artificial Sequence Synthetic oligonucleotide”
ctggcctagatgaccccctg

SEQ ID NO: 534 - DNA, 20 bp, Artificial Sequence
  source: /note=“Description of Artificial Sequence Synthetic oligonucleotide”
tggcctagatgaccccctgg

SEQ ID NO: 535 - DNA, 20 bp, Artificial Sequence
  source: /note=“Description of Artificial Sequence Synthetic oligonucleotide”
tcctgggccagggggtcatc

SEQ ID NO: 536 - DNA, 20 bp, Artificial Sequence
  source: /note=“Description of Artificial Sequence Synthetic oligonucleotide”
ctggcccaggatggagcagg

SEQ ID NO: 537 - DNA, 20 bp, Artificial Sequence
  source: /note=“Description of Artificial Sequence Synthetic oligonucleotide”
tggcccaggatggagcagga

SEQ ID NO: 538 - DNA, 20 bp, Artificial Sequence
  source: /note=“Description of Artificial Sequence Synthetic oligonucleotide”
cgcgagttccgccagacctt

SEQ ID NO: 539 - DNA, 20 bp, Artificial Sequence
  source: /note=“Description of Artificial Sequence Synthetic oligonucleotide”
ccctggggctggtgagtgga

SEQ ID NO: 540 - DNA, 20 bp, Artificial Sequence
  source: /note=“Description of Artificial Sequence Synthetic oligonucleotide”
caggacgatgtgcagcggcc

SEQ ID NO: 541 - DNA, 20 bp, Artificial Sequence
  source: /note=“Description of Artificial Sequence Synthetic oligonucleotide”
accgcacgttcagaagtcgg

SEQ ID NO: 542 - DNA, 20 bp, Artificial Sequence
  source: /note=“Description of Artificial Sequence Synthetic oligonucleotide”
acaactgtgtaaattttgtg

SEQ ID NO: 543 - DNA, 20 bp, Artificial Sequence
  source: /note=“Description of Artificial Sequence Synthetic oligonucleotide”
caactgtgtaaattttgtga

SEQ ID NO: 544 - DNA, 20 bp, Artificial Sequence
  source: /note=“Description of Artificial Sequence Synthetic oligonucleotide”
acctgtgacaaccagaaatc

SEQ ID NO: 545 - DNA, 20 bp, Artificial Sequence
  source: /note=“Description of Artificial Sequence Synthetic oligonucleotide”
cctgtgacaaccagaaatcc

SEQ ID NO: 546 - DNA, 20 bp, Artificial Sequence
  source: /note=“Description of Artificial Sequence Synthetic oligonucleotide”
tgtggcttctcacagatgga

SEQ ID NO: 547 - DNA, 20 bp, Artificial Sequence
  source: /note=“Description of Artificial Sequence Synthetic oligonucleotide”
tctgtgagaagccacaggaa

SEQ ID NO: 548 - DNA, 20 bp, Artificial Sequence
  source: /note=“Description of Artificial Sequence Synthetic oligonucleotide”
aagctcccctaccatgactt

SEQ ID NO: 549 - DNA, 20 bp, Artificial Sequence
  source: /note=“Description of Artificial Sequence Synthetic oligonucleotide”
gaataaagtcatggtagggg

SEQ ID NO: 550 - DNA, 20 bp, Artificial Sequence
  source: /note=“Description of Artificial Sequence Synthetic oligonucleotide”
agaataaagtcatggtaggg

SEQ ID NO: 551 - DNA, 20 bp, Artificial Sequence
  source: /note=“Description of Artificial Sequence Synthetic oligonucleotide”
ctaccatgactttattctgg

SEQ ID NO: 552 - DNA, 20 bp, Artificial Sequence
  source: /note=“Description of Artificial Sequence Synthetic oligonucleotide”
taccatgactttattctgga

SEQ ID NO: 553 - DNA, 20 bp, Artificial Sequence
  source: /note=“Description of Artificial Sequence Synthetic oligonucleotide”
taatgcactttggagaagca

SEQ ID NO: 554 - DNA, 20 bp, Artificial Sequence
  source: /note=“Description of Artificial Sequence Synthetic oligonucleotide”
ttcataatgcactttggaga

SEQ ID NO: 555 - DNA, 20 bp, Artificial Sequence
  source: /note=“Description of Artificial Sequence Synthetic oligonucleotide”
aagtgcattatgaaggaaaa

SEQ ID NO: 556 - DNA, 20 bp, Artificial Sequence
  source: /note=“Description of Artificial Sequence Synthetic oligonucleotide”
tgtgttcctgtagctctgat

SEQ ID NO: 557 - DNA, 20 bp, Artificial Sequence
  source: /note=“Description of Artificial Sequence Synthetic oligonucleotide”
tgtagctctgatgagtgcaa

SEQ ID NO: 558 - DNA, 20 bp, Artificial Sequence
  source: /note=“Description of Artificial Sequence Synthetic oligonucleotide”
agtgacaggcatcagcctcc

SEQ ID NO: 559 - DNA, 20 bp, Artificial Sequence
  source: /note=“Description of Artificial Sequence Synthetic oligonucleotide”
agtggtggcaggaggctgat

SEQ ID NO: 560 - DNA, 20 bp, Artificial Sequence
  source: /note=“Description of Artificial Sequence Synthetic oligonucleotide”
aggttgaactcagcttctgc

SEQ ID NO: 561 - DNA, 20 bp, Artificial Sequence
  source: /note=“Description of Artificial Sequence Synthetic oligonucleotide”
caggttgaactcagcttctg

SEQ ID NO: 562 - DNA, 20 bp, Artificial Sequence
  source: /note=“Description of Artificial Sequence Synthetic oligonucleotide”
acctgggaaaccggcaagac

SEQ ID NO: 563 - DNA, 20 bp, Artificial Sequence
  source: /note=“Description of Artificial Sequence Synthetic oligonucleotide”
cgtcttgccggtttcccagg

SEQ ID NO: 564 - DNA, 20 bp, Artificial Sequence
  source: /note=“Description of Artificial Sequence Synthetic oligonucleotide”
gcgtcttgccggtttcccag

SEQ ID NO: 565 - DNA, 20 bp, Artificial Sequence
  source: /note=“Description of Artificial Sequence Synthetic oligonucleotide”
tgagcttccgcgtcttgccg

SEQ ID NO: 566 - DNA, 20 bp, Artificial Sequence
  source: /note=“Description of Artificial Sequence Synthetic oligonucleotide”
gcgagcactgtgccatcatc

SEQ ID NO: 567 - DNA, 20 bp, Artificial Sequence
  source: /note=“Description of Artificial Sequence Synthetic oligonucleotide”
ggatgatggcacagtgctcg

SEQ ID NO: 568 - DNA, 20 bp, Artificial Sequence
  source: /note=“Description of Artificial Sequence Synthetic oligonucleotide”
aggatgatggcacagtgctc

SEQ ID NO: 569 - DNA, 20 bp, Artificial Sequence
  source: /note=“Description of Artificial Sequence Synthetic oligonucleotide”
cgtgtgccaacaacatcaac

SEQ ID NO: 570 - DNA, 20 bp, Artificial Sequence
  source: /note=“Description of Artificial Sequence Synthetic oligonucleotide”
gctcaatgggcagcagctct

SEQ ID NO: 571 - DNA, 20 bp, Artificial Sequence
  source: /note=“Description of Artificial Sequence Synthetic oligonucleotide”
accagggtgtccagctcaat

SEQ ID NO: 572 - DNA, 20 bp, Artificial Sequence
  source: /note=“Description of Artificial Sequence Synthetic oligonucleotide”
caccagggtgtccagctcaa

SEQ ID NO: 573 - DNA, 20 bp, Artificial Sequence
  source: /note=“Description of Artificial Sequence Synthetic oligonucleotide”
ccaccagggtgtccagctca

SEQ ID NO: 574 - DNA, 20 bp, Artificial Sequence
  source: /note=“Description of Artificial Sequence Synthetic oligonucleotide”
gcttggccttatagacctca

SEQ ID NO: 575 - DNA, 20 bp, Artificial Sequence
  source: /note=“Description of Artificial Sequence Synthetic oligonucleotide”
gagcagtttgagacagtggc

SEQ ID NO: 576 - DNA, 20 bp, Artificial Sequence
  source: /note=“Description of Artificial Sequence Synthetic oligonucleotide”
agaggcatactcctcatagg

SEQ ID NO: 577 - DNA, 20 bp, Artificial Sequence
  source: /note=“Description of Artificial Sequence Synthetic oligonucleotide”
ctatgaggagtatgcctctt

SEQ ID NO: 578 - DNA, 20 bp, Artificial Sequence
  source: /note=“Description of Artificial Sequence Synthetic oligonucleotide”
aagaggcatactcctcatag

SEQ ID NO: 579 - DNA, 20 bp, Artificial Sequence
  source: /note=“Description of Artificial Sequence Synthetic oligonucleotide”
tatgaggagtatgcctcttg

SEQ ID NO: 580 - DNA, 20 bp, Artificial Sequence
  source: /note=“Description of Artificial Sequence Synthetic oligonucleotide”
gattgatgtctgagaagatg

SEQ ID NO: 581 - DNA, 20 bp, Artificial Sequence
  source: /note=“Description of Artificial Sequence Synthetic oligonucleotide”
ctcctcagccgtcaggaact

SEQ ID NO: 582 - DNA, 20 bp, Artificial Sequence
  source: /note=“Description of Artificial Sequence Synthetic oligonucleotide”
gttcctgacggctgaggagc

SEQ ID NO: 583 - DNA, 20 bp, Artificial Sequence
  source: /note=“Description of Artificial Sequence Synthetic oligonucleotide”
gctcctcagccgtcaggaac

SEQ ID NO: 584 - DNA, 20 bp, Artificial Sequence
  source: /note=“Description of Artificial Sequence Synthetic oligonucleotide”
tgacggctgaggagcggaag

SEQ ID NO: 585 - DNA, 20 bp, Artificial Sequence
  source: /note=“Description of Artificial Sequence Synthetic oligonucleotide”
tcttccgctcctcagccgtc

SEQ ID NO: 586 - DNA, 20 bp, Artificial Sequence
  source: /note=“Description of Artificial Sequence Synthetic oligonucleotide”
aactccgtcttccgctcctc

SEQ ID NO: 587 - DNA, 20 bp, Artificial Sequence
  source: /note=“Description of Artificial Sequence Synthetic oligonucleotide”
caactccgtcttccgctcct

SEQ ID NO: 588 - DNA, 20 bp, Artificial Sequence
  source: /note=“Description of Artificial Sequence Synthetic oligonucleotide”
ccaactccgtcttccgctcc

SEQ ID NO: 589 - DNA, 20 bp, Artificial Sequence
  source: /note=“Description of Artificial Sequence Synthetic oligonucleotide”
acgccaagggcaacctacag

SEQ ID NO: 590 - DNA, 20 bp, Artificial Sequence
  source: /note=“Description of Artificial Sequence Synthetic oligonucleotide”
cgccaagggcaacctacagg

SEQ ID NO: 591 - DNA, 20 bp, Artificial Sequence
  source: /note=“Description of Artificial Sequence Synthetic oligonucleotide”
agctgatgacatgccgcgtc

SEQ ID NO: 592 - DNA, 20 bp, Artificial Sequence
  source: /note=“Description of Artificial Sequence Synthetic oligonucleotide”
gggcgagggagctgcccagc

SEQ ID NO: 593 - DNA, 20 bp, Artificial Sequence
  source: /note=“Description of Artificial Sequence Synthetic oligonucleotide”
cgggcgagggagctgcccag

SEQ ID NO: 594 - DNA, 20 bp, Artificial Sequence
  source: /note=“Description of Artificial Sequence Synthetic oligonucleotide”
ccgggcgagggagctgccca

SEQ ID NO: 595 - DNA, 20 bp, Artificial Sequence
  source: /note=“Description of Artificial Sequence Synthetic oligonucleotide”
tcgcccgggggattgctcac

SEQ ID NO: 596 - DNA, 20 bp, Artificial Sequence
  source: /note=“Description of Artificial Sequence Synthetic oligonucleotide”
acatggagtgtgatcactgt

SEQ ID NO: 597 - DNA, 20 bp, Artificial Sequence
  source: /note=“Description of Artificial Sequence Synthetic oligonucleotide”
cagtgatcacactccatgtg

SEQ ID NO: 598 - DNA, 20 bp, Artificial Sequence
  source: /note=“Description of Artificial Sequence Synthetic oligonucleotide”
tgtgggaggcccaagatgcc

SEQ ID NO: 599 - DNA, 20 bp, Artificial Sequence
  source: /note=“Description of Artificial Sequence Synthetic oligonucleotide”
tgtgcacgatgggcatcttg

SEQ ID NO: 600 - DNA, 20 bp, Artificial Sequence
  source: /note=“Description of Artificial Sequence Synthetic oligonucleotide”
cgaggatattggagctcttg

SEQ ID NO: 601 - DNA, 20 bp, Artificial Sequence
  source: /note=“Description of Artificial Sequence Synthetic oligonucleotide”
atatcctcgtgaagaacgac

SEQ ID NO: 602 - DNA, 20 bp, Artificial Sequence
  source: /note=“Description of Artificial Sequence Synthetic oligonucleotide”
gacgcagggaaagcccaaag

SEQ ID NO: 603 - DNA, 20 bp, Artificial Sequence
  source: /note=“Description of Artificial Sequence Synthetic oligonucleotide”
ctgcgtctggaccctactct

SEQ ID NO: 604 - DNA, 20 bp, Artificial Sequence
  source: /note=“Description of Artificial Sequence Synthetic oligonucleotide”
tgcgtctggaccctactctg

SEQ ID NO: 605 - DNA, 20 bp, Artificial Sequence
  source: /note=“Description of Artificial Sequence Synthetic oligonucleotide”
cagacagagtagggtccaga

SEQ ID NO: 606 - DNA, 20 bp, Artificial Sequence
  source: /note=“Description of Artificial Sequence Synthetic oligonucleotide”
gccagcacgatcccaccgca

SEQ ID NO: 607 - DNA, 20 bp, Artificial Sequence
  source: /note=“Description of Artificial Sequence Synthetic oligonucleotide”
aaggaaaaaaaaaagcctgg

SEQ ID NO: 608 - DNA, 20 bp, Artificial Sequence
  source: /note=“Description of Artificial Sequence Synthetic oligonucleotide”
acaccagcaatcctgacttg

SEQ ID NO: 609 - DNA, 20 bp, Artificial Sequence
  source: /note=“Description of Artificial Sequence Synthetic oligonucleotide”
actagcaacaagtcaggatt

SEQ ID NO: 610 - DNA, 20 bp, Artificial Sequence
  source: /note=“Description of Artificial Sequence Synthetic oligonucleotide”
gcaactcccagtggtggcag

SEQ ID NO: 611 - DNA, 20 bp, Artificial Sequence
  source: /note=“Description of Artificial Sequence Synthetic oligonucleotide”
tgtcatcatcatcttctact

SEQ ID NO: 612 - DNA, 20 bp, Artificial Sequence
  source: /note=“Description of Artificial Sequence Synthetic oligonucleotide”
gacctcagcaaagcgacctt

SEQ ID NO: 613 - DNA, 20 bp, Artificial Sequence
  source: /note=“Description of Artificial Sequence Synthetic oligonucleotide”
aggccaagctgaagcagaac

SEQ ID NO: 614 - DNA, 20 bp, Artificial Sequence
  source: /note=“Description of Artificial Sequence Synthetic oligonucleotide”
aggagtatgcctcttggaag

SEQ ID NO: 615 - DNA, 20 bp, Artificial Sequence
  source: /note=“Description of Artificial Sequence Synthetic oligonucleotide”
cctcttggaagacagagaag

SEQ ID NO: 616 - DNA, 20 bp, Artificial Sequence
  source: /note=“Description of Artificial Sequence Synthetic oligonucleotide”
ttctcatgcttcagattgat

SEQ ID NO: 617 - DNA, 20 bp, Artificial Sequence
  source: /note=“Description of Artificial Sequence Synthetic oligonucleotide”
ctcgtgaagaacgacctaac

SEQ ID NO: 618 - DNA, 20 bp, Artificial Sequence
  source: /note=“Description of Artificial Sequence Synthetic oligonucleotide”
ggccgctgcacatcgtcctg

SEQ ID NO: 619 - DNA, 20 bp, Artificial Sequence
  source: /note=“Description of Artificial Sequence Synthetic oligonucleotide”
gcggggtctgccatgggtcg

SEQ ID NO: 620 - DNA, 20 bp, Artificial Sequence
  source: /note=“Description of Artificial Sequence Synthetic oligonucleotide”
agttgctcatgcaggatttc

SEQ ID NO: 621 - DNA, 20 bp, Artificial Sequence
  source: /note=“Description of Artificial Sequence Synthetic oligonucleotide”
ccagaataaagtcatggtag

SEQ ID NO: 622 - DNA, 20 bp, Artificial Sequence
  source: /note=“Description of Artificial Sequence Synthetic oligonucleotide”
cccctaccatgactttattc

SEQ ID NO: 623 - DNA, 20 bp, Artificial Sequence
  source: /note=“Description of Artificial Sequence Synthetic oligonucleotide”
aagtcatggtaggggagctt

SEQ ID NO: 624 - DNA, 20 bp, Artificial Sequence
  source: /note=“Description of Artificial Sequence Synthetic oligonucleotide”
agtcatggtaggggagcttg

SEQ ID NO: 625 - DNA, 20 bp, Artificial Sequence
  source: /note=“Description of Artificial Sequence Synthetic oligonucleotide”
attgcactcatcagagctac

SEQ ID NO: 626 - DNA, 20 bp, Artificial Sequence
  source: /note=“Description of Artificial Sequence Synthetic oligonucleotide”
cctagagtgaagagattcat

SEQ ID NO: 627 - DNA, 20 bp, Artificial Sequence
  source: /note=“Description of Artificial Sequence Synthetic oligonucleotide”
ccaatgaatctcttcactct

SEQ ID NO: 628 - DNA, 20 bp, Artificial Sequence
  source: /note=“Description of Artificial Sequence Synthetic oligonucleotide”
aaagtcatggtaggggagct

SEQ ID NO: 629 - DNA, 20 bp, Artificial Sequence
  source: /note=“Description of Artificial Sequence Synthetic oligonucleotide”
gtgagcaatcccccgggcga

SEQ ID NO: 630 - DNA, 20 bp, Artificial Sequence
  source: /note=“Description of Artificial Sequence Synthetic oligonucleotide”
gtcgttcttcacgaggatat

SEQ ID NO: 631 - DNA, 20 bp, Artificial Sequence
  source: /note=“Description of Artificial Sequence Synthetic oligonucleotide”
gccgcgtcaggtactcctgt

SEQ ID NO: 632 - DNA, 20 bp, Artificial Sequence
  source: /note=“Description of Artificial Sequence Synthetic oligonucleotide”
gacgcggcatgtcatcagct

SEQ ID NO: 633 - DNA, 20 bp, Artificial Sequence
  source: /note=“Description of Artificial Sequence Synthetic oligonucleotide”
gcttctgctgccggttaacg

SEQ ID NO: 634 - DNA, 20 bp, Artificial Sequence
  source: /note=“Description of Artificial Sequence Synthetic oligonucleotide”
gtggatgacctggctaacag

SEQ ID NO: 635 - DNA, 20 bp, Artificial Sequence
  source: /note=“Description of Artificial Sequence Synthetic oligonucleotide”
gtgatcacactccatgtggg

SEQ ID NO: 636 - DNA, 20 bp, Artificial Sequence
  source: /note=“Description of Artificial Sequence Synthetic oligonucleotide”
gcccattgagctggacaccc

SEQ ID NO: 637 - DNA, 20 bp, Artificial Sequence
  source: /note=“Description of Artificial Sequence Synthetic oligonucleotide”
gcggtcatcttccaggatga

SEQ ID NO: 638 - DNA, 20 bp, Artificial Sequence
  source: /note=“Description of Artificial Sequence Synthetic oligonucleotide”
gggagctgcccagcttgcgc

SEQ ID NO: 639 - DNA, 20 bp, Artificial Sequence
  source: /note=“Description of Artificial Sequence Synthetic oligonucleotide”
gttgatgttgttggcacacg

SEQ ID NO: 640 - DNA, 20 bp, Artificial Sequence
  source: /note=“Description of Artificial Sequence Synthetic oligonucleotide”
ggcatcttgggcctcccaca

SEQ ID NO: 641 - DNA, 20 bp, Artificial Sequence
  source: /note=“Description of Artificial Sequence Synthetic oligonucleotide”
gcggcatgtcatcagctggg

SEQ ID NO: 642 - DNA, 20 bp, Artificial Sequence
  source: /note=“Description of Artificial Sequence Synthetic oligonucleotide”
gctcctcagccgtcaggaac

SEQ ID NO: 643 - DNA, 20 bp, Artificial Sequence
  source: /note=“Description of Artificial Sequence Synthetic oligonucleotide”
gctggtgttatattctgatg

SEQ ID NO: 644 - DNA, 20 bp, Artificial Sequence
  source: /note=“Description of Artificial Sequence Synthetic oligonucleotide”
ccgacttctgaacgtgcggt

SEQ ID NO: 645 - DNA, 20 bp, Artificial Sequence
  source: /note=“Description of Artificial Sequence Synthetic oligonucleotide”
tgctggcgatacgcgtccac

SEQ ID NO: 646 - DNA, 20 bp, Artificial Sequence
  source: /note=“Description of Artificial Sequence Synthetic oligonucleotide”
cccgacttctgaacgtgcgg

SEQ ID NO: 647 - DNA, 20 bp, Artificial Sequence
  source: /note=“Description of Artificial Sequence Synthetic oligonucleotide”
ccaccgcacgttcagaagtc

SEQ ID NO: 648 - DNA, 20 bp, Artificial Sequence
  source: /note=“Description of Artificial Sequence Synthetic oligonucleotide”
tcacccgacttctgaacgtg

SEQ ID NO: 649 - DNA, 20 bp, Artificial Sequence
  source: /note=“Description of Artificial Sequence Synthetic oligonucleotide”
cccaccgcacgttcagaagt

SEQ ID NO: 650 - DNA, 20 bp, Artificial Sequence
  source: /note=“Description of Artificial Sequence Synthetic oligonucleotide”
cgagcagcggggtctgccat

SEQ ID NO: 651 - DNA, 20 bp, Artificial Sequence
  source: /note=“Description of Artificial Sequence Synthetic oligonucleotide”
acgagcagcggggtctgcca

SEQ ID NO: 652 - DNA, 20 bp, Artificial Sequence
  source: /note=“Description of Artificial Sequence Synthetic oligonucleotide”
agcggggtctgccatgggtc

SEQ ID NO: 653 - DNA, 20 bp, Artificial Sequence
  source: /note=“Description of Artificial Sequence Synthetic oligonucleotide”
cctgagcagcccccgaccca

SEQ ID NO: 654 - DNA, 20 bp, Artificial Sequence
  source: /note=“Description of Artificial Sequence Synthetic oligonucleotide”
ccatgggtcgggggctgctc

SEQ ID NO: 655 - DNA, 20 bp, Artificial Sequence
  source: /note=“Description of Artificial Sequence Synthetic oligonucleotide”
aacgtgcggtgggatcgtgc

SEQ ID NO: 656 - DNA, 20 bp, Artificial Sequence
  source: /note=“Description of Artificial Sequence Synthetic oligonucleotide”
ggacgatgtgcagcggccac

SEQ ID NO: 657 - DNA, 20 bp, Artificial Sequence
  source: /note=“Description of Artificial Sequence Synthetic oligonucleotide”
gtccacaggacgatgtgcag

SEQ ID NO: 658 - DNA, 20 bp, Artificial Sequence
  source: /note=“Description of Artificial Sequence Synthetic oligonucleotide”
catgggtcgggggctgctca

SEQ ID NO: 659 - DNA, 20 bp, Artificial Sequence
  source: /note=“Description of Artificial Sequence Synthetic oligonucleotide”
cagcggggtctgccatgggt

SEQ ID NO: 660 - DNA, 20 bp, Artificial Sequence
  source: /note=“Description of Artificial Sequence Synthetic oligonucleotide”
atgggtcgggggctgctcag

SEQ ID NO: 661 - DNA, 20 bp, Artificial Sequence
  source: /note=“Description of Artificial Sequence Synthetic oligonucleotide”
cggggtctgccatgggtcgg

SEQ ID NO: 662 - DNA, 20 bp, Artificial Sequence
  source: /note=“Description of Artificial Sequence Synthetic oligonucleotide”
aggaagtctgtgtggctgta

SEQ ID NO: 663 - DNA, 20 bp, Artificial Sequence
  source: /note=“Description of Artificial Sequence Synthetic oligonucleotide”
ctccatctgtgagaagccac

SEQ ID NO: 664 - DNA, 20 bp, Artificial Sequence
  source: /note=“Description of Artificial Sequence Synthetic oligonucleotide”
atgatagtcactgacaacaa

SEQ ID NO: 665 - DNA, 20 bp, Artificial Sequence
  source: /note=“Description of Artificial Sequence Synthetic oligonucleotide”
gatgctgcagttgctcatgc

SEQ ID NO: 666 - DNA, 20 bp, Artificial Sequence
  source: /note=“Description of Artificial Sequence Synthetic oligonucleotide”
acagccacacagacttcctg

SEQ ID NO: 667 - DNA, 20 bp, Artificial Sequence
  source: /note=“Description of Artificial Sequence Synthetic oligonucleotide”
gaagccacaggaagtctgtg

SEQ ID NO: 668 - DNA, 20 bp, Artificial Sequence
  source: /note=“Description of Artificial Sequence Synthetic oligonucleotide”
ttcctgtggcttctcacaga

SEQ ID NO: 669 - DNA, 20 bp, Artificial Sequence
  source: /note=“Description of Artificial Sequence Synthetic oligonucleotide”
ctgtggcttctcacagatgg

SEQ ID NO: 670 - DNA, 20 bp, Artificial Sequence
  source: /note=“Description of Artificial Sequence Synthetic oligonucleotide”
tcacaaaatttacacagttg

SEQ ID NO: 671 - DNA, 20 bp, Artificial Sequence
  source: /note=“Description of Artificial Sequence Synthetic oligonucleotide”
gacaacatcatcttctcaga

SEQ ID NO: 672 - DNA, 20 bp, Artificial Sequence
  source: /note=“Description of Artificial Sequence Synthetic oligonucleotide”
tccagaataaagtcatggta

SEQ ID NO: 673 - DNA, 20 bp, Artificial Sequence
  source: /note=“Description of Artificial Sequence Synthetic oligonucleotide”
ggtaggggagcttggggtca

SEQ ID NO: 674 - DNA, 20 bp, Artificial Sequence
  source: /note=“Description of Artificial Sequence Synthetic oligonucleotide”
ttctccaaagtgcattatga

SEQ ID NO: 675 - DNA, 20 bp, Artificial Sequence
  source: /note=“Description of Artificial Sequence Synthetic oligonucleotide”
catcttccagaataaagtca

SEQ ID NO: 676 - DNA, 20 bp, Artificial Sequence
  source: /note=“Description of Artificial Sequence Synthetic oligonucleotide”
cacatgaagaaagtctcacc

SEQ ID NO: 677 - DNA, 20 bp, Artificial Sequence
  source: /note=“Description of Artificial Sequence Synthetic oligonucleotide”
ttccagaataaagtcatggt

SEQ ID NO: 678 - DNA, 20 bp, Artificial Sequence
  source: /note=“Description of Artificial Sequence Synthetic oligonucleotide”
ttttccttcataatgcactt

SEQ ID NO: 679 - DNA, 20 bp, Artificial Sequence
  source: /note=“Description of Artificial Sequence Synthetic oligonucleotide”
cacagttgtggaaacttgac

SEQ ID NO: 680 - DNA, 20 bp, Artificial Sequence
  source: /note=“Description of Artificial Sequence Synthetic oligonucleotide”
cccaactccgtcttccgctc

SEQ ID NO: 681 - DNA, 20 bp, Artificial Sequence
  source: /note=“Description of Artificial Sequence Synthetic oligonucleotide”
ggctttccctgcgtctggac

SEQ ID NO: 682 - DNA, 20 bp, Artificial Sequence
  source: /note=“Description of Artificial Sequence Synthetic oligonucleotide”
ctgaggtctataaggccaag

SEQ ID NO: 683 - DNA, 20 bp, Artificial Sequence
  source: /note=“Description of Artificial Sequence Synthetic oligonucleotide”
tgatgtgagattttccacct

SEQ ID NO: 684 - DNA, 20 bp, Artificial Sequence
  source: /note=“Description of Artificial Sequence Synthetic oligonucleotide”
cctatgaggagtatgcctct

SEQ ID NO: 685 - DNA, 20 bp, Artificial Sequence
  source: /note=“Description of Artificial Sequence Synthetic oligonucleotide”
aagtgacaggcatcagcctc

SEQ ID NO: 686 - DNA, 20 bp, Artificial Sequence
  source: /note=“Description of Artificial Sequence Synthetic oligonucleotide”
ccatgaccccaagctcccct

SEQ ID NO: 687 - DNA, 20 bp, Artificial Sequence
  source: /note=“Description of Artificial Sequence Synthetic oligonucleotide”
cttcataatgcactttggag

SEQ ID NO: 688 - DNA, 20 bp, Artificial Sequence
  source: /note=“Description of Artificial Sequence Synthetic oligonucleotide”
ttcatgtgttcctgtagctc

SEQ ID NO: 689 - DNA, 20 bp, Artificial Sequence
  source: /note=“Description of Artificial Sequence Synthetic oligonucleotide”
ttctggaagatgctgcttct

SEQ ID NO: 690 - DNA, 20 bp, Artificial Sequence
  source: /note=“Description of Artificial Sequence Synthetic oligonucleotide”
cccaccagggtgtccagctc

SEQ ID NO: 691 - DNA, 20 bp, Artificial Sequence
  source: /note=“Description of Artificial Sequence Synthetic oligonucleotide”
agacagtggcagtcaagatc

SEQ ID NO: 692 - DNA, 20 bp, Artificial Sequence
  source: /note=“Description of Artificial Sequence Synthetic oligonucleotide”
cctgcgtctggaccctactc

SEQ ID NO: 693 - DNA, 20 bp, Artificial Sequence
  source: /note=“Description of Artificial Sequence Synthetic oligonucleotide”
cacaactgtgtaaattttgt

SEQ ID NO: 694 - DNA, 20 bp, Artificial Sequence
  source: /note=“Description of Artificial Sequence Synthetic oligonucleotide”
gagaagcagcatcttccaga

SEQ ID NO: 695 - DNA, 20 bp, Artificial Sequence
  source: /note=“Description of Artificial Sequence Synthetic oligonucleotide”
tggttgtcacaggtggaaaa

SEQ ID NO: 696 - DNA, 20 bp, Artificial Sequence
  source: /note=“Description of Artificial Sequence Synthetic oligonucleotide”
ccaggttgaactcagcttct

SEQ ID NO: 697 - DNA, 21 bp, Artificial Sequence
  source: /note=“Description of Artificial Sequence Synthetic oligonucleotide”
atcacaaaatttacacagttg

SEQ ID NO: 698 - DNA, 21 bp, Artificial Sequence
  source: /note=“Description of Artificial Sequence Synthetic oligonucleotide”
ggcatcagcctcctgccacca

SEQ ID NO: 699 - DNA, 21 bp, Artificial Sequence
  source: /note=“Description of Artificial Sequence Synthetic oligonucleotide”
gttagccaggtcatccacaga

SEQ ID NO: 700 - DNA, 21 bp, Artificial Sequence
  source: /note=“Description of Artificial Sequence Synthetic oligonucleotide”
gctgggcagctccctcgcccg

SEQ ID NO: 701 - DNA, 21 bp, Artificial Sequence
  source: /note=“Description of Artificial Sequence Synthetic oligonucleotide”
caggaggctgatgcctgtcac

SEQ ID NO: 702 - DNA, 21 bp, Artificial Sequence
  source: /note=“Description of Artificial Sequence Synthetic oligonucleotide”
gaggagcggaagacggagttg

SEQ ID NO: 703 - DNA, 21 bp, Artificial Sequence
  source: /note=“Description of Artificial Sequence Synthetic oligonucleotide”
cgtctggaccctactctgtct

SEQ ID NO: 704 - DNA, 21 bp, Artificial Sequence
  source: /note=“Description of Artificial Sequence Synthetic oligonucleotide”
tttttccttcataatgcactt

SEQ ID NO: 705 - DNA, 21 bp, Artificial Sequence
  source: /note=“Description of Artificial Sequence Synthetic oligonucleotide”
ccattgagctggacaccctgg

SEQ ID NO: 706 - DNA, 21 bp, Artificial Sequence
  source: /note=“Description of Artificial Sequence Synthetic oligonucleotide”
cttctccaaagtgcattatga

SEQ ID NO: 707 - DNA, 21 bp, Artificial Sequence
  source: /note=“Description of Artificial Sequence Synthetic oligonucleotide”
gcccaagatgcccatcgtgca

SEQ ID NO: 708 - DNA, 21 bp, Artificial Sequence
  source: /note=“Description of Artificial Sequence Synthetic oligonucleotide”
tcatgtgttcctgtagctctg

SEQ ID NO: 709 - DNA, 21 bp, Artificial Sequence
  source: /note=“Description of Artificial Sequence Synthetic oligonucleotide”
gtgatgctgcagttgctcatg

SEQ ID NO: 710 - DNA, 21 bp, Artificial Sequence
  source: /note=“Description of Artificial Sequence Synthetic oligonucleotide”
tctcatgcttcagattgatgt

SEQ ID NO: 711 - DNA, 21 bp, Artificial Sequence
  source: /note=“Description of Artificial Sequence Synthetic oligonucleotide”
tccctatgaggagtatgcctc

SEQ ID NO: 712 - DNA, 21 bp, Artificial Sequence
  source: /note=“Description of Artificial Sequence Synthetic oligonucleotide”
catcacaaaatttacacagtt

SEQ ID NO: 713 - DNA, 21 bp, Artificial Sequence
  source: /note=“Description of Artificial Sequence Synthetic oligonucleotide”
attgagctggacaccctggtg

SEQ ID NO: 714 - DNA, 21 bp, Artificial Sequence
  source: /note=“Description of Artificial Sequence Synthetic oligonucleotide”
cagtcaagatctttccctatg

SEQ ID NO: 715 - DNA, 21 bp, Artificial Sequence
  source: /note=“Description of Artificial Sequence Synthetic oligonucleotide”
aggatttctggttgtcacagg

SEQ ID NO: 716 - DNA, 21 bp, Artificial Sequence
  source: /note=“Description of Artificial Sequence Synthetic oligonucleotide”
tccacagtgatcacactccat

SEQ ID NO: 717 - DNA, 21 bp, Artificial Sequence
  source: /note=“Description of Artificial Sequence Synthetic oligonucleotide”
agcagaacacttcagagcagt

SEQ ID NO: 718 - DNA, 21 bp, Artificial Sequence
  source: /note=“Description of Artificial Sequence Synthetic oligonucleotide”
ccggcaagacgcggaagctca

SEQ ID NO: 719 - DNA, 21 bp, Artificial Sequence
  source: /note=“Description of Artificial Sequence Synthetic oligonucleotide”
gatgtcagagcggtcatcttc

SEQ ID NO: 720 - DNA, 21 bp, Artificial Sequence
  source: /note=“Description of Artificial Sequence Synthetic oligonucleotide”
tcattgcactcatcagagcta

SEQ ID NO: 721 - DNA, 21 bp, Artificial Sequence
  source: /note=“Description of Artificial Sequence Synthetic oligonucleotide”
cttccagaataaagtcatggt

SEQ ID NO: 722 - DNA, 21 bp, Artificial Sequence
  source: /note=“Description of Artificial Sequence Synthetic oligonucleotide”
agattttccacctgtgacaac

SEQ ID NO: 723 - DNA, 21 bp, Artificial Sequence
  source: /note=“Description of Artificial Sequence Synthetic oligonucleotide”
actgcagcatcacctccatct

SEQ ID NO: 724 - DNA, 21 bp, Artificial Sequence
  source: /note=“Description of Artificial Sequence Synthetic oligonucleotide”
agctgggcagctccctcgccc

SEQ ID NO: 725 - DNA, 21 bp, Artificial Sequence
  source: /note=“Description of Artificial Sequence Synthetic oligonucleotide”
tgacggctgaggagcggaaga

SEQ ID NO: 726 - DNA, 21 bp, Artificial Sequence
  source: /note=“Description of Artificial Sequence Synthetic oligonucleotide”
cattgagctggacaccctggt

SEQ ID NO: 727 - DNA, 21 bp, Artificial Sequence
  source: /note=“Description of Artificial Sequence Synthetic oligonucleotide”
agcaaagcgacctttccccac

SEQ ID NO: 728 - DNA, 21 bp, Artificial Sequence
  source: /note=“Description of Artificial Sequence Synthetic oligonucleotide”
cgcgttaaccggcagcagaag

SEQ ID NO: 729 - DNA, 21 bp, Artificial Sequence
  source: /note=“Description of Artificial Sequence Synthetic oligonucleotide”
gaaatatgactagcaacaagt

SEQ ID NO: 730 - DNA, 21 bp, Artificial Sequence
  source: /note=“Description of Artificial Sequence Synthetic oligonucleotide”
agacagagtagggtccagacg

SEQ ID NO: 731 - DNA, 21 bp, Artificial Sequence
  source: /note=“Description of Artificial Sequence Synthetic oligonucleotide”
caggatttctggttgtcacag

SEQ ID NO: 732 - DNA, 21 bp, Artificial Sequence
  source: /note=“Description of Artificial Sequence Synthetic oligonucleotide”
ctcctgtaggttgcccttggc

SEQ ID NO: 733 - DNA, 21 bp, Artificial Sequence
  source: /note=“Description of Artificial Sequence Synthetic oligonucleotide”
acagagtagggtccagacgca

SEQ ID NO: 734 - DNA, 21 bp, Artificial Sequence
  source: /note=“Description of Artificial Sequence Synthetic oligonucleotide”
gcttctccaaagtgcattatg

SEQ ID NO: 735 - DNA, 21 bp, Artificial Sequence
  source: /note=“Description of Artificial Sequence Synthetic oligonucleotide”
gcagcagaagctgagttcaac

SEQ ID NO: 736 - DNA, 21 bp, Artificial Sequence
  source: /note=“Description of Artificial Sequence Synthetic oligonucleotide”
tgaggagcggaagacggagtt

SEQ ID NO: 737 - DNA, 21 bp, Artificial Sequence
  source: /note=“Description of Artificial Sequence Synthetic oligonucleotide”
ctttggagaagcagcatcttc

SEQ ID NO: 738 - DNA, 21 bp, Artificial Sequence
  source: /note=“Description of Artificial Sequence Synthetic oligonucleotide”
ctcccctaccatgactttatt

SEQ ID NO: 739 - DNA, 21 bp, Artificial Sequence
  source: /note=“Description of Artificial Sequence Synthetic oligonucleotide”
gacagagtagggtccagacgc

SEQ ID NO: 740 - DNA, 21 bp, Artificial Sequence
  source: /note=“Description of Artificial Sequence Synthetic oligonucleotide”
ctgaggagcggaagacggagt

SEQ ID NO: 741 - DNA, 21 bp, Artificial Sequence
  source: /note=“Description of Artificial Sequence Synthetic oligonucleotide”
gggcatcttgggcctcccaca

SEQ ID NO: 742 - DNA, 21 bp, Artificial Sequence
  source: /note=“Description of Artificial Sequence Synthetic oligonucleotide”
ccaagaggcatactcctcata

SEQ ID NO: 743 - DNA, 21 bp, Artificial Sequence
  source: /note=“Description of Artificial Sequence Synthetic oligonucleotide”
agaatgacgagaacataacac

SEQ ID NO: 744 - DNA, 21 bp, Artificial Sequence
  source: /note=“Description of Artificial Sequence Synthetic oligonucleotide”
cctgacgcggcatgtcatcag

SEQ ID NO: 745 - DNA, 21 bp, Artificial Sequence
  source: /note=“Description of Artificial Sequence Synthetic oligonucleotide”
agcgagcactgtgccatcatc

SEQ ID NO: 746 - DNA, 21 bp, Artificial Sequence
  source: /note=“Description of Artificial Sequence Synthetic oligonucleotide”
gcaggttaggtcgttcttcac

SEQ ID NO: 747 - DNA, 21 bp, Artificial Sequence
  source: /note=“Description of Artificial Sequence Synthetic oligonucleotide”
acctccatctgtgagaagcca

SEQ ID NO: 748 - DNA, 21 bp, Artificial Sequence
  source: /note=“Description of Artificial Sequence Synthetic oligonucleotide”
taaagtcatggtaggggagct

SEQ ID NO: 749 - DNA, 21 bp, Artificial Sequence
  source: /note=“Description of Artificial Sequence Synthetic oligonucleotide”
tcagagctacaggaacacatg

SEQ ID NO: 750 - DNA, 21 bp, Artificial Sequence
  source: /note=“Description of Artificial Sequence Synthetic oligonucleotide”
tctcagacatcaatctgaagc

SEQ ID NO: 751 - DNA, 21 bp, Artificial Sequence
  source: /note=“Description of Artificial Sequence Synthetic oligonucleotide”
catcagcctcctgccaccact

SEQ ID NO: 752 - DNA, 21 bp, Artificial Sequence
  source: /note=“Description of Artificial Sequence Synthetic oligonucleotide”
cgctcctcagccgtcaggaac

SEQ ID NO: 753 - DNA, 21 bp, Artificial Sequence
  source: /note=“Description of Artificial Sequence Synthetic oligonucleotide”
aacctgggaaaccggcaagac

SEQ ID NO: 754 - DNA, 21 bp, Artificial Sequence
  source: /note=“Description of Artificial Sequence Synthetic oligonucleotide”
tccacgccaagggcaacctac

SEQ ID NO: 755 - DNA, 21 bp, Artificial Sequence
  source: /note=“Description of Artificial Sequence Synthetic oligonucleotide”
gaggtgagcaatcccccgggc

SEQ ID NO: 756 - DNA, 21 bp, Artificial Sequence
  source: /note=“Description of Artificial Sequence Synthetic oligonucleotide”
cagcagaagctgagttcaacc

SEQ ID NO: 757 - DNA, 21 bp, Artificial Sequence
  source: /note=“Description of Artificial Sequence Synthetic oligonucleotide”
tccaagaggcatactcctcat

SEQ ID NO: 758 - DNA, 21 bp, Artificial Sequence
  source: /note=“Description of Artificial Sequence Synthetic oligonucleotide”
agcagaagctgagttcaacct

SEQ ID NO: 759 - DNA, 21 bp, Artificial Sequence
  source: /note=“Description of Artificial Sequence Synthetic oligonucleotide”
ccagttcctgacggctgagga

SEQ ID NO: 760 - DNA, 21 bp, Artificial Sequence
  source: /note=“Description of Artificial Sequence Synthetic oligonucleotide”
aggagtatgcctcttggaaga

SEQ ID NO: 761 - DNA, 21 bp, Artificial Sequence
  source: /note=“Description of Artificial Sequence Synthetic oligonucleotide”
ttccaagaggcatactcctca

SEQ ID NO: 762 - DNA, 21 bp, Artificial Sequence
  source: /note=“Description of Artificial Sequence Synthetic oligonucleotide”
caactgtgtaaattttgtgat

SEQ ID NO: 763 - DNA, 21 bp, Artificial Sequence
  source: /note=“Description of Artificial Sequence Synthetic oligonucleotide”
tgaaggaaaaaaaaaagcctg

SEQ ID NO: 764 - DNA, 21 bp, Artificial Sequence
  source: /note=“Description of Artificial Sequence Synthetic oligonucleotide”
cgtcttccgctcctcagccgt

SEQ ID NO: 765 - DNA, 21 bp, Artificial Sequence
  source: /note=“Description of Artificial Sequence Synthetic oligonucleotide”
ccaggtcatccacagacagag

SEQ ID NO: 766 - DNA, 21 bp, Artificial Sequence
  source: /note=“Description of Artificial Sequence Synthetic oligonucleotide”
gcctagagtgaagagattcat

SEQ ID NO: 767 - DNA, 21 bp, Artificial Sequence
  source: /note=“Description of Artificial Sequence Synthetic oligonucleotide”
gttctccaaagtgcattatga

SEQ ID NO: 768 - DNA, 21 bp, Artificial Sequence
  source: /note=“Description of Artificial Sequence Synthetic oligonucleotide”
gcatcttccagaataaagtca

SEQ ID NO: 769 - DNA, 20 bp, Artificial Sequence
  source: /note=“Description of Artificial Sequence Synthetic oligonucleotide”
caaccgtctggtggccgacg

SEQ ID NO: 770 - DNA, 20 bp, Artificial Sequence
  source: /note=“Description of Artificial Sequence Synthetic oligonucleotide”
caggatcggggctgtcgctt

SEQ ID NO: 771 - DNA, 20 bp, Artificial Sequence
  source: /note=“Description of Artificial Sequence Synthetic oligonucleotide”
tcgggcctcgctggccgtaa

SEQ ID NO: 772 - DNA, 20 bp, Artificial Sequence
  source: /note=“Description of Artificial Sequence Synthetic oligonucleotide”
gaggtagtcggccatgcgcc

SEQ ID NO: 773 - DNA, 20 bp, Artificial Sequence
  source: /note=“Description of Artificial Sequence Synthetic oligonucleotide”
caggtgttgtcgggcctcgc

SEQ ID NO: 774 - DNA, 20 bp, Artificial Sequence
  source: /note=“Description of Artificial Sequence Synthetic oligonucleotide”
ggaggtagtcggccatgcgc

SEQ ID NO: 775 - DNA, 20 bp, Artificial Sequence
  source: /note=“Description of Artificial Sequence Synthetic oligonucleotide”
ggcatactcaatgcgtacat

SEQ ID NO: 776 - DNA, 20 bp, Artificial Sequence
  source: /note=“Description of Artificial Sequence Synthetic oligonucleotide”
ccgccttgtcatcaaccgtc

SEQ ID NO: 777 - DNA, 20 bp, Artificial Sequence
  source: /note=“Description of Artificial Sequence Synthetic oligonucleotide”
aggatcggggctgtcgcttc

SEQ ID NO: 778 - DNA, 20 bp, Artificial Sequence
  source: /note=“Description of Artificial Sequence Synthetic oligonucleotide”
ccttgtcatcaaccgtctgg

SEQ ID NO: 779 - DNA, 20 bp, Artificial Sequence
  source: /note=“Description of Artificial Sequence Synthetic oligonucleotide”
tactcaatgcgtacattggt

SEQ ID NO: 780 - DNA, 20 bp, Artificial Sequence
  source: /note=“Description of Artificial Sequence Synthetic oligonucleotide”
gggttccattacggccagcg

SEQ ID NO: 781 - DNA, 20 bp, Artificial Sequence
  source: /note=“Description of Artificial Sequence Synthetic oligonucleotide”
ggcactgcttctgcgtacaa

SEQ ID NO: 782 - DNA, 20 bp, Artificial Sequence
  source: /note=“Description of Artificial Sequence Synthetic oligonucleotide”
ggttgatgacaaggcggcac

SEQ ID NO: 783 - DNA, 20 bp, Artificial Sequence
  source: /note=“Description of Artificial Sequence Synthetic oligonucleotide”
tgctggggccttcctcgagg

SEQ ID NO: 784 - DNA, 20 bp, Artificial Sequence
  source: /note=“Description of Artificial Sequence Synthetic oligonucleotide”
ttgctggctgtggagcggac

SEQ ID NO: 785 - DNA, 20 bp, Artificial Sequence
  source: /note=“Description of Artificial Sequence Synthetic oligonucleotide”
ttctcctaccttcgggaatc

SEQ ID NO: 786 - DNA, 20 bp, Artificial Sequence
  source: /note=“Description of Artificial Sequence Synthetic oligonucleotide”
gactggcttgggcagttcca

SEQ ID NO: 787 - DNA, 20 bp, Artificial Sequence
  source: /note=“Description of Artificial Sequence Synthetic oligonucleotide”
catgcagcccttgcctgctg

SEQ ID NO: 788 - DNA, 20 bp, Artificial Sequence
  source: /note=“Description of Artificial Sequence Synthetic oligonucleotide”
agcaaaggacgaggtctaga

SEQ ID NO: 789 - DNA, 20 bp, Artificial Sequence
  source: /note=“Description of Artificial Sequence Synthetic oligonucleotide”
gcctgctggggccttcctcg

SEQ ID NO: 790 - DNA, 20 bp, Artificial Sequence
  source: /note=“Description of Artificial Sequence Synthetic oligonucleotide”
cagactcaccagattcccga

SEQ ID NO: 791 - DNA, 20 bp, Artificial Sequence
  source: /note=“Description of Artificial Sequence Synthetic oligonucleotide”
acctcgtcctttgctggctg

SEQ ID NO: 792 - DNA, 20 bp, Artificial Sequence
  source: /note=“Description of Artificial Sequence Synthetic oligonucleotide”
ctcaccagattcccgaaggt

SEQ ID NO: 793 - DNA, 20 bp, Artificial Sequence
  source: /note=“Description of Artificial Sequence Synthetic oligonucleotide”
tacgcagaagcagtgcccgc

SEQ ID NO: 794 - DNA, 20 bp, Artificial Sequence
  source: /note=“Description of Artificial Sequence Synthetic oligonucleotide”
aggtgtacagcagtggctgg

SEQ ID NO: 795 - DNA, 20 bp, Artificial Sequence
  source: /note=“Description of Artificial Sequence Synthetic oligonucleotide”
ggtgtacagcagtggctggt

SEQ ID NO: 796 - DNA, 20 bp, Artificial Sequence
  source: /note=“Description of Artificial Sequence Synthetic oligonucleotide”
cggatgtggtcagccttgtg

SEQ ID NO: 797 - DNA, 20 bp, Artificial Sequence
  source: /note=“Description of Artificial Sequence Synthetic oligonucleotide”
cactgacagcgtgaacaggt

SEQ ID NO: 798 - DNA, 20 bp, Artificial Sequence
  source: /note=“Description of Artificial Sequence Synthetic oligonucleotide”
actgacagcgtgaacaggta

SEQ ID NO: 799 - DNA, 20 bp, Artificial Sequence
  source: /note=“Description of Artificial Sequence Synthetic oligonucleotide”
gctcactctctgtctgggct

SEQ ID NO: 800 - DNA, 20 bp, Artificial Sequence
  source: /note=“Description of Artificial Sequence Synthetic oligonucleotide”
ctggctgtggagcggactgg

SEQ ID NO: 801 - DNA, 20 bp, Artificial Sequence
  source: /note=“Description of Artificial Sequence Synthetic oligonucleotide”
gctctgactgtacggggcaa

SEQ ID NO: 802 - DNA, 20 bp, Artificial Sequence
  source: /note=“Description of Artificial Sequence Synthetic oligonucleotide”
agctctgactgtacggggca

SEQ ID NO: 803 - DNA, 20 bp, Artificial Sequence
  source: /note=“Description of Artificial Sequence Synthetic oligonucleotide”
acagtaccccttccagctct

SEQ ID NO: 804 - DNA, 20 bp, Artificial Sequence
  source: /note=“Description of Artificial Sequence Synthetic oligonucleotide”
cgtcggccaccagacggttg

SEQ ID NO: 805 - DNA, 20 bp, Artificial Sequence
  source: /note=“Description of Artificial Sequence Synthetic oligonucleotide”
ccagccactgctgtacacct

SEQ ID NO: 806 - DNA, 20 bp, Artificial Sequence
  source: /note=“Description of Artificial Sequence Synthetic oligonucleotide”
accccggccctgcctatgcc

SEQ ID NO: 807 - DNA, 20 bp, Artificial Sequence
  source: /note=“Description of Artificial Sequence Synthetic oligonucleotide”
ggtatcagcagtgcaggagg

SEQ ID NO: 808 - DNA, 20 bp, Artificial Sequence
  source: /note=“Description of Artificial Sequence Synthetic oligonucleotide”
gatgtggtcagccttgtgca

SEQ ID NO: 809 - DNA, 20 bp, Artificial Sequence
  source: /note=“Description of Artificial Sequence Synthetic oligonucleotide”
ggatgtggtcagccttgtgc

SEQ ID NO: 810 - DNA, 20 bp, Artificial Sequence
  source: /note=“Description of Artificial Sequence Synthetic oligonucleotide”
ggccacgcatcctggccttt

SEQ ID NO: 811 - DNA, 20 bp, Artificial Sequence
  source: /note=“Description of Artificial Sequence Synthetic oligonucleotide”
gaaaggccaggatgcgtggc

SEQ ID NO: 812 - DNA, 20 bp, Artificial Sequence
  source: /note=“Description of Artificial Sequence Synthetic oligonucleotide”
actgcttgtccaggccacgc

SEQ ID NO: 813 - DNA, 20 bp, Artificial Sequence
  source: /note=“Description of Artificial Sequence Synthetic oligonucleotide”
tctggactccaactgcttgt

SEQ ID NO: 814 - DNA, 20 bp, Artificial Sequence
  source: /note=“Description of Artificial Sequence Synthetic oligonucleotide”
gtctggactccaactgcttg

SEQ ID NO: 815 - DNA, 20 bp, Artificial Sequence
  source: /note=“Description of Artificial Sequence Synthetic oligonucleotide”
gcttccgtctggactccaac

SEQ ID NO: 816 - DNA, 20 bp, Artificial Sequence
  source: /note=“Description of Artificial Sequence Synthetic oligonucleotide”
gacggaagctggagtcggca

SEQ ID NO: 817 - DNA, 20 bp, Artificial Sequence
  source: /note=“Description of Artificial Sequence Synthetic oligonucleotide”
cgctgtcagtgaaaaccact

SEQ ID NO: 818 - DNA, 20 bp, Artificial Sequence
  source: /note=“Description of Artificial Sequence Synthetic oligonucleotide”
ctgacagcgtgaacaggtag

SEQ ID NO: 819 - DNA, 20 bp, Artificial Sequence
  source: /note=“Description of Artificial Sequence Synthetic oligonucleotide”
ttacggccagcgaggcccga

SEQ ID NO: 820 - DNA, 20 bp, Artificial Sequence
  source: /note=“Description of Artificial Sequence Synthetic oligonucleotide”
attacggccagcgaggcccg

SEQ ID NO: 821 - DNA, 20 bp, Artificial Sequence
  source: /note=“Description of Artificial Sequence Synthetic oligonucleotide”
ggaatctggtgagtctgagg

SEQ ID NO: 822 - DNA, 20 bp, Artificial Sequence
  source: /note=“Description of Artificial Sequence Synthetic oligonucleotide”
ccctcagactcaccagattc

SEQ ID NO: 823 - DNA, 20 bp, Artificial Sequence
  source: /note=“Description of Artificial Sequence Synthetic oligonucleotide”
cgaaggtaggagaaggtctt

SEQ ID NO: 824 - DNA, 20 bp, Artificial Sequence
  source: /note=“Description of Artificial Sequence Synthetic oligonucleotide”
gaaggtaggagaaggtcttg

SEQ ID NO: 825 - DNA, 20 bp, Artificial Sequence
  source: /note=“Description of Artificial Sequence Synthetic oligonucleotide”
gcacctttggctcactctct

SEQ ID NO: 826 - DNA, 20 bp, Artificial Sequence
  source: /note=“Description of Artificial Sequence Synthetic oligonucleotide”
tcgaggaggtggcagagggt

SEQ ID NO: 827 - DNA, 20 bp, Artificial Sequence
  source: /note=“Description of Artificial Sequence Synthetic oligonucleotide”
tggaactgcccaagccagtc

SEQ ID NO: 828 - DNA, 20 bp, Artificial Sequence
  source: /note=“Description of Artificial Sequence Synthetic oligonucleotide”
agggacggggcccacagggg

SEQ ID NO: 829 - DNA, 20 bp, Artificial Sequence
  source: /note=“Description of Artificial Sequence Synthetic oligonucleotide”
gggacggggcccacaggggc

SEQ ID NO: 830 - DNA, 20 bp, Artificial Sequence
  source: /note=“Description of Artificial Sequence Synthetic oligonucleotide”
ctccacagccagcaaaggac

SEQ ID NO: 831 - DNA, 20 bp, Artificial Sequence
  source: /note=“Description of Artificial Sequence Synthetic oligonucleotide”
cagccagcaaaggacgaggt

SEQ ID NO: 832 - DNA, 20 bp, Artificial Sequence
  source: /note=“Description of Artificial Sequence Synthetic oligonucleotide”
ctgccttctagacctcgtcc

SEQ ID NO: 833 - DNA, 20 bp, Artificial Sequence
  source: /note=“Description of Artificial Sequence Synthetic oligonucleotide”
cctaaggaggatgcgcctag

SEQ ID NO: 834 - DNA, 20 bp, Artificial Sequence
  source: /note=“Description of Artificial Sequence Synthetic oligonucleotide”
tggcctcctgcactgctgat

SEQ ID NO: 835 - DNA, 20 bp, Artificial Sequence
  source: /note=“Description of Artificial Sequence Synthetic oligonucleotide”
agcagtgcaggaggccacat

SEQ ID NO: 836 - DNA, 20 bp, Artificial Sequence
  source: /note=“Description of Artificial Sequence Synthetic oligonucleotide”
ccgactccagcttccgtctg

SEQ ID NO: 837 - DNA, 20 bp, Artificial Sequence
  source: /note=“Description of Artificial Sequence Synthetic oligonucleotide”
ggggttccattacggccagc

SEQ ID NO: 838 - DNA, 20 bp, Artificial Sequence
  source: /note=“Description of Artificial Sequence Synthetic oligonucleotide”
cacagcagatcctcctctgg

SEQ ID NO: 839 - DNA, 20 bp, Artificial Sequence
  source: /note=“Description of Artificial Sequence Synthetic oligonucleotide”
attgccccgtacagtcagag

SEQ ID NO: 840 - DNA, 20 bp, Artificial Sequence
  source: /note=“Description of Artificial Sequence Synthetic oligonucleotide”
cccgtacagtcagagctgga

SEQ ID NO: 841 - DNA, 20 bp, Artificial Sequence
  source: /note=“Description of Artificial Sequence Synthetic oligonucleotide”
tggtggaggagcaggcagtg

SEQ ID NO: 842 - DNA, 20 bp, Artificial Sequence
  source: /note=“Description of Artificial Sequence Synthetic oligonucleotide”
tccttaggcataggcagggc

SEQ ID NO: 843 - DNA, 20 bp, Artificial Sequence
  source: /note=“Description of Artificial Sequence Synthetic oligonucleotide”
cggccctgcctatgcctaag

SEQ ID NO: 844 - DNA, 20 bp, Artificial Sequence
  source: /note=“Description of Artificial Sequence Synthetic oligonucleotide”
taggcataggcagggccggg

SEQ ID NO: 845 - DNA, 20 bp, Artificial Sequence
  source: /note=“Description of Artificial Sequence Synthetic oligonucleotide”
aggcagggccggggtgggag

SEQ ID NO: 846 - DNA, 20 bp, Artificial Sequence
  source: /note=“Description of Artificial Sequence Synthetic oligonucleotide”
gcaggatcggggctgtcgct

SEQ ID NO: 847 - DNA, 20 bp, Artificial Sequence
  source: /note=“Description of Artificial Sequence Synthetic oligonucleotide”
ctgcacaaggctgaccacat

SEQ ID NO: 848 - DNA, 20 bp, Artificial Sequence
  source: /note=“Description of Artificial Sequence Synthetic oligonucleotide”
tgcacaaggctgaccacatc

SEQ ID NO: 849 - DNA, 20 bp, Artificial Sequence
  source: /note=“Description of Artificial Sequence Synthetic oligonucleotide”
ctgaccacatccggaaaggc

SEQ ID NO: 850 - DNA, 20 bp, Artificial Sequence
  source: /note=“Description of Artificial Sequence Synthetic oligonucleotide”
ggccacgcatcctggccttt

SEQ ID NO: 851 - DNA, 20 bp, Artificial Sequence
  source: /note=“Description of Artificial Sequence Synthetic oligonucleotide”
gcgtggcctggacaagcagt

SEQ ID NO: 852 - DNA, 20 bp, Artificial Sequence
  source: /note=“Description of Artificial Sequence Synthetic oligonucleotide”
gacaagcagttggagtccag

SEQ ID NO: 853 - DNA, 20 bp, Artificial Sequence
  source: /note=“Description of Artificial Sequence Synthetic oligonucleotide”
gttggagtccagacggaagc

SEQ ID NO: 854 - DNA, 20 bp, Artificial Sequence
  source: /note=“Description of Artificial Sequence Synthetic oligonucleotide”
atgcgtacattggtggggcc

SEQ ID NO: 855 - DNA, 20 bp, Artificial Sequence
  source: /note=“Description of Artificial Sequence Synthetic oligonucleotide”
tggccccaccaatgtacgca

SEQ ID NO: 856 - DNA, 20 bp, Artificial Sequence
  source: /note=“Description of Artificial Sequence Synthetic oligonucleotide”
gctacctgttcacgctgtca

SEQ ID NO: 857 - DNA, 20 bp, Artificial Sequence
  source: /note=“Description of Artificial Sequence Synthetic oligonucleotide”
tgacagcgtgaacaggtagc

SEQ ID NO: 858 - DNA, 20 bp, Artificial Sequence
  source: /note=“Description of Artificial Sequence Synthetic oligonucleotide”
gtcgggcctcgctggccgta

SEQ ID NO: 859 - DNA, 20 bp, Artificial Sequence
  source: /note=“Description of Artificial Sequence Synthetic oligonucleotide”
gcacttgcctaggctggtat

SEQ ID NO: 860 - DNA, 20 bp, Artificial Sequence
  source: /note=“Description of Artificial Sequence Synthetic oligonucleotide”
gggaatctggtgagtctgag

SEQ ID NO: 861 - DNA, 20 bp, Artificial Sequence
  source: /note=“Description of Artificial Sequence Synthetic oligonucleotide”
ctcaccagattcccgaaggt

SEQ ID NO: 862 - DNA, 20 bp, Artificial Sequence
  source: /note=“Description of Artificial Sequence Synthetic oligonucleotide”
ctcctaccttcgggaatctg

SEQ ID NO: 863 - DNA, 20 bp, Artificial Sequence
  source: /note=“Description of Artificial Sequence Synthetic oligonucleotide”
caagaccttctcctaccttc

SEQ ID NO: 864 - DNA, 20 bp, Artificial Sequence
  source: /note=“Description of Artificial Sequence Synthetic oligonucleotide”
ccaagaccttctcctacctt

SEQ ID NO: 865 - DNA, 20 bp, Artificial Sequence
  source: /note=“Description of Artificial Sequence Synthetic oligonucleotide”
gccaagaccttctcctacct

SEQ ID NO: 866 - DNA, 20 bp, Artificial Sequence
  source: /note=“Description of Artificial Sequence Synthetic oligonucleotide”
tatgcacagcagatcctcct

SEQ ID NO: 867 - DNA, 20 bp, Artificial Sequence
  source: /note=“Description of Artificial Sequence Synthetic oligonucleotide”
caaaggtgctggacccagag

SEQ ID NO: 868 - DNA, 20 bp, Artificial Sequence
  source: /note=“Description of Artificial Sequence Synthetic oligonucleotide”
ggctcactctctgtctgggc

SEQ ID NO: 869 - DNA, 20 bp, Artificial Sequence
  source: /note=“Description of Artificial Sequence Synthetic oligonucleotide”
agggtaccccagcccagaca

SEQ ID NO: 870 - DNA, 20 bp, Artificial Sequence
  source: /note=“Description of Artificial Sequence Synthetic oligonucleotide”
agagggtaccccagcccaga

SEQ ID NO: 871 - DNA, 20 bp, Artificial Sequence
  source: /note=“Description of Artificial Sequence Synthetic oligonucleotide”
gtaccctctgccacctcctc

SEQ ID NO: 872 - DNA, 20 bp, Artificial Sequence
  source: /note=“Description of Artificial Sequence Synthetic oligonucleotide”
ccttcctcgaggaggtggca

SEQ ID NO: 873 - DNA, 20 bp, Artificial Sequence
  source: /note=“Description of Artificial Sequence Synthetic oligonucleotide”
atgactggcttgggcagttc

SEQ ID NO: 874 - DNA, 20 bp, Artificial Sequence
  source: /note=“Description of Artificial Sequence Synthetic oligonucleotide”
ggcccctgtgggccccgtcc

SEQ ID NO: 875 - DNA, 20 bp, Artificial Sequence
  source: /note=“Description of Artificial Sequence Synthetic oligonucleotide”
aggacgaggtctagaaggca

SEQ ID NO: 876 - DNA, 20 bp, Artificial Sequence
  source: /note=“Description of Artificial Sequence Synthetic oligonucleotide”
tataagtggaggcgtcgcgc

SEQ ID NO: 877 - DNA, 20 bp, Artificial Sequence
  source: /note=“Description of Artificial Sequence Synthetic oligonucleotide”
gggcacgcgtttaatataag

SEQ ID NO: 878 - DNA, 20 bp, Artificial Sequence
  source: /note=“Description of Artificial Sequence Synthetic oligonucleotide”
actcacgctggatagcctcc

SEQ ID NO: 879 - DNA, 20 bp, Artificial Sequence
  source: /note=“Description of Artificial Sequence Synthetic oligonucleotide”
ggccgagatgtctcgctccg

SEQ ID NO: 880 - DNA, 20 bp, Artificial Sequence
  source: /note=“Description of Artificial Sequence Synthetic oligonucleotide”
cacgcgtttaatataagtgg

SEQ ID NO: 881 - DNA, 20 bp, Artificial Sequence
  source: /note=“Description of Artificial Sequence Synthetic oligonucleotide”
aagtggaggcgtcgcgctgg

SEQ ID NO: 882 - DNA, 20 bp, Artificial Sequence
  source: /note=“Description of Artificial Sequence Synthetic oligonucleotide”
gagtagcgcgagcacagcta

SEQ ID NO: 883 - DNA, 20 bp, Artificial Sequence
  source: /note=“Description of Artificial Sequence Synthetic oligonucleotide”
agtggaggcgtcgcgctggc

SEQ ID NO: 884 - DNA, 20 bp, Artificial Sequence
  source: /note=“Description of Artificial Sequence Synthetic oligonucleotide”
gcccgaatgctgtcagcttc

SEQ ID NO: 885 - DNA, 20 bp, Artificial Sequence
  source: /note=“Description of Artificial Sequence Synthetic oligonucleotide”
cgcgagcacagctaaggcca

SEQ ID NO: 886 - DNA, 20 bp, Artificial Sequence
  source: /note=“Description of Artificial Sequence Synthetic oligonucleotide”
ctcgcgctactctctctttc

SEQ ID NO: 887 - DNA, 20 bp, Artificial Sequence
  source: /note=“Description of Artificial Sequence Synthetic oligonucleotide”
ggccacggagcgagacatct

SEQ ID NO: 888 - DNA, 20 bp, Artificial Sequence
  source: /note=“Description of Artificial Sequence Synthetic oligonucleotide”
cgtgagtaaacctgaatctt

SEQ ID NO: 889 - DNA, 20 bp, Artificial Sequence
  source: /note=“Description of Artificial Sequence Synthetic oligonucleotide”
agtcacatggttcacacggc

SEQ ID NO: 890 - DNA, 20 bp, Artificial Sequence
  source: /note=“Description of Artificial Sequence Synthetic oligonucleotide”
aagtcaacttcaatgtcgga

SEQ ID NO: 891 - DNA, 20 bp, Artificial Sequence
  source: /note=“Description of Artificial Sequence Synthetic oligonucleotide”
cagtaagtcaacttcaatgt

SEQ ID NO: 892 - DNA, 20 bp, Artificial Sequence
  source: /note=“Description of Artificial Sequence Synthetic oligonucleotide”
acccagacacatagcaattc

SEQ ID NO: 893 - DNA, 20 bp, Artificial Sequence
  source: /note=“Description of Artificial Sequence Synthetic oligonucleotide”
gcatactcatctttttcagt

SEQ ID NO: 894 - DNA, 20 bp, Artificial Sequence
  source: /note=“Description of Artificial Sequence Synthetic oligonucleotide”
acagcccaagatagttaagt

SEQ ID NO: 895 - DNA, 20 bp, Artificial Sequence
  source: /note=“Description of Artificial Sequence Synthetic oligonucleotide”
ggcatactcatctttttcag

SEQ ID NO: 896 - DNA, 20 bp, Artificial Sequence
  source: /note=“Description of Artificial Sequence Synthetic oligonucleotide”
ttcctgaagctgacagcatt

SEQ ID NO: 897 - DNA, 20 bp, Artificial Sequence
  source: /note=“Description of Artificial Sequence Synthetic oligonucleotide”
tcacgtcatccagcagagaa

SEQ ID NO: 898 - DNA, 20 bp, Artificial Sequence
  source: /note=“Description of Artificial Sequence Synthetic oligonucleotide”
cagcccaagatagttaagtg

SEQ ID NO: 899 - RNA, 18 nt, Artificial Sequence
  source: /note=“Description of Artificial Sequence Synthetic oligonucleotide”
aauucucucuccauucuu

SEQ ID NO: 900 - RNA, 19 nt, Artificial Sequence
  source: /note=“Description of Artificial Sequence Synthetic oligonucleotide”
aauucucucuccauucuuc

SEQ ID NO: 901 - RNA, 20 nt, Artificial Sequence
  source: /note=“Description of Artificial Sequence Synthetic oligonucleotide”
aauucucucuccauucuuca

SEQ ID NO: 902 - RNA, 21 nt, Artificial Sequence
  source: /note=“Description of Artificial Sequence Synthetic oligonucleotide”
aauucucucuccauucuucag

SEQ ID NO: 903 - RNA, 22 nt, Artificial Sequence
  source: /note=“Description of Artificial Sequence Synthetic oligonucleotide”
aauucucucuccauucuucagu

SEQ ID NO: 904 - RNA, 23 nt, Artificial Sequence
  source: /note=“Description of Artificial Sequence Synthetic oligonucleotide”
aauucucucuccauucuucagua

SEQ ID NO: 905 - RNA, 24 nt, Artificial Sequence
  source: /note=“Description of Artificial Sequence Synthetic oligonucleotide”
aauucucucuccauucuucaguaa

SEQ ID NO: 906 - RNA, 18 nt, Artificial Sequence
  source: /note=“Description of Artificial Sequence Synthetic oligonucleotide”
acuuuccauucucugcug

SEQ ID NO: 907 - RNA, 19 nt, Artificial Sequence
  source: /note=“Description of Artificial Sequence Synthetic oligonucleotide”
acuuuccauucucugcugg

SEQ ID NO: 908 - RNA, 20 nt, Artificial Sequence
  source: /note=“Description of Artificial Sequence Synthetic oligonucleotide”
acuuuccauucucugcugga

SEQ ID NO: 909 - RNA, 21 nt, Artificial Sequence
  source: /note=“Description of Artificial Sequence Synthetic oligonucleotide”
acuuuccauucucugcuggau

SEQ ID NO: 910 - RNA, 22 nt, Artificial Sequence
  source: /note=“Description of Artificial Sequence Synthetic oligonucleotide”
acuuuccauucucugcuggaug

SEQ ID NO: 911 - RNA, 23 nt, Artificial Sequence
  source: /note=“Description of Artificial Sequence Synthetic oligonucleotide”
acuuuccauucucugcuggauga

SEQ ID NO: 912 - RNA, 24 nt, Artificial Sequence
  source: /note=“Description of Artificial Sequence Synthetic oligonucleotide”
acuuuccauucucugcuggaugac

SEQ ID NO: 913 - RNA, 18 nt, Artificial Sequence
  source: /note=“Description of Artificial Sequence Synthetic oligonucleotide”
agcaaggacuggucuuuc

SEQ ID NO: 914 - RNA, 19 nt, Artificial Sequence
  source: /note=“Description of Artificial Sequence Synthetic oligonucleotide”
agcaaggacuggucuuucu

SEQ ID NO: 915 - RNA, 20 nt, Artificial Sequence
  source: /note=“Description of Artificial Sequence Synthetic oligonucleotide”
agcaaggacuggucuuucua

SEQ ID NO: 916 - RNA, 21 nt, Artificial Sequence
  source: /note=“Description of Artificial Sequence Synthetic oligonucleotide”
agcaaggacuggucuuucuau

SEQ ID NO: 917 - RNA, 22 nt, Artificial Sequence
  source: /note=“Description of Artificial Sequence Synthetic oligonucleotide”
agcaaggacuggucuuucuauc

SEQ ID NO: 918 - RNA, 23 nt, Artificial Sequence
  source: /note=“Description of Artificial Sequence Synthetic oligonucleotide”
agcaaggacuggucuuucuaucu

SEQ ID NO: 919 - RNA, 24 nt, Artificial Sequence
  source: /note=“Description of Artificial Sequence Synthetic oligonucleotide”
agcaaggacuggucuuucuaucuc

SEQ ID NO: 920 - RNA, 18 nt, Artificial Sequence
  source: /note=“Description of Artificial Sequence Synthetic oligonucleotide”
agugggggugaauucagu

SEQ ID NO: 921 - RNA, 19 nt, Artificial Sequence
  source: /note=“Description of Artificial Sequence Synthetic oligonucleotide”
agugggggugaauucagug

SEQ ID NO: 922 - RNA, 20 nt, Artificial Sequence
  source: /note=“Description of Artificial Sequence Synthetic oligonucleotide”
agugggggugaauucagugu

SEQ ID NO: 923 - RNA, 21 nt, Artificial Sequence
  source: /note=“Description of Artificial Sequence Synthetic oligonucleotide”
agugggggugaauucagugua

SEQ ID NO: 924 - RNA, 22 nt, Artificial Sequence
  source: /note=“Description of Artificial Sequence Synthetic oligonucleotide”
agugggggugaauucaguguag

SEQ ID NO: 925 - RNA, 23 nt, Artificial Sequence
  source: /note=“Description of Artificial Sequence Synthetic oligonucleotide”
agugggggugaauucaguguagu

SEQ ID NO: 926 - RNA, 24 nt, Artificial Sequence
  source: /note=“Description of Artificial Sequence Synthetic oligonucleotide”
agugggggugaauucaguguagua

SEQ ID NO: 927 - RNA, 18 nt, Artificial Sequence
  source: /note=“Description of Artificial Sequence Synthetic oligonucleotide”
auccauccgacauugaag

SEQ ID NO: 928 - RNA, 19 nt, Artificial Sequence
  source: /note=“Description of Artificial Sequence Synthetic oligonucleotide”
auccauccgacauugaagu

SEQ ID NO: 929 - RNA, 20 nt, Artificial Sequence
  source: /note=“Description of Artificial Sequence Synthetic oligonucleotide”
auccauccgacauugaaguu

SEQ ID NO: 930 - RNA, 21 nt, Artificial Sequence
  source: /note=“Description of Artificial Sequence Synthetic oligonucleotide”
auccauccgacauugaaguug

SEQ ID NO: 931 - RNA, 22 nt, Artificial Sequence
  source: /note=“Description of Artificial Sequence Synthetic oligonucleotide”
auccauccgacauugaaguuga

SEQ ID NO: 932 - RNA, 23 nt, Artificial Sequence
  source: /note=“Description of Artificial Sequence Synthetic oligonucleotide”
auccauccgacauugaaguugac

SEQ ID NO: 933 - RNA, 24 nt, Artificial Sequence
  source: /note=“Description of Artificial Sequence Synthetic oligonucleotide”
auccauccgacauugaaguugacu

SEQ ID NO: 934 - RNA, 18 nt, Artificial Sequence
  source: /note=“Description of Artificial Sequence Synthetic oligonucleotide”
caauucucucuccauucu

SEQ ID NO: 935 - RNA, 19 nt, Artificial Sequence
  source: /note=“Description of Artificial Sequence Synthetic oligonucleotide”
caauucucucuccauucuu

SEQ ID NO: 936 - RNA, 20 nt, Artificial Sequence
  source: /note=“Description of Artificial Sequence Synthetic oligonucleotide”
caauucucucuccauucuuc

SEQ ID NO: 937 - RNA, 21 nt, Artificial Sequence
  source: /note=“Description of Artificial Sequence Synthetic oligonucleotide”
caauucucucuccauucuuca

SEQ ID NO: 938 - RNA, 22 nt, Artificial Sequence
  source: /note=“Description of Artificial Sequence Synthetic oligonucleotide”
caauucucucuccauucuucag

SEQ ID NO: 939 - RNA, 23 nt, Artificial Sequence
  source: /note=“Description of Artificial Sequence Synthetic oligonucleotide”
caauucucucuccauucuucagu

SEQ ID NO: 940 - RNA, 24 nt, Artificial Sequence
  source: /note=“Description of Artificial Sequence Synthetic oligonucleotide”
caauucucucuccauucuucagua

SEQ ID NO: 941 - RNA, 18 nt, Artificial Sequence
  source: /note=“Description of Artificial Sequence Synthetic oligonucleotide”
cagugggggugaauucag

SEQ ID NO: 942 - RNA, 19 nt, Artificial Sequence
  source: /note=“Description of Artificial Sequence Synthetic oligonucleotide”
cagugggggugaauucagu

SEQ ID NO: 943 - RNA, 20 nt, Artificial Sequence
  source: /note=“Description of Artificial Sequence Synthetic oligonucleotide”
cagugggggugaauucagug

SEQ ID NO: 944 - RNA, 21 nt, Artificial Sequence
  source: /note=“Description of Artificial Sequence Synthetic oligonucleotide”
cagugggggugaauucagugu

SEQ ID NO: 945 - RNA, 22 nt, Artificial Sequence
  source: /note=“Description of Artificial Sequence Synthetic oligonucleotide”
cagugggggugaauucagugua

SEQ ID NO: 946 - RNA, 23 nt, Artificial Sequence
  source: /note=“Description of Artificial Sequence Synthetic oligonucleotide”
cagugggggugaauucaguguag

SEQ ID NO: 947 - RNA, 24 nt, Artificial Sequence
  source: /note=“Description of Artificial Sequence Synthetic oligonucleotide”
cagugggggugaauucaguguagu

SEQ ID NO: 948 - RNA, 18 nt, Artificial Sequence
  source: /note=“Description of Artificial Sequence Synthetic oligonucleotide”
cauucucugcuggaugac

SEQ ID NO: 949 - RNA, 19 nt, Artificial Sequence
  source: /note=“Description of Artificial Sequence Synthetic oligonucleotide”
cauucucugcuggaugacg

SEQ ID NO: 950 - RNA, 20 nt, Artificial Sequence
  source: /note=“Description of Artificial Sequence Synthetic oligonucleotide”
cauucucugcuggaugacgu

SEQ ID NO: 951 - RNA, 21 nt, Artificial Sequence
  source: /note=“Description of Artificial Sequence Synthetic oligonucleotide”
cauucucugcuggaugacgug

SEQ ID NO: 952 - RNA, 22 nt, Artificial Sequence
  source: /note=“Description of Artificial Sequence Synthetic oligonucleotide”
cauucucugcuggaugacguga

SEQ ID NO: 953 - RNA, 23 nt, Artificial Sequence
  source: /note=“Description of Artificial Sequence Synthetic oligonucleotide”
cauucucugcuggaugacgugag

SEQ ID NO: 954 - RNA, 24 nt, Artificial Sequence
  source: /note=“Description of Artificial Sequence Synthetic oligonucleotide”
cauucucugcuggaugacgugagu

SEQ ID NO: 955 - RNA, 18 nt, Artificial Sequence
  source: /note=“Description of Artificial Sequence Synthetic oligonucleotide”
cccgauauuccucaggua

SEQ ID NO: 956 - RNA, 19 nt, Artificial Sequence
  source: /note=“Description of Artificial Sequence Synthetic oligonucleotide”
cccgauauuccucagguac

SEQ ID NO: 957 - RNA, 20 nt, Artificial Sequence
  source: /note=“Description of Artificial Sequence Synthetic oligonucleotide”
cccgauauuccucagguacu

SEQ ID NO: 958 - RNA, 21 nt, Artificial Sequence
  source: /note=“Description of Artificial Sequence Synthetic oligonucleotide”
cccgauauuccucagguacuc

SEQ ID NO: 959 - RNA, 22 nt, Artificial Sequence
  source: /note=“Description of Artificial Sequence Synthetic oligonucleotide”
cccgauauuccucagguacucc

SEQ ID NO: 960 - RNA, 23 nt, Artificial Sequence
  source: /note=“Description of Artificial Sequence Synthetic oligonucleotide”
cccgauauuccucagguacucca

SEQ ID NO: 961 - RNA, 24 nt, Artificial Sequence
  source: /note=“Description of Artificial Sequence Synthetic oligonucleotide”
cccgauauuccucagguacuccaa

SEQ ID NO: 962 - RNA, 18 nt, Artificial Sequence
  source: /note=“Description of Artificial Sequence Synthetic oligonucleotide”
ccgauauuccucagguac

SEQ ID NO: 963 - RNA, 19 nt, Artificial Sequence
  source: /note=“Description of Artificial Sequence Synthetic oligonucleotide”
ccgauauuccucagguacu

SEQ ID NO: 964 - RNA, 20 nt, Artificial Sequence
  source: /note=“Description of Artificial Sequence Synthetic oligonucleotide”
ccgauauuccucagguacuc

SEQ ID NO: 965 - RNA, 21 nt, Artificial Sequence
  source: /note=“Description of Artificial Sequence Synthetic oligonucleotide”
ccgauauuccucagguacucc

SEQ ID NO: 966 - RNA, 22 nt, Artificial Sequence
  source: /note=“Description of Artificial Sequence Synthetic oligonucleotide”
ccgauauuccucagguacucca

SEQ ID NO: 967 - RNA, 23 nt, Artificial Sequence
  source: /note=“Description of Artificial Sequence Synthetic oligonucleotide”
ccgauauuccucagguacuccaa

SEQ ID NO: 968 - RNA, 24 nt, Artificial Sequence
  source: /note=“Description of Artificial Sequence Synthetic oligonucleotide”
ccgauauuccucagguacuccaaa

SEQ ID NO: 969 - RNA, 18 nt, Artificial Sequence
  source: /note=“Description of Artificial Sequence Synthetic oligonucleotide”
cucacgucauccagcaga

SEQ ID NO: 970 - RNA, 19 nt, Artificial Sequence
  source: /note=“Description of Artificial Sequence Synthetic oligonucleotide”
cucacgucauccagcagag

SEQ ID NO: 971 - RNA, 20 nt, Artificial Sequence
  source: /note=“Description of Artificial Sequence Synthetic oligonucleotide”
cucacgucauccagcagaga

SEQ ID NO: 972 - RNA, 21 nt, Artificial Sequence
  source: /note=“Description of Artificial Sequence Synthetic oligonucleotide”
cucacgucauccagcagagaa

SEQ ID NO: 973 - RNA, 22 nt, Artificial Sequence
  source: /note=“Description of Artificial Sequence Synthetic oligonucleotide”
cucacgucauccagcagagaau

SEQ ID NO: 974 - RNA, 23 nt, Artificial Sequence
  source: /note=“Description of Artificial Sequence Synthetic oligonucleotide”
cucacgucauccagcagagaaug

SEQ ID NO: 975 - RNA, 24 nt, Artificial Sequence
  source: /note=“Description of Artificial Sequence Synthetic oligonucleotide”
cucacgucauccagcagagaaugg

SEQ ID NO: 976 - RNA, 18 nt, Artificial Sequence
  source: /note=“Description of Artificial Sequence Synthetic oligonucleotide”
cugaauugcuaugugucu

SEQ ID NO: 977 - RNA, 19 nt, Artificial Sequence
  source: /note=“Description of Artificial Sequence Synthetic oligonucleotide”
cugaauugcuaugugucug

SEQ ID NO: 978 - RNA, 20 nt, Artificial Sequence
  source: /note=“Description of Artificial Sequence Synthetic oligonucleotide”
cugaauugcuaugugucugg

SEQ ID NO: 979 - RNA, 21 nt, Artificial Sequence
  source: /note=“Description of Artificial Sequence Synthetic oligonucleotide”
cugaauugcuaugugucuggg

SEQ ID NO: 980 - RNA, 22 nt, Artificial Sequence
  source: /note=“Description of Artificial Sequence Synthetic oligonucleotide”
cugaauugcuaugugucugggu

SEQ ID NO: 981 - RNA, 23 nt, Artificial Sequence
  source: /note=“Description of Artificial Sequence Synthetic oligonucleotide”
cugaauugcuaugugucuggguu

SEQ ID NO: 982 - RNA, 24 nt, Artificial Sequence
  source: /note=“Description of Artificial Sequence Synthetic oligonucleotide”
cugaauugcuaugugucuggguuu

SEQ ID NO: 983 - RNA, 18 nt, Artificial Sequence
  source: /note=“Description of Artificial Sequence Synthetic oligonucleotide”
gaguaccugaggaauauc

SEQ ID NO: 984 - RNA, 19 nt, Artificial Sequence
  source: /note=“Description of Artificial Sequence Synthetic oligonucleotide”
gaguaccugaggaauaucg

SEQ ID NO: 985 - RNA, 20 nt, Artificial Sequence
  source: /note=“Description of Artificial Sequence Synthetic oligonucleotide”
gaguaccugaggaauaucgg

SEQ ID NO: 986 - RNA, 21 nt, Artificial Sequence
  source: /note=“Description of Artificial Sequence Synthetic oligonucleotide”
gaguaccugaggaauaucggg

SEQ ID NO: 987 - RNA, 22 nt, Artificial Sequence
  source: /note=“Description of Artificial Sequence Synthetic oligonucleotide”
gaguaccugaggaauaucggga

SEQ ID NO: 988 - RNA, 23 nt, Artificial Sequence
  source: /note=“Description of Artificial Sequence Synthetic oligonucleotide”
gaguaccugaggaauaucgggaa

SEQ ID NO: 989 - RNA, 24 nt, Artificial Sequence
  source: /note=“Description of Artificial Sequence Synthetic oligonucleotide”
gaguaccugaggaauaucgggaaa

SEQ ID NO: 990 - RNA, 18 nt, Artificial Sequence
  source: /note=“Description of Artificial Sequence Synthetic oligonucleotide”
uaucucuuguacuacacu

SEQ ID NO: 991 - RNA, 19 nt, Artificial Sequence
  source: /note=“Description of Artificial Sequence Synthetic oligonucleotide”
uaucucuuguacuacacug

SEQ ID NO: 992 - RNA, 20 nt, Artificial Sequence
  source: /note=“Description of Artificial Sequence Synthetic oligonucleotide”
uaucucuuguacuacacuga

SEQ ID NO: 993 - RNA, 21 nt, Artificial Sequence
  source: /note=“Description of Artificial Sequence Synthetic oligonucleotide”
uaucucuuguacuacacugaa

SEQ ID NO: 994 - RNA, 22 nt, Artificial Sequence
  source: /note=“Description of Artificial Sequence Synthetic oligonucleotide”
uaucucuuguacuacacugaau

SEQ ID NO: 995 - RNA, 23 nt, Artificial Sequence
  source: /note=“Description of Artificial Sequence Synthetic oligonucleotide”
uaucucuuguacuacacugaauu

SEQ ID NO: 996 - RNA, 24 nt, Artificial Sequence
  source: /note=“Description of Artificial Sequence Synthetic oligonucleotide”
uaucucuuguacuacacugaauuc

SEQ ID NO: 997 - RNA, 18 nt, Artificial Sequence
  source: /note=“Description of Artificial Sequence Synthetic oligonucleotide”
ucaauucucucuccauuc

SEQ ID NO: 998 - RNA, 19 nt, Artificial Sequence
  source: /note=“Description of Artificial Sequence Synthetic oligonucleotide”
ucaauucucucuccauucu

SEQ ID NO: 999 - RNA, 20 nt, Artificial Sequence
  source: /note=“Description of Artificial Sequence Synthetic oligonucleotide”
ucaauucucucuccauucuu

SEQ ID NO: 1000 - RNA, 21 nt, Artificial Sequence
  source: /note=“Description of Artificial Sequence Synthetic oligonucleotide”
ucaauucucucuccauucuuc

SEQ ID NO: 1001 - RNA, 22 nt, Artificial Sequence
  source: /note=“Description of Artificial Sequence Synthetic oligonucleotide”
ucaauucucucuccauucuuca

SEQ ID NO: 1002 - RNA, 23 nt, Artificial Sequence
  source: /note=“Description of Artificial Sequence Synthetic oligonucleotide”
ucaauucucucuccauucuucag

SEQ ID NO: 1003 - RNA, 24 nt, Artificial Sequence
  source: /note=“Description of Artificial Sequence Synthetic oligonucleotide”
ucaauucucucuccauucuucagu

SEQ ID NO: 1004 - RNA, 18 nt, Artificial Sequence
  source: /note=“Description of Artificial Sequence Synthetic oligonucleotide”
ucacagcccaagauaguu

SEQ ID NO: 1005 - RNA, 19 nt, Artificial Sequence
  source: /note=“Description of Artificial Sequence Synthetic oligonucleotide”
ucacagcccaagauaguua

SEQ ID NO: 1006 - RNA, 20 nt, Artificial Sequence
  source: /note=“Description of Artificial Sequence Synthetic oligonucleotide”
ucacagcccaagauaguuaa

SEQ ID NO: 1007 - RNA, 21 nt, Artificial Sequence
  source: /note=“Description of Artificial Sequence Synthetic oligonucleotide”
ucacagcccaagauaguuaag

SEQ ID NO: 1008 - RNA, 22 nt, Artificial Sequence
  source: /note=“Description of Artificial Sequence Synthetic oligonucleotide”
ucacagcccaagauaguuaagu

SEQ ID NO: 1009 - RNA, 23 nt, Artificial Sequence
  source: /note=“Description of Artificial Sequence Synthetic oligonucleotide”
ucacagcccaagauaguuaagug

SEQ ID NO: 1010 - RNA, 24 nt, Artificial Sequence
  source: /note=“Description of Artificial Sequence Synthetic oligonucleotide”
ucacagcccaagauaguuaagugg

SEQ ID NO: 1011 - RNA, 18 nt, Artificial Sequence
  source: /note=“Description of Artificial Sequence Synthetic oligonucleotide”
ucagugggggugaauuca

SEQ ID NO: 1012 - RNA, 19 nt, Artificial Sequence
  source: /note=“Description of Artificial Sequence Synthetic oligonucleotide”
ucagugggggugaauucag

SEQ ID NO: 1013 - RNA, 20 nt, Artificial Sequence
  source: /note=“Description of Artificial Sequence Synthetic oligonucleotide”
ucagugggggugaauucagu

SEQ ID NO: 1014 - RNA, 21 nt, Artificial Sequence
  source: /note=“Description of Artificial Sequence Synthetic oligonucleotide”
ucagugggggugaauucagug

SEQ ID NO: 1015 - RNA, 22 nt, Artificial Sequence
  source: /note=“Description of Artificial Sequence Synthetic oligonucleotide”
ucagugggggugaauucagugu

SEQ ID NO: 1016 - RNA, 23 nt, Artificial Sequence
  source: /note=“Description of Artificial Sequence Synthetic oligonucleotide”
ucagugggggugaauucagugua

SEQ ID NO: 1017 - RNA, 24 nt, Artificial Sequence
  source: /note=“Description of Artificial Sequence Synthetic oligonucleotide”
ucagugggggugaauucaguguag

SEQ ID NO: 1018 - RNA, 18 nt, Artificial Sequence
  source: /note=“Description of Artificial Sequence Synthetic oligonucleotide”
uggccuggaggcuaucca

SEQ ID NO: 1019 - RNA, 19 nt, Artificial Sequence
  source: /note=“Description of Artificial Sequence Synthetic oligonucleotide”
uggccuggaggcuauccag

SEQ ID NO: 1020 - RNA, 20 nt, Artificial Sequence
  source: /note=“Description of Artificial Sequence Synthetic oligonucleotide”
uggccuggaggcuauccagc

SEQ ID NO: 1021 - RNA, 21 nt, Artificial Sequence
  source: /note=“Description of Artificial Sequence Synthetic oligonucleotide”
uggccuggaggcuauccagcg

SEQ ID NO: 1022 - RNA, 22 nt, Artificial Sequence
  source: /note=“Description of Artificial Sequence Synthetic oligonucleotide”
uggccuggaggcuauccagcgu

SEQ ID NO: 1023 - RNA, 23 nt, Artificial Sequence
  source: /note=“Description of Artificial Sequence Synthetic oligonucleotide”
uggccuggaggcuauccagcgug

SEQ ID NO: 1024 - RNA, 24 nt, Artificial Sequence
  source: /note=“Description of Artificial Sequence Synthetic oligonucleotide”
uggccuggaggcuauccagcguga

SEQ ID NO: 1025 - RNA, 18 nt, Artificial Sequence
  source: /note=“Description of Artificial Sequence Synthetic oligonucleotide”
auagaucgagacauguaa

SEQ ID NO: 1026 - RNA, 19 nt, Artificial Sequence
  source: /note=“Description of Artificial Sequence Synthetic oligonucleotide”
auagaucgagacauguaag

SEQ ID NO: 1027 - RNA, 20 nt, Artificial Sequence
  source: /note=“Description of Artificial Sequence Synthetic oligonucleotide”
auagaucgagacauguaagc

SEQ ID NO: 1028 - RNA, 21 nt, Artificial Sequence
  source: /note=“Description of Artificial Sequence Synthetic oligonucleotide”
auagaucgagacauguaagca

SEQ ID NO: 1029 - RNA, 22 nt, Artificial Sequence
  source: /note=“Description of Artificial Sequence Synthetic oligonucleotide”
auagaucgagacauguaagcag

SEQ ID NO: 1030 - RNA, 23 nt, Artificial Sequence
  source: /note=“Description of Artificial Sequence Synthetic oligonucleotide”
auagaucgagacauguaagcagc

SEQ ID NO: 1031 - RNA, 24 nt, Artificial Sequence
  source: /note=“Description of Artificial Sequence Synthetic oligonucleotide”
auagaucgagacauguaagcagca

SEQ ID NO: 1032 - RNA, 18 nt, Artificial Sequence
  source: /note=“Description of Artificial Sequence Synthetic oligonucleotide”
cauagaucgagacaugua

SEQ ID NO: 1033 - RNA, 19 nt, Artificial Sequence
  source: /note=“Description of Artificial Sequence Synthetic oligonucleotide”
cauagaucgagacauguaa

SEQ ID NO: 1034 - RNA, 20 nt, Artificial Sequence
  source: /note=“Description of Artificial Sequence Synthetic oligonucleotide”
cauagaucgagacauguaag

SEQ ID NO: 1035 - RNA, 21 nt, Artificial Sequence
  source: /note=“Description of Artificial Sequence Synthetic oligonucleotide”
cauagaucgagacauguaagc

SEQ ID NO: 1036 - RNA, 22 nt, Artificial Sequence
  source: /note=“Description of Artificial Sequence Synthetic oligonucleotide”
cauagaucgagacauguaagca

SEQ ID NO: 1037 - RNA, 23 nt, Artificial Sequence
  source: /note=“Description of Artificial Sequence Synthetic oligonucleotide”
cauagaucgagacauguaagcag

SEQ ID NO: 1038 - RNA, 24 nt, Artificial Sequence
  source: /note=“Description of Artificial Sequence Synthetic oligonucleotide”
cauagaucgagacauguaagcagc

SEQ ID NO: 1039 - RNA, 18 nt, Artificial Sequence
  source: /note=“Description of Artificial Sequence Synthetic oligonucleotide”
cuccacugucuuuuucau

SEQ ID NO: 1040 - RNA, 19 nt, Artificial Sequence
  source: /note=“Description of Artificial Sequence Synthetic oligonucleotide”
cuccacugucuuuuucaua

SEQ ID NO: 1041 - RNA, 20 nt, Artificial Sequence
  source: /note=“Description of Artificial Sequence Synthetic oligonucleotide”
cuccacugucuuuuucauag

SEQ ID NO: 1042 - RNA, 21 nt, Artificial Sequence
  source: /note=“Description of Artificial Sequence Synthetic oligonucleotide”
cuccacugucuuuuucauaga

SEQ ID NO: 1043 - RNA, 22 nt, Artificial Sequence
  source: /note=“Description of Artificial Sequence Synthetic oligonucleotide”
cuccacugucuuuuucauagau

SEQ ID NO: 1044 - RNA, 23 nt, Artificial Sequence
  source: /note=“Description of Artificial Sequence Synthetic oligonucleotide”
cuccacugucuuuuucauagauc

SEQ ID NO: 1045 - RNA, 24 nt, Artificial Sequence
  source: /note=“Description of Artificial Sequence Synthetic oligonucleotide”
cuccacugucuuuuucauagaucg

SEQ ID NO: 1046 - RNA, 18 nt, Artificial Sequence
  source: /note=“Description of Artificial Sequence Synthetic oligonucleotide”
ucauagaucgagacaugu

SEQ ID NO: 1047 - RNA, 19 nt, Artificial Sequence
  source: /note=“Description of Artificial Sequence Synthetic oligonucleotide”
ucauagaucgagacaugua

SEQ ID NO: 1048 - RNA, 20 nt, Artificial Sequence
  source: /note=“Description of Artificial Sequence Synthetic oligonucleotide”
ucauagaucgagacauguaa

SEQ ID NO: 1049 - RNA, 21 nt, Artificial Sequence
  source: /note=“Description of Artificial Sequence Synthetic oligonucleotide”
ucauagaucgagacauguaag

SEQ ID NO: 1050 - RNA, 22 nt, Artificial Sequence
  source: /note=“Description of Artificial Sequence Synthetic oligonucleotide”
ucauagaucgagacauguaagc

SEQ ID NO: 1051 - RNA, 23 nt, Artificial Sequence
  source: /note=“Description of Artificial Sequence Synthetic oligonucleotide”
ucauagaucgagacauguaagca

SEQ ID NO: 1052 - RNA, 24 nt, Artificial Sequence
  source: /note=“Description of Artificial Sequence Synthetic oligonucleotide”
ucauagaucgagacauguaagcag

SEQ ID NO: 1053 - RNA, 18 nt, Artificial Sequence
  source: /note=“Description of Artificial Sequence Synthetic oligonucleotide”
uccacugucuuuuucaua

SEQ ID NO: 1054 - RNA, 19 nt, Artificial Sequence
  source: /note=“Description of Artificial Sequence Synthetic oligonucleotide”
uccacugucuuuuucauag

SEQ ID NO: 1055 - RNA, 20 nt, Artificial Sequence
  source: /note=“Description of Artificial Sequence Synthetic oligonucleotide”
uccacugucuuuuucauaga

SEQ ID NO: 1056 - RNA, 21 nt, Artificial Sequence
  source: /note=“Description of Artificial Sequence Synthetic oligonucleotide”
uccacugucuuuuucauagau

SEQ ID NO: 1057 - RNA, 22 nt, Artificial Sequence
  source: /note=“Description of Artificial Sequence Synthetic oligonucleotide”
uccacugucuuuuucauagauc

SEQ ID NO: 1058 - RNA, 23 nt, Artificial Sequence
  source: /note=“Description of Artificial Sequence Synthetic oligonucleotide”
uccacugucuuuuucauagaucg

SEQ ID NO: 1059 - RNA, 24 nt, Artificial Sequence
  source: /note=“Description of Artificial Sequence Synthetic oligonucleotide”
uccacugucuuuuucauagaucga

SEQ ID NO: 1060 - RNA, 18 nt, Artificial Sequence
  source: /note=“Description of Artificial Sequence Synthetic oligonucleotide”
ucuccacugucuuuuuca

SEQ ID NO: 1061 - RNA, 19 nt, Artificial Sequence
  source: /note=“Description of Artificial Sequence Synthetic oligonucleotide”
ucuccacugucuuuuucau

SEQ ID NO: 1062 - RNA, 20 nt, Artificial Sequence
  source: /note=“Description of Artificial Sequence Synthetic oligonucleotide”
ucuccacugucuuuuucaua

SEQ ID NO: 1063 - RNA, 21 nt, Artificial Sequence
  source: /note=“Description of Artificial Sequence Synthetic oligonucleotide”
ucuccacugucuuuuucauag

SEQ ID NO: 1064 - RNA, 22 nt, Artificial Sequence
  source: /note=“Description of Artificial Sequence Synthetic oligonucleotide”
ucuccacugucuuuuucauaga

SEQ ID NO: 1065 - RNA, 23 nt, Artificial Sequence
  source: /note=“Description of Artificial Sequence Synthetic oligonucleotide”
ucuccacugucuuuuucauagau

SEQ ID NO: 1066 - RNA, 24 nt, Artificial Sequence
  source: /note=“Description of Artificial Sequence Synthetic oligonucleotide”
ucuccacugucuuuuucauagauc

SEQ ID NO: 1067 - RNA, 18 nt, Artificial Sequence
  source: /note=“Description of Artificial Sequence Synthetic oligonucleotide”
uucuccacugucuuuuuc

SEQ ID NO: 1068 - RNA, 19 nt, Artificial Sequence
  source: /note=“Description of Artificial Sequence Synthetic oligonucleotide”
uucuccacugucuuuuuca

SEQ ID NO: 1069 - RNA, 20 nt, Artificial Sequence
  source: /note=“Description of Artificial Sequence Synthetic oligonucleotide”
uucuccacugucuuuuucau

SEQ ID NO: 1070 - RNA, 21 nt, Artificial Sequence
  source: /note=“Description of Artificial Sequence Synthetic oligonucleotide”
uucuccacugucuuuuucaua

SEQ ID NO: 1071 - RNA, 22 nt, Artificial Sequence
  source: /note=“Description of Artificial Sequence Synthetic oligonucleotide”
uucuccacugucuuuuucauag

SEQ ID NO: 1072 - RNA, 23 nt, Artificial Sequence
  source: /note=“Description of Artificial Sequence Synthetic oligonucleotide”
uucuccacugucuuuuucauaga

SEQ ID NO: 1073 - RNA, 24 nt, Artificial Sequence
  source: /note=“Description of Artificial Sequence Synthetic oligonucleotide”
uucuccacugucuuuuucauagau

SEQ ID NO: 1074 - RNA, 18 nt, Artificial Sequence
  source: /note=“Description of Artificial Sequence Synthetic oligonucleotide”
uuucuccacugucuuuuu

SEQ ID NO: 1075 - RNA, 19 nt, Artificial Sequence
  source: /note=“Description of Artificial Sequence Synthetic oligonucleotide”
uuucuccacugucuuuuuc

SEQ ID NO: 1076 - RNA, 20 nt, Artificial Sequence
  source: /note=“Description of Artificial Sequence Synthetic oligonucleotide”
uuucuccacugucuuuuuca

SEQ ID NO: 1077 - RNA, 21 nt, Artificial Sequence
  source: /note=“Description of Artificial Sequence Synthetic oligonucleotide”
uuucuccacugucuuuuucau

SEQ ID NO: 1078 - RNA, 22 nt, Artificial Sequence
  source: /note=“Description of Artificial Sequence Synthetic oligonucleotide”
uuucuccacugucuuuuucaua

SEQ ID NO: 1079 - RNA, 23 nt, Artificial Sequence
  source: /note=“Description of Artificial Sequence Synthetic oligonucleotide”
uuucuccacugucuuuuucauag

SEQ ID NO: 1080 - RNA, 24 nt, Artificial Sequence
  source: /note=“Description of Artificial Sequence Synthetic oligonucleotide”
uuucuccacugucuuuuucauaga

SEQ ID NO: 1081 - RNA, 18 nt, Artificial Sequence
  source: /note=“Description of Artificial Sequence Synthetic oligonucleotide”
uuuucuccacugucuuuu

SEQ ID NO: 1082 - RNA, 19 nt, Artificial Sequence
  source: /note=“Description of Artificial Sequence Synthetic oligonucleotide”
uuuucuccacugucuuuuu

SEQ ID NO: 1083 - RNA, 20 nt, Artificial Sequence
  source: /note=“Description of Artificial Sequence Synthetic oligonucleotide”
uuuucuccacugucuuuuuc

SEQ ID NO: 1084 - RNA, 21 nt, Artificial Sequence
  source: /note=“Description of Artificial Sequence Synthetic oligonucleotide”
uuuucuccacugucuuuuuca

SEQ ID NO: 1085 - RNA, 22 nt, Artificial Sequence
  source: /note=“Description of Artificial Sequence Synthetic oligonucleotide”
uuuucuccacugucuuuuucau

SEQ ID NO: 1086 - RNA, 23 nt, Artificial Sequence
  source: /note=“Description of Artificial Sequence Synthetic oligonucleotide”
uuuucuccacugucuuuuucaua

SEQ ID NO: 1087 - RNA, 24 nt, Artificial Sequence
  source: /note=“Description of Artificial Sequence Synthetic oligonucleotide”
uuuucuccacugucuuuuucauag

SEQ ID NO: 1088 - DNA, 21 bp, Artificial Sequence
  source: /note=“Description of Artificial Sequence Synthetic oligonucleotide”
gaggtaaagcgtttgcatttg

SEQ ID NO: 1089 - DNA, 21 bp, Artificial Sequence
  source: /note=“Description of Artificial Sequence Synthetic oligonucleotide”
cctctaaagcttatgcttaca

SEQ ID NO: 1090 - DNA, 21 bp, Artificial Sequence
  source: /note=“Description of Artificial Sequence Synthetic oligonucleotide”
agtcgatttacttgtagcact

SEQ ID NO: 1091 - DNA, 21 bp, Artificial Sequence
  source: /note=“Description of Artificial Sequence Synthetic oligonucleotide”
cttgtagcactgcacagttaa

SEQ ID NO: 1092 - DNA, 21 bp, Artificial Sequence
  source: /note=“Description of Artificial Sequence Synthetic oligonucleotide”
tccattacaggataaaagact

SEQ ID NO: 1093 - DNA, 21 bp, Artificial Sequence
  source: /note=“Description of Artificial Sequence Synthetic oligonucleotide”
ctccattacaggataaaagac

SEQ ID NO: 1094 - DNA, 21 bp, Artificial Sequence
  source: /note=“Description of Artificial Sequence Synthetic oligonucleotide”
tctccattacaggataaaaga

SEQ ID NO: 1095 - DNA, 21 bp, Artificial Sequence
  source: /note=“Description of Artificial Sequence Synthetic oligonucleotide”
atcctgtaatggagaaaaatc

SEQ ID NO: 1096 - DNA, 21 bp, Artificial Sequence
  source: /note=“Description of Artificial Sequence Synthetic oligonucleotide”
tcctgtaatggagaaaaatcc

SEQ ID NO: 1097 - DNA, 21 bp, Artificial Sequence
  source: /note=“Description of Artificial Sequence Synthetic oligonucleotide”
aaacatgagtaagttgttttg

SEQ ID NO: 1098 - DNA, 21 bp, Artificial Sequence
  source: /note=“Description of Artificial Sequence Synthetic oligonucleotide”
gctttcaaacatgagtaagtt

SEQ ID NO: 1099 - DNA, 21 bp, Artificial Sequence
  source: /note=“Description of Artificial Sequence Synthetic oligonucleotide”
aaagccaaaccattcattgtc

SEQ ID NO: 1100 - DNA, 21 bp, Artificial Sequence
  source: /note=“Description of Artificial Sequence Synthetic oligonucleotide”
gtaacagcagtcatcatccat

SEQ ID NO: 1101 - DNA, 21 bp, Artificial Sequence
  source: /note=“Description of Artificial Sequence Synthetic oligonucleotide”
accatcctcatggattggtgt

SEQ ID NO: 1102 - DNA, 21 bp, Artificial Sequence
  source: /note=“Description of Artificial Sequence Synthetic oligonucleotide”
tgtccatcatttcaccatcct

SEQ ID NO: 1103 - DNA, 21 bp, Artificial Sequence
  source: /note=“Description of Artificial Sequence Synthetic oligonucleotide”
gaaatttctgtccatcatttc

SEQ ID NO: 1104 - DNA, 21 bp, Artificial Sequence
  source: /note=“Description of Artificial Sequence Synthetic oligonucleotide”
agaaatttctgtccatcattt

SEQ ID NO: 1105 - DNA, 21 bp, Artificial Sequence
  source: /note=“Description of Artificial Sequence Synthetic oligonucleotide”
ttttagaaatttctgtccatc

SEQ ID NO: 1106 - DNA, 21 bp, Artificial Sequence
  source: /note=“Description of Artificial Sequence Synthetic oligonucleotide”
cttttagaaatttctgtccat

SEQ ID NO: 1107 - DNA, 21 bp, Artificial Sequence
  source: /note=“Description of Artificial Sequence Synthetic oligonucleotide”
ttttcttttagaaatttctgt

SEQ ID NO: 1108 - DNA, 21 bp, Artificial Sequence
  source: /note=“Description of Artificial Sequence Synthetic oligonucleotide”
taaaagaaaagaaagaatttt

SEQ ID NO: 1109 - DNA, 21 bp, Artificial Sequence
  source: /note=“Description of Artificial Sequence Synthetic oligonucleotide”
aaacatttacatcttaccatt

SEQ ID NO: 1110 - DNA, 21 bp, Artificial Sequence
  source: /note=“Description of Artificial Sequence Synthetic oligonucleotide”
catcttaccatttcttcttca

SEQ ID NO: 1111 - DNA, 21 bp, Artificial Sequence
  source: /note=“Description of Artificial Sequence Synthetic oligonucleotide”
tatagataatgaagaagaaat

SEQ ID NO: 1112 - DNA, 21 bp, Artificial Sequence
  source: /note=“Description of Artificial Sequence Synthetic oligonucleotide”
ttcttcattatctatagaaag

SEQ ID NO: 1113 - DNA, 21 bp, Artificial Sequence
  source: /note=“Description of Artificial Sequence Synthetic oligonucleotide”
ctggcctgtacttcgaagaac

SEQ ID NO: 1114 - DNA, 21 bp, Artificial Sequence
  source: /note=“Description of Artificial Sequence Synthetic oligonucleotide”
cttaccaatgtagtaacaact

SEQ ID NO: 1115 - DNA, 21 bp, Artificial Sequence
  source: /note=“Description of Artificial Sequence Synthetic oligonucleotide”
gcacgtcattgtggccattgt

SEQ ID NO: 1116 - DNA, 21 bp, Artificial Sequence
  source: /note=“Description of Artificial Sequence Synthetic oligonucleotide”
tttagcacgtcattgtggcca

SEQ ID NO: 1117 - DNA, 21 bp, Artificial Sequence
  source: /note=“Description of Artificial Sequence Synthetic oligonucleotide”
ccatcagctccagagaagctc

SEQ ID NO: 1118 - DNA, 21 bp, Artificial Sequence
  source: /note=“Description of Artificial Sequence Synthetic oligonucleotide”
tctccctgcagatttaccatc

SEQ ID NO: 1119 - DNA, 21 bp, Artificial Sequence
  source: /note=“Description of Artificial Sequence Synthetic oligonucleotide”
aaatgctttacctttgcagtg

SEQ ID NO: 1120 - DNA, 21 bp, Artificial Sequence
  source: /note=“Description of Artificial Sequence Synthetic oligonucleotide”
aatgctttacctttgcagtga

SEQ ID NO: 1121 - DNA, 21 bp, Artificial Sequence
  source: /note=“Description of Artificial Sequence Synthetic oligonucleotide”
cctttgcagtgataggttttg

SEQ ID NO: 1122 - DNA, 21 bp, Artificial Sequence
  source: /note=“Description of Artificial Sequence Synthetic oligonucleotide”
cagtgataggttttgtcattc

SEQ ID NO: 1123 - DNA, 21 bp, Artificial Sequence
  source: /note=“Description of Artificial Sequence Synthetic oligonucleotide”
aagggaatgacaaaacctatc

SEQ ID NO: 1124 - DNA, 21 bp, Artificial Sequence
  source: /note=“Description of Artificial Sequence Synthetic oligonucleotide”
caagggaatgacaaaacctat

SEQ ID NO: 1125 - DNA, 21 bp, Artificial Sequence
  source: /note=“Description of Artificial Sequence Synthetic oligonucleotide”
gtcattcccttgaaaatcctg

SEQ ID NO: 1126 - DNA, 21 bp, Artificial Sequence
  source: /note=“Description of Artificial Sequence Synthetic oligonucleotide”
tcattcccttgaaaatcctga

SEQ ID NO: 1127 - DNA, 21 bp, Artificial Sequence
  source: /note=“Description of Artificial Sequence Synthetic oligonucleotide”
tgaaggtttaattccgcatag

SEQ ID NO: 1128 - DNA, 21 bp, Artificial Sequence
  source: /note=“Description of Artificial Sequence Synthetic oligonucleotide”
gaaggtttaattccgcatagg

SEQ ID NO: 1129 - DNA, 21 bp, Artificial Sequence
  source: /note=“Description of Artificial Sequence Synthetic oligonucleotide”
aaggtttaattccgcataggt

SEQ ID NO: 1130 - DNA, 21 bp, Artificial Sequence
  source: /note=“Description of Artificial Sequence Synthetic oligonucleotide”
attccgcataggttatttcct

SEQ ID NO: 1131 - DNA, 21 bp, Artificial Sequence
  source: /note=“Description of Artificial Sequence Synthetic oligonucleotide”
gcaactgaacaggaaataacc

SEQ ID NO: 1132 - DNA, 21 bp, Artificial Sequence
  source: /note=“Description of Artificial Sequence Synthetic oligonucleotide”
agcaactgaacaggaaataac

SEQ ID NO: 1133 - DNA, 21 bp, Artificial Sequence
  source: /note=“Description of Artificial Sequence Synthetic oligonucleotide”
ctgttcagttgctaaaatgga

SEQ ID NO: 1134 - DNA, 21 bp, Artificial Sequence
  source: /note=“Description of Artificial Sequence Synthetic oligonucleotide”
tattgcctttaggttttcgtt

SEQ ID NO: 1135 - DNA, 21 bp, Artificial Sequence
  source: /note=“Description of Artificial Sequence Synthetic oligonucleotide”
attgcctttaggttttcgttg

SEQ ID NO: 1136 - DNA, 21 bp, Artificial Sequence
  source: /note=“Description of Artificial Sequence Synthetic oligonucleotide”
ttgcctttaggttttcgttgc

SEQ ID NO: 1137 - DNA, 21 bp, Artificial Sequence
  source: /note=“Description of Artificial Sequence Synthetic oligonucleotide”
ggttttcgttgctgcctcttt

SEQ ID NO: 1138 - DNA, 21 bp, Artificial Sequence
  source: /note=“Description of Artificial Sequence Synthetic oligonucleotide”
cgttgctgcctctttgggttt

SEQ ID NO: 1139 - DNA, 21 bp, Artificial Sequence
  source: /note=“Description of Artificial Sequence Synthetic oligonucleotide”
gttgctgcctctttgggtttg

SEQ ID NO: 1140 - DNA, 21 bp, Artificial Sequence
  source: /note=“Description of Artificial Sequence Synthetic oligonucleotide”
ggtttgggggcagattcaggt

SEQ ID NO: 1141 - DNA, 21 bp, Artificial Sequence
  source: /note=“Description of Artificial Sequence Synthetic oligonucleotide”
ggggcagattcaggtctgagt

SEQ ID NO: 1142 - protein, 1334 aa, Artificial Sequence
  source: /note=“Description of Artificial Sequence Synthetic polypeptide”
MGHHHHHHGSTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAARLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRNGSPKKKRKVGSPKKKRKV

SEQ ID NO: 1143 - protein, 1363 aa, Artificial Sequence
  source: /note=“Description of Artificial Sequence Synthetic polypeptide”
MTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQRPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFLFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRNGRSSDDEATADSQHAAPPKKKRKVGGSGGSGGSGGSGGSGGSGGSGGSLEHHHHHH

SEQ ID NO: 1144 - protein, 1363 aa, Artificial Sequence
  source: /note=“Description of Artificial Sequence Synthetic polypeptide”
MTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRNGRSSDDEATADSQHAAPPKKKRKVGGSGGSGGSGGSGGSGGSGGSGGSLEHHHHHH

SEQ ID NO: 1145 - protein, 1363 aa, Artificial Sequence
  source: /note=“Description of Artificial Sequence Synthetic polypeptide”
MTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQRPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAARLGEKMLNKKLKDQKTPIPDTLYQELYDYVNHRLSHDLSDEARALLPNVITKEVSHEIIKDRRFTSDKFLFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRNGRSSDDEATADSQHAAPPKKKRKVGGSGGSGGSGGSGGSGGSGGSGGSLEHHHHHH

SEQ ID NO: 1146 - protein, 1331 aa, Artificial Sequence
  source: /note=“Description of Artificial Sequence Synthetic polypeptide”
MTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQRPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAARLGEKMLNKKLKDQKTPIPDTLYQELYDYVNHRLSHDLSDEARALLPNVITKEVSHEIIKDRRFTSDKFLFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRNGRSSDDEATADSQHAAPPKKKRKV

SEQ ID NO: 1147 - protein, 1331 aa, Artificial Sequence
  source: /note=“Description of Artificial Sequence Synthetic polypeptide”
MTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQRPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFLFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRNGRSSDDEATADSQHAAPPKKKRKV

SEQ ID NO: 1148 - protein, 1363 aa, Artificial Sequence; identical to SEQ ID NO: 1143
  source: /note=“Description of Artificial Sequence Synthetic polypeptide”

SEQ ID NO: 1149 - protein, 1361 aa, Artificial Sequence
  source: /note=“Description of Artificial Sequence Synthetic polypeptide”
MGRDPGKPIPNPLLGLDSTAPKKKRKVGIHGVPAATQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRNPKKKRKVKLAAALEHHHHHH

SEQ ID NO: 1150 - protein, 1307 aa, Acidaminococcus sp.
MTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRN

SEQ ID NO: 1151 - RNA, 20 nt, Artificial Sequence
  source: /note=“Description of Artificial Sequence Synthetic oligonucleotide”
uagauguucucaucuuuaau

SEQ ID NO: 1152 - DNA, 25 bp, Artificial Sequence
  source: /note=“Description of Artificial Sequence Synthetic oligonucleotide”
atgtgtttttgtcaaaagacctttt

SEQ ID NO: 1153 - DNA, 20 bp, Artificial Sequence
  source: /note=“Description of Artificial Sequence Synthetic oligonucleotide”
ggtgtacagcagtggctggt

SEQ ID NO: 1154 - DNA, 65 bp, Artificial Sequence
  source: /note=“Description of Artificial Sequence Synthetic oligonucleotide”
  source: /note=“Description of Combined DNA/RNA Molecule Synthetic oligonucleotide”
atgtgtttttgtcaaaagaccttttuaauuucuacucuuguagaugguguacagcaguggcuggu

SEQ ID NO: 1155 - DNA, 20 bp, Artificial Sequence
  source: /note=“Description of Artificial Sequence Synthetic oligonucleotide”
tgatgtgagattttccacct

SEQ ID NO: 1156 - DNA, 65 bp, Artificial Sequence
  source: /note=“Description of Artificial Sequence Synthetic oligonucleotide”
  source: /note=“Description of Combined DNA/RNA Molecule Synthetic oligonucleotide”
atgtgtttttgtcaaaagaccttttuaauuucuacucuuguagauugaugugagauuuuccaccu

SEQ ID NO: 1157 - DNA, 20 bp, Artificial Sequence
  source: /note=“Description of Artificial Sequence Synthetic oligonucleotide”
tgcagagaaaggtggctcta

SEQ ID NO: 1158 - DNA, 65 bp, Artificial Sequence
  source: /note=“Description of Artificial Sequence Synthetic oligonucleotide”
  source: /note=“Description of Combined DNA/RNA Molecule Synthetic oligonucleotide”
atgtgtttttgtcaaaagaccttttuaauuucuacucuuguagauugcagagaaagguggcucua

SEQ ID NO: 1159 - DNA, 21 bp, Artificial Sequence
  source: /note=“Description of Artificial Sequence Synthetic oligonucleotide”
gcaactgaacaggaaataacc

SEQ ID NO: 1160 - RNA, 41 nt, Artificial Sequence
  source: /note=“Description of Artificial Sequence Synthetic oligonucleotide”
uaauuucuacucuuguagaugcaacugaacaggaaauaacc

SEQ ID NO: 1161 - DNA, 20 bp, Artificial Sequence
  source: /note=“Description of Artificial Sequence Synthetic oligonucleotide”
cctgtgtgctggtgcccctg

SEQ ID NO: 1162 - DNA, 65 bp, Artificial Sequence
  source: /note=“Description of Artificial Sequence Synthetic oligonucleotide”
  source: /note=“Description of Combined DNA/RNA Molecule Synthetic oligonucleotide”
atgtgtttttgtcaaaagaccttttuaauuucuacucuuguagauccugugugcuggugccccug

SEQ ID NO: 1163 - DNA, 21 bp, Artificial Sequence
  source: /note=“Description of Artificial Sequence Synthetic oligonucleotide”
actgacagcgtgaacaggtag

SEQ ID NO: 1164 - DNA, 66 bp, Artificial Sequence
  source: /note=“Description of Artificial Sequence Synthetic oligonucleotide”
  source: /note=“Description of Combined DNA/RNA Molecule Synthetic oligonucleotide”
atgtgtttttgtcaaaagaccttttuaauuucuacucuuguagauacugacagcgugaacagguag

SEQ ID NO: 1165 - DNA, 20 bp, Artificial Sequence
  source: /note=“Description of Artificial Sequence Synthetic oligonucleotide”
tgatgtgagattttccacct

SEQ ID NO: 1166 - DNA, 65 bp, Artificial Sequence; identical to SEQ ID NO: 1156
  source: /note=“Description of Artificial Sequence Synthetic oligonucleotide”
  source: /note=“Description of Combined DNA/RNA Molecule Synthetic oligonucleotide”

SEQ ID NO: 1167 - DNA, 20 bp, Artificial Sequence
  source: /note=“Description of Artificial Sequence Synthetic oligonucleotide”
tgcagagaaaggtggctcta

SEQ ID NO: 1168 - DNA, 65 bp, Artificial Sequence; identical to SEQ ID NO: 1158
  source: /note=“Description of Artificial Sequence Synthetic oligonucleotide”
  source: /note=“Description of Combined DNA/RNA Molecule Synthetic oligonucleotide”